SEQUENCE LISTING (207 sequences)

SEQ ID NO: 1 - DNA, 135 bp, Homo sapiens
  misc_feature 1..135: note = CD8alpha hinge - DNA
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 2 - protein, 45 aa, Homo sapiens
  REGION 1..45: note = MISC_FEATURE - CD8a hinge - protein
TTTPAPRPPTPAPTIASQPLSLRPEACRPAAGGAVHTRGLDFACD

SEQ ID NO: 3 - DNA, 63 bp, Homo sapiens
  misc_feature 1..63: note = CD8 TM - DNA
  source 1..63: mol_type = other DNA
atctacatctgggcgcccttggccgggacttgtggggtccttctcctgtcactggttatcacc

SEQ ID NO: 4 - protein, 21 aa, Homo sapiens
  REGION 1..21: note = MISC_FEATURE - CD8 TM - protein
IYIWAPLAGTCGVLLLSLVIT

SEQ ID NO: 5 - DNA, 111 bp, Homo sapiens
  misc_feature 1..111: note = OX40 - DNA
  source 1..111: mol_type = other DNA
cggagggaccagaggctgccccccgatgcccacaagccccctgggggaggcagtttccggacccccatccaagaggagcaggccgacgcccactccaccctggccaagatc

SEQ ID NO: 6 - protein, 37 aa, Homo sapiens
  REGION 1..37: note = MISC_FEATURE - OX40 - protein
RRDQRLPPDAHKPPGGGSFRTPIQEEQADAHSTLAKI

SEQ ID NO: 7 - DNA, 336 bp, Homo sapiens
  misc_feature 1..336: note = CD3zeta - DNA
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 112 aa, Homo sapiens
  REGION 1..112: note = MISC_FEATURE - CD3zeta - protein
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, Homo sapiens
  misc_feature 1..63: note = T2A - DNA
  source 1..63: mol_type = other DNA
ggctctggcgagggaaggggttccctgcttacttgcggcgacgtcgaagagaatcccggtccg

SEQ ID NO: 10 - protein, 21 aa, Homo sapiens
  REGION 1..21: note = MISC_FEATURE - T2A - protein
GSGEGRGSLLTCGDVEENPGP

SEQ ID NO: 11 - DNA, 342 bp, Homo sapiens
  misc_feature 1..342: note = IL15 - DNA
  source 1..342: mol_type = other DNA
aactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtcc

SEQ ID NO: 12 - protein, 114 aa, Homo sapiens
  REGION 1..114: note = MISC_FEATURE - IL15 - protein
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 207 bp, Homo sapiens
  misc_feature 1..207: note = CD8hinge/TM - DNA
  source 1..207: mol_type = other DNA
actaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 14 - protein, 66 aa, Homo sapiens
  REGION 1..66: note = MISC_FEATURE - CD8hinge/TM - protein
TTTPAPRPPTPAPTIASQPLSLRPEACRPAAGGAVHTRGLDFACDIYIWAPLAGTCGVLLLSLVIT

SEQ ID NO: 15 - DNA, 30 bp, Homo sapiens
  misc_feature 1..30: note = Linker 1 (used after FLAG tag) - DNA
  source 1..30: mol_type = other DNA
ggcggtggtggctctggtggtggcggcagc

SEQ ID NO: 16 - protein, 10 aa, Homo sapiens
  REGION 1..10: note = MISC_FEATURE - Linker 1 (used after FLAG tag) - protein
GGGGSGGGGS

SEQ ID NO: 17 - DNA, 30 bp, Homo sapiens
  misc_feature 1..30: note = Linker 2 - after neg. control binding domain - DNA
  source 1..30: mol_type = other DNA
ggccaggccggctccggaggaggaggatcc

SEQ ID NO: 18 - protein, 10 aa, Homo sapiens
  REGION 1..10: note = MISC_FEATURE - Linker 2 - after neg. control binding domain - Protein
GQAGSGGGGS

SEQ ID NO: 19 - DNA, 12 bp, Homo sapiens
  misc_feature 1..12: note = Linker post scFv - DNA
  source 1..12: mol_type = other DNA
ggccaggccggc

SEQ ID NO: 20 - protein, 4 aa, Homo sapiens
  REGION 1..4: note = MISC_FEATURE - Linker post scFv - protein
GQAG

SEQ ID NO: 21 - DNA, 33 bp, Homo sapiens
  misc_feature 1..33: note = Linker - DNA
  misc_feature 1..33: note = Misc Linker - DNA
  source 1..33: mol_type = other DNA
ggcggcggcggtagcggtggtggcggctccgga

SEQ ID NO: 22 - protein, 11 aa, Homo sapiens
  REGION 1..11: note = MISC_FEATURE - Misc Linker - protein
GGGGSGGGGSG

SEQ ID NO: 23 - DNA, 18 bp, Homo sapiens
  misc_feature 1..18: note = Linker - DNA
  source 1..18: mol_type = other DNA
ggccaggccggctccgga

SEQ ID NO: 24 - protein, 6 aa, Homo sapiens
  REGION 1..6: note = MISC_FEATURE - Linker - protein
GQAGSG

SEQ ID NO: 25 - DNA, 405 bp, Homo sapiens
  misc_feature 1..405: note = NKG2D Extracellular Fragment - DNA
  source 1..405: mol_type = other DNA
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 26 - protein, 135 aa, Homo sapiens
  REGION 1..135: note = MISC_FEATURE - NKG2D Extracellular Fragment - protein
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 27 - DNA, 645 bp, Homo sapiens
  misc_feature 1..645: note = Full Human NKG2D - DNA
  source 1..645: mol_type = other DNA
gggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 28 - DNA, 405 bp, Homo sapiens
  misc_feature 1..405: note = Codon optimized human NKG2D fragment - DNA
  source 1..405: mol_type = other DNA
ctgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtg

SEQ ID NO: 29 - DNA, 126 bp, Homo sapiens
  misc_feature 1..126: note = 4-1BB
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 30 - protein, 29 aa, Homo sapiens
  REGION 1..29: note = MISC_FEATURE - Amino Acid Sequence CD28 Transmembrane domain
FWVLVVVGGVLACYSLLVTVAFIIFWVRS

SEQ ID NO: 31 - protein, 39 aa, Homo sapiens
  REGION 1..39: note = MISC_FEATURE - Amino Acid Sequence CD28 IC domain
KRSRLLHSDYMNMTPRRPGPTRKHYQPYAPPRDFAAYRS

SEQ ID NO: 32 - protein, 123 aa, Homo sapiens
  REGION 1..123: note = MISC_FEATURE - VL Nicholson et al.
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 33 - protein, 120 aa, Homo sapiens
  REGION 1..120: note = MISC_FEATURE - VH Nicholson et al.
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 34 - protein, 634 aa, Homo sapiens
  REGION 1..634: note = MISC_FEATURE - PRT Artificial Sequence CD19R zeta chimeric receptor
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKMALIVLGGVAGLLLFIGLGIFFRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 120 aa, Homo sapiens
  REGION 1..120: note = MISC_FEATURE - anti-CD19 scFv HCV
EVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSS

SEQ ID NO: 36 - protein, 107 aa, Homo sapiens
  REGION 1..107: note = MISC_FEATURE - anti-CD19 scFv HCV
DIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIK

SEQ ID NO: 37 - protein, 11 aa, Homo sapiens
  REGION 1..11: note = MISC_FEATURE - LC CDR1
RASQDISKYLN

SEQ ID NO: 38 - protein, 7 aa, Homo sapiens
  REGION 1..7: note = MISC_FEATURE - LC CDR2
HTSRLHS

SEQ ID NO: 39 - protein, 9 aa, Homo sapiens
  REGION 1..9: note = MISC_FEATURE - LC CDR3
QQGNTLPYT

SEQ ID NO: 40 - protein, 10 aa, Homo sapiens
  REGION 1..10: note = MISC_FEATURE - HC CDR1
GVSLPDYGVS

SEQ ID NO: 41 - protein, 16 aa, Homo sapiens
  REGION 1..16: note = MISC_FEATURE - HC CDR2
VIWGSETTYYSSSLKS

SEQ ID NO: 42 - protein, 16 aa, Homo sapiens
  REGION 1..16: note = MISC_FEATURE - HC CDR2 - alternative
VIWGSETTYYQSSLKS

SEQ ID NO: 43 - protein, 16 aa, Homo sapiens
  REGION 1..16: note = MISC_FEATURE - HC CDR2 - alternative 2
VIWGSETTYYNSSLKS

SEQ ID NO: 44 - protein, 12 aa, Homo sapiens
  REGION 1..12: note = MISC_FEATURE - HC CDR3
HYYYGGSYAMDY

SEQ ID NO: 45 - protein, 107 aa, Homo sapiens
  REGION 1..107: note = MISC_FEATURE - Light chain variable region 1
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 46 - protein, 107 aa, Homo sapiens
  REGION 1..107: note = MISC_FEATURE - Light chain variable region 2
DIQMTQSPSSLSASVGDRVTITCRASQDISKYLNWYQQKPGKAVKLLIYHTSRLHSGVPSRFSGSGSGTDYTLTISSLQPEDIATYFCQQGNTLPYTFGGGTKLEIK

SEQ ID NO: 47 - protein, 107 aa, Homo sapiens
  REGION 1..107: note = MISC_FEATURE - Light chain variable region 3
DIQMTQSPSFLSASVGDRVTITCRASQDISKYLNWYQQKPDQAPKLLIKHTSRLHSGVPSRFSGSGSGTDFTFTISSLQPEDIATYYCQQGNTLPYTFGQGTKLEIK

SEQ ID NO: 48 - protein, 107 aa, Homo sapiens
  REGION 1..107: note = MISC_FEATURE - Light chain variable region 4
DIQMTQSPSTLSASVGDRVTITCRASQDISKYLNWYQQKPDQAPKLLIKHTSRLHSGVPSRFSGSGSGTDFTFTISSLQPEDIATYYCQQGNTLPYTFGQGTKLEIK

SEQ ID NO: 49 - protein, 120 aa, Homo sapiens
  REGION 1..120: note = MISC_FEATURE - Heavy chain variable region 1
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 50 - protein, 120 aa, Homo sapiens
  REGION 1..120: note = MISC_FEATURE - Heavy chain variable region 2
QVQLQESGPGLVKPSETLSVTCTVSGVSLPDYGVSWIRQPPGKGLEWLGVIWGSETTYYNSALKSRLTISKDTSKNQVFLKMSSLTAADTAIYYCAKHYYYGGSYAMDYWGQGTLVTVSS

SEQ ID NO: 51 - protein, 120 aa, Homo sapiens
  REGION 1..120: note = MISC_FEATURE - Heavy chain variable region 3
EVQLVESGGGLVQPGRSLRLSCTASGVSLPDYGVSWVRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 52 - protein, 120 aa, Homo sapiens
  REGION 1..120: note = MISC_FEATURE - Heavy chain variable region 4
QVQLQESGPGLVKPSGTLSLTCAVSGVSLPDYGVSWIRQPPGKGLEWIGVIWGSETTYYNSALKSRVTISRDNSKNTLYLQMNSLRAEDTAVYYCAKHYYYGGSYAMDYWGQGTLVTVSS

SEQ ID NO: 53 - protein, 8 aa, Homo sapiens
  REGION 1..8: note = MISC_FEATURE - LC CDR1
RASQDISK

SEQ ID NO: 54 - protein, 7 aa, Homo sapiens
  REGION 1..7: note = MISC_FEATURE - LC CDR2
IYHTSRL

SEQ ID NO: 55 - protein, 9 aa, Homo sapiens
  REGION 1..9: note = MISC_FEATURE - LC CDR3
QQGNTLPYT

SEQ ID NO: 56 - protein, 10 aa, Homo sapiens
  REGION 1..10: note = MISC_FEATURE - HC CDR1
GVSLPDYGVS

SEQ ID NO: 57 - protein, 14 aa, Homo sapiens
  REGION 1..14: note = MISC_FEATURE - HC CDR2
VIWGSETTYYNSAL

SEQ ID NO: 58 - protein, 12 aa, Homo sapiens
  REGION 1..12: note = MISC_FEATURE - HC CDR3
HYYYGGSYAMDY

SEQ ID NO: 59 - DNA, 2210 bp, Homo sapiens
  misc_feature 1..2210: note = NK19-1b DNA
  source 1..2210: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 60 - protein, 724 aa, Homo sapiens
  REGION 1..724: note = MISC_FEATURE - NK19-1b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 61 - DNA, 2231 bp, Homo sapiens
  misc_feature 1..2231: note = NK19-2b DNA
  source 1..2231: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 62 - protein, 731 aa, Homo sapiens
  REGION 1..731: note = MISC_FEATURE - NK19-2b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 63 - DNA, 2213 bp, Homo sapiens
  misc_feature 1..2213: note = NK19-3b DNA
  source 1..2213: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 64 - protein, 725 aa, Homo sapiens
  REGION 1..725: note = MISC_FEATURE - NK19-3b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 65 - DNA, 2357 bp, Homo sapiens
  misc_feature 1..2357: note = NK19-4b DNA
  source 1..2357: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 66 - protein, 773 aa, Homo sapiens
  REGION 1..773: note = MISC_FEATURE - NK19-4b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 67 - DNA, 2201 bp, Homo sapiens
  misc_feature 1..2201: note = NK19-5b DNA
  source 1..2201: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatccattttttttctgctgcttcatcgctgtagccatgggaatccgtttcattattatggtaacacggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 68 - protein, 721 aa, Homo sapiens
  REGION 1..721: note = MISC_FEATURE - NK19-5b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDPFFFCCFIAVAMGIRFIIMVTRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 69 - DNA, 1805 bp, Homo sapiens
  misc_feature 1..1805: note = NK19-6b DNA
  source 1..1805: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 70 - protein, 589 aa, Homo sapiens
  REGION 1..589: note = MISC_FEATURE - NK19-6b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 71 - DNA, 3347 bp, Homo sapiens
  misc_feature 1..3347: note = NK19-7b DNA
  source 1..3347: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgcggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgcgtcgac

SEQ ID NO: 72 - protein, 1103 aa, Homo sapiens
  REGION 1..1103: note = MISC_FEATURE - NK19-7b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYCGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 73 - DNA, 1844 bp, Homo sapiens
  misc_feature 1..1844: note = NK19-8b DNA
  source 1..1844: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 74 - protein, 602 aa, Homo sapiens
  REGION 1..602: note = MISC_FEATURE - NK19-8b Protein
MALPVTALLLPLALLLHAARPDYKDDDDKGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 75 - DNA, 2243 bp, Homo sapiens
  misc_feature 1..2243: note = NK19-9b DNA
  source 1..2243: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctcccccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 76 - protein, 735 aa, Homo sapiens
  REGION 1..735: note = MISC_FEATURE - NK19-9b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 77 - DNA, 2150 bp, Homo sapiens
  misc_feature 1..2150: note = NK19-10b DNA
  source 1..2150: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcatgccggaggagggttcgggctgctcggtgcggcgcaggccctatgggtgcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 78 - protein, 704 aa, Homo sapiens
  REGION 1..704: note = MISC_FEATURE - NK19-10b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCMPEEGSGCSVRRRPYGCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 79 - DNA, 2234 bp, Homo sapiens
  misc_feature 1..2234: note = NK19-11b DNA
  source 1..2234: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcatggaccaacaagcaatatatgctgagttaaacttacccacagactcaggcccagaaagttcttcaccttcatctcttcctcgggatgtctgtcagggttcaccttggcatcaatttgccctgaaacttagctgtagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 80 - protein, 732 aa, Homo sapiens
  REGION 1..732: note = MISC_FEATURE - NK19-11b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCMDQQAIYAELNLPTDSGPESSSPSSLPRDVCQGSPWHQFALKLSCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 81 - DNA, 2165 bp, Homo sapiens
  misc_feature 1..2165: note = NK19-12b DNA
  source 1..2165: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcatgatcgaaacatacaaccaaacttctccccgatctgcggccactggactgcccatcagcatgaaaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 82 - protein, 709 aa, Homo sapiens
  REGION 1..709: note = MISC_FEATURE - NK19-12b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCMIETYNQTSPRSAATGLPISMKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 83 - DNA, 2315 bp, Homo sapiens
  misc_feature 1..2315: note = NK19-13b DNA
  source 1..2315: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 84 - protein, 759 aa, Homo sapiens
  REGION 1..759: note = MISC_FEATURE - NK19-13b Protein
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCNSRRRCGQKKKLVINSGNGAVEDRKPSGLNGEASKSQEMVHLVNKESSETPDQFMTADETRNLQNVDMKIGVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 85 - DNA, 6488 bp, Homo sapiens
  misc_feature 1..6488: note = NK19 DNA
  source 1..6488: mol_type = other DNA
atgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgttaacctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggga

SEQ ID NO: 86 - protein, 486 aa, Homo sapiens
  REGION 1..486: note = MISC_FEATURE - NK19 PROTEIN
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 6488 bp, Homo sapiens; identical to SEQ ID NO: 85
  misc_feature 1..6488: note = MSCV-IRES-GFP expression plasmid
  source 1..6488: mol_type = other DNA

SEQ ID NO: 88 - protein, 117 aa, Homo sapiens
  REGION 1..117: note = MISC_FEATURE - anti CLDN6 heavy chain
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSS

SEQ ID NO: 89 - protein, 107 aa, Homo sapiens
  REGION 1..107: note = MISC_FEATURE - anti CLDN6 light chain 1
QIVLTQSPAIMSASPGEKVTITCSASSSVSYLHWFQQKPGTSPKLWVYSTSNLPSGVPARFGGSGSGTSYSLTISRMEAEDAATYYCQQRSIYPPWTFGGGTKLEIK

SEQ ID NO: 90 - protein, 107 aa, Homo sapiens
  REGION 1..107: note = MISC_FEATURE - anti CLDN6 light chain 2
QIVLTQSPSIMSVSPGEKVTITCSASSSVSYMHWFQQKPGTSPKLGIYSTSNLASGVPARFSGRGSGTSYSLTISRVAAEDAATYYCQQRSNYPPWTFGGGTKLEIK

SEQ ID NO: 91 - protein, 107 aa, Homo sapiens
  REGION 1..107: note = MISC_FEATURE - anti CLDN6 light chain 3
QIVLTQSPSIMSVSPGEKVTITCSASSSVSYMHWFQQKPGTSPKLSIYSTSNLASGVPARFSGRGSGTSYSLTISRVAAEDAATYYCQQRSNYPPWTFGGGTKLEIK

SEQ ID NO: 92 - protein, 8 aa, Homo sapiens
  REGION 1..8: note = MISC_FEATURE - anti CLDN6 heavy chain CDR1
GYSFTGYT

SEQ ID NO: 93 - protein, 8 aa, Homo sapiens
  REGION 1..8: note = MISC_FEATURE - anti CLDN6 heavy chain CDR2
INPYNGGT

SEQ ID NO: 94 - protein, 10 aa, Homo sapiens
  REGION 1..10: note = MISC_FEATURE - anti CLDN6 heavy chain CDR3
ARDYGFVLDY

SEQ ID NO: 95 - protein, 5 aa, Homo sapiens
  REGION 1..5: note = MISC_FEATURE - anti CLDN6 light chain 1 CDR1
SSVSY

SEQ ID NO: 96 - skipped

SEQ ID NO: 97 - protein, 8 aa, Homo sapiens
  REGION 1..8: note = MISC_FEATURE - anti CLDN6 light chain 1 CDR3
QQRSIYPP

SEQ ID NO: 98 - protein, 5 aa, Homo sapiens
  REGION 1..5: note = MISC_FEATURE - anti CLDN6 light chain 2 CDR1
SSVSY

SEQ ID NO: 99 - skipped

SEQ ID NO: 100 - protein, 8 aa, Homo sapiens
  REGION 1..8: note = MISC_FEATURE - anti CLDN6 light chain 2 CDR3
QQRSNYPP

SEQ ID NO: 101 - protein, 5 aa, Homo sapiens
  REGION 1..5: note = MISC_FEATURE - anti CLDN6 LC CDR1
SSVSY

SEQ ID NO: 102 - skipped

SEQ ID NO: 103 - protein, 8 aa, Homo sapiens
  REGION 1..8: note = MISC_FEATURE - anti CLDN6 LC3 CDR3
QQRSNYPP

SEQ ID NO: 104 - protein, 120 aa, Homo sapiens
  REGION 1..120: note = MISC_FEATURE - anti CD19 heavy chain variable region
QVQLVQSGGGVVQPGRSLRLSCAASGVSLPDYGVSWVRQAPGKGLEWVAVIWGSETTYYNSALKSRFTISRDNSKNTLYLQMNSLRAEDTAVYYCAKHYYYGGSYAMDYWGQGTLVTVSS

SEQ ID NO: 105 - protein, 107 aa, Homo sapiens
  REGION 1..107: note = MISC_FEATURE - anti CD19 light chain variable region
DIQMTQSPSSLSASVGDRVTITCRASQDISKYLNWYQQKPGKAIKLLIYHTSRLHSGVPSRFSGSGSGTDFTLTISSLQPEDFATYYCQQGATLPYTFGPGTKVDIK

SEQ ID NO: 106 - protein, 450 aa, Homo sapiens
  REGION 1..450: note = MISC_FEATURE - anti CD19 heavy chain variable
QVQLVQSGGGVVQPGRSLRLSCAASGVSLPDYGVSWVRQAPGKGLEWVAVIWGSETTYYNSALKSRFTISRDNSKNTLYLQMNSLRAEDTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 214 aa, Homo sapiens
  REGION 1..214: note = MISC_FEATURE - anti CD19 light chain variable
DIQMTQSPSSLSASVGDRVTITCRASQDISKYLNWYQQKPGKAIKLLIYHTSRLHSGVPSRFSGSGSGTDFTLTISSLQPEDFATYYCQQGATLPYTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 11 aa, Homo sapiens
  REGION 1..11: note = MISC_FEATURE - anti CD19 LCCDR1
RASQDISKYLN

SEQ ID NO: 109 - protein, 7 aa, Homo sapiens
  REGION 1..7: note = MISC_FEATURE - anti CD19 LCCDR2
HTSRLHS

SEQ ID NO: 110 - protein, 9 aa, Homo sapiens
  REGION 1..9: note = MISC_FEATURE - anti CD19 LCCDR3
QQGNTLPYT

SEQ ID NO: 111 - protein, 10 aa, Homo sapiens
  REGION 1..10: note = MISC_FEATURE - anti CD19 HCCDR1
GVSLPDYGVS

SEQ ID NO: 112 - protein, 16 aa, Homo sapiens
  REGION 1..16: note = MISC_FEATURE - anti CD19 HCCDR2
VIWGSETTYYSSSLKS

SEQ ID NO: 113 - protein, 16 aa, Homo sapiens
  REGION 1..16: note = MISC_FEATURE - anti CD19 HCCDR2
VIWGSETTYYQSSLKS

SEQ ID NO: 114 - protein, 16 aa, Homo sapiens
  REGION 1..16: note = MISC_FEATURE - anti CD19 HCCDR2
VIWGSETTYYNSSLKS

SEQ ID NO: 115 - protein, 12 aa, Homo sapiens
  REGION 1..12: note = MISC_FEATURE - anti CD19 HCCDR3
HYYYGGSYAMDY

SEQ ID NO: 116 - protein, 242 aa, Homo sapiens
  REGION 1..242: note = MISC_FEATURE - antiCD19CAR
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 117 - protein, 107 aa, Homo sapiens
  REGION 1..107: note = MISC_FEATURE - huFMC63VLv1
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEIT

SEQ ID NO: 118 - protein, 107 aa, Homo sapiens
  REGION 1..107: note = MISC_FEATURE - HUFMC63VLV2
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEIT

SEQ ID NO: 119 - protein, 107 aa, Homo sapiens
  REGION 1..107: note = MISC_FEATURE - HUFMC63VLV3
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEIT

SEQ ID NO: 120 - protein, 120 aa, Homo sapiens
  REGION 1..120: note = MISC_FEATURE - HUFMC63VHV1
QVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSS

SEQ ID NO: 121 - protein, 120 aa, Homo sapiens
  REGION 1..120: note = MISC_FEATURE - HUFMC63VHV2
QVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSS

SEQ ID NO: 122 - protein, 120 aa, Homo sapiens
  REGION 1..120: note = MISC_FEATURE - HUFMC63VHV3
QVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSS

SEQ ID NO: 123 - protein, 120 aa, Homo sapiens
  REGION 1..120: note = MISC_FEATURE - HUFMC63VHV4
QVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSS

SEQ ID NO: 124 - protein, 11 aa, Homo sapiens
  REGION 1..11: note = MISC_FEATURE - huFMC63VLv1 CDR1
RASQDISKYLN

SEQ ID NO: 125 - protein, 7 aa, Homo sapiens
  REGION 1..7: note = MISC_FEATURE - huFMC63VLv1 CDR2
HTSRLHS

SEQ ID NO: 126 - skipped

SEQ ID NO: 127 - protein, 11 aa, Homo sapiens
  REGION 1..11: note = MISC_FEATURE - HUFMC63VLV2 CDR1
RASQDISKYLN

SEQ ID NO: 128 - protein, 7 aa, Homo sapiens
  REGION 1..7: note = MISC_FEATURE - HUFMC63VLV2 CDR2
HTSRLHS

SEQ ID NO: 129 - protein, 9 aa, Homo sapiens
  REGION 1..9: note = MISC_FEATURE - HUFMC63VLV2 CDR3
QQGNTLPYT

SEQ ID NO: 130 - protein, 11 aa, Homo sapiens
  REGION 1..11: note = MISC_FEATURE - HUFMC63VLV3 CDR1
RASQDISKYLN

SEQ ID NO: 131 - protein, 7 aa, Homo sapiens
  REGION 1..7: note = MISC_FEATURE - HUFMC63VLV3 CDR2
HTSRLHS

SEQ ID NO: 132 - protein, 9 aa, Homo sapiens
  REGION 1..9: note = MISC_FEATURE - HUFMC63VLV3 CDR3
QQGNTLPYT

SEQ ID NO: 133 - protein, 10 aa, Homo sapiens
  REGION 1..10: note = MISC_FEATURE - HUFMC63VHV1 CDR1
GVSLPDYGVS

SEQ ID NO: 134 - protein, 16 aa, Homo sapiens
  REGION 1..16: note = MISC_FEATURE - HUFMC63VHV1 CDR2
VIWGSETTYYNSALKS

SEQ ID NO: 135 - protein, 12 aa, Homo sapiens
  REGION 1..12: note = MISC_FEATURE - HUFMC63VHV1 CDR3
HYYYGGSYAMDY

SEQ ID NO: 136 - protein, 10 aa, Homo sapiens
  REGION 1..10: note = MISC_FEATURE - HUFMC63VHV2 CDR1
GVSLPDYGVS

SEQ ID NO: 137 - protein, 16 aa, Homo sapiens
  REGION 1..16: note = MISC_FEATURE - HUFMC63VHV2 CDR2
VIWGSETTYYNSALKS

SEQ ID NO: 138 - protein, 12 aa, Homo sapiens
  REGION 1..12: note = MISC_FEATURE - HUFMC63VHV2 CDR3
HYYYGGSYAMDY

SEQ ID NO: 139 - protein, 10 aa, Homo sapiens
  REGION 1..10: note = MISC_FEATURE - HUFMC63VHV3 CDR1
GVSLPDYGVS

SEQ ID NO: 140 - protein, 16 aa, Homo sapiens
  REGION 1..16: note = MISC_FEATURE - HUFMC63VHV3 CDR2
VIWGSETTYYNSALKS

SEQ ID NO: 141 - protein, 12 aa, Homo sapiens
  REGION 1..12: note = MISC_FEATURE - HUFMC63VHV3 CDR3
HYYYGGSYAMDY

SEQ ID NO: 142 - protein, 10 aa, Homo sapiens
  REGION 1..10: note = MISC_FEATURE - HUFMC63VHV4 CDR1
GVSLPDYGVS

SEQ ID NO: 143 - protein, 16 aa, Homo sapiens
  REGION 1..16: note = MISC_FEATURE - HUFMC63VHV4 CDR2
VIWGSETTYYNSALKS

SEQ ID NO: 144 - protein, 12 aa, Homo sapiens
  REGION 1..12: note = MISC_FEATURE - HUFMC63VHV4 CDR3
HYYYGGSYAMDY

SEQ ID NO: 145 - DNA, 1122 bp, Homo sapiens
  misc_feature 1..1122: note = DNA NKG2D-Ox40-CD3z
  source 1..1122: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 146 - protein, 341 aa, Homo sapiens
  REGION 1..341: note = MISC_FEATURE - Amino Acid NKG2D-OX40-CD3z
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVESKYGPPCPSCPIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - DNA, 1467 bp, Homo sapiens
  misc_feature 1..1467: note = DNA Sequence FMC63_CD28_CAR
  source 1..1467: mol_type = other DNA
atgcttctcctggtgacaagccttctttaccacacccagcattcctcctgatcccagacatccagatacatcctccctgtctgcctctctgggagacagagtcaccgggcaagtcaggacattagtaaatatttaaattggtatcagctctgtgagtgacacagacatcagttgcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 148 - DNA, 331 bp, Homo sapiens
  misc_feature 1..331: note = DNA Sequence FCM63 Light Chain
  source 1..331: mol_type = other DNA
gacatccagatacatcctccctgtctgcctctctgggagacagagtcaccgggcaagtcaggacattagtaaatatttaaattggtatcagctctgtgagtgacacagacatcagttgcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 149 - DNA, 54 bp, Homo sapiens
  misc_feature 1..54: note = DNA Sequence FMC63 Linker
  source 1..54: mol_type = other DNA
ggctccacctctggatccggcaagcccggatctggcgagggatccaccaagggc

SEQ ID NO: 150 - DNA, 360 bp, Homo sapiens
  misc_feature 1..360: note = DNA Sequence FMC63 Heavy Chain
  source 1..360: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 151 - DNA, 117 bp, Homo sapiens
  misc_feature 1..117: note = DNA Sequence CD28 spacer
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 152 - DNA, 81 bp, Homo sapiens
  misc_feature 1..81: note = DNA Sequence transmembrane region
  source 1..81: mol_type = other DNA
ttttgggtgctggtggtggttgggggagtcctggcttgctatagcttgctagtaacagtggcctttattattttctgggtg

SEQ ID NO: 153 - DNA, 123 bp, Homo sapiens
  misc_feature 1..123: note = DNA Sequence CD28 Co-stim
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 154 - DNA, 336 bp, Homo sapiens; identical to SEQ ID NO: 7
  misc_feature 1..336: note = CD3zeta [repeat]
  source 1..336: mol_type = other DNA

SEQ ID NO: 155 - DNA, 342 bp, Homo sapiens; identical to SEQ ID NO: 11
  misc_feature 1..342: note = Coding sequence for mbIL15
  source 1..342: mol_type = other DNA

SEQ ID NO: 156 - DNA, 135 bp, Homo sapiens
  misc_feature 1..135: note = DNA seq of cd hinge rev. trans from No: 157
  source 1..135: mol_type = other DNA
accaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgat

SEQ ID NO: 157 - protein, 45 aa, Homo sapiens; identical to SEQ ID NO: 2
  REGION 1..45: note = MISC_FEATURE - Amino Acid CD8 Hinge

SEQ ID NO: 158 - DNA, 72 bp, Homo sapiens
  misc_feature 1..72: note = DNA seq of CD8 TM rev trans from No: 159
  source 1..72: mol_type = other DNA
atttatatttgggcgccgctggcgggcacctgcggcgtgctgctgctgagcctggtgattaccctgtattgc

SEQ ID NO: 159 - protein, 24 aa, Homo sapiens
  REGION 1..24: note = MISC_FEATURE - AA Sequence CD8 TM
IYIWAPLAGTCGVLLLSLVITLYC

SEQ ID NO: 160 - DNA, 2204 bp, Homo sapiens
  misc_feature 1..2204: note = DNA NK19H-1
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 161 - protein, 724 aa, Homo sapiens
  REGION 1..724: note = MISC_FEATURE - AA NK19H-1
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 162 - DNA, 2187 bp, Homo sapiens
  misc_feature 1..2187: note = DNA NK19H-2
  source 1..2187: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 163 - protein, 724 aa, Homo sapiens
  REGION 1..724: note = MISC_FEATURE - AA NK19H-2
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 164 - DNA, 2204 bp, Homo sapiens
  misc_feature 1..2204: note = DNA NK19H-3
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 165 - protein, 724 aa, Homo sapiens
  REGION 1..724: note = MISC_FEATURE - AA NK19H-3
MALPVTALLLPLALLLHAARPDYKDDDDKGGGGSGGGGSDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 166 - DNA, 2204 bp, Homo sapiens
  misc_feature 1..2204: note = DNA NK19H-4
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggctgggcgtgatttggggcagcgaaaccacctattataacagcgcgctgaaaagccgcctgaccattagcaaagataacagcaaaagccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 167 - protein, 724 aa, Homo sapiens
  REGION 1..724: note = MISC_FEATURE - AA NK19H-4
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 168 - DNA, 2204 bp, Homo sapiens
  misc_feature 1..2204: note = DNA NK19H-5
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggctgggcgtgatttggggcagcgaaaccacctattataacagcgcgctgaaaagccgcctgaccattagcaaagataacagcaaaagccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 169 - protein, 724 aa, Homo sapiens
  REGION 1..724: note = MISC_FEATURE - AA NK19H-5
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 170 - DNA, 2204 bp, Homo sapiens
  misc_feature 1..2204: note = DNA NK19H-6
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 171 - protein, 724 aa, Homo sapiens
  REGION 1..724: note = MISC_FEATURE - AA NK19H-6
MALPVTALLLPLALLLHAARPDYKDDDDKGGGGSGGGGSDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 172 - DNA, 2204 bp, Homo sapiens
  misc_feature 1..2204: note = DNA NK19H-7
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttactactgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 173 - protein, 724 aa, Homo sapiens
  REGION 1..724: note = MISC_FEATURE - AA NK19H-7
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 174 - DNA, 2204 bp, Homo sapiens
  misc_feature 1..2204: note = DNA NK19H-8
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 175 - protein, 724 aa, Homo sapiens
  REGION 1..724: note = MISC_FEATURE - AA NK19H-8
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 176 - DNA, 2204 bp, Homo sapiens
  misc_feature 1..2204: note = DNA NK19H-9
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 177 - protein, 724 aa, Homo sapiens
  REGION 1..724: note = MISC_FEATURE - AA NK19H-9
MALPVTALLLPLALLLHAARPDYKDDDDKGGGGSGGGGSDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 178 - DNA, 2204 bp, Homo sapiens
  misc_feature 1..2204: note = DNA NK19H-10
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 179 - protein, 724 aa, Homo sapiens
  REGION 1..724: note = MISC_FEATURE - AA NK19H-10
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 180 - DNA, 2204 bp, Homo sapiens
  misc_feature 1..2204: note = DNA NK19H-11
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 181 - protein, 724 aa, Homo sapiens
  REGION 1..724: note = MISC_FEATURE - AA NK19H-11
MALPVTALLLPLALLLHAARPDYKDDDDKGGGGSGGGGSDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 182 - DNA, 2204 bp, Homo sapiens
  misc_feature 1..2204: note = DNA NK19H-12
  source 1..2204: mol_type = other DNA
gaattcgccgccaccatggccttaccagtgaccgccttgctcctgccgctggccttgctgctccacgccgccaggccggactacaaagacgatgacgataaaggcggtggtggctctggtggtggcggcagcgatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 183 - protein, 724 aa, Homo sapiens
  REGION 1..724: note = MISC_FEATURE - AA NK19H-12
MALPVTALLLPLALLLHAARPDYKDDDDKGGGGSGGGGSDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 184 - DNA, 2150 bp, Homo sapiens
  misc_feature 1..2150: note = DNA NK19H-NF-1
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 185 - protein, 706 aa, Homo sapiens
  REGION 1..706: note = MISC_FEATURE - AA NK19H-NF-1
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 186 - DNA, 2150 bp, Homo sapiens
  misc_feature 1..2150: note = DNA NK19H-NF-2
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 187 - protein, 706 aa, Homo sapiens
  REGION 1..706: note = MISC_FEATURE - AA NK19H-NF-2
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 188 - DNA, 2150 bp, Homo sapiens
  misc_feature 1..2150: note = DNA NK19H-NF-3
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 189 - protein, 706 aa, Homo sapiens
  REGION 1..706: note = MISC_FEATURE - AA NK19H-NF-3
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 190 - DNA, 2150 bp, Homo sapiens
  misc_feature 1..2150: note = DNA NK19H-NF-4
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggctgggcgtgatttggggcagcgaaaccacctattataacagcgcgctgaaaagccgcctgaccattagcaaagataacagcaaaagccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 191 - protein, 706 aa, Homo sapiens
  REGION 1..706: note = MISC_FEATURE - AA NK19H-NF-4
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 192 - DNA, 2153 bp, Homo sapiens
  misc_feature 1..2153: note = DNA NK19H-NF-5
  source 1..2153: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcagatctagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 193 - protein, 707 aa, Homo sapiens
  REGION 1..707: note = MISC_FEATURE - AA NK19H-NF-5
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 194 - DNA, 2150 bp, Homo sapiens
  misc_feature 1..2150: note = DNA NK19H-NF-6
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 195 - protein, 706 aa, Homo sapiens
  REGION 1..706: note = MISC_FEATURE - AA NK19H-NF-6
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 196 - DNA, 2150 bp, Homo sapiens
  misc_feature 1..2150: note = DNA NK19H-NF-7
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttactactgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 197 - protein, 706 aa, Homo sapiens
  REGION 1..706: note = MISC_FEATURE - AA NK19H-NF-7
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 198 - DNA, 2150 bp, Homo sapiens
  misc_feature 1..2150: note = DNA NK19H-NF-8
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 199 - protein, 706 aa, Homo sapiens
  REGION 1..706: note = MISC_FEATURE - AA NK19H-NF-8
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 200 - DNA, 2150 bp, Homo sapiens
  misc_feature 1..2150: note = DNA NK19H-NF-9
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 201 - protein, 706 aa, Homo sapiens
  REGION 1..706: note = MISC_FEATURE - AA NK19H-NF-9
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 202 - DNA, 2150 bp, Homo sapiens
  misc_feature 1..2150: note = DNA NK19H-NF-10
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 203 - protein, 706 aa, Homo sapiens
  REGION 1..706: note = MISC_FEATURE - AA NK19H-NF-10
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 204 - DNA, 2150 bp, Homo sapiens
  misc_feature 1..2150: note = DNA NK19H-NF-11
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 205 - protein, 706 aa, Homo sapiens
  REGION 1..706: note = MISC_FEATURE - AA NK19H-NF-11
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 206 - DNA, 2150 bp, Homo sapiens
  misc_feature 1..2150: note = DNA NK19H-NF-12
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 207 - protein, 706 aa, Homo sapiens
  REGION 1..706: note = MISC_FEATURE - AA NK19H-NF-12
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

(19) United States
(12) Patent Application Publication
(10) Pub. No.: US 2025/0296970 A1
(43) Pub. Date: Sep. 25, 2025

(54) CD19-DIRECTED CHIMERIC ANTIGEN RECEPTORS AND USES THEREOF IN IMMUNOTHERAPY

(71) Applicant: Nkarta, Inc., South San Francisco, CA (US)

(72) Inventors: James Barnaby Trager, Albany, CA (US); Luxuan Guo Buren, San Francisco, CA (US); Chao Guo, San Francisco, CA (US); Mira Tohmé, San Francisco, CA (US); Ivan Chan, Millbrae, CA (US); Alexandra Leida Liana Lazetic, San Jose, CA (US)

(21) Appl. No.: 19/080,060

(22) Filed: Mar. 14, 2025

Related U.S. Application Data
(63) Continuation of application No. 17/571,325, filed on Jan. 7, 2022.
Application No. 17/571,325 is a continuation of application No. 17/089,449, filed on Nov. 4, 2020, now Pat. No. 11,253,547.
Application No. 17/089,449 is a continuation of application No. PCT/US2020/020824, filed on Mar. 3, 2020.
(60) Provisional application No. 62/814,180, filed on Mar. 5, 2019.
(60) Provisional application No. 62/895,910, filed on Sep. 4, 2019.
(60) Provisional application No. 62/932,165, filed on Nov. 7, 2019.

(51) Int. Cl. C07K 14/55; A61K 40/15; A61K 40/31; A61K 40/42; A61P 35/00; C07K 14/54; C07K 14/705; C07K 14/725; C07K 16/28; C12N 5/0783

(52) U.S. Cl. CPC C07K 14/55; A61K 40/15; A61K 40/31; A61K 40/4211; A61P 35/00; C07K 14/5443; C07K 14/7051; C07K 14/70517; C07K 14/70578; C07K 16/2803; C07K 16/2878; C12N 5/0646; A61K 2239/38; A61K 2239/48; C07K 2317/56; C07K 2317/622; C07K 2319/02; C07K 2319/03

(57) ABSTRACT

Provided for herein in several embodiments are immune cell-based (e.g., natural killer (NK) cell) compositions comprising CD19-directed chimeric antigen receptors. In some, embodiments the anti-CD19 binder portion of the CAR is humanized. In several embodiments, the humanized anti-CD19 CAR expressing cells exhibit enhanced expression of the CAR as well as enhanced cytotoxicity and/or persistence. Several embodiments include methods of using of the anti-CD19 CAR expressing immune cells in immunotherapy.

### RELATED CASES

This application is a continuation of U.S. patent application Ser. No. 17/571,325, filed Jan. 7, 2022, which is a continuation of U.S. patent application Ser. No. 17/089,449 (issued as U.S. Pat. No. 11,253,547), filed Nov. 4, 2020, which is a continuation of International Patent Application No. PCT/US2020/020824, filed Mar. 3, 2020, which claims the priority to U.S. Provisional Patent Application Nos. 62/814,180, filed Mar. 5, 2019, 62/895,910, filed Sep. 4, 2019, and 62/932,165, filed Nov. 7, 2019, the entire contents of each of which is incorporated by reference herein.

### FIELD

Some embodiments of the methods and compositions provided herein relate to CD19-directed receptors. In some embodiments the receptors are chimeric. Some embodiments include methods of use of the chimeric receptors in immunotherapy.

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered immune cells are one approach to treating cancers.

### INCORPORATION BY REFERENCE OF MATERIAL IN SEQUENCE LISTING FILE

The present application is being filed along with a Sequence Listing in electronic format. The Sequence Listing is provided as a file entitled NKT.033C5_ST26.xml, which was created on Mar. 14, 2025, which is 389,802 bytes in size. The information in the electronic Sequence Listing is hereby incorporated by reference in its entirety.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric receptors in immune cells to achieve the targeted recognition and destruction of aberrant cells of interest.

In several embodiments, there is provided herein an immune cell, and also populations of immune cells, that expresses a CD19-directed chimeric receptor, the chimeric receptor comprising an extracellular anti-CD19 binding moiety, a hinge and/or transmembrane domain, and an intracellular signaling domain. Also provided for herein are polynucleotides (as well as vectors for transfecting cells with the same) encoding a CD19-directed chimeric antigen receptor, the chimeric antigen receptor comprising an extracellular anti-CD19 binding moiety, a hinge and/or transmembrane domain, and an intracellular signaling domain.

In several embodiments, there is provided a polynucleotide encoding a CD19-directed chimeric antigen receptor, the chimeric antigen receptor comprising an extracellular anti-CD19 binding moiety, wherein the anti-CD19 binding moiety comprises a variable heavy (VH) domain of a single chain Fragment variable (scFv) and a variable light (VL) domain of a scFv, a hinge, a transmembrane domain, and an intracellular signaling domain, and wherein the intracellular signaling domain comprises an OX40 subdomain, a CD3 zeta subdomain.

In several embodiments, there is provided a polynucleotide encoding a CD19-directed chimeric antigen receptor, the chimeric antigen receptor comprising an extracellular anti-CD19 binding moiety, wherein the anti-CD19 binding moiety comprises a variable heavy (VH) domain of a single chain Fragment variable (scFv) and a variable light (VL) domain of a scFv, wherein the encoded VH domain comprises at least one heavy chain complementarity determining region (CDR) selected from the group consisting of SEQ ID NO: 133, SEQ ID NO: 134, and SEQ ID NO: 135, wherein the encoded VL domain comprises at least one light chain complementarity determining region (CDR) selected from the group consisting of SEQ ID NO: 127, SEQ ID NO: 128, and SEQ ID NO: 129, a hinge domain, a transmembrane domain, an intracellular signaling domain, and wherein the intracellular signaling domain comprises an OX40 subdomain and a CD3 zeta subdomain.

In several embodiments, the polynucleotide also encodes membrane-bound interleukin-15 (mbIL15). However, in some embodiments, a separate polynucleotide is used to encode the mbIL15. In several embodiments, the transmembrane domain is derived from or comprises a CD8 alpha transmembrane domain. In several embodiments, the CD8 alpha transmembrane domain is encoded by SEQ ID NO: 3. In several embodiments, the hinge is derived from or comprises a CD8 alpha hinge. In several embodiments, the CD8 alpha hinge is encoded by SEQ ID NO: 1. In several embodiments, the OX40 subdomain is encoded by a sequence having at least 90% (e.g., 90-95%, 95%, 96%, 97%, 98%, or 99%) sequence identity to SEQ ID NO. 5. In several embodiments, the CD3 zeta subdomain is encoded by a sequence having at least 90% (e.g., 90-95%, 95%, 96%, 97%, 98%, or 99%) sequence identity to SEQ ID NO. 7. In several embodiments, the mbIL15 is encoded by a sequence having at least 90% (e.g., 90-95%, 95%, 96%, 97%, 98%, or 99%) sequence identity to SEQ ID NO. 11. In several embodiments, the OX40 domain is encoded by SEQ ID NO: 5, the CD3 zeta subdomain is encoded by SEQ ID NO. 7 and/or mbIL15 (whether encoded separately or bicistronically) is encoded by SEQ ID NO: 11. In several embodiments, the encoded OX40 subdomain comprises the amino acid sequence of SEQ ID NO: 6, the encoded CD3 zeta subdomain comprises the amino acid sequence of SEQ ID NO: 8, and/or the encoded mbIL15 (whether encoded separately or bicistronically) comprises the amino acid sequence of SEQ ID NO: 12.

In several embodiments, the VH domain comprises a VH domain selected from SEQ ID NO: 120, SEQ ID NO: 121, SEQ ID NO: 122, and SEQ ID NO: 123, and wherein the VL domain comprises a VL domain selected from SEQ ID NO: 117, SEQ ID NO: 118, and SEQ ID NO: 119. In several embodiments, the polynucleotide encodes a VL domain comprising at least one light chain complementarity determining region (CDR) selected from the group consisting of SEQ ID NO: 127, SEQ ID NO: 128, and SEQ ID NO: 129. In several embodiments, the polynucleotide encodes a VH domain comprising at least one heavy chain complementarity determining region (CDR) selected from the group consisting of SEQ ID NO: 133, SEQ ID NO: 134, and SEQ ID NO: 135. In several embodiments, the polynucleotide is designed (e.g., engineered) to reduce potential antigenicity of the encoded protein and/or enhance one or more characteristics of the encoded protein (e.g., target recognition and/or binding characteristics) Thus, according to several embodiments, the anti-CD19 binding moiety does not comprise certain sequences. For example, according to several embodiments the polynucleotide does not encode one or more of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52. SEQ ID NO: 53, SEQ ID NO: 54, or SEQ ID NO: 55. In several embodiments, the encoded VH domain comprises an amino acid sequence at having at least 90% (e.g., 90-95%, 95%, 96%, 97%, 98%, or 99%) sequence identity to SEQ ID NO: 120. In several embodiments, the encoded VH domain comprises the amino acid sequence of SEQ ID NO: 120. In several embodiments, the encoded VL domain comprises an amino acid sequence at having at least 90% (e.g., 90-95%, 95%, 96%, 97%, 98%, or 99%) sequence identity to SEQ ID NO: 118. In several embodiments, the encoded VL domain comprises the amino acid sequence of SEQ ID NO: 118. In several embodiments, VH domain is derived from a parent amino acid sequence and is modified from the parent sequence. For example, mutations, truncations, extensions, conservative substitutions, or other modifications are introduced to increase the affinity of the domain for its target, increase the avidity for the target, and/or reduce potential antigenicity of the sequence. In several embodiments, the VH domain results from humanization of the VH domain amino acid sequence set forth in SEQ ID NO: 33. Likewise, in several embodiments, the VL domain results from humanization of the VL domain amino acid sequence set forth in SEQ ID NO: 32. In several embodiments, the polynucleotide encodes a CD19-directed chimeric antigen receptor having at least is encoded by a sequence having at least 90% (e.g., 90-95%, 95%, 96%, 97%, 98%, or 99%) sequence identity to the amino acid sequence set forth in SEQ ID NO: 187. In several embodiments, the polynucleotide encodes a CD19-directed chimeric antigen receptor comprising the amino acid sequence set forth in SEQ ID NO: 187.

In several embodiments, the polynucleotide does not encode or otherwise comprise a DAP10 domain. In several embodiments, the polynucleotide does not encode or otherwise comprise a DAP12 domain. In several embodiments, the intracellular signaling domain comprises additional subdomains, that advantageously enhance generation of cytotoxic signals by cells expressing the constructs. In several embodiments, the polynucleotide further encodes one or more of CD44 and CD27 as signaling subdomains. In several embodiments, the polynucleotide optionally further encodes a detection tag or other moiety (e.g., marker) that allows for detection of expression of the protein(s) encoded by the polynucleotide by host cells.

There are also provided for herein uses of the disclosed polynucleotides in the manufacture of a medicament for enhancing NK cell cytotoxicity in a mammal in need thereof, in the manufacture of a medicament for treating cancer in a mammal in need thereof and/or for the treatment of cancer in a mammal in need thereof.

There are also provided for herein engineered immune cells that express the CD19-directed chimeric antigen receptors encoded by the polynucleotides disclosed herein. In several embodiments, the engineered immune cells are natural killer (NK) cell. In some embodiments, the engineered cells are T cells, though combinations of NK cell and T cells (and optionally other immune cell types) are used in some embodiments. In several embodiments, the immune cells are allogeneic with respect to a subject receiving the cells. There are also provided for herein the use of immune cells that express the CD19-directed chimeric antigen receptors encoded by the polynucleotides disclosed herein for the treatment of cancer in a mammal in need thereof. There are also provided for herein the use of immune cells that express the CD19-directed chimeric antigen receptors encoded by the polynucleotides disclosed herein for the manufacture of a medicament for the treatment of cancer in a mammal in need thereof.

In several embodiments, there are provided methods for treating cancer using the polynucleotides disclosed herein. For example, in several embodiments the methods comprise administering to a subject having a cancer a composition comprising a population of immune cells expressing CD19-directed chimeric antigen receptors as disclosed herein. In several embodiments, the CAR comprises an extracellular anti-CD19 binding moiety, wherein the anti-CD19 binding moiety comprises a variable heavy (VH) domain of a single chain Fragment variable (scFv) and a variable light (VL) domain of a scFv, a hinge, such as a CD8 alpha hinge, a transmembrane domain, such as a CD8 alpha transmembrane domain; and an intracellular signaling domain comprising an OX40 subdomain and a CD3 zeta subdomain, and wherein the cell also expresses membrane-bound interleukin-15 (mbIL15). In several embodiments, the OX40 subdomain is encoded by a sequence having at least 95% sequence identity to SEQ ID NO. 5, the CD3 zeta subdomain is encoded by a sequence having at least 95% sequence identity to SEQ ID NO. 7, and/or the mbIL15 is encoded by a sequence having at least 95% sequence identity to SEQ ID NO. 11. In several embodiments, the encoded VH domain comprises an amino acid sequence having at least 95% sequence identity to SEQ ID NO: 120, wherein the encoded VL domain comprises an amino acid sequence having at least 95% sequence identity to SEQ ID NO: 118. In several embodiments, the polynucleotide encodes a CD19-directed chimeric antigen receptor having at least 95% sequence identity to the amino acid sequence set forth in SEQ ID NO: 187. As discussed above, in several embodiments, the immune cells expressing such CD19 CAR constructs are natural killer (NK) cells. In some embodiments, the immune cells are T cells. In several embodiments, combinations of NK and T cells (and optionally other immune cells) are used. In several embodiments, the cells are allogeneic cells originating from a donor that is not the subject. In several embodiments, the cells are autologous cells originating the subject. Mixtures of allogeneic and autologous cells may also be used, in some embodiments. In several embodiments, the administered population comprises about 2×10⁶ cells per kilogram of body weight of the subject. In several embodiments, the administration is intravenous. In several embodiments, the method further comprises administering one or more doses of interleukin 2 to the subject. In several embodiments, the methods also involve the administration of another therapy to the subject. For example, in several embodiments, the methods involve administering a chemotherapy treatment to the subject prior to the administration of the cells. In several embodiments, the chemotherapy treatment induces lymphodepletion in the subject. In some embodiments, the subject is administered a combination of cyclophosphamide and fludarabine prior to administration of the cells. In several embodiments, the cyclophosphamide is administered in a dose between about 400 and about 600 mg/m². In several embodiments, the fludarabine is administered in a dose between about 25 and 25 mg/m². In several embodiments, the lymphodepleting chemotherapy is administered several days prior to administration of engineered immune cells and optionally administered multiple times. For example, in several embodiments the lymphodepleting chemotherapy is administered on at least the fifth, fourth, and/or third day prior to administration of engineered immune cells disclosed herein.

In several embodiments there is provided a polynucleotide encoding a humanized CD19-directed chimeric antigen receptor, the chimeric antigen receptor comprising a humanized anti-CD19 binding moiety, a co-stimulatory domain, and a signaling domain. In several embodiments, the co-stimulatory domain comprises OX40. In several embodiments, the humanized anti-CD19-binding moiety comprises a humanized scFv, wherein one or more of the heavy and light chains have been humanized. In several embodiments, one or more of the CDRs on the heavy and/or light chains have been humanized. For example, in several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a heavy chain variable (VH) domain and a and a light chain variable (VL) domain, the VH domain comprising a VH domain selected from SEQ ID NO: 120, SEQ ID NO: 121, SEQ ID NO: 122, and SEQ ID NO: 123 and the VL domain comprising a VL domain selected from SEQ ID NO: 117, SEQ ID NO: 118, and SEQ ID NO: 119, a hinge and/or transmembrane domain, an intracellular signaling domain.

In several embodiments, there is provided a polynucleotide encoding a humanized chimeric antigen receptor (CAR), wherein the CAR comprises a single chain antibody or single chain antibody fragment which comprises a humanized anti-CD19 binding domain, a transmembrane domain, a primary intracellular signaling domain comprising a native intracellular signaling domain of CD3-zeta, or a functional fragment thereof, and a costimulatory domain comprising a native intracellular signaling domain of a protein selected from the group consisting of OX40, CD27, CD28, ICOS, and 4-1BB, or a functional fragment thereof, wherein said anti-CD19 binding domain comprises a light chain complementary determining region 1 (LC CDR1) of SEQ ID NO: 124, 127, or 130, a light chain complementary determining region 2 (LC CDR2) of SEQ ID NO: 125, 128, or 131, and a light chain complementary determining region 3 (LC CDR3) of SEQ ID NO: 126, 129, or 132, and a heavy chain complementary determining region 1 (HC CDR1) of SEQ ID NO: 133, 136, 139, or 142, a heavy chain complementary determining region 2 (HC CDR2) of SEQ ID NO: 134, 137, 140, or 143, and a heavy chain complementary determining region 3 (HC CDR3) of SEQ ID NO: 135, 138, 141, or 144.

In several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable light (VL) domain of SEQ ID NO: 117, a hinge and/or transmembrane domain, and an intracellular signaling domain. In several embodiments, the polynucleotide encodes the humanized chimeric antigen receptor of SEQ ID NO: 161, SEQ ID NO: 167, SEQ ID NO: 173, SEQ ID NO: 179, SEQ ID NO: 185, SEQ ID NO: 191, SEQ ID NO: 197, or SEQ ID NO: 203.

In several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable light (VL) domain of SEQ ID NO: 118, a hinge and/or transmembrane domain, and an intracellular signaling domain. In several embodiments, the polynucleotide encodes the humanized chimeric antigen receptor of SEQ ID NO: 163, SEQ ID NO: 169, SEQ ID NO: 175, SEQ ID NO: 181, SEQ ID NO: 187, SEQ ID NO: 193, SEQ ID NO: 199, or SEQ ID NO: 205.

In several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable light (VL) domain of SEQ ID NO: 119, a hinge and/or transmembrane domain, and an intracellular signaling domain. In several embodiments, the polynucleotide encodes the humanized chimeric antigen receptor of SEQ ID NO: 165, SEQ ID NO: 171, SEQ ID NO: 177, SEQ ID NO: 183, SEQ ID NO: 189, SEQ ID NO: 195, SEQ ID NO: 201, or SEQ ID NO: 207.

In several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 120, a hinge and/or transmembrane domain, and an intracellular signaling domain. In several embodiments, the polynucleotide encodes the humanized chimeric antigen receptor of SEQ ID NO: 161, SEQ ID NO: 163, SEQ ID NO: 165, SEQ ID NO: 185, SEQ ID NO: 187, or SEQ ID NO: 189.

In several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 121, a hinge and/or transmembrane domain, and an intracellular signaling domain. In several embodiments, the polynucleotide encodes the humanized chimeric antigen receptor of SEQ ID NO: 167, SEQ ID NO: 169, SEQ ID NO: 171, SEQ ID NO: 191, SEQ ID NO: 193, or SEQ ID NO: 195.

In several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 122, a hinge and/or transmembrane domain, and an intracellular signaling domain. In several embodiments, the polynucleotide encodes the humanized chimeric antigen receptor of SEQ ID NO: 173, SEQ ID NO: 175, SEQ ID NO: 177, SEQ ID NO: 197, SEQ ID NO: 199, or SEQ ID NO: 201.

In several embodiments, there is provided a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 123, a hinge and/or transmembrane domain, and an intracellular signaling domain. In several embodiments, the polynucleotide encodes the humanized chimeric antigen receptor of SEQ ID NO: 179, SEQ ID NO: 181, SEQ ID NO: 183, SEQ ID NO: 203, SEQ ID NO: 205, or SEQ ID NO: 207.

In several embodiments, the provided polynucleotides also encode membrane-bound interleukin-15 (mbIL15).

In several embodiments, the intracellular signaling domain comprises an OX40 subdomain. However, in several embodiments the intracellular signaling domain comprises one or more of an OX40 subdomain, a CD28 subdomain, an iCOS subdomain, a CD28-41BB subdomain, a CD27 subdomain, a CD44 subdomain, or combinations thereof.

In several embodiments, the chimeric antigen receptor comprises a hinge and a transmembrane domain, wherein the hinge is a CD8 alpha hinge, wherein the transmembrane domain is either a CD8 alpha or an NKG2D transmembrane domain. In several embodiments, the intracellular signaling domain comprises a CD3zeta domain.

In several embodiments, the polynucleotide does not encode SEQ ID NO: 112, 113, or 114. In several embodiments the polynucleotide does not encode SEQ ID NO: 116.

In several embodiments, there are provided engineered NK cells, engineered T cells, and/or mixed populations of NK cells and T cells that express one or more of the humanized CD19-directed chimeric antigen receptors provided for herein.

Also provided are methods for treating cancer in a subject comprising administering to a subject having cancer the engineered NK and/or T cells expressing chimeric antigen receptors as disclosed herein. Also provided for are the use of the polynucleotides provided for herein for the treatment of cancer as well as use of the polynucleotides provided for herein in the manufacture of a medicament for the treatment of cancer.

Also provided for herein, in several embodiments, is a polynucleotide encoding a CD19-directed chimeric antigen receptor, the chimeric antigen receptor comprising an extracellular anti-CD19 binding moiety, wherein the anti-CD19 binding moiety comprises a scFv, a transmembrane domain, and an intracellular signaling domain, wherein the intracellular signaling domain comprises a CD28 co-stimulatory domain and a CD3 zeta signaling domain.

Also provided for herein, in several embodiments, is a polynucleotide encoding a CD19-directed chimeric antigen receptor, the chimeric antigen receptor comprising an extracellular anti-CD19 binding moiety, wherein the anti-CD19 binding moiety comprises a scFv, a hinge, wherein the hinge is a CD8 alpha hinge, a transmembrane domain, and an intracellular signaling domain, wherein the intracellular signaling domain comprises a CD3 zeta ITAM.

Also provided for herein, in several embodiments, is a polynucleotide encoding a CD19-directed chimeric antigen receptor, the chimeric antigen receptor comprising an extracellular anti-CD19 binding moiety, wherein the anti-CD19 binding moiety comprises a variable heavy chain of a scFv or a variable light chain of a scFv, a hinge, wherein the hinge is a CD8 alpha hinge, a transmembrane domain, wherein the transmembrane domain comprises a CD8 alpha transmembrane domain, and an intracellular signaling domain, wherein the intracellular signaling domain comprises a CD3 zeta ITAM.

In several embodiments, the transmembrane domain comprises a CD8 alpha transmembrane domain. In several embodiments, the transmembrane domain comprises an NKG2D transmembrane domain. In several embodiments, the transmembrane domain comprises a CD28 transmembrane domain.

In several embodiments the intracellular signaling domain comprises or further comprises a CD28 signaling domain. In several embodiments, the intracellular signaling domain comprises or further comprises a 4-1BB signaling domain. In several embodiments, the intracellular signaling domain comprises an or further comprises OX40 domain. In several embodiments, the intracellular signaling domain comprises or further comprises a 4-1BB signaling domain. In several embodiments, the intracellular signaling domain comprises or further comprises a domain selected from ICOS, CD70, CD161, CD40L, CD44, and combinations thereof.

In several embodiments, the polynucleotide also encodes a truncated epidermal growth factor receptor (EGFRt). In several embodiments, the EGFRt is expressed in a cell as a soluble factor. In several embodiments, the EGFRt is expressed in a membrane bound form. In several embodiments, the EGFRt operates to provide a “suicide switch” function in the engineered NK cells. In several embodiments, the polynucleotide also encodes membrane-bound interleukin-15 (mbIL15). Also provided for herein are engineered immune cells (e.g., NK or T cells, or mixtures thereof) that express a CD19-directed chimeric antigen receptor encoded by a polynucleotide disclosed herein. Further provided are methods for treating cancer in a subject comprising administering to a subject having cancer engineered immune cells expressing the chimeric antigen receptors disclosed herein. In several embodiments, there is provided the use of the polynucleotides disclosed herein in the treatment of cancer and/or in the manufacture of a medicament for the treatment of cancer.

In several embodiments, the anti-CD19 binding moiety comprises a heavy chain variable (VH) domain and a light chain variable (VL) domain. In several embodiments, the VH domain has at least 95% (e.g., 95, 96, 97, 98, or 99%) identity to the VH domain amino acid sequence set forth in SEQ ID NO: 33. In several embodiments, the VL domain has at least 95% (e.g., 95, 96, 97, 98, or 99%) identity to the VL domain amino acid sequence set forth in SEQ ID NO: 32. In several embodiments, the anti-CD19 binding moiety is derived from the VH and/or VL sequences of SEQ ID NO: 33 or 32. For example, in several embodiments, the VH and VL sequences for SEQ ID NO: 33 and/or 32 are subject to a humanization campaign and therefore are expressed more readily and/or less immunogenic when administered to human subjects. Thus, in several embodiments, the anti-CD19 binding moiety does not comprise SEQ ID NO: 32 and/or SEQ ID NO: 33. In several embodiments, the anti-CD19 binding moiety comprises a scFv that targets CD19 wherein the scFv comprises a heavy chain variable region comprising the sequence of SEQ ID NO. 35 or a sequence at least 95% (e.g., 95, 96, 97, 98, or 99%) identical to SEQ ID NO: 35. In several embodiments, the anti-CD19 binding moiety comprises an scFv that targets CD19 comprises a light chain variable region comprising the sequence of SEQ ID NO. 36 or a sequence at least 95% identical (e.g., 95, 96, 97, 98, or 99%) to SEQ ID NO: 36. In several embodiments, the anti-CD19 binding moiety comprises a light chain CDR comprising a first, second and third complementarity determining region (LC CDR1, LC CDR2, and LC CDR3, respectively) and/or a heavy chain CDR comprising a first, second and third complementarity determining region (HC CDR1, HC CDR2, and HC CDR3, respectively). Depending on the embodiment, various combinations of the LC CDRs and HC CDRs are used. For example, in one embodiment the anti-CD19 binding moiety comprises LC CDR1, LC CDR3, HC CD2, and HC, CDR3. Other combinations are used in some embodiments. In several embodiments, the LC CDR1 comprises the sequence of SEQ ID NO. 37 or a sequence at least about 95% homologous to the sequence of SEQ NO. 37. In several embodiments, the LC CDR2 comprises the sequence of SEQ ID NO. 38 or a or a sequence at least about 95% (e.g., 96, 97, 98, or 99%) homologous to the sequence of SEQ NO. 38. In several embodiments, the LC CDR3 comprises the sequence of SEQ ID NO. 39 or a sequence at least about 95% homologous to the sequence of SEQ NO. 39. In several embodiments, the HC CDR1 comprises the sequence of SEQ ID NO. 40 or a sequence at least about 95% homologous to the sequence of SEQ NO. 40. In several embodiments, the HC CDR2 comprises the sequence of SEQ ID NO. 41, 42, or 43 or a sequence at least about 95% homologous to the sequence of SEQ NO. 41, 42, or 43. In several embodiments, the HC CDR3 comprises the sequence of SEQ ID NO. 44 or a sequence at least about 95% (e.g., 96, 97, 98, 99 or 99%) homologous to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48 or a sequence at least about 95% (e.g., 96, 97, 98, 99 or 99%) homologous to the sequence of SEQ NO. 45, 46, 47, or 48. In several embodiments, the VH region comprises the sequence of SEQ ID NO. 49, 50, 51 or 52 or a sequence at least about 95% (e.g., 96, 97, 98, 99 or 99%) homologous to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53 or a sequence at least about 95% homologous to the sequence of SEQ NO. 53. In several embodiments, the LC CDR2 comprises the sequence of SEQ ID NO. 54 or a sequence at least about 95% homologous to the sequence of SEQ NO. 54. In several embodiments, the LC CDR3 comprises the sequence of SEQ ID NO. 55 or a sequence at least about 95% homologous to the sequence of SEQ NO. 55. In several embodiments, the HC CDR1 comprises the sequence of SEQ ID NO. 56 or a sequence at least about 95% homologous to the sequence of SEQ NO. 56. In several embodiments, the HC CDR2 comprises the sequence of SEQ ID NO. 57 or a sequence at least about 95% homologous to the sequence of SEQ NO. 57. In several embodiments, the HC CDR3 comprises the sequence of SEQ ID NO. 58 or a sequence at least about 95% homologous to the sequence of SEQ NO. 58. In several embodiments, the anti-CD19 binding moiety (and thus the resultant CAR) is engineered to not include certain sequences, such as, for example, those that may cause increased risk of immunogenicity and/or side effects, such as cytokine release syndrome. Thus, according to several embodiments, the anti-CD19 binding moiety does not comprise one or more of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52. SEQ ID NO: 53, SEQ ID NO: 54, SEQ ID NO: 55, SEQ ID NO: 32, or SEQ ID NO: 33.

In several embodiments, the intracellular signaling domain of the chimeric receptor comprises an OX40 subdomain. In several embodiments, the intracellular signaling domain further comprises a CD3zeta subdomain. In several embodiments, the OX40 subdomain comprises the amino acid sequence of SEQ ID NO: 16 (or a sequence at least about 95% homologous to the sequence of SEQ ID NO. 16) and the CD3zeta subdomain comprises the amino acid sequence of SEQ ID NO: 8 (or a sequence at least about 95% homologous to the sequence of SEQ ID NO: 8).

In several embodiments, the hinge domain comprises a CD8a hinge domain. In several embodiments, the CD8a hinge domain, comprises the amino acid sequence of SEQ ID NO: 2 or a sequence at least about 95% homologous to the sequence of SEQ ID NO: 2).

In several embodiments, the immune cell also expresses membrane-bound interleukin-15 (mbIL15). In several embodiments, the mbIL15 comprises the amino acid sequence of SEQ ID NO: 12 or a sequence at least about 95% homologous to the sequence of SEQ ID NO: 12.

In several embodiments, wherein the chimeric receptor further comprises an extracellular domain of an NKG2D receptor. In several embodiments, the immune cell expresses a second chimeric receptor comprising an extracellular domain of an NKG2D receptor, a transmembrane domain, a cytotoxic signaling complex and optionally, mbIL15. In several embodiments, the extracellular domain of the NKG2D receptor comprises a functional fragment of NKG2D comprising the amino acid sequence of SEQ ID NO: 26 or a sequence at least about 95% homologous to the sequence of SEQ ID NO: 26. In various embodiments, the immune cell engineered to express the chimeric antigen receptor and/or chimeric receptors disclosed herein is an NK cell. In some embodiments, T cells are used. In several embodiments, combinations of NK and T cells (and/or other immune cells) are used.

In several embodiments, there are provided herein methods of treating cancer in a subject comprising administering to the subject having an engineered immune cell targeting CD19 as disclosed herein. Also provided for herein is the use of an immune cell targeting CD19 as disclosed herein for the treatment of cancer. Likewise, there is provided for herein the use of an immune cell targeting CD19 as disclosed herein in the preparation of a medicament for the treatment of cancer. In several embodiments, the cancer treated is acute lymphocytic leukemia.

Some embodiments of the methods and compositions described herein relate to an immune cell. In some embodiments, the immune cell expresses a CD19-directed chimeric receptor comprising an extracellular anti-CD19 moiety, a hinge and/or transmembrane domain, and/or an intracellular signaling domain. In some embodiments, the immune cell is a natural killer (NK) cell. In some embodiments, the immune cell is a T cell.

In some embodiments, the hinge domain comprises a CD8a hinge domain. In some embodiments, the hinge domain comprises an Ig4 SH domain.

In some embodiments, the transmembrane domain comprises a CD8a transmembrane domain. In some embodiments, the transmembrane domain comprises a CD28 transmembrane domain. In some embodiments, the transmembrane domain comprises a CD3 transmembrane domain.

In some embodiments, the signaling domain comprises an OX40 signaling domain. In some embodiments, the signaling domain comprises a 4-1BB signaling domain. In some embodiments, the signaling domain comprises a CD28 signaling domain. In some embodiments, the signaling domain comprises an NKp80 signaling domain. In some embodiments, the signaling domain comprises a CD16 IC signaling domain. In some embodiments, the signaling domain comprises a CD3zeta or CD3ζ ITAM signaling domain. In some embodiments, the signaling domain comprises an mbIL-15 signaling domain. In some embodiments, the signaling domain comprises a 2A cleavage domain. In some embodiments, the mIL-15 signaling domain is separated from the rest or another portion of the CD19-directed chimeric receptor by a 2A cleavage domain.

Some embodiments relate to a method comprising administering an immune cell as described herein to a subject in need. In some embodiments, the subject has cancer. In some embodiments, the administration treats, inhibits, or prevents progression of the cancer.

### BRIEF DESCRIPTION OF THE DRAWINGS

FIG. 1A includes depictions of non-limiting examples of CD19-directed chimeric receptors.

FIG. 1B includes depictions of additional non-limiting examples of CD19-directed chimeric receptors.

FIG. 2 also includes depictions of non-limiting examples of CD19-directed chimeric receptors.

FIG. 3A also includes depictions of non-limiting examples of CD19-directed chimeric receptors.

FIG. 3B also includes depictions of non-limiting examples of CD19-directed chimeric receptors.

FIG. 3C also includes depictions of non-limiting examples of CD19-directed chimeric receptors.

FIG. 3D also includes depictions of non-limiting examples of CD19-directed chimeric receptors comprising humanized CD19 binding domains.

FIG. 3E also includes depictions of non-limiting examples of CD19-directed chimeric receptors comprising humanized CD19 binding domains.

FIG. 3F also includes depictions of non-limiting examples of CD19-directed chimeric receptors comprising humanized CD19 binding domains.

FIG. 3G also includes depictions of non-limiting examples of CD19-directed chimeric receptors comprising humanized CD19 binding domains without a tag sequence.

FIG. 3H also includes depictions of non-limiting examples of CD19-directed chimeric receptors comprising humanized CD19 binding domains without a tag sequence.

FIG. 3I also includes depictions of non-limiting examples of CD19-directed chimeric receptors comprising humanized CD19 binding domains without a tag sequence.

FIG. 4 depicts a schematic of a non-limiting NK cell expansion protocol.

FIG. 5 shows a schematic of an experimental protocol assessing the effectiveness of a CD19-directed chimeric antigen receptor in accordance with several embodiments disclosed herein.

FIG. 6A depicts in vivo data related to the anti-tumor effect of various non-limiting CD19-directed chimeric receptors.

FIG. 6B depicts summary data related to the anti-tumor effect of various non-limiting CD19-directed chimeric receptors.

FIG. 7 depicts data related to the expression of various CD19-directed chimeric receptor constructs by NK cells.

FIG. 8A depicts raw fluorescence data (mean fluorescence intensity, MFI) related to the expression of selected CD19-directed chimeric receptors.

FIG. 8B depicts data related to the expression of selected CD19-directed chimeric receptors, displayed as percent of NK cells expressing the indicated receptor.

FIGS. 9A-9D depicts data related to the cytotoxicity (against Nalm6 or Raji cells) for the indicated CD19-directed chimeric receptors.

FIG. 9E depicts summary data related to the cytotoxicity (against Nalm6 cells) for the indicated CD19-directed chimeric receptors at various effector:target ratios.

FIG. 9F depicts summary data related to the cytotoxicity (against Raji cells) for the indicated CD19-directed chimeric receptors at various effector:target ratios.

FIG. 10A depicts data related to the degree of enhanced cytotoxicity (against Nalm6 cells) for the indicated CD19-directed chimeric receptors at 7 days post transduction.

FIG. 10B depicts data related to the degree of enhanced cytotoxicity (against Raji cells) for the indicated CD19-directed chimeric receptors at 7 days post transduction.

FIG. 10C depicts data related to the degree of enhanced cytotoxicity (against Nalm6 cells) for the indicated CD19-directed chimeric receptors at 14 days post transduction.

FIG. 10D depicts data related to the degree of enhanced cytotoxicity (against Raji cells) for the indicated CD19-directed chimeric receptors at 14 days post transduction.

FIGS. 11A-11E depict data related to cytokine release by NK cells expressing various CD19-directed chimeric receptors when co-cultured with Nalm6 cells. FIG. 11A depicts Granzyme B release. FIG. 11B depicts perforin release. FIG. 11C depicts TNF-alpha release.

FIG. 11D depicts GM-CSF release. FIG. 11E depicts interferon gamma release.

FIGS. 12A-12E depict data related to cytokine release by NK cells expressing various CD19-directed chimeric receptors when co-cultured with Raji cells. FIG. 12A depicts Granzyme B release. FIG. 12B depicts perforin release. FIG. 12C depicts TNF-alpha release.

FIG. 12D depicts GM-CSF release. FIG. 12E depicts interferon gamma release.

FIG. 13 depicts in vivo imaging data related to tumor burden over time in mice treated with PBS, non-transduced NK cells, or NK cells expressing the indicated CD19-directed chimeric receptors, with the indicated number of engineered NK cells administered (M=million cells).

FIG. 14A shows raw data related to the detected fluorescent signal from the in vivo data shown in FIG. 13, with data being tracked for 25 days post-administration of Nalm6 cells.

FIG. 14B shows the data from FIG. 14A on a logarithmic scale.

FIG. 14C shows data related to CD3 expression by the indicated cells expressing the indicated constructs.

FIG. 14D shows data related to CD56 expression by the indicated cells expressing the indicated constructs.

FIG. 14E shows data related to GFP-expressing tumor cells when contacted with the indicated cells expressing the indicated constructs.

FIG. 14F shows the data related to CD19-expressing tumor cells when contacts with the indicated cells expressing the indicated constructs.

FIG. 15 depicts data related to selected functional characteristics of selected humanized anti-CD19 CAR constructs.

FIG. 16 depicts data related to the expression of various humanized anti-CD19 CAR constructs disclosed in embodiments herein.

FIGS. 17A-17E show cytotoxicity data. 17A shows data related to the cytotoxicity of NK cells from a first donor engineered to express selected humanized CD19 CAR constructs disclosed herein against Nalm6 cells. 17A shows data related to the cytotoxicity of NK cells from a first donor engineered to express selected humanized CD19 CAR constructs disclosed herein against Raji cells. 17C shows data related to the cytotoxicity of NK cells from a second donor engineered to express selected humanized CD19 CAR constructs disclosed herein against Nalm6 cells. 17D shows data related to the cytotoxicity of NK cells from a third donor engineered to express selected humanized CD19 CAR constructs disclosed herein against Raji cells. 17E shows data related to the cytotoxicity of NK cells from a third donor engineered to express selected humanized CD19 CAR constructs disclosed herein against Nalm6 cells.

FIG. 18 shows summary (from 3 donors) expression data for NK cells expressing the indicated non-limiting anti-CD19 CAR constructs at 3 days post-transduction.

FIGS. 19A-19D show cytotoxicity rechallenge data. FIG. 19A shows data related to the cytotoxicity of NK cells from a first donor engineered to express selected humanized CD19 CAR constructs disclosed herein against Raji cells with addition of the Raji cells to the NK cell culture at day 7 and again at day 14. FIG. 19B shows data related to the cytotoxicity of NK cells from a first donor engineered to express selected humanized CD19 CAR constructs disclosed herein against Nalm6 cells with addition of the Nalm6 cells to the NK cell culture at day 7 and again at day 14. FIG. 19C shows data related to the cytotoxicity of NK cells from a second donor engineered to express selected humanized CD19 CAR constructs disclosed herein against Raji cells with addition of the Raji cells to the NK cell culture at day 7 and again at day 14. FIG. 19D shows data related to the cytotoxicity of NK cells from a second donor engineered to express selected humanized CD19 CAR constructs disclosed herein against Nalm6 cells with addition of the Nalm6 cells to the NK cell culture at day 7 and again at day 14.

FIGS. 20A-20B show expression data. FIG. 20A shows fluorescence data from expression of non-limiting examples of humanized anti-CD19 CAR constructs from two donors 10 days post-transduction. FIG. 20B shows data related to the percentage of cells expressing CD19 (indicative of the efficiency of expression of the given anti-CD19 CAR.

FIGS. 21A-21B show cytotoxicity data. FIG. 21A shows data related to the cytotoxicity of NK cells (from the first of the two donors of FIG. 20) engineered to express selected humanized CD19 CAR constructs disclosed herein against Raji cells with addition of the Raji cells to the NK cell culture at day 7 and again at day 14. FIG. 21B shows data related to the cytotoxicity of NK cells (from the second of the two donors of FIG. 20) engineered to express selected humanized CD19 CAR constructs disclosed herein against Raji cells with addition of the Raji cells to the NK cell culture at day 7 and again at day 14.

FIGS. 22A-22E show data related to cytokine release by NK cells expressing various CD19-directed chimeric receptors when co-cultured with Raji cells. FIG. 22A shows interferon gamma release. FIG. 22B shows GM-CSF release. FIG. 22C shows tumor necrosis factor release. FIG. 22D shows perforin release. FIG. 22E shows granzyme release.

FIGS. 23A-23D show data related to NK cell survival. FIG. 23A shows the analysis of survival of NK cells from a first donor engineered to express the indicated non-limiting embodiments of anti-CD19 CAR constructs at days 7, 13, 19, and 26 post-transduction. FIG. 23B shows the analysis of survival of NK cells from a second donor engineered to express the indicated non-limiting embodiments of anti-CD19 CAR constructs at days 7, 13, 19, and 26 post-transduction. FIG. 23C shows the analysis of survival of NK cells from a third donor engineered to express the indicated non-limiting embodiments of anti-CD19 CAR constructs at days 11, 19, and 26 post-transduction. FIG. 23D shows the analysis of survival of NK cells from a fourth donor engineered to express the indicated non-limiting embodiments of anti-CD19 CAR constructs at days 11, 19, and 26 post-transduction. For each experiment, media was changed twice per week.

FIG. 24 shows data related to the percent of NK cells expressing the indicated non-limiting embodiments of anti-CD19 CAR constructs at 11 days post-transduction.

FIGS. 25A-25I show data related to the expression of CD19 by NK cells transduced with various non-limiting embodiments of anti-CD19 CAR constructs disclosed herein. FIG. 25A shows GFP transduced NK cells as a control. FIG. 25B shows CD19 expression in NK cells transduced with NK19-1. FIG. 25C shows CD19 expression in NK cells transduced with NK19H-1. FIG. 25D shows CD19 expression in NK cells transduced with NK19H-2.

FIG. 25E shows CD19 expression in NK cells transduced with NK19H-3. FIG. 25F shows CD19 expression in NK cells transduced with NK19H-4. FIG. 25G shows CD19 expression in NK cells transduced with NK19H-5. FIG. 25H shows CD19 expression in NK cells transduced with NK19H-11. FIG. 25I shows CD19 expression in NK cells transduced with NK19H-12.

FIGS. 26A-26D show cytotoxicity data. FIG. 26A shows data related to the cytotoxicity of NK cells (from a first donor) engineered to express selected humanized CD19 CAR constructs disclosed herein against Raji cells with addition of the Raji cells to the NK cell culture at day 7 and again at day 14. FIG. 26B shows data related to the cytotoxicity of NK cells (from the first donor) engineered to express selected humanized CD19 CAR constructs disclosed herein against Nalm6 cells with addition of the Nalm6 cells to the NK cell culture at day 7 and again at day 14. FIG. 26C shows data related to the cytotoxicity of NK cells (from a second donor) engineered to express selected humanized CD19 CAR constructs disclosed herein against Raji cells with addition of the Raji cells to the NK cell culture at day 7 and again at day 14. FIG. 26D shows data related to the cytotoxicity of NK cells (from the second donor) engineered to express selected humanized CD19 CAR constructs disclosed herein against Nalm6 cells with addition of the Nalm6 cells to the NK cell culture at day 7 and again at day 14.

FIGS. 27A-27B show data related to CD19 expression by engineered NK cells. FIG. 27A shows flow cytometry data for NK cells from a donor that were engineered to express various non-limiting anti-CD19 CAR constructs disclosed herein. FIG. 27B shows corresponding data from NK cells isolated from an additional donor.

FIGS. 28A-28B show cytotoxicity data. FIG. 28A shows the Raji cell count over time when exposed (at 1:1 E:T ratio) to NK cells (from the first donor of FIG. 27) expressing the non-limiting examples of anti-CD19 CAR constructs indicated. FIG. 28B shows the Raji cell count over time when exposed (at 1:1 E:T ratio) to NK cells (from the second donor of FIG. 27) expressing the non-limiting examples of anti-CD19 CAR constructs indicated.

FIGS. 29A-29E show data related to various humanized CD19-directed CAR constructs and their efficacy in an in vivo model. FIG. 29A shows a schematic depiction of an experimental protocol for assessing the effectiveness of CD19-directed CAR constructs in vivo.

FIG. 29B shows in vivo bioluminescent imaging of mice having been administered NALM6 tumor cells and treated with the indicated construct. FIG. 29C shows a line graph of the bioluminescent data collected from FIG. 29B. FIG. 29D is a survival curve showing the days that animals in each treatment group survived. FIG. 29E shows the relative expression level of the indicated constructs by NK cells, as measured by the MFI of a tag included in the CD19 CAR construct.

FIGS. 30A-30F relate to expression of the indicated CD19-directed CAR constructs. FIG. 30A shows data related to the expression of the indicated constructs as a percentage of all CD56 positive cells in a blood sample from mice 15 days post-NK cell administration (e.g., as per the schematic in FIG. 29A). FIG. 30B shows data related to expression of the indicated constructs as a percentage of cells that are both CD56 positive and express a Flag tag (as part of the CD19 CAR construct), also at 15 days post-NK cell administration. FIG. 30C shows data related to detection of GFP+tumor cells in samples from animals treated with the indicated constructs, also at day 15 post-NK cell administration. FIGS. 30D, 30E, and 30F show corresponding data at 32 days post-NK cell administration.

FIGS. 31A-31B show data related to the expression of non-tagged CD19 CAR constructs. As discussed herein, in several embodiments the CD19 CAR constructs comprise a Flag, or other tag, for detection purposes. However, all constructs disclosed herein with a tag are also included herein without a tag. FIG. 31A shows data related to the expressing of selected non-flag tagged humanized anti CD19 CAR constructs over time. FIG. 31B shows corresponding data in terms of the detected mean fluorescent intensity for the indicated constructs.

FIGS. 32A-32D show data related to an in vitro re-challenge experiment in which a first population of tumor cells were co-cultured with NK cells expressing the indicated constructs and the NK cells were “rechallenged” with an additional bolus of tumor cells. FIG. 32A shows data at 10 days after inception of co-culture with Raji cells. FIG. 32B shows data related to Raji at the final time point, 14 days. FIG. 32C shows data related to Nalm6 cells at 10 days. FIG. 32D shows data related to Nalm6 cells at 14 days.

FIGS. 33A-33J relate to evaluation of cytokine production by NK cells expressing the indicated constructs. FIG. 33A shows expression of interferon gamma by NK cells expressing the indicated constructs after co-culturing with Raji cells. FIG. 33B shows expression of GM-CSF by NK cells expressing the indicated constructs after co-culturing with Raji cells. FIG. 33C shows expression of tumor necrosis factor alpha by NK cells expressing the indicated constructs after co-culturing with Raji cells. FIG. 33D shows expression of perforin by NK cells expressing the indicated constructs after co-culturing with Raji cells. FIG. 33E shows expression of granzyme B by NK cells expressing the indicated constructs after co-culturing with Raji cells. FIG. 33F shows expression of interferon gamma by NK cells expressing the indicated constructs after co-culturing with Nalm6 cells. FIG. 33G shows expression of GM-CSF by NK cells expressing the indicated constructs after co-culturing with Nalm6cells. FIG. 33H shows expression of tumor necrosis factor alpha by NK cells expressing the indicated constructs after co-culturing with Nalm6cells. FIG. 33I shows expression of perforin by NK cells expressing the indicated constructs after co-culturing with Nalm6 cells. FIG. 33J shows expression of granzyme B by NK cells expressing the indicated constructs after co-culturing with Nalm6 cells.

FIG. 34 shows data related to the viability of NK cells expressing humanized non-tagged CD19 CAR constructs over four weeks post-transduction.

FIGS. 35A-35D show data related to various humanized, non-tagged, CD19-directed CAR constructs and their efficacy in an in vivo model. FIG. 35A shows a schematic depiction of an experimental protocol for assessing the effectiveness of humanized, non-tagged, CD19-directed CAR constructs in vivo. FIG. 35B shows in vivo bioluminescent imaging of mice having been administered NALM6 tumor cells and treated with the indicated construct.

FIG. 35C shows a line graph of the bioluminescent data collected from FIG. 35B. FIG. 35D shows the relative expression level of the indicated constructs by NK cells, as measured by detection of a CD19-Fc fusion protein that binds to the CD19 CAR construct.

FIGS. 36A-36F relate to expression of the indicated CD19-directed CAR constructs. FIG. 36A shows data related to the expression of the indicated constructs as a percentage of all CD56 positive cells in a blood sample from mice 13 days post-NK cell administration (e.g., as per the schematic in FIG. 35A). FIG. 36B shows data related to detection of CD19+ tumor cells in samples from animals treated with the indicated constructs, also at 13 days post-NK cell administration. FIG. 36C shows data related to detection of GFP+tumor cells in samples from animals treated with the indicated constructs, also at 13 days post-NK cell administration. FIGS. 36D, 36E, and 36F show corresponding data at 27 days post-NK cell administration.

FIGS. 37A-37C relate to the in vivo efficacy of various CD19-directed CAR according to embodiments disclosed herein. FIG. 37A shows a schematic depiction of an experimental protocol for assessing the effectiveness of humanized, NK cells expressing various CD19-directed CAR constructs in vivo. The various experimental groups tested are as indicated. For cells with an “IL12/IL18” designation, the cells were expanded in the presence of soluble IL12 and/or IL18, as described in in U.S. Provisional Patent Application No. 62/881,311, filed Jul. 31, 2019 and Application No. 62/932,342, filed Nov. 7, 2019, each of which is incorporated in its entirety by reference herein. FIGS. 37B and 37C show bioluminescence data from animals dosed with Nalm6 tumor cells and treated with the indicated construct.

FIGS. 38A-38J show graphical depictions of the bioluminescence data from FIG. 37B. FIG. 38A shows bioluminescence (as photon/second flux) from animals receiving untransduced NK cells. FIG. 38B shows flux measured in animals receiving PBS as a vehicle. FIG. 38C shows flux measured in animals receiving previously frozen NK cells expressing the NK19 NF2 CAR (as a non-limiting example of a CAR). FIG. 38D shows flux measured in animals receiving previously frozen NK cells expressing the NK19 NF2 CAR (as a non-limiting example of a CAR) expanded using IL12 and/or IL18. FIG. 38E and FIG. 38F show flux measured in animals receiving fresh NK cells expressing the NK19 NF2 CAR (as a non-limiting example of a CAR). FIG. 38G and FIG. 38H show flux measured in animals receiving previously fresh NK cells expressing the NK19 NF2 CAR (as a non-limiting example of a CAR) expanded using IL12 and/or IL18. FIG. 38I shows a line graph depicting the bioluminescence measured in the various groups over the first 30 days post-tumor inoculation. FIG. 38J shows a line graph depicting the bioluminescence measured in the various groups over the first 56 days post-tumor inoculation.

FIG. 39 shows data related to the body mass of mice over time when receiving the indicated therapy.

### DETAILED DESCRIPTION

Some embodiments of the methods and compositions provided herein relate to CD19-directed chimeric receptors. In some embodiments, the receptors are expressed on a cell as described herein. Some embodiments include methods of use of the compositions or cells in immunotherapy.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anticancer effect” can also be manifested by the ability of the SIRs in prevention of the occurrence of cancer in the first place.

### Cell Types

Some embodiments of the methods and compositions provided herein relate to a cell such as an immune cell. For example, an immune cell may be engineered to include a chimeric receptor such as a CD19-directed chimeric receptor, or engineered to include a nucleic acid encoding said chimeric receptor as described herein.

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells or Natural Killer (NK cells), as described in more detail below.

To facilitate cancer immunotherapies, there are provided for herein polynucleotides, polypeptides, and vectors that encode chimeric antigen receptors (CAR) that comprise a target binding moiety (e.g., an extracellular binder of a ligand, or a CD19-directed chimeric receptor, expressed by a cancer cell) and a cytotoxic signaling complex. For example, some embodiments include a polynucleotide, polypeptide, or vector that encodes a CD19-directed chimeric receptor to facilitate targeting of an immune cell to a cancer and exerting cytotoxic effects on the cancer cell. Also provided are engineered immune cells (e.g., T cells or NK cells) expressing such CARs. There are also provided herein, in several embodiments, polynucleotides, polypeptides, and vectors that encode a construct comprising an extracellular domain comprising two or more subdomains, e.g., first CD19-targeting subdomain comprising a CD19 binding moiety as disclosed herein and a second subdomain comprising a C-type lectin-like receptor and a cytotoxic signaling complex. Also provided are engineered immune cells (e.g., T cells or NK cells) expressing such bi-specific constructs. Engineered immune cells (e.g., T cells or NK cells) expressing multi-specific constructs and/or having the ability to bind a plurality of target markers are also provided. Methods of treating cancer and other uses of such cells for cancer immunotherapy are also provided for herein.

### Engineered Cells for Immunotherapy

In several embodiments, cells of the immune system are engineered to have enhanced cytotoxic effects against target cells, such as tumor cells. For example, a cell of the immune system may be engineered to include a CD19-directed chimeric receptor as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric receptor such as a CD19-directed chimeric receptor, or a nucleic acid encoding the chimeric receptor and/or engineered to co-express a membrane-bound interleukin 15 (mbIL15) co-stimulatory domain.

### Monocytes for Immunotherapy

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake cellular material, or entire cells, followed by digestion and destruction of the engulfed cellular material. In several embodiments, monocytes are used in connection with one or more additional engineered cells as disclosed herein. Some embodiments of the methods and compositions described herein relate to a monocyte that includes a CD19-directed chimeric receptor, or a nucleic acid encoding the CD19-directed chimeric receptor. Several embodiments of the methods and compositions disclosed herein relate to monocytes engineered to express a CD19-directed chimeric receptor and a membrane-bound interleukin 15 (mbIL15) co-stimulatory domain.

### Lymphocytes for Immunotherapy

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments). Some embodiments of the methods and compositions described herein relate to a lymphocyte that includes a CD19-directed chimeric receptor, or a nucleic acid encoding the CD19-directed chimeric receptor. Several embodiments of the methods and compositions disclosed herein relate to lymphocytes engineered to express a CD19-directed chimeric receptor and a membrane-bound interleukin 15 (mbIL15) co-stimulatory domain.

### T Cells for Immunotherapy

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Some embodiments of the methods and compositions described herein relate to a T cell that includes a CD19-directed chimeric receptor, or a nucleic acid encoding the CD19-directed chimeric receptor. Several embodiments of the methods and compositions disclosed herein relate to T-cells engineered to express a CD19-directed chimeric receptor and a membrane-bound interleukin 15 (mbIL15) co-stimulatory domain.

### NK Cells for Immunotherapy

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). Some embodiments of the methods and compositions described herein relate to an NK that includes a CD19-directed chimeric receptor, or a nucleic acid encoding the CD19-directed chimeric receptor. Several embodiments of the methods and compositions disclosed herein relate to NK cells engineered to express a CD19-directed chimeric receptor and a membrane-bound interleukin 15 (mbIL15) co-stimulatory domain.

### Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell, that includes a CD19-directed chimeric receptor, or a nucleic acid encoding the CD19-directed chimeric receptor. Several embodiments of the methods and compositions disclosed herein relate to stem cells, such as hematopoietic stem cells that are engineered to express a CD19-directed chimeric receptor and a membrane-bound interleukin 15 (mbIL15) co-stimulatory domain.

### Extracellular Domains (Tumor Binder)

Some embodiments of the compositions and methods described herein relate to a chimeric receptor, such as a CD19-directed chimeric receptor, that includes an extracellular domain. In some embodiments, the extracellular domain comprises a tumor-binding domain (also referred to as an antigen-binding protein or antigen-binding domain) as described herein. in some embodiments, the antigen-binding domain is derived from or comprises wild-type or non-wild-type sequence of an antibody, an antibody fragment, an scFv, a Fv, a Fab, a (Fab′)2, a single domain antibody (SDAB), a vH or vL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the tumor-binding domain contains more than one antigen binding domain. In embodiments, the antigen-binding domain is operably linked directly or via an optional linker to the NH2-terminal end of a TCR domain (e.g. constant chains of TCR-alpha, TCR-beta1, TCR-beta2, preTCR-alpha, pre-TCR-alpha-Del48, TCR-gamma, or TCR-delta)

### Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen (e.g., CD19) or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Nonlimiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g. enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (k) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (μ), delta (A), gamma (γ), alpha (a), and epsilon (F), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3)(see U.S. Pat. No. 6,703,199, which describes fibronectin polypeptide mini bodies). An antibody fragment may include a Fab, Fab′, F(ab′)2, and/or Fv fragment that contains at least one CDR of an immunoglobulin that is sufficient to confer specific antigen binding to a cancer antigen (e.g., CD19). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it an antigen-binding protein. In several embodiments, the antigen-binding proteins provided herein comprise a heavy chain variable region selected from SEQ ID NO: 104 and SEQ ID NO: 106. In several embodiments, the antigen-binding proteins provided herein comprise a light chain variable region selected from SEQ ID NO: 105 and SEQ ID NO: 107.

In several embodiments, the antigen-binding protein has been modified from its original sequence, for example for purposes of improving expression, function, or reducing a potential immune response to the antigen-binding protein by a host. In several embodiments, the antigen-binding protein comprises a light chain variable region selected from SEQ ID NO: 117, SEQ ID NO: 118, and SEQ ID NO: 119 and/or sequences having at least 90% identity and/or homology (e.g., 90-95%, 95%, 96%, 97%, 98%, 99%). In several embodiments, the light chain variable region differs in sequence from SEQ ID NO: 117, SEQ ID NO: 118, or SEQ ID NO: 119 by more than 5% (e.g., by 5-7%, 5-10%, 10-20% or higher) with ligand-binding function (or other functionality) being similar, substantially similar or the same as SEQ ID NO: 117, SEQ ID NO: 118, or SEQ ID NO: 119. In several embodiments, the antigen-binding protein comprises a heavy chain variable region selected from SEQ ID NO: 120, SEQ ID NO: 121, SEQ ID NO: 122, and SEQ ID NO: 123 and/or sequences having at least 90% identity and/or homology (e.g., 90-95%, 95%, 96%, 97%, 98%, 99%). In several embodiments, the heavy chain variable region differs in sequence from SEQ ID NO: 120, SEQ ID NO: 121, SEQ ID NO: 122, or SEQ ID NO: 123 by more than 5% (e.g., by 5-7%, 5-10%, 10-20% or higher) with ligand-binding function (or other functionality) being similar, substantially similar or the same as SEQ ID NO: 120, SEQ ID NO: 121, SEQ ID NO: 122, or SEQ ID NO: 123. Depending on the embodiment, any combination of heavy and light chain regions may be used (e.g., in assembling a scFv). In several embodiments, the antigen-binding protein comprises one or more CDRs selected from SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 134, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, and SEQ ID NO: 144.

In additional embodiments, the CDRs are selected from SEQ ID NO: 108, SEQ ID NO: 109, SEQ ID NO: 110, SEQ ID NO: 111, SEQ ID NO: 112, SEQ ID NO: 113, SEQ ID NO: 114, and SEQ ID NO: 115, in any combination. In one embodiment, the CDRs are assembled to generate a CAR directed to CD19 and comprising SEQ ID NO: 116.

In several embodiments, the antigen-binding protein comprises a heavy chain having the sequence of SEQ ID NO: 88. In several embodiments, that heavy chain is coupled with (e.g., as an scFv), one of the light chains of SEQ ID NO: 89, SEQ ID NO: 90, and/or SEQ ID NO: 91. In several embodiments, the antigen-binding protein comprises one of more CDRs selected from SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, and SEQ ID NO: 100.

In several embodiments, the antigen-binding protein comprises a light chain region of an FMC63 antibody that has the sequence of SEQ ID NO: 150. In several embodiments, the antigen-binding protein comprises a light chain region of an FMC63 antibody that has the sequence of SEQ ID NO: 148. In several embodiments, linkers are used between heavy and light chains, and in some embodiments, the linker comprises the sequence of SEQ ID NO: 149. In several embodiments, such heavy and light chains are used in conjunction with a CD28 co-stimulatory domain, such as that of SEQ ID NO: 153. Often a spacer is used to separate component parts of a CAR. For example, in several embodiments, a spacer is used comprising the sequence of SEQ ID NO: 151. In several embodiments, a transmembrane domain having the sequence of SEQ ID NO: 152 is used. In several embodiments, the CAR comprises a nucleic acid sequence that shares at least about 90%, at least about 94%, at least about 95%, at least about 96%, at least about 97%, at least about 98%, or at least about 99%, sequence identity, homology and/or functional equivalence with SEQ ID NO: 147.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

There is also provided, in some embodiments, antigen-binding proteins with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets. In several embodiments, this is particularly advantageous, as a bispecific chimeric antigen receptor can impart to an engineered cell the ability to target multiple tumor markers. For example, CD19 and an additional tumor marker, such as CD123, NKG2D or any other marker disclosed herein or appreciated in the art as a tumor specific antigen or tumor associated antigen.

As used herein, the term “chimeric antibody” shall be given its ordinary meaning, and shall also refer to an antibody that contains one or more regions from one antibody and one or more regions from one or more other antibodies. In some embodiments, one or more of the CDRs are derived from an anti-cancer antigen (e.g., CD19) antibody. In several embodiments, all of the CDRs are derived from an anti-cancer antigen antibody (such as an anti-CD19 antibody). In some embodiments, the CDRs from more than one anti-cancer antigen antibodies are mixed and matched in a chimeric antibody. For instance, a chimeric antibody may comprise a CDR1 from the light chain of a first anti-cancer antigen antibody, a CDR2 and a CDR3 from the light chain of a second anti-cancer antigen antibody, and the CDRs from the heavy chain from a third anti-cancer antigen antibody. Further, the framework regions of antigen-binding proteins disclosed herein may be derived from one of the same anti-cancer antigen (e.g., CD19) antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also provided herein are fragments of such antibodies that exhibit the desired biological activity.

In some embodiments, an antigen-binding protein is provided comprising a heavy chain variable domain having at least 90% identity to the VH domain amino acid sequence set forth in SEQ ID NO: 33. In some embodiments, the antigen-binding protein comprises a heavy chain variable domain having at least 95% identity to the VH domain amino acid sequence set forth in SEQ ID NO: 33. In some embodiments, the antigen-binding protein comprises a heavy chain variable domain having at least 96, 97, 98, or 99% identity to the VH domain amino acid sequence set forth in SEQ ID NO: 33. In several embodiments, the heavy chain variable domain may have one or more additional mutations (e.g., for purposes of humanization) in the VH domain amino acid sequence set forth in SEQ ID NO: 33, but retains specific binding to a cancer antigen (e.g., CD19). In several embodiments, the heavy chain variable domain may have one or more additional mutations in the VH domain amino acid sequence set forth in SEQ ID NO: 33, but has improved specific binding to a cancer antigen (e.g., CD19).

In some embodiments, the antigen-binding protein comprises a light chain variable domain having at least 90% identity to the VL domain amino acid sequence set forth in SEQ ID NO: 32. In some embodiments, the antigen-binding protein comprises a light chain variable domain having at least 95% identity to the VL domain amino acid sequence set forth in SEQ ID NO: 32. In some embodiments, the antigen-binding protein comprises a light chain variable domain having at least 96, 97, 98, or 99% identity to the VL domain amino acid sequence set forth in SEQ ID NO: 32. In several embodiments, the light chain variable domain may have one or more additional mutations (e.g., for purposes of humanization) in the VL domain amino acid sequence set forth in SEQ ID NO: 32, but retains specific binding to a cancer antigen (e.g., CD19). In several embodiments, the light chain variable domain may have one or more additional mutations in the VL domain amino acid sequence set forth in SEQ ID NO: 32, but has improved specific binding to a cancer antigen (e.g., CD19).

In some embodiments, the antigen-binding protein comprises a heavy chain variable domain having at least 90% identity to the VH domain amino acid sequence set forth in SEQ ID NO: 33, and a light chain variable domain having at least 90% identity to the VL domain amino acid sequence set forth in SEQ ID NO: 32. In some embodiments, the antigen-binding protein comprises a heavy chain variable domain having at least 95% identity to the VH domain amino acid sequence set forth in SEQ ID NO: 33, and a light chain variable domain having at least 95% identity to the VL domain amino acid sequence set forth in SEQ ID NO: 32. In some embodiments, the antigen-binding protein comprises a heavy chain variable domain having at least 96, 97, 98, or 99% identity to the VH domain amino acid sequence set forth in SEQ ID NO: 33, and a light chain variable domain having at least 96, 97, 98, or 99% identity to the VL domain amino acid sequence set forth in SEQ ID NO: 32.

In some embodiments, the antigen-binding protein comprises a heavy chain variable domain having the VH domain amino acid sequence set forth in SEQ ID NO: 33, and a light chain variable domain having the VL domain amino acid sequence set forth in SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a light chain variable domain of SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a heavy chain variable domain in accordance with SEQ ID NO: 33.

In some embodiments, the light chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99% or 100% identical to the polynucleotide sequence SEQ ID NO: 32. In some embodiments, the light chain variable domain comprises a sequence of amino acids that is encoded by a polynucleotide that hybridizes under moderately stringent conditions to the complement of a polynucleotide that encodes a light chain variable domain in accordance with the sequence in SEQ ID NO: 32. In some embodiments, the light chain variable domain comprises a sequence of amino acids that is encoded by a polynucleotide that hybridizes under stringent conditions to the complement of a polynucleotide that encodes a light chain variable domain in accordance with the sequence in SEQ ID NO: 32.

In some embodiments, the heavy chain variable domain comprises a sequence of amino acids that is at least 70%, 75%, 80%, 85%, 90%, 91%, 92%, 93%, 94%, 95%, 96%, 97%, 98%, 99% or 100% identical to the sequence of a heavy chain variable domain in accordance with the sequence of SEQ ID NO: 33. In some embodiments, the heavy chain variable domain comprises a sequence of amino acids that is encoded by a polynucleotide that hybridizes under moderately stringent conditions to the complement of a polynucleotide that encodes a heavy chain variable domain in accordance with the sequence of SEQ ID NO: 33. In some embodiments, the heavy chain variable domain comprises a sequence of amino acids that is encoded by a polynucleotide that hybridizes under stringent conditions to the complement of a polynucleotide that encodes a heavy chain variable domain in accordance with the sequence of SEQ ID NO: 33.

In several embodiments, additional anti-CD19 binding constructs are provided. For example, in several embodiments, there is provided an scFv that targets CD19 wherein the scFv comprises a heavy chain variable region comprising the sequence of SEQ ID NO. 35. In some embodiments, the antigen-binding protein comprises a heavy chain variable domain having at least 95% identity to the HCV domain amino acid sequence set forth in SEQ ID NO: 35. In some embodiments, the antigen-binding protein comprises a heavy chain variable domain having at least 96, 97, 98, or 99% identity to the HCV domain amino acid sequence set forth in SEQ ID NO: 35. In several embodiments, the heavy chain variable domain may have one or more additional mutations (e.g., for purposes of humanization) in the HCV domain amino acid sequence set forth in SEQ ID NO: 35, but retains specific binding to a cancer antigen (e.g., CD19). In several embodiments, the heavy chain variable domain may have one or more additional mutations in the HCV domain amino acid sequence set forth in SEQ ID NO: 35, but has improved specific binding to a cancer antigen (e.g., CD19).

Additionally, in several embodiments, an scFv that targets CD19 comprises a light chain variable region comprising the sequence of SEQ ID NO. 36. In some embodiments, the antigen-binding protein comprises a light chain variable domain having at least 95% identity to the LCV domain amino acid sequence set forth in SEQ ID NO: 36. In some embodiments, the antigen-binding protein comprises a light chain variable domain having at least 96, 97, 98, or 99% identity to the LCV domain amino acid sequence set forth in SEQ ID NO: 36. In several embodiments, the light chain variable domain may have one or more additional mutations (e.g., for purposes of humanization) in the LCV domain amino acid sequence set forth in SEQ ID NO: 36, but retains specific binding to a cancer antigen (e.g., CD19). In several embodiments, the light chain variable domain may have one or more additional mutations in the LCV domain amino acid sequence set forth in SEQ ID NO: 36, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 37. In several embodiments, the LC CDR1 comprises an amino acid sequence with at least about 85%, about 90%, about 95%, or about 98% homology to the sequence of SEQ NO. 37. In several embodiments, the LC CDR2 comprises the sequence of SEQ ID NO. 38. In several embodiments, the LC CDR2 comprises an amino acid sequence with at least about 85%, about 90%, about 95%, or about 98% homology to the sequence of SEQ NO. 38. In several embodiments, the LC CDR3 comprises the sequence of SEQ ID NO. 39. In several embodiments, the LC CDR3 comprises an amino acid sequence with at least about 85%, about 90%, about 95%, or about 98% homology to the sequence of SEQ NO. 39. In several embodiments, the HC CDR1 comprises the sequence of SEQ ID NO. 40. In several embodiments, the HC CDR1 comprises an amino acid sequence with at least about 85%, about 90%, about 95%, or about 98% homology to the sequence of SEQ NO. 40. In several embodiments, the HC CDR2 comprises the sequence of SEQ ID NO. 41, 42, or 43. In several embodiments, the HC CDR2 comprises an amino acid sequence with at least about 85%, about 90%, about 95%, or about 98% homology to the sequence of SEQ NO. 41, 42, or 43. In several embodiments, the HC CDR3 comprises the sequence of SEQ ID NO. 44. In several embodiments, the HC CDR3 comprises an amino acid sequence with at least about 85%, about 90%, about 95%, or about 98% homology to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48. In several embodiments, the VL region comprises an amino acid sequence with at least about 85%, about 90%, about 95%, or about 98% homology to the sequence of SEQ NO. 45, 46, 47, or 48. In several embodiments, the VH region comprises the sequence of SEQ ID NO. 49, 50, 51 or 52. In several embodiments, the VH region comprises an amino acid sequence with at least about 85%, about 90%, about 95%, or about 98% homology to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53. In several embodiments, the LC CDR1 comprises an amino acid sequence with at least about 85%, about 90%, about 95%, or about 98% homology to the sequence of SEQ NO. 53. In several embodiments, the LC CDR2 comprises the sequence of SEQ ID NO. 54. In several embodiments, the LC CDR2 comprises an amino acid sequence with at least about 85%, about 90%, about 95%, or about 98% homology to the sequence of SEQ NO. 54. In several embodiments, the LC CDR3 comprises the sequence of SEQ ID NO. 55. In several embodiments, the LC CDR3 comprises an amino acid sequence with at least about 85%, about 90%, about 95%, or about 98% homology to the sequence of SEQ NO. 55. In several embodiments, the HC CDR1 comprises the sequence of SEQ ID NO. 56. In several embodiments, the HC CDR1 comprises an amino acid sequence with at least about 85%, about 90%, about 95%, or about 98% homology to the sequence of SEQ NO. 56. In several embodiments, the HC CDR2 comprises the sequence of SEQ ID NO. 57. In several embodiments, the HC CDR2 comprises an amino acid sequence with at least about 85%, about 90%, about 95%, or about 98% homology to the sequence of SEQ NO. 57. In several embodiments, the HC CDR3 comprises the sequence of SEQ ID NO. 58. In several embodiments, the HC CDR3 comprises an amino acid sequence with at least about 85%, about 90%, about 95%, or about 98% homology to the sequence of SEQ NO. 58.

Additional anti-CD19 binding moieties are known in the art, such as those disclosed in, for example, U.S. Pat. No. 8,399,645, US Patent Publication No. 2018/0153977, US Patent Publication No. 2014/0271635, US Patent Publication No. 2018/0251514, and US Patent Publication No. 2018/0312588, the entirety of each of which is incorporated by reference herein.

Natural Killer Group Domains that Bind Tumor Ligands

In several embodiments, engineered immune cells such as NK cells are leveraged for their ability to recognize and destroy tumor cells. For example, an engineered NK cell may include a CD19-directed chimeric receptor or a nucleic acid encoding said chimeric receptor. NK cells express both inhibitory and activating receptors on the cell surface. Inhibitory receptors bind self-molecules expressed on the surface of healthy cells (thus preventing immune responses against “self” cells), while the activating receptors bind ligands expressed on abnormal cells, such as tumor cells. When the balance between inhibitory and activating receptor activation is in favor of activating receptors, NK cell activation occurs and target (e.g., tumor) cells are lysed.

Natural killer Group 2 member D (NKG2D) is an NK cell activating receptor that recognizes a variety of ligands expressed on cells. The surface expression of various NKG2D ligands is generally low in healthy cells but is upregulated upon, for example, malignant transformation. Non-limiting examples of ligands recognized by NKG2D include, but are not limited to, MICA, MICB, ULBP1, ULBP2, ULBP3, ULBP4, ULBP5, and ULBP6, as well as other molecules expressed on target cells that control the cytolytic or cytotoxic function of NK cells. In several embodiments, T cells are engineered to express an extracellular domain to binds to one or more tumor ligands and activate the T cell. For example, in several embodiments, T cells are engineered to express an NKG2D receptor as the binder/activation moiety. In several embodiments, engineered cells as disclosed herein are engineered to express another member of the NKG2 family, e.g., NKG2A, NKG2C, and/or NKG2E. Combinations of such receptors are engineered in some embodiments. Moreover, in several embodiments, other receptors are expressed, such as the Killer-cell immunoglobulin-like receptors (KIRs).

In several embodiments, cells are engineered to express a cytotoxic receptor complex comprising a full length NKG2D as an extracellular component to recognize ligands on the surface of tumor cells (e.g., liver cells). In one embodiment, full length NKG2D has the nucleic acid sequence of SEQ ID NO: 27. In several embodiments, the full length NKG2D, or functional fragment thereof is human NKG2D.

In several embodiments, cells are engineered to express a cytotoxic receptor complex comprising a functional fragment of NKG2D as an extracellular component to recognize ligands on the surface of tumor cells or other diseased cells. In one embodiment, the functional fragment of NKG2D has the nucleic acid sequence of SEQ ID NO: 25. In several embodiments, the fragment of NKG2D is at least 70%, at least 75%, at least 80%, at least 85%, at least 90%, or at least 95% homologous with full-length wild-type NKG2D. In several embodiments, the fragment may have one or more additional mutations from SEQ ID NO: 25, but retains, or in some embodiments, has enhanced, ligand-binding function. In several embodiments, the functional fragment of NKG2D comprises the amino acid sequence of SEQ ID NO: 26. In several embodiments, the NKG2D fragment is provided as a dimer, trimer, or other concatameric format, such embodiments providing enhanced ligand-binding activity. In several embodiments, the sequence encoding the NKG2D fragment is optionally fully or partially codon optimized. In one embodiment, a sequence encoding a codon optimized NKG2D fragment comprises the sequence of SEQ ID NO: 28. Advantageously, according to several embodiments, the functional fragment lacks its native transmembrane or intracellular domains but retains its ability to bind ligands of NKG2D as well as transduce activation signals upon ligand binding. A further advantage of such fragments is that expression of DAP10 to localize NKG2D to the cell membrane is not required. Thus, in several embodiments, the cytotoxic receptor complex encoded by the polypeptides disclosed herein does not comprise DAP10. In several embodiments, immune cells, such as NK or T cells, are engineered to express one or more chimeric receptors that target CD19 and an NGG2D ligand. Such cells, in several embodiments, also co-express mbIL15.

In several embodiments, the cytotoxic receptor complexes are configured to dimerize. Dimerization may comprise homodimers or heterodimers, depending on the embodiment. In several embodiments, dimerization results in improved ligand recognition by the cytotoxic receptor complexes (and hence the NK cells expressing the receptor), resulting in a reduction in (or lack) of adverse toxic effects. In several embodiments, the cytotoxic receptor complexes employ internal dimers, or repeats of one or more component subunits. For example, in several embodiments, the cytotoxic receptor complexes may optionally comprise a first NKG2D extracellular domain coupled to a second NKG2D extracellular domain, and a transmembrane/signaling region (or a separate transmembrane region along with a separate signaling region).

In several embodiments, the various domains/subdomains are separated by a linker such as, a GS3 linker (SEQ ID NO: 15 and 16, nucleotide and protein, respectively) is used (or a GSn linker). Other linkers used according to various embodiments disclosed herein include, but are not limited to those encoded by SEQ ID NO: 17, 19, 21 or 23. This provides the potential to separate the various component parts of the receptor complex along the polynucleotide, which can enhance expression, stability, and/or functionality of the receptor complex.

### Cytotoxic Signaling Complex

Some embodiments of the compositions and methods described herein relate to a chimeric receptor, such as a CD19-directed chimeric receptor, that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells. Certain embodiments disclosed herein relate to chimeric antigen receptor constructs wherein the tumor-targeting domain (or CD19-directed domain) is coupled to a cytotoxic signaling complex.

In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. Moreover, in some embodiments, a domain may serve multiple functions, for example, a transmembrane domain may also serve to provide signaling function.

### Transmembrane Domains

Some embodiments of the compositions and methods described herein relate to a chimeric receptor, such as a CD19-directed chimeric receptor, that includes a transmembrane domain. Some embodiments include a transmembrane domain from NKG2D or another transmembrane protein. In several embodiments in which a transmembrane domain is employed, the portion of the transmembrane protein employed retains at least a portion of its normal transmembrane domain.

In several embodiments, however, the transmembrane domain comprises at least a portion of CD8, a transmembrane glycoprotein normally expressed on both T cells and NK cells. In several embodiments, the transmembrane domain comprises CD8a. In several embodiments, the transmembrane domain is referred to as a “hinge”. In several embodiments, the “hinge” of CD8a has the nucleic acid sequence of SEQ ID NO: 1. In several embodiments, the CD8a hinge is truncated or modified and is at least 70%, at least 75%, at least 80%, at least 85%, at least 90%, at least 95% homologous with the CD8a having the sequence of SEQ ID NO: 1. In several embodiments, the “hinge” of CD8a comprises the amino acid sequence of SEQ ID NO: 2. In several embodiments, the CD8a can be truncated or modified, such that it is at least 70%, at least 75%, at least 80%, at least 85%, at least 90%, at least 95% homologous with the sequence of SEQ ID NO: 2.

In several embodiments, the transmembrane domain comprises a CD8a transmembrane region. In several embodiments, the CD8a transmembrane domain has the nucleic acid sequence of SEQ ID NO: 3. In several embodiments, the CD8a hinge is truncated or modified and is at least 70%, at least 75%, at least 80%, at least 85%, at least 90%, at least 95% homologous with the CD8a having the sequence of SEQ ID NO: 3. In several embodiments, the CD8a transmembrane domain comprises the amino acid sequence of SEQ ID NO: 4. In several embodiments, the CD8a hinge is truncated or modified and is at least 70%, at least 75%, at least 80%, at least 85%, at least 90%, at least 95% homologous with the CD8a having the sequence of SEQ ID NO: 4.

Taken together in several embodiments, the CD8 hinge/transmembrane complex is encoded by the nucleic acid sequence of SEQ ID NO: 13. In several embodiments, the CD8 hinge/transmembrane complex is truncated or modified and is at least 70%, at least 75%, at least 80%, at least 85%, at least 90%, at least 95% homologous with the CD8 hinge/transmembrane complex having the sequence of SEQ ID NO: 13. In several embodiments, the CD8 hinge/transmembrane complex comprises the amino acid sequence of SEQ ID NO: 14. In several embodiments, the CD8 hinge/transmembrane complex hinge is truncated or modified and is at least 70%, at least 75%, at least 80%, at least 85%, at least 90%, at least 95% homologous with the CD8 hinge/transmembrane complex having the sequence of SEQ ID NO: 14.

In some embodiments, the transmembrane domain comprises a CD28 transmembrane domain or a fragment thereof. In several embodiments, the CD28 transmembrane domain comprises the amino acid sequence of SEQ ID NO: 30. In several embodiments, the CD28 transmembrane domain complex hinge is truncated or modified and is at least 70%, at least 75%, at least 80%, at least 85%, at least 90%, at least 95% homologous with the CD28 transmembrane domain having the sequence of SEQ ID NO: 30.

### Co-Stimulatory Domains

Some embodiments of the compositions and methods described herein relate to a chimeric receptor, such as a CD19-directed chimeric receptor, that includes a co-stimulatory domain. In addition the various the transmembrane domains and signaling domain (and the combination transmembrane/signaling domains), additional co-activating molecules can be provided, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such co-stimulatory domains are engineered to be membrane bound, acting as autocrine stimulatory molecules (or even as paracrine stimulators to neighboring cells delivered). In several embodiments, NK cells are engineered to express membrane-bound interleukin 15 (mbIL15). In such embodiments, mbIL15 expression on the NK enhances the cytotoxic effects of the engineered NK cell by enhancing the proliferation and/or longevity of the NK cells. In several embodiments, mbIL15 has the nucleic acid sequence of SEQ ID NO: 11. In several embodiments, mbIL15 can be truncated or modified, such that it is at least 70%, at least 75%, at least 80%, at least 85%, at least 90%, at least 95% homologous with the sequence of SEQ ID NO: 11. In several embodiments, the mbIL15 comprises the amino acid sequence of SEQ ID NO: 12. In several embodiments, the mbIL15 is truncated or modified and is at least 70%, at least 75%, at least 80%, at least 85%, at least 90%, at least 95% homologous with the mbIL15 having the sequence of SEQ ID NO: 12.

In some embodiments, the CD19-directed chimeric receptor or engineered cytotoxic receptor complex is encoded by a polynucleotide that includes one or more cytosolic protease cleavage sites, for example a T2A cleavage site, a P2A cleavage site, an E2A cleavage site, and/or a F2A cleavage site. Such sites are recognized and cleaved by a cytosolic protease, which can result in separation (and separate expression) of the various component parts of the receptor encoded by the polynucleotide. As a result, depending on the embodiment, the various constituent parts of a CD19-directed chimeric receptor or engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site has the nucleic acid sequence of SEQ ID NO: 9. In several embodiments, T2A cleavage site can be truncated or modified, such that it is at least 70%, at least 75%, at least 80%, at least 85%, at least 90%, at least 95% homologous with the sequence of SEQ ID NO: 9. In several embodiments, the T2A cleavage site comprises the amino acid sequence of SEQ ID NO: 10. In several embodiments, the T2A cleavage site is truncated or modified and is at least 70%, at least 75%, at least 80%, at least 85%, at least 90%, at least 95% homologous with the T2A cleavage site having the sequence of SEQ ID NO: 10.

### Signaling Domains

Some embodiments of the compositions and methods described herein relate to a chimeric receptor, such as a CD19-directed chimeric receptor, that includes a signaling domain. For example, immune cells engineered according to several embodiments disclosed herein may comprise at least one subunit of the CD3 T cell receptor complex (or a fragment thereof). In several embodiments, the signaling domain comprises the CD3 zeta subunit. In several embodiments, the CD3 zeta is encoded by the nucleic acid sequence of SEQ ID NO: 7. In several embodiments, the CD3 zeta can be truncated or modified, such that it is at least 70%, at least 75%, at least 80%, at least 85%, at least 90%, at least 95% homologous with the CD3 zeta having the sequence of SEQ ID NO: 7. In several embodiments, the CD3 zeta domain comprises the amino acid sequence of SEQ ID NO: 8. In several embodiments, the CD3 zeta domain is truncated or modified and is at least 70%, at least 75%, at least 80%, at least 85%, at least 90%, at least 95% homologous with the CD3 zeta domain having the sequence of SEQ ID NO: 8.

In several embodiments, unexpectedly enhanced signaling is achieved through the use of multiple signaling domains whose activities act synergistically. For example, in several embodiments, the signaling domain further comprises an OX40 domain. In several embodiments, the OX40 domain is an intracellular signaling domain. In several embodiments, the OX40 intracellular signaling domain has the nucleic acid sequence of SEQ ID NO: 5. In several embodiments, the OX40 intracellular signaling domain can be truncated or modified, such that it is at least 70%, at least 75%, at least 80%, at least 85%, at least 90%, at least 95% homologous with the OX40 having the sequence of SEQ ID NO: 5. In several embodiments, the OX40 intracellular signaling domain comprises the amino acid sequence of SEQ ID NO: 16. In several embodiments, the OX40 intracellular signaling domain is truncated or modified and is at least 70%, at least 75%, at least 80%, at least 85%, at least 90%, at least 95% homologous with the OX40 intracellular signaling domain having the sequence of SEQ ID NO: 6. In several embodiments, OX40 is used as the sole transmembrane/signaling domain in the construct, however, in several embodiments, OX40 can be used with one or more other domains. For example, combinations of OX40 and CD3zeta are used in some embodiments. By way of further example, combinations of CD28, OX40, 4-1BB, and/or CD3zeta are used in some embodiments.

In several embodiments, the signaling domain comprises a 4-1BB domain. In several embodiments, the 4-1BB domain is an intracellular signaling domain. In several embodiments, the 4-1BB intracellular signaling domain comprises the amino acid sequence of SEQ ID NO: 29. In several embodiments, the 4-1BB intracellular signaling domain is truncated or modified and is at least 70%, at least 75%, at least 80%, at least 85%, at least 90%, at least 95% homologous with the 4-1BB intracellular signaling domain having the sequence of SEQ ID NO: 29. In several embodiments, 4-1BB is used as the sole transmembrane/signaling domain in the construct, however, in several embodiments, 4-1BB can be used with one or more other domains. For example, combinations of 4-1BB and CD3zeta are used in some embodiments. By way of further example, combinations of CD28, OX40, 4-1BB, and/or CD3zeta are used in some embodiments.

In several embodiments, the signaling domain comprises a CD28 domain. In several embodiments the CD28 domain is an intracellular signaling domain. In several embodiments, the CD28 intracellular signaling domain comprises the amino acid sequence of SEQ ID NO: 31. In several embodiments, the CD28 intracellular signaling domain is truncated or modified and is at least 70%, at least 75%, at least 80%, at least 85%, at least 90%, at least 95% homologous with the CD28 intracellular signaling domain having the sequence of SEQ ID NO: 31. In several embodiments, CD28 is used as the sole transmembrane/signaling domain in the construct, however, in several embodiments, CD28 can be used with one or more other domains. For example, combinations of CD28 and CD3zeta are used in some embodiments. By way of further example, combinations of CD28, OX40, 4-1BB, and/or CD3zeta are used in some embodiments.

### Cytotoxic Receptor Complex Constructs

Some embodiments of the compositions and methods described herein relate to a chimeric receptor, such as a CD19-directed chimeric receptor, that comprises a cytotoxic receptor complex or cytotoxic receptor complex construct. In line with the above, a variety of cytotoxic receptor complexes (also referred to as cytotoxic receptors) are provided for herein. The expression of these complexes in immune cells, such as T cells and/or NK cells, allows the targeting and destruction of particular target cells, such as cancerous cells. Non-limiting examples of such cytotoxic receptor complexes are discussed in more detail below.

### Chimeric Antigen Receptor Cytotoxic Receptor Complex Constructs

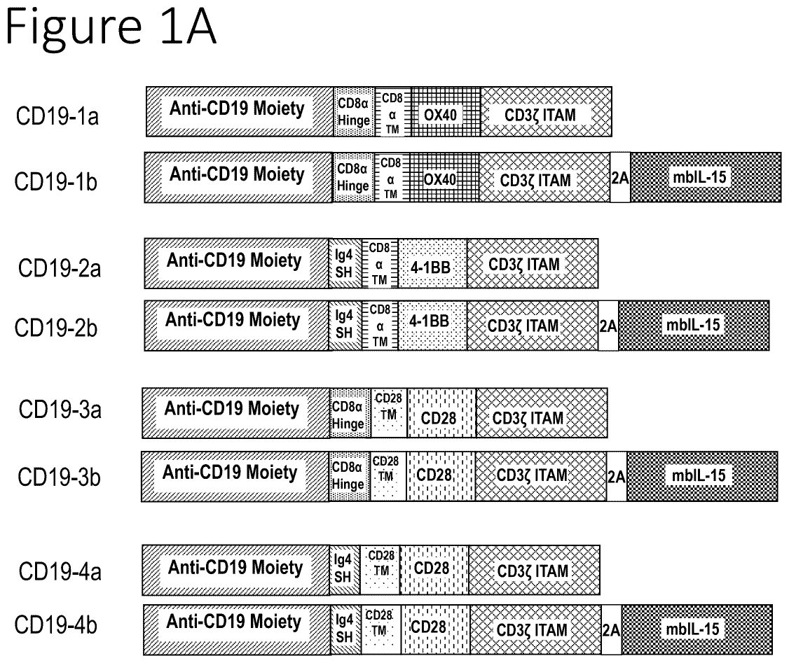

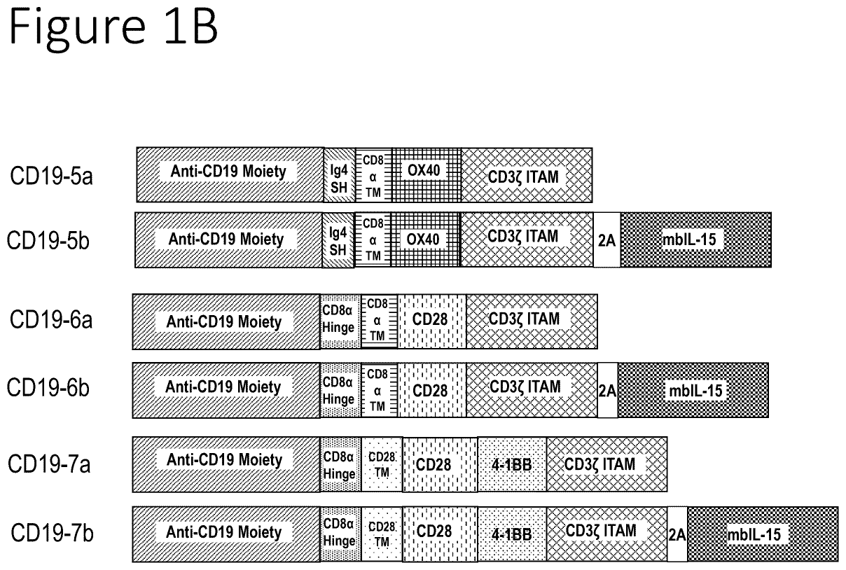

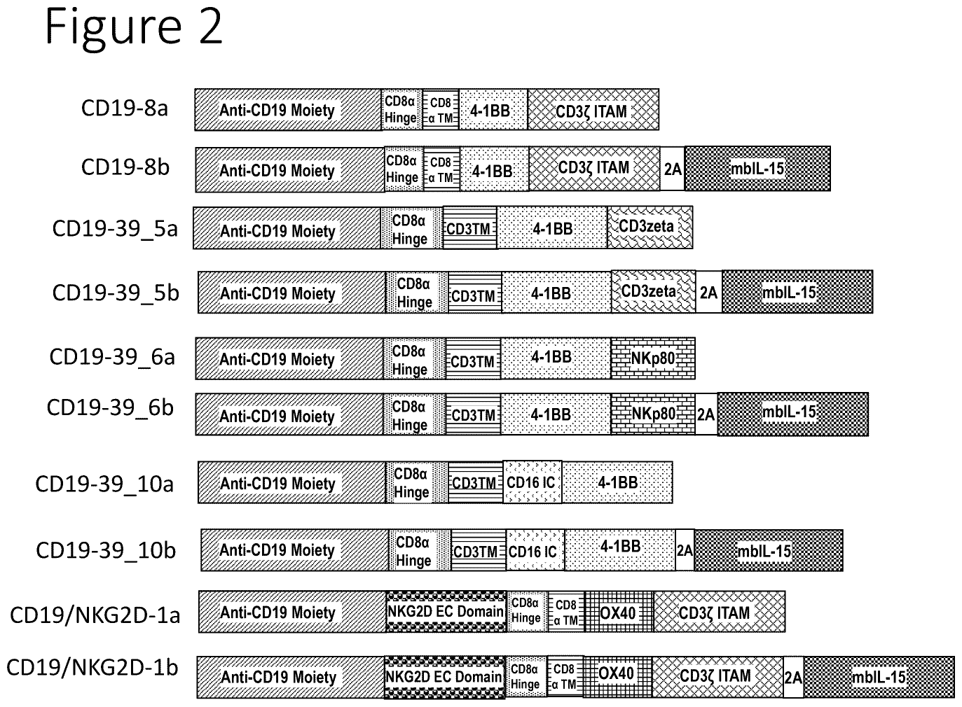

In several embodiments, there are provided for herein a variety of cytotoxic receptor complexes (also referred to as cytotoxic receptors) are provided for herein with the general structure of a chimeric antigen receptor. FIGS. 1A, 1B, and 2 schematically depict non-limiting schematics of constructs that include an anti-CD19 moiety that binds to tumor antigens or tumor-associated antigens expressed on the surface of cancer cells and activates the engineered cell expressing the chimeric antigen receptor. As shown in the figures, several embodiments of the chimeric receptor include an anti-CD19 moiety, a CD8a hinge domain, an Ig4 SH domain (or hinge), a CD8a transmembrane domain, a CD28 transmembrane domain, an OX40 domain, a 4-1BB domain, a CD28 domain, a CD3ζ ITAM domain or subdomain, a CD3zeta domain, an NKp80 domain, a CD16 IC domain, a 2A cleavage site, and a membrane-bound IL-15 domain (though, as above, in several embodiments soluble IL-15 is used). In several embodiments, the binding and activation functions are engineered to be performed by separate domains. Several embodiments relate to complexes with more than one anti-CD19 moiety or other binder/activation moiety. In some embodiments, the binder/activation moiety targets other markers besides CD19, such as a cancer target described herein. In several embodiments, the general structure of the chimeric antigen receptor construct includes a hinge and/or transmembrane domain. These may, in some embodiments, be fulfilled by a single domain, or a plurality of subdomains may be used, in several embodiments. The receptor complex further comprises a signaling domain, which transduces signals after binding of the homing moiety to the target cell, ultimately leading to the cytotoxic effects on the target cell. In several embodiments, the complex further comprises a co-stimulatory domain, which operates, synergistically, in several embodiments, to enhance the function of the signaling domain. Expression of these complexes in immune cells, such as T cells and/or NK cells, allows the targeting and destruction of particular target cells, such as cancerous cells that express CD19. Some such receptor complexes comprise an extracellular domain comprising an anti-CD19 moiety, or CD19-binding moiety, that binds CD19 on the surface of target cells and activates the engineered cell. The CD3zeta ITAM subdomain may act in concert as a signaling domain. The IL-15 domain, e.g., mbIL-15 domain, may acting as a co-stimulatory domain. The IL-15 domain, e.g. mbIL-15 domain, may render immune cells (e.g., NK or T cells) expressing it particularly efficacious against target tumor cells. It shall be appreciated that the IL-15 domain, such as an mbIL-15 domain, can, in accordance with several embodiments, be encoded on a separate construct. Additionally, each of the components may be encoded in one or more separate constructs. In some embodiments, the cytotoxic receptor or CD19-directed receptor comprises a sequence of amino acids that is at least 70%, 75%, 80%, 85%, 90%, 91%, 92%, 93%, 94%, 95%, 96%, 97%, 98%, 99% or 100%, or a range defined by any two of the aforementioned percentages, identical to the sequence of SEQ ID NO: 34.

In one embodiment, there is provided a polynucleotide encoding an anti-CD19moiety/CD8hinge-CD8TM/OX40/CD3zeta chimeric antigen receptor complex (see FIG. 1A, CD19-1a). The polynucleotide comprises or is composed of an anti-CD19 moiety, a CD8a hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8hinge-CD8TM/OX40/CD3zeta/2A/mIL-15 chimeric antigen receptor complex (see FIG. 1A, CD19-1b). The polynucleotide comprises or is composed of an anti-CD19 moiety, a CD8a hinge, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/Ig4SH-CD8TM/4-1BB/CD3zeta chimeric antigen receptor complex (see FIG. 1A, CD19-2a). The polynucleotide comprises or is composed of an anti-CD19 moiety, a Ig4 SH domain, a CD8a transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/Ig4SH-CD8TM/4-1BB/CD3zeta/2A/mIL-15 chimeric antigen receptor complex (see FIG. 1A, CD19-2b). The polynucleotide comprises or is composed of an anti-CD19 moiety, a Ig4 SH domain, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8hinge-CD28TM/CD28/CD3zeta chimeric antigen receptor complex (see FIG. 1A, CD19-3a). The polynucleotide comprises or is composed of an anti-CD19 moiety, a CD8a hinge, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8hinge-CD28TM/CD28/CD3zeta/2A/mIL-15 chimeric antigen receptor complex (see FIG. 1A, CD19-3b). The polynucleotide comprises or is composed of an anti-CD19 moiety, a CD8a hinge, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/Ig4SH-CD28TM/CD28/CD3zeta chimeric antigen receptor complex (see FIG. 1A, CD19-4a). The polynucleotide comprises or is composed of an anti-CD19 moiety, an Ig4 SH domain, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/Ig4SH-CD28TM/CD28/CD3zeta/2A/mIL-15 chimeric antigen receptor complex (see FIG. 1A, CD19-4b). The polynucleotide comprises or is composed of an anti-CD19 moiety, an Ig4 SH domain, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/Ig4SH-CD8TM/OX40/CD3zeta chimeric antigen receptor complex (see FIG. 2A, CD19-5a). The polynucleotide comprises or is composed of an anti-CD19 moiety, a Ig4 SH domain, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/Ig4SH-CD8TM/OX40/CD3zeta/2A/mIL-15 chimeric antigen receptor complex (see FIG. 2A, CD19-5b). The polynucleotide comprises or is composed of an anti-CD19 moiety, a Ig4 SH domain, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8hinge-CD3aTM/CD28/CD3zeta chimeric antigen receptor complex (see FIG. 1B, CD19-6a). The polynucleotide comprises or is composed of an anti-CD19 moiety, a CD8a hinge, a CD3a transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8hinge-CD3αTM/CD28/CD3zeta/2A/mIL-15 chimeric antigen receptor complex (see FIG. 1B, CD19-6b). The polynucleotide comprises or is composed of an anti-CD19 moiety, a CD8a hinge, a CD3a transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8hinge-CD28TM/CD28/4-1BB/CD3zeta chimeric antigen receptor complex (see FIG. 1B, CD19-7a). The polynucleotide comprises or is composed of an anti-CD19 moiety, a CD8a hinge, a CD28 transmembrane domain, a CD28 domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8hinge-CD28TM/CD28/4-1BB/CD3zeta/2A/mIL-15 chimeric antigen receptor complex (see FIG. 1B, CD19-7b). The polynucleotide comprises or is composed of an anti-CD19 moiety, a CD8a hinge, a CD28 transmembrane domain, a CD28 domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD8 alpha TM/4-1BB/CD3zeta chimeric antigen receptor complex (see FIG. 2, CD19-8a). The polynucleotide comprises or is composed of an anti-CD19 moiety, a CD8a hinge, a CD8a transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD8 alpha TM/4-1BB/CD3zeta/2A/mIL-15 chimeric antigen receptor complex (see FIG. 2, CD19-8b). The polynucleotide comprises or is composed of an anti-CD19 moiety, a CD8a hinge, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD3 TM/4-1BB/CD3zeta chimeric antigen receptor complex (see FIG. 2, CD19-39_5a). The polynucleotide comprises or is composed of an anti-CD19 moiety, a CD8a hinge, a CD3 transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD3 TM/4-1BB/CD3zeta/2A/mIL-15 chimeric antigen receptor complex (see FIG. 2, CD19-39_5b). The polynucleotide comprises or is composed of an anti-CD19 moiety, a CD8a hinge, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD3 TM/4-1BB/NKp80 chimeric antigen receptor complex (see FIG. 2, CD19-39_6a). The polynucleotide comprises or is composed of an anti-CD19 moiety, a CD8a hinge, a CD3 transmembrane domain, a 4-1BB domain, and an NKp80 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD3 TM/4-1BB/NKp80/2A/mIL-15 chimeric antigen receptor complex (see FIG. 2, CD19-39_6b). The polynucleotide comprises or is composed of an anti-CD19 moiety, a CD8a hinge, a CD8a transmembrane domain, a 4-1BB domain, an NKp80 domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD3 TM/CD16 intracellular domain/4-1BB chimeric antigen receptor complex (see FIG. 2, CD19-39_10a). The polynucleotide comprises or is composed of an anti-CD19 moiety, a CD8a hinge, a CD3 transmembrane domain, CD16 intracellular domain, and a 4-1BB domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/CD8 alpha hinge/CD3 TM/CD16/4-1BB/2A/mIL-15 chimeric antigen receptor complex (see FIG. 2, CD19-39_10b). The polynucleotide comprises or is composed of an anti-CD19 moiety, a CD8a hinge, a CD8a transmembrane domain, a CD16 intracellular domain, a 4-1BB domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/NKG2D Extracellular Domain/CD8hinge-CD8TM/OX40/CD3zeta chimeric antigen receptor complex (see FIG. 2, CD19/NKG2D-1a). The polynucleotide comprises or is composed of an anti-CD19 moiety, an NKG2D extracellular domain (either full length or a fragment), a CD8a hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

In several embodiments, there is provided a polynucleotide encoding an anti-CD19moiety/NKG2D EC Domain/CD8hinge-CD8TM/OX40/CD3zeta/2A/mIL-15 chimeric antigen receptor complex (see FIG. 2, CD19/NKG2D-1b). The polynucleotide comprises or is composed of an anti-CD19 moiety, an NKG2D extracellular domain (either full length or a fragment), a CD8a hinge, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein.

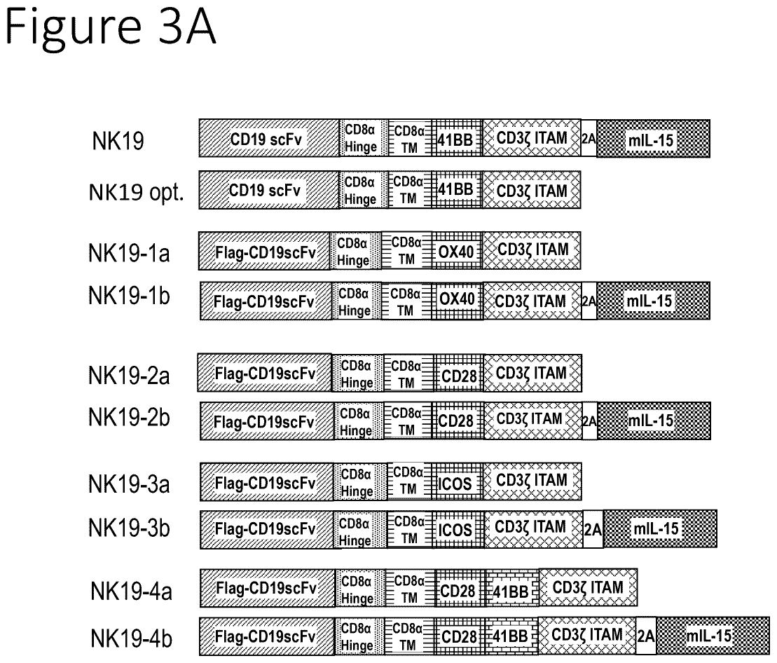

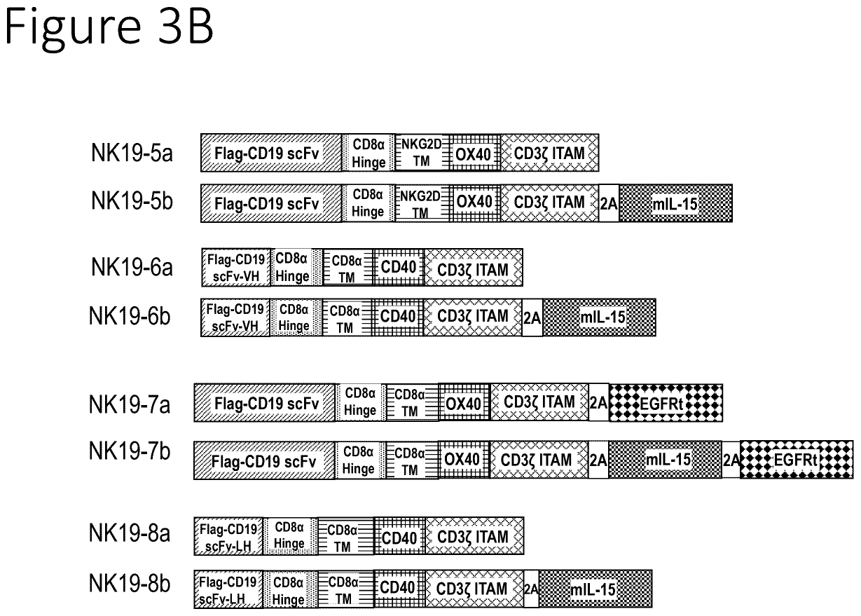

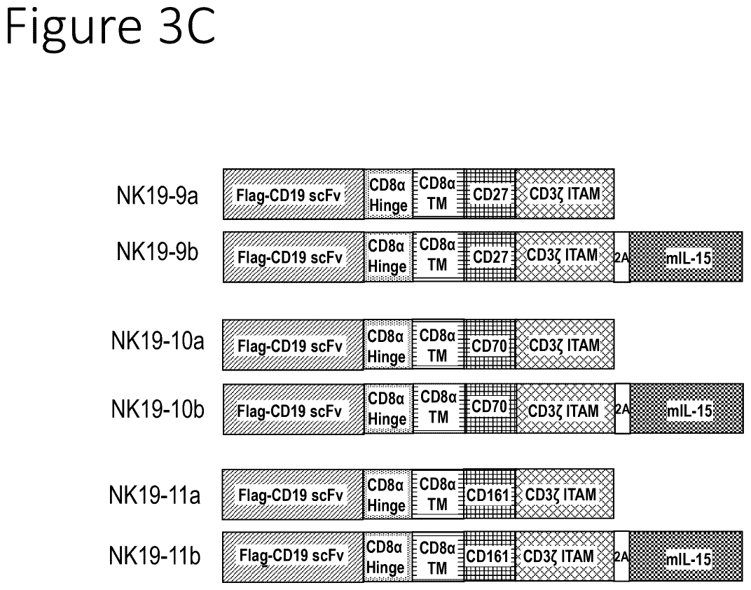

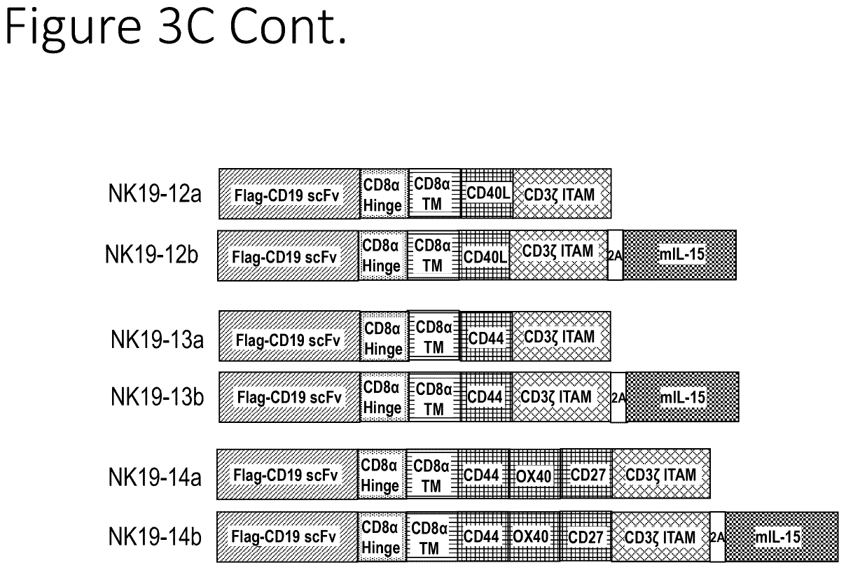

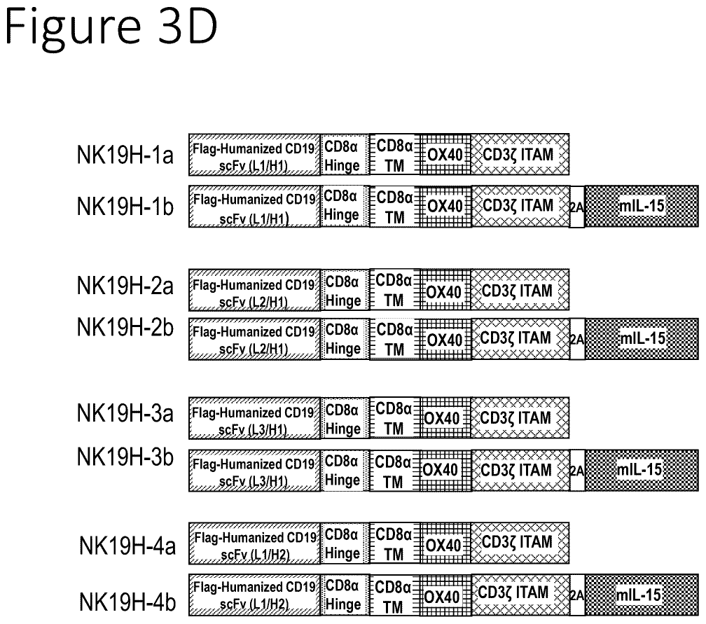

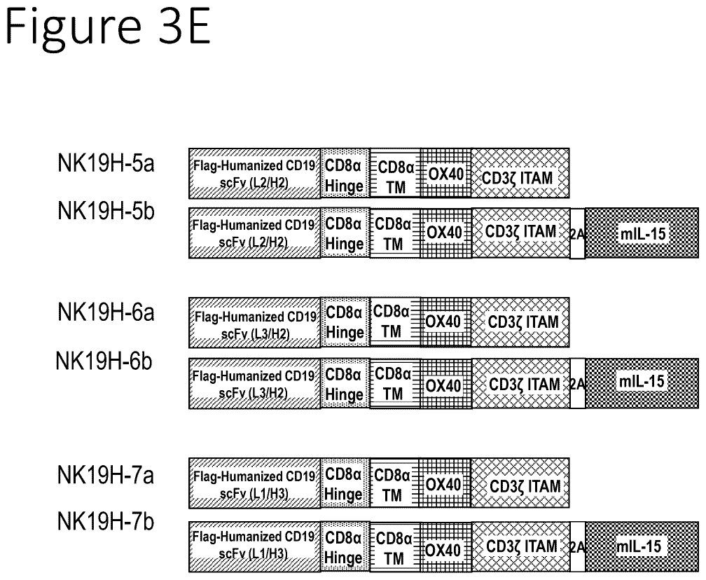

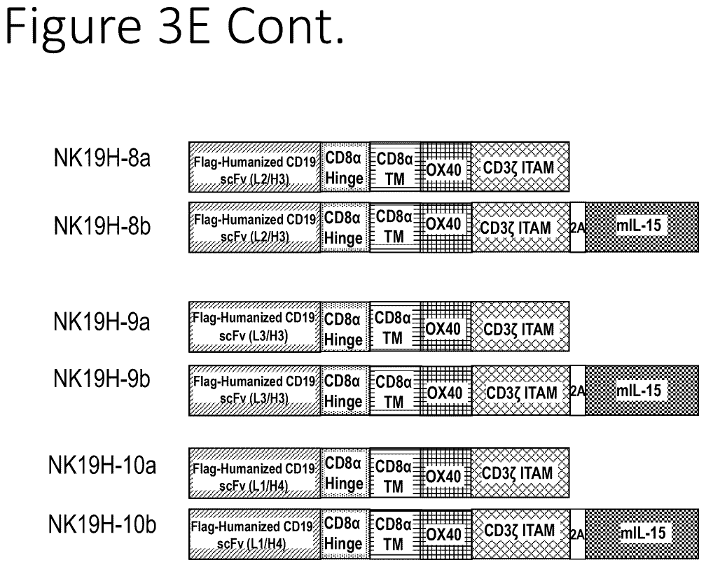

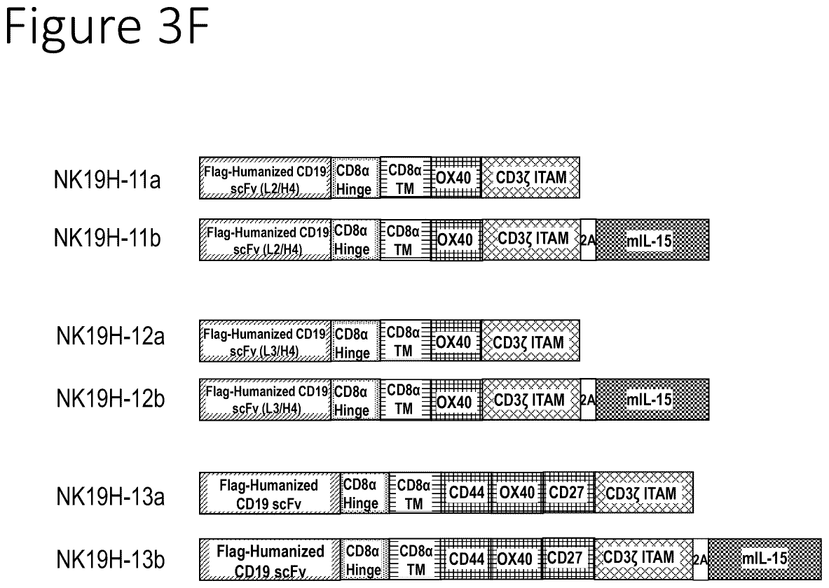

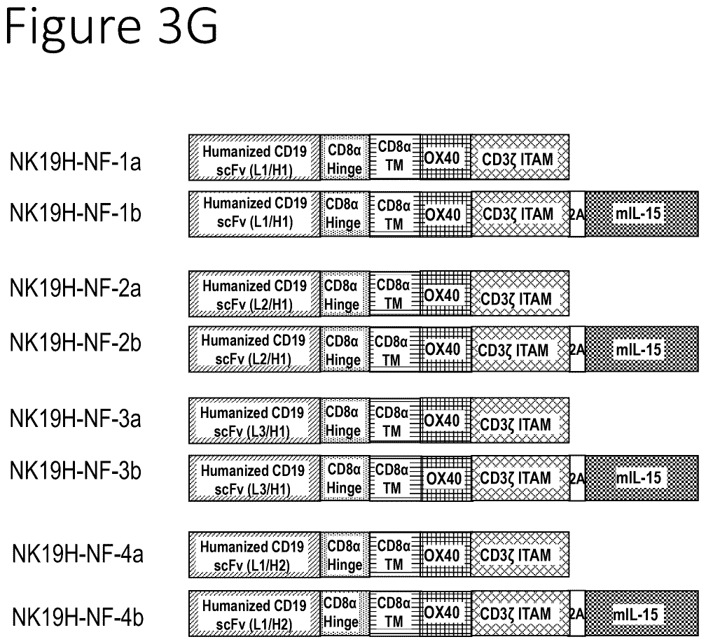

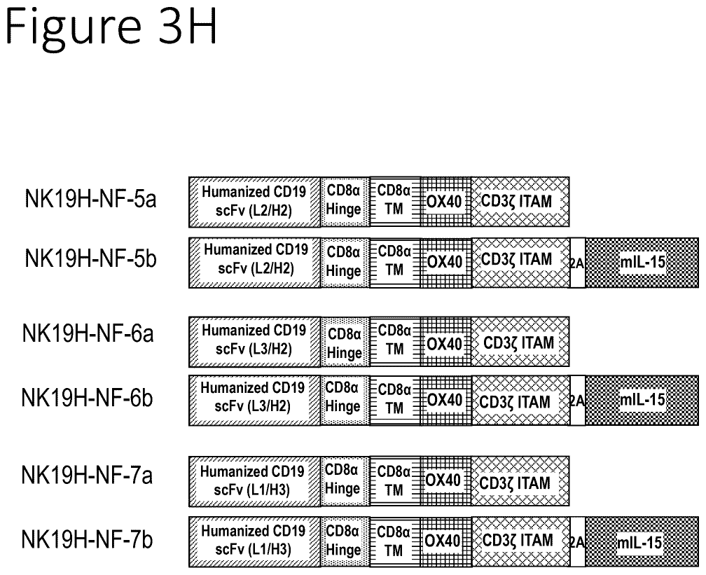

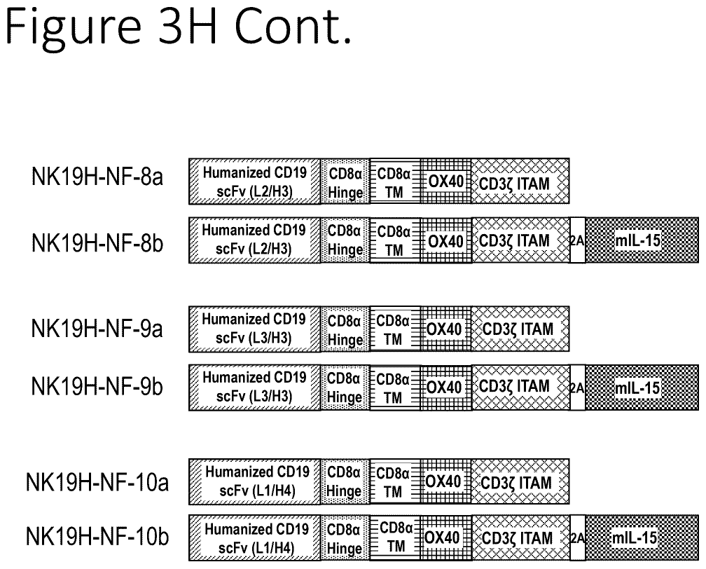

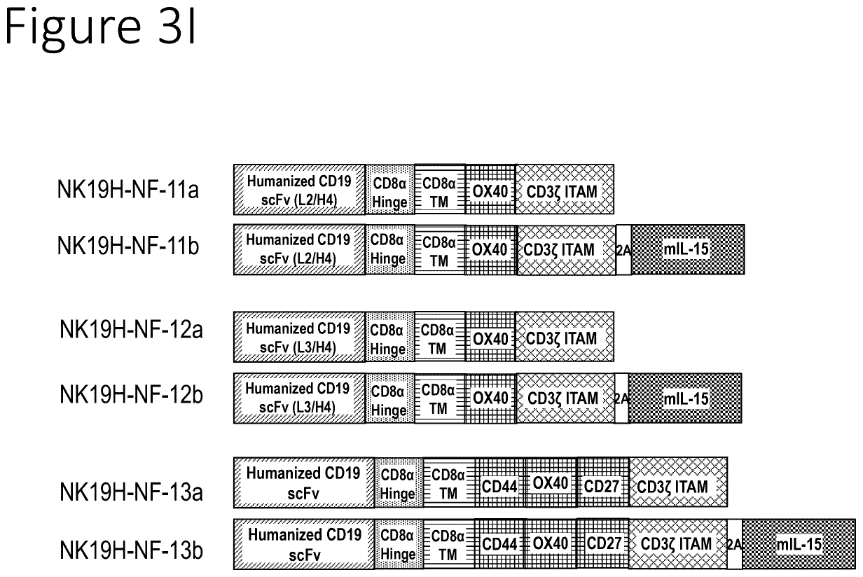

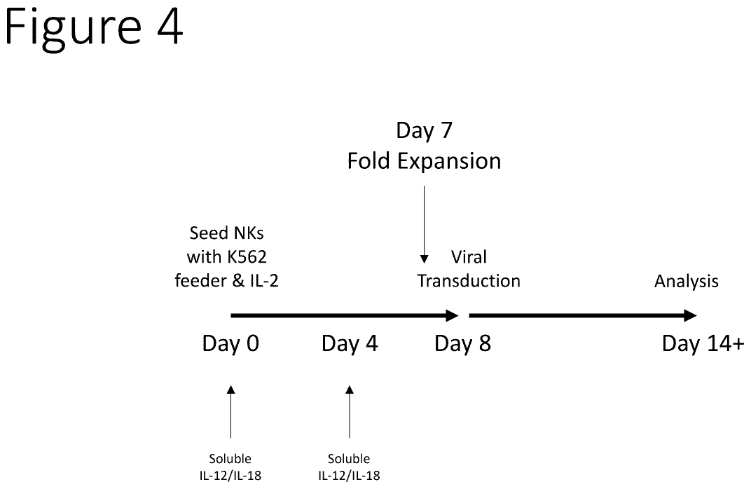

In several embodiments, there is provided a polynucleotide encoding an anti-CD19moiety/CD8hinge/CD8TM/4-1BB/CD3zeta/mbIL15 chimeric antigen receptor complex (see FIG. 3A, NK19). The polynucleotide comprises or is composed of an anti-CD19 scFv, a CD8a hinge, a CD8a transmembrane domain, a 4-1BB domain, and a CD3zeta domain. In several embodiments, this receptor complex is encoded by a nucleic acid molecule having the sequence of SEQ ID NO: 85. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 85. In several embodiments, the chimeric receptor comprises the amino acid sequence of SEQ ID NO: 86. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 86. Schematically depicted and used in several embodiments, there is provided an NK19 construct that lacks an mbIL15 domain (FIG. 3A, NK19 opt.)

In several embodiments, there is provided a polynucleotide encoding an anti-CD19moiety/CD8hinge/CD8TM/OX40/CD3zeta chimeric antigen receptor complex (see FIG. 3A, NK19-1a). The polynucleotide comprises or is composed of an anti-CD19 scFv, a CD8a hinge, a CD8a transmembrane domain, an OX40 domain, and a CD3zeta domain. In several embodiments, the chimeric antigen receptor further comprises mbIL15 (see FIG. 3A, NK19-1b). In such embodiments, the polynucleotide comprises or is composed of an anti-CD19 scFv, a CD8a hinge, a CD8a transmembrane domain, an OX40 domain, a CD3zeta domain, a 2A cleavage site, and an mbIL-15 domain as described herein. In several embodiments, this receptor complex is encoded by a nucleic acid molecule having the sequence of SEQ ID NO: 59. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 59. In several embodiments, the chimeric receptor comprises the amino acid sequence of SEQ ID NO: 60. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 60. In several embodiments, the CD19 scFv does not comprise a Flag tag.

In several embodiments, there is provided a polynucleotide encoding an anti-CD19moiety/CD8hinge/CD28TM/CD28/CD3zeta chimeric antigen receptor complex (see FIG. 3A, NK19-2a). The polynucleotide comprises or is composed of an anti-CD19 scFv, a CD8a hinge, a CD28 transmembrane domain, CD28 signaling domain, and a CD3zeta domain. In several embodiments, the chimeric antigen receptor further comprises mbIL15 (see FIG. 3A, NK19-2b). In such embodiments, the polynucleotide comprises or is composed of an anti-CD19 scFv, a CD8a hinge, a CD28 transmembrane domain, CD28 signaling domain, a CD3zeta domain a 2A cleavage site, and an mbIL-15 domain as described herein. In several embodiments, this receptor complex is encoded by a nucleic acid molecule having the sequence of SEQ ID NO: 61. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 61. In several embodiments, the chimeric receptor comprises the amino acid sequence of SEQ ID NO: 62. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 62. In several embodiments, the CD19 scFv does not comprise a Flag tag.

In several embodiments, there is provided a polynucleotide encoding an anti-CD19moiety/CD8hinge/CD8aTM/ICOS/CD3zeta chimeric antigen receptor complex (see FIG. 3A, NK19-3a). The polynucleotide comprises or is composed of an anti-CD19 scFv, a CD8a hinge, a CD8a transmembrane domain, inducible costimulator (ICOS) signaling domain, and a CD3zeta domain. In several embodiments, the chimeric antigen receptor further comprises mbIL15 (see FIG. 3A, NK19-3b). In such embodiments, the polynucleotide comprises or is composed of an anti-CD19 scFv, a CD8a hinge, a CD8a transmembrane domain, inducible costimulator (ICOS) signaling domain, a CD3zeta domain, a 2A cleavage site, and an mbIL-15 domain as described herein. In several embodiments, this receptor complex is encoded by a nucleic acid molecule having the sequence of SEQ ID NO: 63. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 63. In several embodiments, the chimeric receptor comprises the amino acid sequence of SEQ ID NO: 64. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 64. In several embodiments, the CD19 scFv does not comprise a Flag tag.

In several embodiments, there is provided a polynucleotide encoding an anti-CD19moiety/CD8hinge/CD8aTM/CD28/4-1BB/CD3zeta chimeric antigen receptor complex (see FIG. 3A, NK19-4a). The polynucleotide comprises or is composed of an anti-CD19 scFv, a CD8a hinge, a CD8a transmembrane domain, a CD28 signaling domain, a 4-1BB signaling domain, and a CD3zeta domain. In several embodiments, the chimeric antigen receptor further comprises mbIL15 (see FIG. 3A, NK19-4b). In such embodiments, the polynucleotide comprises or is composed of an anti-CD19 scFv, a CD8a hinge, a CD8a transmembrane domain, a CD28 signaling domain, a 4-1BB signaling domain, a CD3zeta domain, a 2A cleavage site, and an mbIL-15 domain as described herein. In several embodiments, this receptor complex is encoded by a nucleic acid molecule having the sequence of SEQ ID NO: 65. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 65. In several embodiments, the chimeric receptor comprises the amino acid sequence of SEQ ID NO: 66. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 66. In several embodiments, the CD19 scFv does not comprise a Flag tag.

In several embodiments, there is provided a polynucleotide encoding an anti-CD19moiety/CD8hinge/NKG2DTM/OX40/CD3zeta chimeric antigen receptor complex (see FIG. 3B, NK19-5a). The polynucleotide comprises or is composed of an anti-CD19 scFv, a CD8a hinge, a NKG2D transmembrane domain, an OX40 signaling domain, and a CD3zeta domain. In several embodiments, the chimeric antigen receptor further comprises mbIL15 (see FIG. 3B, NK19-5b). In such embodiments, the polynucleotide comprises or is composed of an anti-CD19 scFv, a CD8a hinge, a NKG2D transmembrane domain, an OX40 signaling domain, a CD3zeta domain, a 2A cleavage site, and an mbIL-15 domain as described herein. In several embodiments, this receptor complex is encoded by a nucleic acid molecule having the sequence of SEQ ID NO: 67. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 67. In several embodiments, the chimeric receptor comprises the amino acid sequence of SEQ ID NO: 68. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 68. In several embodiments, the CD19 scFv does not comprise a Flag tag.

In several embodiments, there is provided a polynucleotide encoding an anti-CD19moiety/CD8hinge/CD8aTM/CD40/CD3zeta chimeric antigen receptor complex (see FIG. 3B, NK19-6a). The polynucleotide comprises or is composed of an anti-CD19 scFv variable heavy chain, a CD8a hinge, a CD8a transmembrane domain, a CD40 signaling domain, and a CD3zeta domain. In several embodiments, the chimeric antigen receptor further comprises mbIL15 (see FIG. 3B, NK19-6b). In such embodiments, the polynucleotide comprises or is composed of an anti-CD19 scFv variable heavy chain, a CD8a hinge, a CD8a transmembrane domain, a CD40 signaling domain, a CD3zeta domain, a 2A cleavage site, and an mbIL-15 domain as described herein. In several embodiments, this receptor complex is encoded by a nucleic acid molecule having the sequence of SEQ ID NO: 69. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 69. In several embodiments, the chimeric receptor comprises the amino acid sequence of SEQ ID NO: 70. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 70. In several embodiments, the CD19 scFv does not comprise a Flag tag.

In several embodiments, there is provided a polynucleotide encoding an anti-CD19moiety/CD8hinge/CD8aTM/OX40/CD3zeta/2A/EGFRt chimeric antigen receptor complex (see FIG. 3B, NK19-7a). The polynucleotide comprises or is composed of an anti-CD19 scFv, a CD8a hinge, a CD8a transmembrane domain, an OX40 signaling domain, a CD3zeta domain, a 2A cleavage side, and a truncated version of the epidermal growth factor receptor (EGFRt). In several embodiments, the chimeric antigen receptor further comprises mbIL15 (see FIG. 3B, NK19-7b). In such embodiments, the polynucleotide comprises or is composed of an anti-CD19 scFv, a CD8a hinge, a CD8a transmembrane domain, an OX40 signaling domain, a CD3zeta domain, a 2A cleavage side, a truncated version of the epidermal growth factor receptor (EGFRt), an additional 2A cleavage site, and an mbIL-15 domain as described herein. In several embodiments, this receptor complex is encoded by a nucleic acid molecule having the sequence of SEQ ID NO: 71. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 71. In several embodiments, the chimeric receptor comprises the amino acid sequence of SEQ ID NO: 72. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 72. In several embodiments, the CD19 scFv does not comprise a Flag tag.

In several embodiments, there is provided a polynucleotide encoding an anti-CD19moiety/CD8hinge/CD8aTM/CD40/CD3zeta chimeric antigen receptor complex (see FIG. 3B, NK19-8a). The polynucleotide comprises or is composed of an anti-CD19 scFv variable light chain, a CD8a hinge, a CD8a transmembrane domain, a CD40 signaling domain, and a CD3zeta domain. In several embodiments, the chimeric antigen receptor further comprises mbIL15 (see FIG. 3B, NK19-7b). In such embodiments, the polynucleotide comprises or is composed of an anti-CD19 scFv variable light chain, a CD8a hinge, a CD8a transmembrane domain, a CD40 signaling domain, a CD3zeta domain, a 2A cleavage site, and an mbIL-15 domain as described herein. In several embodiments, this receptor complex is encoded by a nucleic acid molecule having the sequence of SEQ ID NO: 73. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 73. In several embodiments, the chimeric receptor comprises the amino acid sequence of SEQ ID NO: 74. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 74. In several embodiments, the CD19 scFv does not comprise a Flag tag.

In several embodiments, there is provided a polynucleotide encoding an anti-CD19moiety/CD8hinge/CD8aTM/CD40/CD3zeta chimeric antigen receptor complex (see FIG. 3B, NK19-8a). The polynucleotide comprises or is composed of an anti-CD19 scFv variable light chain, a CD8a hinge, a CD8a transmembrane domain, a CD40 signaling domain, and a CD3zeta domain. In several embodiments, the chimeric antigen receptor further comprises mbIL15 (see FIG. 3B, NK19-7b). In such embodiments, the polynucleotide comprises or is composed of an anti-CD19 scFv variable light chain, a CD8a hinge, a CD8a transmembrane domain, a CD40 signaling domain, a CD3zeta domain, a 2A cleavage site, and an mbIL-15 domain as described herein. In several embodiments, this receptor complex is encoded by a nucleic acid molecule having the sequence of SEQ ID NO: 73. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 73. In several embodiments, the chimeric receptor comprises the amino acid sequence of SEQ ID NO: 74. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 74. In several embodiments, the CD19 scFv does not comprise a Flag tag.

In several embodiments, there is provided a polynucleotide encoding an anti-CD19moiety/CD8hinge/CD8aTM/CD27/CD3zeta chimeric antigen receptor complex (see FIG. 3C, NK19-9a). The polynucleotide comprises or is composed of an anti-CD19 scFv, a CD8a hinge, a CD8a transmembrane domain, a CD27 signaling domain, and a CD3zeta domain. In several embodiments, the chimeric antigen receptor further comprises mbIL15 (see FIG. 3C, NK19-9b). In such embodiments, the polynucleotide comprises or is composed of an anti-CD19 scFv, a CD8a hinge, a CD8a transmembrane domain, a CD27 signaling domain, a CD3zeta domain, a 2A cleavage site, and an mbIL-15 domain as described herein. In several embodiments, this receptor complex is encoded by a nucleic acid molecule having the sequence of SEQ ID NO: 75. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 75. In several embodiments, the chimeric receptor comprises the amino acid sequence of SEQ ID NO: 76. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 76. In several embodiments, the CD19 scFv does not comprise a Flag tag.

In several embodiments, there is provided a polynucleotide encoding an anti-CD19moiety/CD8hinge/CD8aTM/CD70/CD3zeta chimeric antigen receptor complex (see FIG. 3C, NK19-10a). The polynucleotide comprises or is composed of an anti-CD19 scFv, a CD8a hinge, a CD8a transmembrane domain, a CD70 signaling domain, and a CD3zeta domain. In several embodiments, the chimeric antigen receptor further comprises mbIL15 (see FIG. 3C, NK19-10b). In such embodiments, the polynucleotide comprises or is composed of an anti-CD19 scFv, a CD8a hinge, a CD8a transmembrane domain, a CD70 signaling domain, a CD3zeta domain, a 2A cleavage site, and an mbIL-15 domain as described herein. In several embodiments, this receptor complex is encoded by a nucleic acid molecule having the sequence of SEQ ID NO: 77. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 77. In several embodiments, the chimeric receptor comprises the amino acid sequence of SEQ ID NO: 78. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 78. In several embodiments, the CD19 scFv does not comprise a Flag tag.

In several embodiments, there is provided a polynucleotide encoding an anti-CD19moiety/CD8hinge/CD8aTM/CD161/CD3zeta chimeric antigen receptor complex (see FIG. 3C, NK19-11a). The polynucleotide comprises or is composed of an anti-CD19 scFv, a CD8a hinge, a CD8a transmembrane domain, a CD161 signaling domain, and a CD3zeta domain. In several embodiments, the chimeric antigen receptor further comprises mbIL15 (see FIG. 3C, NK19-11b). In such embodiments, the polynucleotide comprises or is composed of an anti-CD19 scFv, a CD8a hinge, a CD8a transmembrane domain, a CD161 signaling domain, a CD3zeta domain, a 2A cleavage site, and an mbIL-15 domain as described herein. In several embodiments, this receptor complex is encoded by a nucleic acid molecule having the sequence of SEQ ID NO: 79. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 79. In several embodiments, the chimeric receptor comprises the amino acid sequence of SEQ ID NO: 80. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 80. In several embodiments, the CD19 scFv does not comprise a Flag tag.

In several embodiments, there is provided a polynucleotide encoding an anti-CD19moiety/CD8hinge/CD8aTM/CD40L/CD3zeta chimeric antigen receptor complex (see FIG. 3C, NK19-12a). The polynucleotide comprises or is composed of an anti-CD19 scFv, a CD8a hinge, a CD8a transmembrane domain, a CD40L signaling domain, and a CD3zeta domain. In several embodiments, the chimeric antigen receptor further comprises mbIL15 (see FIG. 3C, NK19-12b). In such embodiments, the polynucleotide comprises or is composed of an anti-CD19 scFv, a CD8a hinge, a CD8a transmembrane domain, a CD40L signaling domain, a CD3zeta domain, a 2A cleavage site, and an mbIL-15 domain as described herein. In several embodiments, this receptor complex is encoded by a nucleic acid molecule having the sequence of SEQ ID NO: 81. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 81. In several embodiments, the chimeric receptor comprises the amino acid sequence of SEQ ID NO: 82. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 82. In several embodiments, the CD19 scFv does not comprise a Flag tag.

In several embodiments, there is provided a polynucleotide encoding an anti-CD19moiety/CD8hinge/CD8aTM/CD44/CD3zeta chimeric antigen receptor complex (see FIG. 3C, NK19-13). The polynucleotide comprises or is composed of an anti-CD19 scFv, a CD8a hinge, a CD8a transmembrane domain, a CD44 signaling domain, and a CD3zeta domain. In several embodiments, the chimeric antigen receptor further comprises mbIL15 (see FIG. 3C, NK19-13b). In such embodiments, the polynucleotide comprises or is composed of an anti-CD19 scFv, a CD8a hinge, a CD8a transmembrane domain, a CD44 signaling domain, a CD3zeta domain, a 2A cleavage site, and an mbIL-15 domain as described herein. In several embodiments, this receptor complex is encoded by a nucleic acid molecule having the sequence of SEQ ID NO: 83. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 83. In several embodiments, the chimeric receptor comprises the amino acid sequence of SEQ ID NO: 84. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 84. In several embodiments, the CD19 scFv does not comprise a Flag tag.

In several embodiments, there is provided a polynucleotide encoding a Flag-tag humanized anti-CD19moiety/CD8hinge/CD8TM/OX40/CD3zeta chimeric antigen receptor complex (see FIG. 3D, NK19H-1a). The polynucleotide comprises or is composed of an anti-CD19 scFv that has been humanized and comprises a first humanized light chain and a first humanized heavy chain (L1/H1), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3D, NK19H-1b). In such embodiments, the polynucleotide comprises or is composed of an anti-CD19 scFv that has been humanized and comprises a first humanized light chain and a first humanized heavy chain (L1/H1), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 160. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 160. In several embodiments, the chimeric receptor comprises the amino acid sequence of SEQ ID NO: 161. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 161.

In several embodiments, there is provided a polynucleotide encoding a Flag-tag humanized anti-CD19moiety/CD8hinge/CD8TM/OX40/CD3zeta chimeric antigen receptor complex (see FIG. 3D, NK19H-2a). The polynucleotide comprises or is composed of an anti-CD19 scFv that has been humanized, and comprises a second humanized light chain and a first humanized heavy chain (L2/H1), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3D, NK19H-2b). In such embodiments, the polynucleotide comprises or is composed of an anti-CD19 scFv that has been humanized comprises a second humanized light chain and a first humanized heavy chain (L2/H1), and comprises a Flag tag, a CD8a hinge, a CD8a transmembrane domain, an OX40 signaling domain, a CD3zeta domain, a 2A cleavage site, and an mbIL-15 domain as described herein. In several embodiments, this receptor complex is encoded by a nucleic acid molecule having the sequence of SEQ ID NO: 162. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 162. In several embodiments, the chimeric receptor comprises the amino acid sequence of SEQ ID NO: 163. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 162.

In several embodiments, there is provided a polynucleotide encoding a Flag-tag humanized anti-CD19moiety/CD8hinge/CD8TM/OX40/CD3zeta chimeric antigen receptor complex (see FIG. 3D, NK19H-3a). The polynucleotide comprises or is composed of an anti-CD19 scFv that has been humanized and comprises a third humanized light chain and a first humanized heavy chain (L3/H1), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3D, NK19H-3b). In such embodiments, the polynucleotide comprises or is composed of an anti-CD19 scFv that has been humanized and comprises a third humanized light chain and a first humanized heavy chain (L3/H1), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 164. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 164. In several embodiments, the chimeric receptor comprises the amino acid sequence of SEQ ID NO: 165. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 165.

In several embodiments, there is provided a polynucleotide encoding a Flag-tag humanized anti-CD19moiety/CD8hinge/CD8TM/OX40/CD3zeta chimeric antigen receptor complex (see FIG. 3D, NK19H-4a). The polynucleotide comprises or is composed of an anti-CD19 scFv that has been humanized and comprises a first humanized light chain and a second humanized heavy chain (L1/H2), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3D, NK19H-4b). In such embodiments, the polynucleotide comprises or is composed of an anti-CD19 scFv that has been humanized and comprises a first humanized light chain and a second humanized heavy chain (L1/H2), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 166. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 166. In several embodiments, the chimeric receptor comprises the amino acid sequence of SEQ ID NO: 167. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 167.

In several embodiments, there is provided a polynucleotide encoding a Flag-tag humanized anti-CD19moiety/CD8hinge/CD8TM/OX40/CD3zeta chimeric antigen receptor complex (see FIG. 3E, NK19H-5a). The polynucleotide comprises or is composed of an anti-CD19 scFv that has been humanized and comprises a second humanized light chain and a second humanized heavy chain (L2/H2), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3E, NK19H-5b). In such embodiments, the polynucleotide comprises or is composed of an anti-CD19 scFv that has been humanized and comprises a second humanized light chain and a second humanized heavy chain (L2/H2), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 168. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 168. In several embodiments, the chimeric receptor comprises the amino acid sequence of SEQ ID NO: 169. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 169.

In several embodiments, there is provided a polynucleotide encoding a Flag-tag humanized anti-CD19moiety/CD8hinge/CD8TM/OX40/CD3zeta chimeric antigen receptor complex (see FIG. 3E, NK19H-6a). The polynucleotide comprises or is composed of an anti-CD19 scFv that has been humanized and comprises a third humanized light chain and a second humanized heavy chain (L3/H2), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3E, NK19H-6b). In such embodiments, the polynucleotide comprises or is composed of an anti-CD19 scFv that has been humanized and comprises a third humanized light chain and a second humanized heavy chain (L3/H2) and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 170. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 170. In several embodiments, the chimeric receptor comprises the amino acid sequence of SEQ ID NO: 171. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 171.

In several embodiments, there is provided a polynucleotide encoding an Flag-tag humanized anti-CD19moiety/CD8hinge/CD8TM/OX40/CD3zeta chimeric antigen receptor complex (see FIG. 3E, NK19H-7a). The polynucleotide comprises or is composed of an anti-CD19 scFv that has been humanized and comprises a first humanized light chain and a third humanized heavy chain (L1/H3), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3E, NK19H-7b). In such embodiments, the polynucleotide comprises or is composed of an anti-CD19 scFv that has been humanized and comprises a first humanized light chain and a third humanized heavy chain (L1/H3), and comprises a Flag tag, a CD8a hinge, a CD8a transmembrane domain, an OX40 signaling domain, and a CD3zeta domain, a 2A cleavage side, a truncated version of the epidermal growth factor receptor (EGFRt), an additional 2A cleavage site, and an mbIL-15 domain as described herein. In several embodiments, this receptor complex is encoded by a nucleic acid molecule having the sequence of SEQ ID NO: 172. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 172. In several embodiments, the chimeric receptor comprises the amino acid sequence of SEQ ID NO: 173. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 174.

In several embodiments, there is provided a polynucleotide encoding a Flag-tag humanized anti-CD19moiety/CD8hinge/CD8TM/OX40/CD3zeta chimeric antigen receptor complex (see FIG. 3E, NK19H-8a). The polynucleotide comprises or is composed of an anti-CD19 scFv that has been humanized and comprises a second humanized light chain and a third humanized heavy chain (L2/H3), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3E, NKH19-8b). In such embodiments, the polynucleotide comprises or is composed of an anti-CD19 scFv that has been humanized and comprises a second humanized light chain and a third humanized heavy chain (L2/H3), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 174. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 174. In several embodiments, the chimeric receptor comprises the amino acid sequence of SEQ ID NO: 175. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 175.

In several embodiments, there is provided a polynucleotide encoding a Flag-tag humanized anti-CD19moiety/CD8hinge/CD8TM/OX40/CD3zeta chimeric antigen receptor complex (see FIG. 3E, NK19H-9a). The polynucleotide comprises or is composed of an anti-CD19 scFv that has been humanized and comprises a third humanized light chain and a third humanized heavy chain (L3/H3), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3E, NKH19-9b). In such embodiments, the polynucleotide comprises or is composed of an anti-CD19 scFv that has been humanized and comprises a third humanized light chain and a third humanized heavy chain (L3/H3), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 176. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 176. In several embodiments, the chimeric receptor comprises the amino acid sequence of SEQ ID NO: 177. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 177.

In several embodiments, there is provided a polynucleotide encoding a Flag-tag humanized anti-CD19moiety/CD8hinge/CD8TM/OX40/CD3zeta chimeric antigen receptor complex (see FIG. 3E, NKH19-10a). The polynucleotide comprises or is composed of an anti-CD19 scFv that has been humanized and comprises a first humanized light chain and a fourth humanized heavy chain (L1/H4), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3E, NK19H-10b). In such embodiments, the polynucleotide comprises or is composed of an anti-CD19 scFv that has been humanized and comprises a first humanized light chain and a fourth humanized heavy chain (L1/H4), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 178. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 178. In several embodiments, the chimeric receptor comprises the amino acid sequence of SEQ ID NO: 179. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 179.

In several embodiments, there is provided a polynucleotide encoding a Flag-tag humanized anti-CD19moiety/CD8hinge/CD8TM/OX40/CD3zeta chimeric antigen receptor complex (see FIG. 3F, NK19H-11a). The polynucleotide comprises or is composed of an anti-CD19 scFv that has been humanized and comprises a second humanized light chain and a fourth humanized heavy chain (L2/H4), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3F, NK19H-11b). In such embodiments, the polynucleotide comprises or is composed of an anti-CD19 scFv that has been humanized and comprises a second humanized light chain and a fourth humanized heavy chain (L2/H4), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 180. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 180. In several embodiments, the chimeric receptor comprises the amino acid sequence of SEQ ID NO: 181. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 181.

In several embodiments, there is provided a polynucleotide encoding a Flag-tag, humanized anti-CD19moiety/CD8hinge/CD8TM/OX40/CD3zeta chimeric antigen receptor complex (see FIG. 3F NK19H-12a). The polynucleotide comprises or is composed of an anti-CD19 scFv that has been humanized and comprises a third humanized light chain and a fourth humanized heavy chain (L3/H4), and comprises a Flag tag, a CD8a hinge, a CD8a transmembrane domain, an OX40 signaling domain, and a CD3zeta domain. In several embodiments, the chimeric antigen receptor further comprises mbIL15 (see FIG. 3F, NK19H-12b). In such embodiments, the polynucleotide comprises or is composed of an anti-CD19 scFv that has been humanized and comprises a third humanized light chain and a fourth humanized heavy chain (L3/H4), and comprises a Flag tag, a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 182. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 182. In several embodiments, the chimeric receptor comprises the amino acid sequence of SEQ ID NO: 183. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 183.

In several embodiments, there is provided a polynucleotide encoding chimeric antigen receptor that comprises a Flag-tag, humanized anti-CD19moiety and multiple co-stimulatory domains. For example, a schematic architecture is anti-CD19 moiety/transmembrane domain/co-stimulatory domain 1/co-stimulatory domain 2/co-stimulatory domain 3/signaling domain. The co-stimulatory domains vary in order, depending on the embodiment. For example, in several embodiments the co-stimulatory domains (“CSD”) may be positioned as: CSD1/CSD2, CSD2/CSD1, CSD1/CSD2/CSD3, CSD1/CSD2/CSD3, CSD3/CSD2/CSD1, etc. In several embodiments, there is provided a polynucleotide encoding a Flag-tag, humanized anti-CD19moiety/CD8hinge/CD8aTM/CD44/OX40/CD27/CD3zeta chimeric antigen receptor complex (see FIG. 3F, NK19H-13a). The polynucleotide comprises or is composed of an anti-CD19 scFv that has been humanized and comprises a Flag tag, a CD8a hinge, a CD8a transmembrane domain, a CD44 co-stimulatory domain, an OX40 co-stimulatory domain, a CD27 co-stimulatory domain, and a CD3zeta domain. In several embodiments, the chimeric antigen receptor further comprises mbIL15 (see FIG. 3F, NK19H-13b). In such embodiments, the polynucleotide comprises or is composed of an anti-CD19 scFv that has been humanized and comprises a Flag tag, a CD8a hinge, a CD8a transmembrane domain, a CD44 co-stimulatory domain, an OX40 co-stimulatory domain, a CD27 co-stimulatory domain, a CD3zeta domain, a 2A cleavage site, and an mbIL-15 domain as described herein.

In several embodiments, there is provided a polynucleotide encoding a humanized anti-CD19moiety/CD8hinge/CD8TM/OX40/CD3zeta chimeric antigen receptor complex (see FIG. 3G, NK19H-NF-1a). The polynucleotide comprises or is composed of an anti-CD19 scFv that has been humanized and comprises a first humanized light chain and a first humanized heavy chain (L1/H1), a CD8a hinge, a CD8a transmembrane domain, an OX40 signaling domain, and a CD3zeta domain. In several embodiments, the chimeric antigen receptor further comprises mbIL15 (see FIG. 3G, NK19H-NF-1b). In such embodiments, the polynucleotide comprises or is composed of an anti-CD19 scFv that has been humanized and comprises a first humanized light chain and a first humanized heavy chain (L1/H1), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 184. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 184. In several embodiments, the chimeric receptor comprises the amino acid sequence of SEQ ID NO: 185. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 185.

In several embodiments, there is provided a polynucleotide encoding a humanized anti-anti-CD19moiety/CD8hinge/CD8TM/OX40/CD3zeta chimeric antigen receptor complex (see FIG. 3G, NK19H-NF-2a). The polynucleotide comprises or is composed of an anti-CD19 scFv that has been humanized and comprises a second humanized light chain and a first humanized heavy chain (L2/H1), a CD8a hinge, a CD8a transmembrane domain, an OX40 signaling domain, and a CD3zeta domain. In several embodiments, the chimeric antigen receptor further comprises mbIL15 (see FIG. 3G, NK19H-NF-2b). In such embodiments, the polynucleotide comprises or is composed of an anti-CD19 scFv that has been humanized and comprises a second humanized light chain and a first humanized heavy chain (L2/H1), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 186. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 186. In several embodiments, the chimeric receptor comprises the amino acid sequence of SEQ ID NO: 187. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 187.

In several embodiments, there is provided a polynucleotide encoding a humanized anti-anti-CD19moiety/CD8hinge/CD8TM/OX40/CD3zeta chimeric antigen receptor complex (see FIG. 3G, NK19H-NF-3a). The polynucleotide comprises or is composed of an anti-CD19 scFv that has been humanized and comprises a third humanized light chain and a first humanized heavy chain (L3/H1), a CD8a hinge, a CD8a transmembrane domain, an OX40 signaling domain, and a CD3zeta domain. In several embodiments, the chimeric antigen receptor further comprises mbIL15 (see FIG. 3G, NK19H-NF-3b). In such embodiments, the polynucleotide comprises or is composed of an anti-CD19 scFv that has been humanized and comprises a third humanized light chain and a first humanized heavy chain (L3/H1), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 188. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 188. In several embodiments, the chimeric receptor comprises the amino acid sequence of SEQ ID NO: 189. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 189.

In several embodiments, there is provided a polynucleotide encoding a humanized anti-anti-CD19moiety/CD8hinge/CD8TM/OX40/CD3zeta chimeric antigen receptor complex (see FIG. 3G, NK19H-NF-4a). The polynucleotide comprises or is composed of an anti-CD19 scFv that has been humanized and comprises a first humanized light chain and a second humanized heavy chain (L1/H2), a CD8a hinge, a CD8a transmembrane domain, an OX40 signaling domain, and a CD3zeta domain. In several embodiments, the chimeric antigen receptor further comprises mbIL15 (see FIG. 3G, NK19H-NF-4b). In such embodiments, the polynucleotide comprises or is composed of an anti-CD19 scFv that has been humanized and comprises a first humanized light chain and a second humanized heavy chain (L1/H2), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 190. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 190. In several embodiments, the chimeric receptor comprises the amino acid sequence of SEQ ID NO: 191. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 191.

In several embodiments, there is provided a polynucleotide encoding a humanized anti-CD19moiety/CD8hinge/CD8TM/OX40/CD3zeta chimeric antigen receptor complex (see FIG. 3H, NK19H-NF-5a). The polynucleotide comprises or is composed of an anti-CD19 scFv that has been humanized and comprises a second humanized light chain and a second humanized heavy chain (L2/H2), a CD8a hinge, a CD8a transmembrane domain, an OX40 signaling domain, and a CD3zeta domain. In several embodiments, the chimeric antigen receptor further comprises mbIL15 (see FIG. 3H, NK19H-NF-5b). In such embodiments, the polynucleotide comprises or is composed of an anti-CD19 scFv that has been humanized and comprises a second humanized light chain and a second humanized heavy chain (L2/H2), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 192. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 192. In several embodiments, the chimeric receptor comprises the amino acid sequence of SEQ ID NO: 193. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 193.

In several embodiments, there is provided a polynucleotide encoding a humanized anti-CD19moiety/CD8hinge/CD8TM/OX40/CD3zeta chimeric antigen receptor complex (see FIG. 3H, NK19H-NF-6a). The polynucleotide comprises or is composed of an anti-CD19 scFv that has been humanized and comprises a third humanized light chain and a second humanized heavy chain (L3/H2), a CD8a hinge, a CD8a transmembrane domain, an OX40 signaling domain, and a CD3zeta domain. In several embodiments, the chimeric antigen receptor further comprises mbIL15 (see FIG. 3H, NK19H-NF-6b). In such embodiments, the polynucleotide comprises or is composed of an anti-CD19 scFv that has been humanized and comprises a third humanized light chain and a second humanized heavy chain (L3/H2), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 194. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 194. In several embodiments, the chimeric receptor comprises the amino acid sequence of SEQ ID NO: 195. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 195.

In several embodiments, there is provided a polynucleotide encoding a humanized anti-CD19moiety/CD8hinge/CD8TM/OX40/CD3zeta chimeric antigen receptor complex (see FIG. 3G, NK19H-NF-7a). The polynucleotide comprises or is composed of an anti-CD19 scFv that has been humanized and comprises a first humanized light chain and a third humanized heavy chain (L1/H3), a CD8a hinge, a CD8a transmembrane domain, an OX40 signaling domain, and a CD3zeta domain. In several embodiments, the chimeric antigen receptor further comprises mbIL15 (see FIG. 3H, NK19H-NF-7b). In such embodiments, the polynucleotide comprises or is composed of an anti-CD19 scFv that has been humanized and comprises a first humanized light chain and a third humanized heavy chain (L1/H3), a CD8a hinge, a CD8a transmembrane domain, an OX40 signaling domain, a CD3zeta domain, a 2A cleavage site, and an mbIL-15 domain as described herein. In several embodiments, this receptor complex is encoded by a nucleic acid molecule having the sequence of SEQ ID NO: 196. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 196. In several embodiments, the chimeric receptor comprises the amino acid sequence of SEQ ID NO: 197. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 197.

In several embodiments, there is provided a polynucleotide encoding a humanized anti-CD19moiety/CD8hinge/CD8TM/OX40/CD3zeta chimeric antigen receptor complex (see FIG. 3H, NK19H-NF-8a). The polynucleotide comprises or is composed of an anti-CD19 scFv that has been humanized and comprises a second humanized light chain and a third humanized heavy chain (L2/H3), a CD8a hinge, a CD8a transmembrane domain, an OX40 signaling domain, and a CD3zeta domain. In several embodiments, the chimeric antigen receptor further comprises mbIL15 (see FIG. 3H, NKH19-NF-8b). In such embodiments, the polynucleotide comprises or is composed of an anti-CD19 scFv variable light chain that has been humanized and comprises a second humanized light chain and a third humanized heavy chain (L2/H3), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 198. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 198. In several embodiments, the chimeric receptor comprises the amino acid sequence of SEQ ID NO: 199. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 199.

In several embodiments, there is provided a polynucleotide encoding a humanized anti-anti-CD19moiety/CD8hinge/CD8TM/OX40/CD3zeta chimeric antigen receptor complex (see FIG. 3H, NK19H-NF-9a). The polynucleotide comprises or is composed of an anti-CD19 scFv that has been humanized and comprises a third humanized light chain and a third humanized heavy chain (L3/H3), a CD8a hinge, a CD8a transmembrane domain, an OX40 signaling domain, and a CD3zeta domain. In several embodiments, the chimeric antigen receptor further comprises mbIL15 (see FIG. 3H, NKH19-NF-9b). In such embodiments, the polynucleotide comprises or is composed of an anti-CD19 scFv that has been humanized and comprises a third humanized light chain and a third humanized heavy chain (L3/H3), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 200. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 200. In several embodiments, the chimeric receptor comprises the amino acid sequence of SEQ ID NO: 201. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 201.

In several embodiments, there is provided a polynucleotide encoding a humanized anti-CD19moiety/CD8hinge/CD8TM/OX40/CD3zeta chimeric antigen receptor complex (see FIG. 3H, NKH19-NF-10a). The polynucleotide comprises or is composed of an anti-CD19 scFv that has been humanized and comprises a first humanized light chain and a fourth humanized heavy chain (L1/H4), a CD8a hinge, a CD8a transmembrane domain, an OX40 signaling domain, and a CD3zeta domain. In several embodiments, the chimeric antigen receptor further comprises mbIL15 (see FIG. 3H, NK19H-NF-10b). In such embodiments, the polynucleotide comprises or is composed of an anti-CD19 scFv that has been humanized and comprises a first humanized light chain and a fourth humanized heavy chain (L1/H4), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 202. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 202. In several embodiments, the chimeric receptor comprises the amino acid sequence of SEQ ID NO: 203. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 203.

In several embodiments, there is provided a polynucleotide encoding a humanized anti-CD19moiety/CD8hinge/CD8TM/OX40/CD3zeta chimeric antigen receptor complex (see FIG. 3I, NK19H-NF-11a). The polynucleotide comprises or is composed of an anti-CD19 scFv that has been humanized and comprises a second humanized light chain and a fourth humanized heavy chain (L2/H4), a CD8a hinge, a CD8a transmembrane domain, an OX40 signaling domain, and a CD3zeta domain. In several embodiments, the chimeric antigen receptor further comprises mbIL15 (see FIG. 3I, NK19H-NF-11b). In such embodiments, the polynucleotide comprises or is composed of an anti-CD19 scFv that has been humanized, and comprises a second humanized light chain and a fourth humanized heavy chain (L2/H4), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 204. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 204. In several embodiments, the chimeric receptor comprises the amino acid sequence of SEQ ID NO: 205. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 205.

In several embodiments, there is provided a polynucleotide encoding a humanized anti-CD19moiety/CD8hinge/CD8TM/OX40/CD3zeta chimeric antigen receptor complex (see FIG. 3I NK19H-12a). The polynucleotide comprises or is composed of an anti-CD19 scFv that has been humanized and comprises a third humanized light chain and a fourth humanized heavy chain (L3/H4), a CD8a hinge, a CD8a transmembrane domain, an OX40 signaling domain, and a CD3zeta domain. In several embodiments, the chimeric antigen receptor further comprises mbIL15 (see FIG. 3I, NK19H-NF-12b). In such embodiments, the polynucleotide comprises or is composed of an anti-CD19 scFv that has been humanized and comprises a third humanized light chain and a fourth humanized heavy chain (L3/H4), a CD8a hinge, a CD8a transmembrane domain, an OX40 signaling domain, and a CD3zeta domain, a 2A cleavage site, and an mbIL-15 domain as described herein. In several embodiments, this receptor complex is encoded by a nucleic acid molecule having the sequence of SEQ ID NO: 206. In several embodiments, a nucleic acid sequence encoding an NK19 chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 206. In several embodiments, the chimeric receptor comprises the amino acid sequence of SEQ ID NO: 207. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 207.

In several embodiments, there is provided a polynucleotide encoding chimeric antigen receptor that comprises a Flag-tag, humanized anti-CD19moiety and multiple co-stimulatory domains. For example, a schematic architecture is anti-CD19 moiety/transmembrane domain/co-stimulatory domain 1/co-stimulatory domain 2/co-stimulatory domain 3/signaling domain. The co-stimulatory domains vary in order, depending on the embodiment. For example, in several embodiments the co-stimulatory domains (“CSD”) may be positioned as: CSD1/CSD2, CSD2/CSD1, CSD1/CSD2/CSD3, CSD1/CSD2/CSD3, CSD3/CSD2/CSD1, etc. In several embodiments, there is provided a polynucleotide encoding a Flag-tag, humanized anti-CD19moiety/CD8hinge/CD8aTM/CD44/OX40/CD27/CD3zeta chimeric antigen receptor complex (see FIG. 3I, NK19H-NF-13a). The polynucleotide comprises or is composed of an anti-CD19 scFv that has been humanized and comprises a Flag tag, a CD8a hinge, a CD8a transmembrane domain, a CD44 co-stimulatory domain, an OX40 co-stimulatory domain, a CD27 co-stimulatory domain, and a CD3zeta domain. In several embodiments, the chimeric antigen receptor further comprises mbIL15 (see FIG. 3I, NK19H-NF-13b). In such embodiments, the polynucleotide comprises or is composed of an anti-CD19 scFv that has been humanized and comprises a Flag tag, a CD8a hinge, a CD8a transmembrane domain, a CD44 co-stimulatory domain, an OX40 co-stimulatory domain, a CD27 co-stimulatory domain, a CD3zeta domain, a 2A cleavage site, and an mbIL-15 domain as described herein.

In several embodiments, there is provided a polynucleotide encoding chimeric antigen receptor that comprises a Flag-tag, humanized anti-CD19moiety and multiple co-stimulatory domains. For example, a schematic architecture is anti-CD19 moiety/transmembrane domain/co-stimulatory domain 1/co-stimulatory domain 2/co-stimulatory domain 3/signaling domain. The co-stimulatory domains vary in order, depending on the embodiment. For example, in several embodiments the co-stimulatory domains (“CSD”) may be positioned as: CSD1/CSD2, CSD2/CSD1, CSD1/CSD2/CSD3, CSD1/CSD2/CSD3, CSD3/CSD2/CSD1, etc. In several embodiments, there is provided a polynucleotide encoding a Flag-tag, humanized anti-CD19moiety/CD8hinge/CD8aTM/CD44/OX40/CD27/CD3zeta chimeric antigen receptor complex (see FIG. 3F, NK19H-13a). The polynucleotide comprises or is composed of an anti-CD19 scFv that has been humanized and comprises a Flag tag, a CD8a hinge, a CD8a transmembrane domain, a CD44 co-stimulatory domain, an OX40 co-stimulatory domain, a CD27 co-stimulatory domain, and a CD3zeta domain. In several embodiments, the chimeric antigen receptor further comprises mbIL15 (see FIG. 3F, NK19H-13b). In such embodiments, the polynucleotide comprises or is composed of an anti-CD19 scFv that has been humanized and comprises a Flag tag, a CD8a hinge, a CD8a transmembrane domain, a CD44 co-stimulatory domain, an OX40 co-stimulatory domain, a CD27 co-stimulatory domain, a CD3zeta domain, a 2A cleavage site, and an mbIL-15 domain as described herein.

It shall be appreciated that, for any receptor construct described herein, certain sequence variability, extensions, and/or truncations of the disclosed sequences may result when combining sequences, as a result of, for example, ease or efficiency in cloning (e.g., for creation of a restriction site).

### Methods of Treatment

Some embodiments relate to a method of treating, ameliorating, inhibiting, or preventing cancer with a cell or immune cell comprising a chimeric receptor such as a CD19-directed chimeric receptor. In some embodiments, the method includes treating or preventing cancer. In some embodiments, the method includes administering a therapeutically effective amount of immune cells expressing a CD19-directed chimeric receptor as described herein. Examples of types of cancer that may be treated as such are described herein.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Administration can be by a variety of routes, including, without limitation, intravenous, intra-arterial, subcutaneous, intramuscular, intrahepatic, intraperitoneal and/or local delivery to an affected tissue.

### Administration and Dosing

Further provided herein are methods of treating a subject having cancer, comprising administering to the subject a composition comprising immune cells (such as NK and/or T cells) engineered to express a cytotoxic receptor complex as disclosed herein. For example, some embodiments of the compositions and methods described herein relate to use of a CD19-directed chimeric receptor, or use of cells expressing the CD19-directed chimeric receptor, for treating a cancer patient. Uses of such engineered immune cells for treating cancer are also provided.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein.

Administration can be by a variety of routes, including, without limitation, intravenous, intra-arterial, subcutaneous, intramuscular, intrahepatic, intraperitoneal and/or local delivery to an affected tissue. Doses of immune cells such as NK and/or T cells can be readily determined for a given subject based on their body mass, disease type and state, and desired aggressiveness of treatment, but range, depending on the embodiments, from about 10¹ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg. In several embodiments, the dosage ranges from about 2×10⁵ cells/kg to about 2×10⁸ cells/kg, including about 2×10⁶ and 2×10⁷ cells/kg. In several embodiments, a dose is determined by the maximum number of viable engineered cells at the time of dosing. For example, in some embodiments, a single dose comprises a maximum of between about 2×10⁵ and about 2×10⁹ viable engineered cells, including about 2×10⁶, about 2×10⁷, or about 2×10⁸ viable engineered cells. Depending on the embodiment, various types of cancer can be treated. In several embodiments, hepatocellular carcinoma is treated. Additional embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, also provided herein are nucleic acid and amino acid sequences that have sequence identity or homology of at least 80%, 85%, 90%, 95%, 96%, 97%, 98%, 99% (and ranges therein) as compared with the respective nucleic acid or amino acid sequences of SEQ ID NOS. 1-207 (or combinations of two or more of SEQ ID NOS: 1-207) and that also exhibit one or more of the functions as compared with the respective SEQ ID NOS. 1-207 (or combinations of two or more of SEQ ID NOS: 1-207) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes or CD19-directed chimeric receptors are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein.

Doses of immune cells such as NK cells or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of NK cells is administered, for example between about 1×10⁶ cells/kg to about 1×10⁸ cells/kg. Depending on the embodiment, various types of cancer or infection disease can be treated.

### Cancer Types

Some embodiments of the compositions and methods described herein relate to administering immune cells comprising a chimeric receptor, such as a CD19-directed chimeric receptor, to a subject with cancer. Various embodiments provided for herein include treatment or prevention of the following non-limiting examples of cancers. Examples of cancer include, but are not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

### Cancer Targets

Some embodiments of the compositions and methods described herein relate to immune cells comprising a chimeric receptor that targets a cancer antigen. Non-limiting examples of target antigens include: CD5, CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ES0-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMl, PTK7, gpNMB, CDH1-CD324, DLL3, CD276/B7H3, ILl 1Ra, IL13Ra2, CD179b-IGLll, TCRgamma-delta, NKG2D, CD32 (FCGR2A), Tn ag, Timl-/HVCR1, CSF2RA (GM-CSFR-alpha), TGFbetaR2, Lews Ag, TCR-beta1 chain, TCR-beta2 chain, TCR-gamma chain, TCR-delta chain, FITC, Luteinizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVl-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudin 18.2 (CLD18A2 or CLDN18A.2)), P-glycoprotein, STEAPI, Livl, Nectin-4, Cripto, gpA33, BST1/CD157, low conductance chloride channel, and the antigen recognized by TNT antibody.

Additionally, in several embodiments there is provided an immune cell that expresses a CD19-directed chimeric antigen receptor, the chimeric antigen receptor comprising an extracellular anti-CD19 binding moiety, wherein the anti-CD19 binding moiety comprises a heavy chain variable (VH) domain and a and a light chain variable (VL) domain, the VH domain comprising a VH domain having at least 95% identity to the VH domain amino acid sequence set forth in SEQ ID NO: 33, the VL domain having at least 95% identity to the VL domain amino acid sequence set forth in SEQ ID NO: 32, a hinge and/or transmembrane domain, an intracellular signaling domain, wherein the intracellular signaling domain comprises an OX40 subdomain, and wherein the cell also expresses membrane-bound interleukin-15 (mbIL15). In several embodiments, the intracellular signaling domain further comprises a CD3zeta subdomain. In several embodiments, the OX40 subdomain comprises the amino acid sequence of SEQ ID NO: 6 and the CD3zeta subdomain comprises the amino acid sequence of SEQ ID NO: 7. In several embodiments, the hinge domain comprises a CD8a hinge domain. In several embodiments, the CD8a hinge domain, comprises the amino acid sequence of SEQ ID NO: 2. In several embodiments, the mbIL15 comprises the amino acid sequence of SEQ ID NO: 12. In several embodiments, the chimeric receptor further comprises an extracellular domain of an NKG2D receptor. In several embodiments, the extracellular domain of the NKG2D receptor comprises a functional fragment of NKG2D comprising the amino acid sequence of SEQ ID NO: 26. In several embodiments, the immune cell is a natural killer (NK) cell. In several embodiments, the immune cell is a T cell. In several embodiments, the such immune cells are administered to a subject in a method of treating cancer, or are otherwise used to the treatment of cancer, such as in the preparation of a medicament for the treatment of cancer. In several embodiments, the cancer is acute lymphocytic leukemia.

In several embodiments there is provided a polynucleotide encoding a CD19-directed chimeric antigen receptor, the chimeric antigen receptor comprising an extracellular anti-CD19 binding moiety, wherein the anti-CD19 binding moiety comprises a heavy chain variable (VH) domain and a and a light chain variable (VL) domain, the VH domain having at least 95% identity to the VH domain amino acid sequence set forth in SEQ ID NO: 33, the VL domain having at least 95% identity to the VL domain amino acid sequence set forth in SEQ ID NO: 32, a hinge and/or transmembrane domain, an intracellular signaling domain, wherein the intracellular signaling domain comprises an OX40 subdomain, and wherein the polynucleotide also encodes membrane-bound interleukin-15 (mbIL15). In several embodiments, the intracellular signaling domain further comprises a CD3zeta subdomain. In several embodiments, the encoded OX40 subdomain comprises the amino acid sequence of SEQ ID NO: 16 and the encoded CD3zeta subdomain comprises the amino acid sequence of SEQ ID NO: 8. In several embodiments, the hinge domain comprises a CD8a hinge domain and comprises the amino acid sequence of SEQ ID NO: 2. In several embodiments, the encoded mbIL15 comprises the amino acid sequence of SEQ ID NO: 12. In several embodiments, the chimeric receptor further comprises an extracellular domain of an NKG2D receptor. In several embodiments, the encoded extracellular domain of the NKG2D receptor comprises a functional fragment of NKG2D comprising the amino acid sequence of SEQ ID NO: 26.

Also provided herein is an immune cell that expresses a CD19-directed chimeric receptor comprising an extracellular anti-CD19 moiety, a hinge and/or transmembrane domain, and an intracellular signaling domain. In several embodiments, the immune cell is an NK cell. In several embodiments, the immune cell is a T cell. In several embodiments, the hinge domain comprises a CD8a hinge domain or an Ig4 SH domain. In several embodiments, the transmembrane domain comprises a CD8a transmembrane domain, a CD28 transmembrane domain and/or a CD3 transmembrane domain. In several embodiments, the signaling domain comprises an OX40 signaling domain, a 4-1BB signaling domain, a CD28 signaling domain, an NKp80 signaling domain, a CD16 IC signaling domain, a CD3zeta or CD3ζ ITAM signaling domain, and/or a mIL-15 signaling domain. In several embodiments, the signaling domain comprises a 2A cleavage domain. In several embodiments, the mIL-15 signaling domain is separated from the rest or another portion of the CD19-directed chimeric receptor by a 2A cleavage domain. In several embodiments, such immune cells are administered to a subject having cancer in order to treat, inhibit or prevent progression of the cancer.

Provided herein is also an engineered NK or T cell that expresses a CD19-directed chimeric antigen receptor, the chimeric antigen receptor comprising an extracellular anti-CD19 binding moiety, wherein the anti-CD19 binding moiety comprises a heavy chain variable (VH) domain and a and a light chain variable (VL) domain, the VH domain comprising a VH domain resulting from humanization of the VH domain amino acid sequence set forth in SEQ ID NO: 33, the VL domain comprising a VL domain resulting from humanization the VL domain amino acid sequence set forth in SEQ ID NO: 32, a hinge and/or transmembrane domain, an intracellular signaling domain, wherein the intracellular signaling domain comprises an OX40 subdomain, and wherein the cell also expresses membrane-bound interleukin-15 (mbIL15).

Provided herein is a polynucleotide encoding a CD19-directed chimeric antigen receptor, the chimeric antigen receptor comprising an extracellular anti-CD19 binding moiety, wherein the anti-CD19 binding moiety comprises a heavy chain variable (VH) domain and a and a light chain variable (VL) domain, the VH domain comprising a VH domain resulting from humanization of the VH domain amino acid sequence set forth in SEQ ID NO: 33, the VL domain comprising a VL domain resulting from humanization the VL domain amino acid sequence set forth in SEQ ID NO: 32; a hinge and/or transmembrane domain,
an intracellular signaling domain, wherein the intracellular signaling domain comprises an OX40 subdomain, and wherein the polynucleotide also encodes membrane-bound interleukin-15 (mbIL15).

Provided here is a polynucleotide encoding a CD19-directed chimeric antigen receptor, the chimeric antigen receptor comprising an extracellular anti-CD19 binding moiety, wherein the anti-CD19 binding moiety comprises a scFv; a hinge, wherein the hinge is a CD8 alpha hinge; a transmembrane domain; and an intracellular signaling domain, wherein the intracellular signaling domain comprises a CD3 zeta ITAM. In several embodiments, the transmembrane domain comprises a CD8 alpha transmembrane domain, an NKG2D transmembrane domain, and/or a CD28 transmembrane domain. In several embodiments, the intracellular signaling domain comprises a CD28 signaling domain, a 4-1BB signaling domain, and/or an OX40 domain. In several embodiments, the intracellular signaling domain may also comprise a domain selected from ICOS, CD70, CD161, CD40L, CD44, and combinations thereof.

Provided herein is a polynucleotide encoding a CD19-directed chimeric antigen receptor, the chimeric antigen receptor comprising an extracellular anti-CD19 binding moiety, wherein the anti-CD19 binding moiety comprises a variable heavy chain of a scFv or a variable light chain of a scFv; a hinge, wherein the hinge is a CD8 alpha hinge; a transmembrane domain, wherein the transmembrane domain comprises a CD8 alpha transmembrane domain; and an intracellular signaling domain, wherein the intracellular signaling domain comprises a CD3 zeta ITAM. In several embodiments, the polynucleotide also encodes a truncated epidermal growth factor receptor (EGFRt). In several embodiments, the polynucleotide also encodes membrane-bound interleukin-15 (mbIL15). Provided herein is an engineered NK or T cell that expresses such a CD19-directed chimeric antigen, as well as methods of treating cancer by administering such an NK cell or T cell. Also provided for is the use of such polynucleotides in the treatment of cancer, for example, in the manufacture of a medicament for the treatment of cancer.

Provided herein is a polynucleotide encoding a humanized CD19-directed chimeric antigen receptor, the chimeric antigen receptor comprising an extracellular anti-CD19 binding moiety, wherein the anti-CD19 binding moiety comprises a heavy chain variable (VH) domain and a and a light chain variable (VL) domain, the VH domain comprising a VH domain selected from SEQ ID NO: 120, SEQ ID NO: 121, SEQ ID NO: 122, and SEQ ID NO: 123, the VL domain comprising a VL domain selected from SEQ ID NO: 117, SEQ ID NO: 118, and SEQ ID NO: 119; a hinge and/or transmembrane domain, an intracellular signaling domain, and wherein the polynucleotide also encodes membrane-bound interleukin-15 (mbIL15). In several embodiments, the intracellular signaling domain comprises an OX40 subdomain, a CD28 subdomain, an iCOS subdomain, a CD28-41BB subdomain, a CD27 subdomain, a CD44 subdomain, or combinations thereof. In several embodiments, the chimeric antigen receptor comprises a hinge and a transmembrane domain, wherein the hinge is a CD8 alpha hinge, wherein the transmembrane domain is either a CD8 alpha or an NKG2D transmembrane domain. In several embodiments, the intracellular signaling domain comprises a CD3zeta domain.

Provided for herein is a polynucleotide encoding a humanized chimeric antigen receptor (CAR), wherein the CAR comprises a single chain antibody or single chain antibody fragment which comprises a humanized anti-CD19 binding domain, a transmembrane domain, a primary intracellular signaling domain comprising a native intracellular signaling domain of CD3-zeta, or a functional fragment thereof, and a costimulatory domain comprising a native intracellular signaling domain of a protein selected from the group consisting of OX40, CD27, CD28, ICOS, and 4-1BB, or a functional fragment thereof, wherein said anti-CD19 binding domain comprises a light chain complementary determining region 1 (LC CDR1) of SEQ ID NO: 124, 127, or 130, a light chain complementary determining region 2 (LC CDR2) of SEQ ID NO: 125, 128, or 131, and a light chain complementary determining region 3 (LC CDR3) of SEQ ID NO: 126, 129, or 132, and a heavy chain complementary determining region 1 (HC CDR1) of SEQ ID NO: 133, 136, 139, or 142, a heavy chain complementary determining region 2 (HC CDR2) of SEQ ID NO: 134, 137, 140, or 143, and a heavy chain complementary determining region 3 (HC CDR3) of SEQ ID NO: 135, 138, 141, or 144. In several embodiments, the polynucleotide further comprises a region encoding membrane-bound interleukin 15 (mbIL15).

Provided for herein is a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable light (VL) domain of SEQ ID NO: 117, a hinge and/or transmembrane domain, an intracellular signaling domain, and wherein the polynucleotide also encodes membrane-bound interleukin-15 (mbIL15). In several embodiments, the polynucleotide encodes the humanized chimeric antigen receptor of SEQ ID NO: 161, SEQ ID NO: 167, SEQ ID NO: 173, SEQ ID NO: 179, SEQ ID NO: 185, SEQ ID NO: 191, SEQ ID NO: 197, or SEQ ID NO: 203.

Provided for is a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable light (VL) domain of SEQ ID NO: 118, a hinge and/or transmembrane domain, an intracellular signaling domain, and wherein the polynucleotide also encodes membrane-bound interleukin-15 (mbIL15). In several embodiments, the polynucleotide encodes the humanized chimeric antigen receptor of SEQ ID NO: 163, SEQ ID NO: 169, SEQ ID NO: 175, SEQ ID NO: 181, SEQ ID NO: 187, SEQ ID NO: 193, SEQ ID NO: 199, or SEQ ID NO: 205.

Provided for is a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable light (VL) domain of SEQ ID NO: 119, a hinge and/or transmembrane domain, an intracellular signaling domain, and wherein the polynucleotide also encodes membrane-bound interleukin-15 (mbIL15). In several embodiments, the polynucleotide encodes the humanized chimeric antigen receptor of SEQ ID NO: 165, SEQ ID NO: 171, SEQ ID NO: 177, SEQ ID NO: 183, SEQ ID NO: 189, SEQ ID NO: 195, SEQ ID NO: 201, or SEQ ID NO: 207.

Provided for is a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 120, a hinge and/or transmembrane domain, an intracellular signaling domain, and wherein the polynucleotide also encodes membrane-bound interleukin-15 (mbIL15). In several embodiments, the polynucleotide encodes the humanized chimeric antigen receptor of SEQ ID NO: 161, SEQ ID NO: 163, SEQ ID NO: 165, SEQ ID NO: 185, SEQ ID NO: 187, or SEQ ID NO: 189.

Provided for is a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 121, a hinge and/or transmembrane domain, an intracellular signaling domain, and wherein the polynucleotide also encodes membrane-bound interleukin-15 (mbIL15). In several embodiments, the polynucleotide encodes the humanized chimeric antigen receptor of SEQ ID NO: 167, SEQ ID NO: 169, SEQ ID NO: 171, SEQ ID NO: 191, SEQ ID NO: 193, or SEQ ID NO: 195.

Provided for is a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 122, a hinge and/or transmembrane domain, an intracellular signaling domain, and wherein the polynucleotide also encodes membrane-bound interleukin-15 (mbIL15). In several embodiments, the polynucleotide encodes the humanized chimeric antigen receptor of SEQ ID NO: 173, SEQ ID NO: 175, SEQ ID NO: 177, SEQ ID NO: 197, SEQ ID NO: 199, or SEQ ID NO: 201.

Provided for is a polynucleotide encoding a humanized CD19-directed chimeric antigen receptor, the chimeric antigen receptor comprising an extracellular anti-CD19 binding moiety, wherein the anti-CD19 binding moiety comprises a humanized scFv sequence comprising a variable heavy (VH) domain of SEQ ID NO: 123, a hinge and/or transmembrane domain, an intracellular signaling domain, and wherein the polynucleotide also encodes membrane-bound interleukin-15 (mbIL15). In several embodiments, the polynucleotide encodes the humanized chimeric antigen receptor of SEQ ID NO: 179, SEQ ID NO: 181, SEQ ID NO: 183, SEQ ID NO: 203, SEQ ID NO: 205, or SEQ ID NO: 207.

In several embodiments, the provided for polynucleotides do not encode SEQ ID NO: 112, 113, 114, or 116.

Provided for is a polynucleotide encoding a humanized CD19-directed chimeric antigen receptor, the chimeric antigen receptor comprising an extracellular anti-CD19 binding moiety; a hinge and/or transmembrane domain; an intracellular signaling domain, and wherein the polynucleotide also encodes membrane-bound interleukin-15 (mbIL15), and wherein the polynucleotide is selected from the group consisting of polynucleotides having at least 95% identity to SEQ ID NO: 184, SEQ ID NO: 186, SEQ ID NO: 192, or SEQ ID NO: 200. In several embodiments, the polynucleotide has the sequence of SEQ ID NO: 184, SEQ ID NO: 186, SEQ ID NO: 192, or SEQ ID NO: 200. In several embodiments, there are provided engineered NK or T cells that express such a humanized CD19-directed chimeric antigen receptor. Also provided for is a method of treating cancer in a subject comprising administering to a subject having cancer such engineered NK or T cells. Also provided is the use of such polynucleotides in the treatment of cancer, such as in the manufacture of a medicament for the treatment of cancer.

### EXAMPLES

The materials and methods disclosed herein are non-limiting examples that are employed according to certain embodiments disclosed herein.

According to several embodiments, NK cells are isolated from peripheral blood mononuclear cells and expanded through the use of a feeder cell line. As discussed in more detail below, in several embodiments, the feeder cells are engineered to express certain stimulatory molecules (e.g. interleukins, CD3, 4-1BBL, etc.) to promote immune cell expansion and activation. Engineered feeder cells are disclosed in, for example, International Patent Application PCT/SG2018/050138, which is incorporated in its entirety by reference herein. In several embodiments, the stimulatory molecules, such as interleukin 12, 18, and/or 21 are separately added to the co-culture media, for example at defined times and in particular amounts, to effect an enhanced expansion of a desired sub-population(s) of immune cells.

NK cells isolated from PBMC were cocultured with K562 cells expressing membrane-bound IL15 and 4-1BBL, with the media being supplemented with IL2. For one group of engineered NK cells, they were expanded in media was supplemented (at Day 0) with a combination of soluble IL12 and soluble IL18. The media was refreshed with additional soluble IL12 and soluble IL18 at Day 4. Additional details on embodiments of such culture methodology is disclosed in U.S. Provisional Patent Application No. 62/881,311, filed Jul. 31, 2019 and incorporated in its entirety by reference here. Viral transduction, with a CD19-directed chimeric receptor construct, was performed at Day 7. The resultant engineered NK cells were evaluated at 14, or more, days of total culture time.

### Example 1

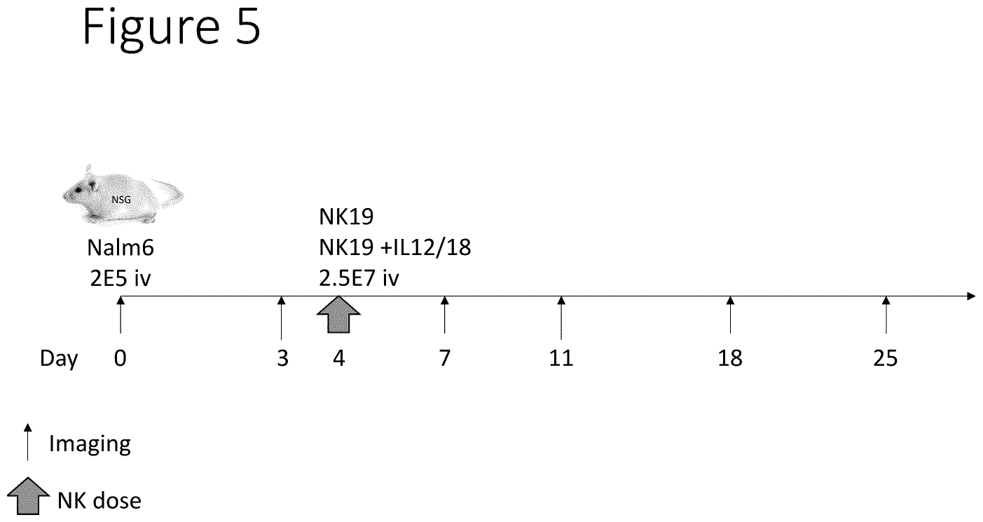

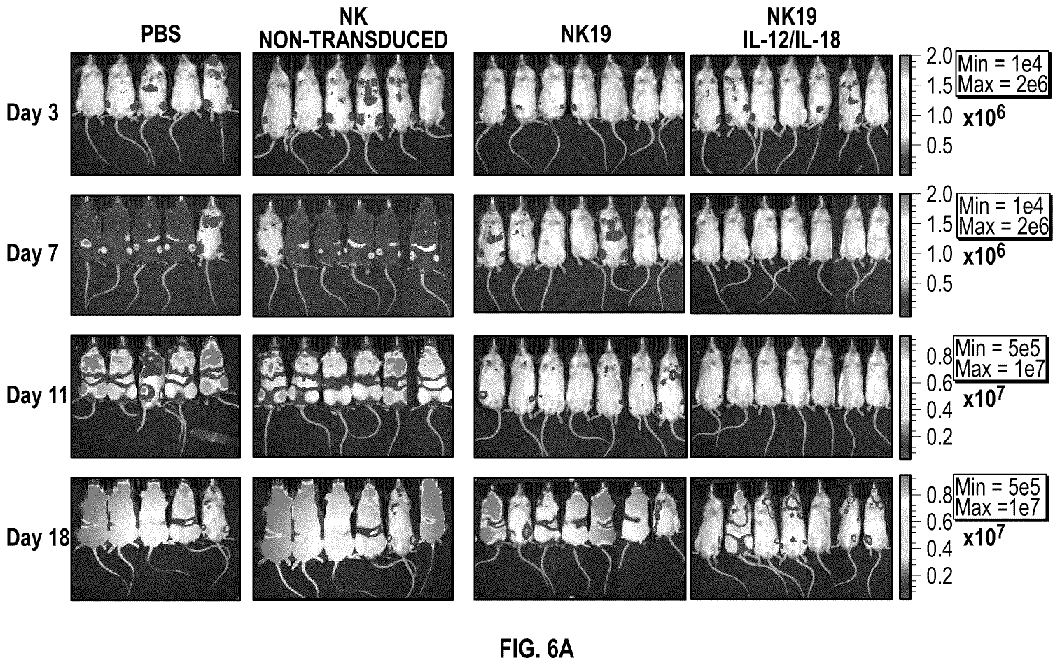

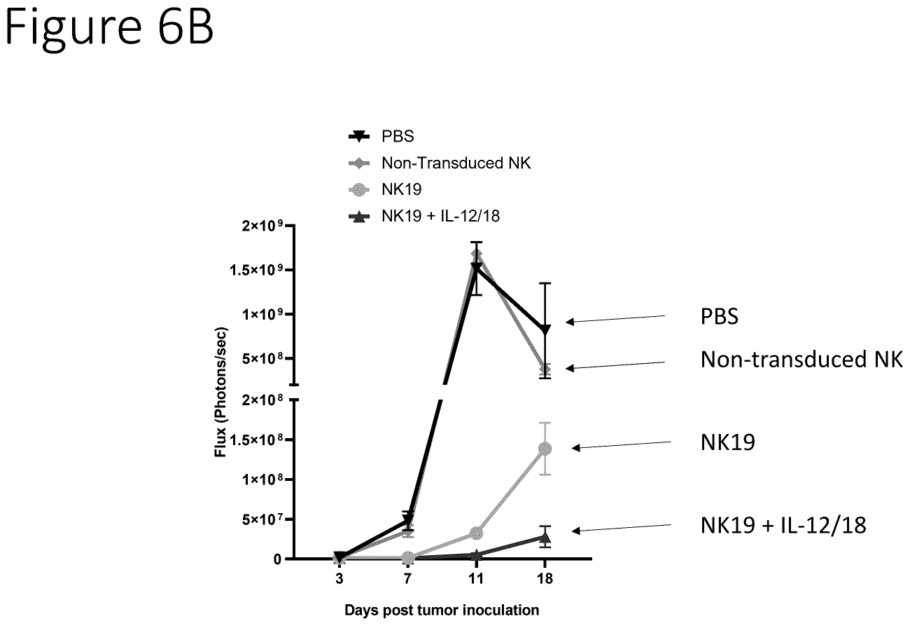

FIG. 5 depicts a schematic experimental model for evaluating the anti-tumor efficacy of engineered NK cells generated according to methods disclosed herein. NOD-scid IL2Rgamma^{null} mice were administered 2×10′ Nalm6 cells (B cell precursor leukemia cell line) intravenously at Day 0. At Day 4, one group of mice received 2.7×10⁷ NK cells expressing an NK19 CAR (see FIG. 3A, though it shall be appreciated that other CD19-directed chimeric receptors could be used) while another group received NK cells that had been expanded using soluble IL12/IL18 (identified as NK19-IL12/18 cells). Leukemic tumor burden was assessed by fluorescent imaging performed at Days 3, 7, 11, 18, and 25. FIG. 6A shows the imaging results from Days 3, 7, 11, and 18. As can be seen, mice receiving either NK19 or NK19 IL12/18 had significantly less tumor burden than mice receiving either non-transduced NK cells or PBS as a control. FIG. 6B shows a summary of the imaging data in a line graph (greater values of Flux (photons/second) indicates more fluorescent signal detection and greater tumor burden). Both NK19 and NK19 IL12/18 groups have less tumor burden as early as Day 7 post-injection of the Nalm6 leukemia cells. This difference is even more pronounced at Day 11, when the PBS and NT NK cell groups are exhibiting large amounts of tumor growth. Even as far out as Day 18, when tumor burden in the PBS and NT NK groups is extensive, the NK19 and NK19 IL12/18 groups show much less tumor burden. Unexpectedly, the NK19 IL12/18 groups show less tumor burden than this receiving the NK19 construct. This is surprising not only because NK19 cells are quite efficacious at preventing leukemia cell growth, so the further enhancement of this effect is unexpected, but also because an upstream methodology of expanding the cells has not only impacted the cell number itself, but also the activity level of those expanded cells.

### Example 2

Experiments were undertaken to determine if a given stimulatory domain (also referred to as co-stimulatory domains, given that many construct employ multiple “signaling” domains in tandem, triplet or other multiplex fashion) utilized in a CAR impacted expression (as well as activity). NK cells were generated by transduction with viruses encoding various CARs depicted in FIGS. 3A-3C (though other constructs are used, in several embodiments). By way of non-limiting example, NK cells were cells were generated by transduction with a bicistronic virus encoding an anti-CD19 scFv, an intracellular OX40 costimulatory domain, CD3ζ signaling domain, and membrane-bound IL-15 (see NK19, FIG. 3A) which supports prolonged cell survival and proliferation. The other CAR constructs tested, NK19-1, 2, 3, 4, 5, 8, 9, 10, 11, 12, and NK19-13, were transduced into NK cells in similar fashion. It shall be appreciated that, for those constructs that employ a Flag domain to determine expression, analogous constructs not including the Flag (or other tag domain) are provided for, in several embodiments.

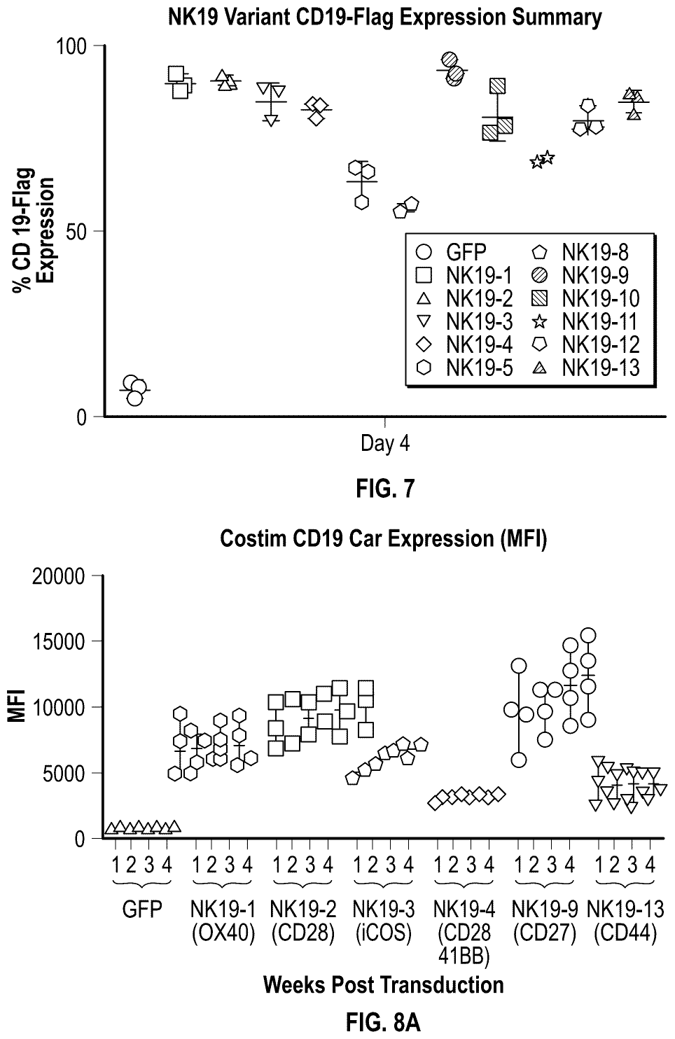

FIG. 7 summarizes the expression data of NK 19-1 to NK5 and NK8 to NK19-13 as evidenced by detection of CD19Flag. Data are presented as percentage of NK cells expressing CD19-Flag relative to the total number of NK cells presented. The data were collected at 4 days after transduction with the relevant virus encoding the NK19-“X” CAR. As evidenced by the expression data, all constructs were expressed by at least 55% of the NK cells. In fact, for eight of the eleven constructs for which data was generated expression was detected in approximately 75% or more of the total number of NK cells, with several constructs expressed at over 80% efficiency. This expression data indicates that, according to several embodiments, selection of specific stimulatory domains can enable a more efficient expression of the CAR by the NK cell. This is advantageous, in several embodiments, because a greater portion of a given NK cell preparation is useful clinically (e.g., fewer input NK cells needed to generate a clinically relevant engineered NK cell dose).

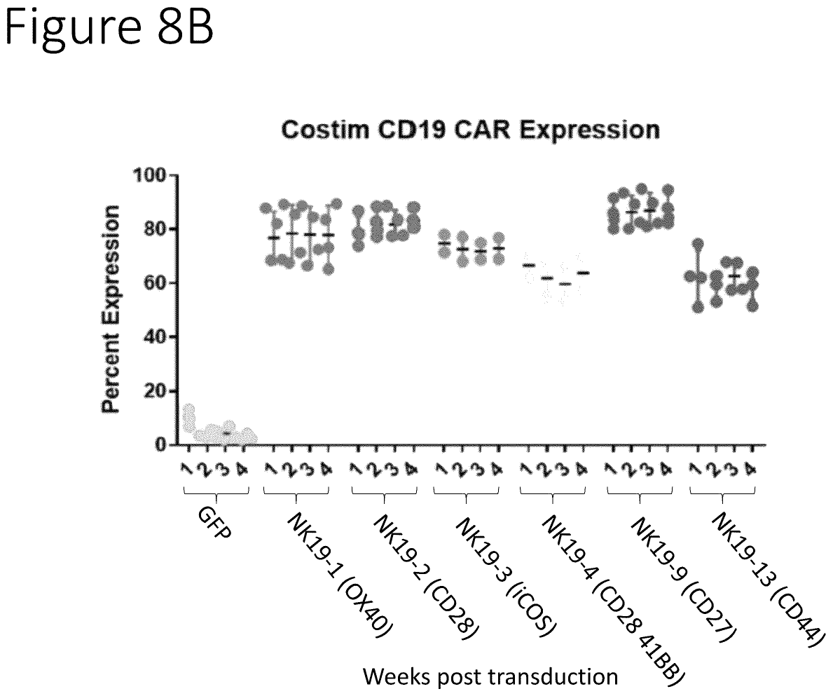

As discussed herein, various co-stimulatory domains can be employed in chimeric antigen receptors that Target CD19 (or other tumor markers). FIG. 8A depicts data related to the expression of CARs targeting CD19 using various co-stimulatory domains. By way of non-limiting example, co-stimulatory domains can include, but are not limited to, OX40, CD28, iCOS, CD28/41BB, CS27, and CD44). FIG. 8A shows mean fluorescence intensity data representing expression of the indicated CAR constructs by NK cells. As evidenced by the low MFI detected for the GFP control, these data indicate that these construct (a) are expressed by NK cells, and (b) are expressed relatively stably by NK cells over a 4 week period post-transduction. FIG. 8B shows the efficiency of expression of the CARs with the indicated co-stimulatory domains. While there is some variability in efficiency of expression, ranging from about 60% to about 80% efficiency, each of the CARs with the indicated co-stimulatory domains expressed well, and also expressed relatively consistently over at least 4 weeks.

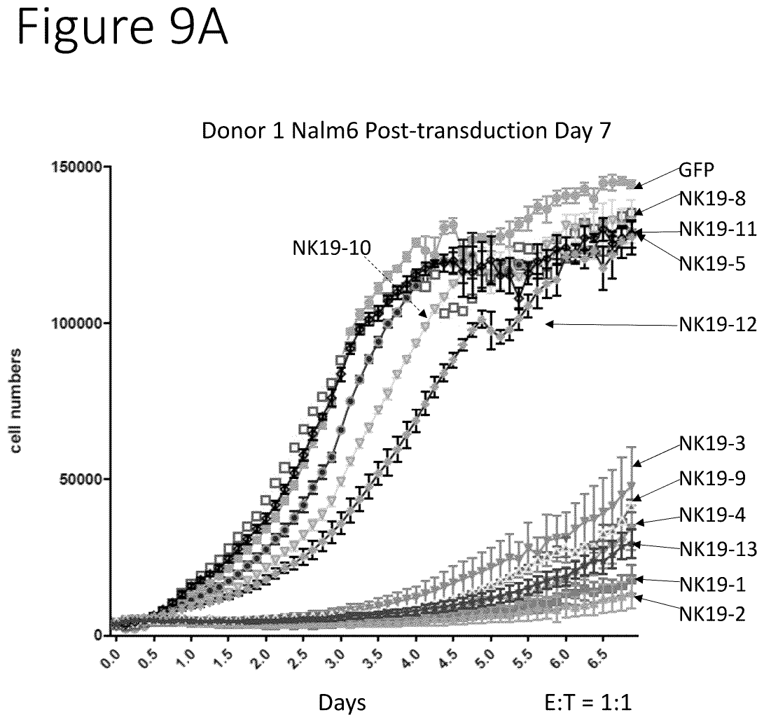

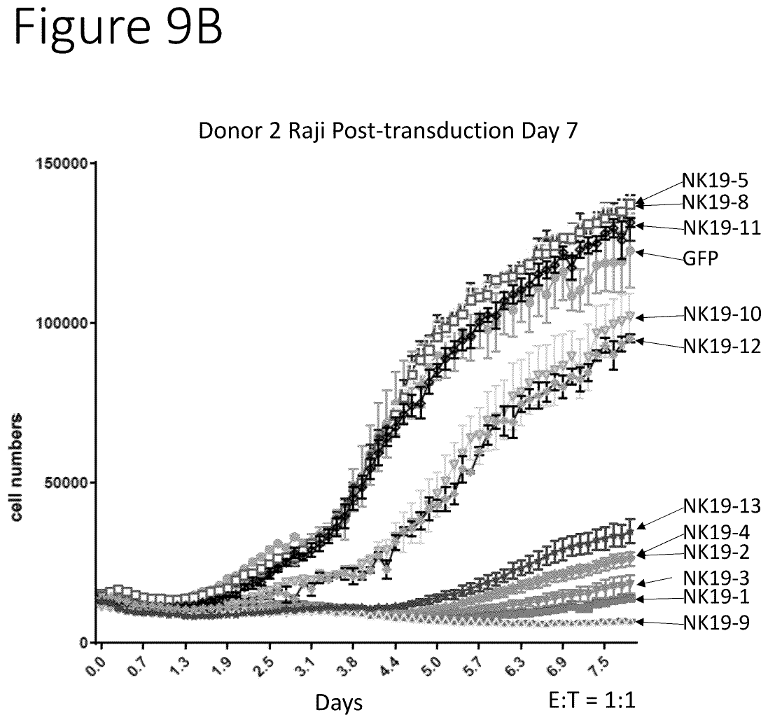

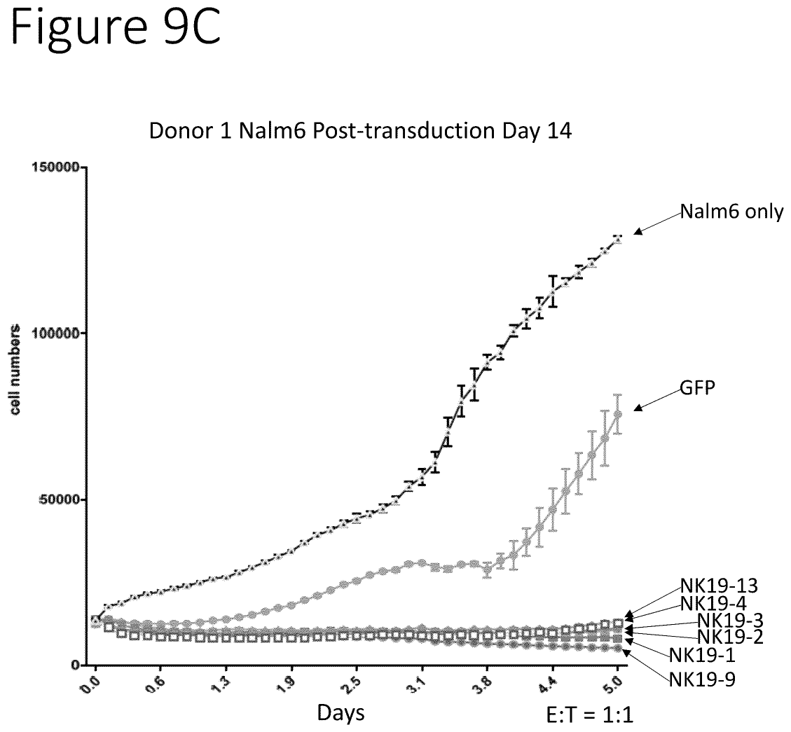

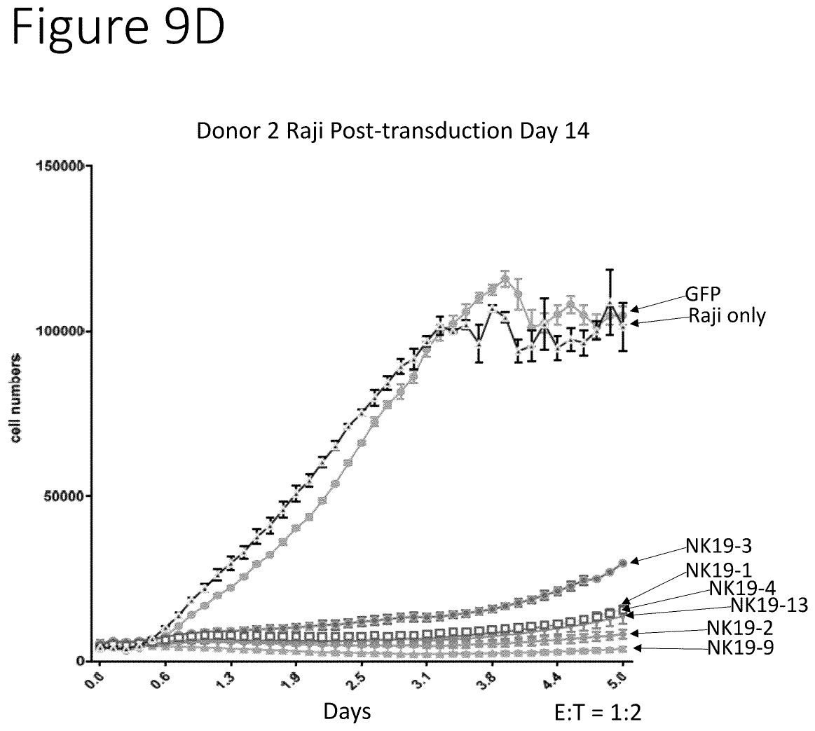

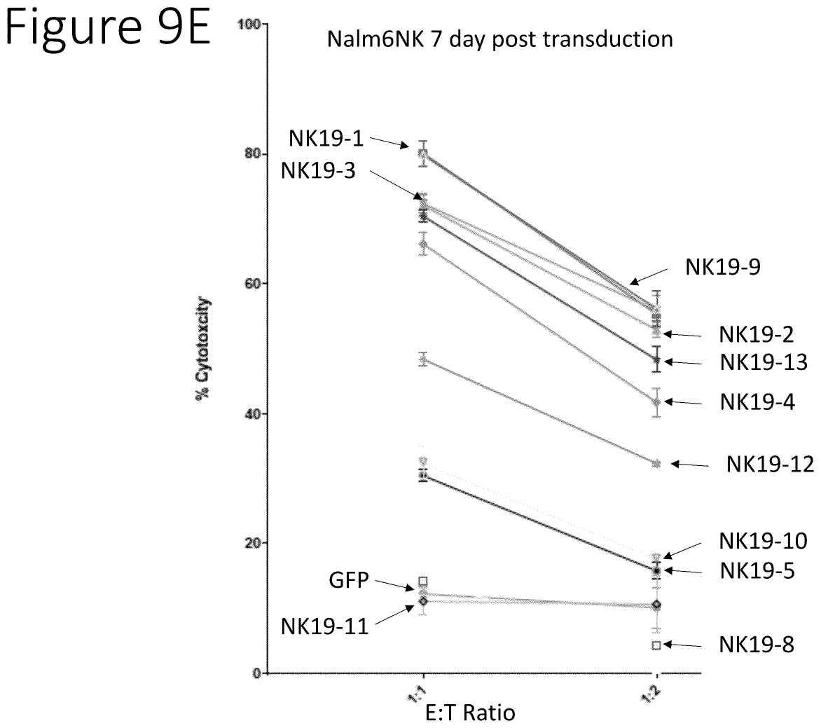

With respect to the cytotoxic efficacy of the NK cells expressing the CD19-directed CARs utilizing the various co-stimulatory domains, that data is shown in FIGS. 9A-9F. Cultured Nalm6 or Raji cells were exposed to engineered NK cells expressing the indicated NK19 constructs and co-cultured for the indicated number of days (X axis represents days) of exposure to NK19-X-expressing NK cells. FIG. 9A shows the cytotoxic effects of the indicated constructs against Nalm6 cells 7 days after NK cells from a first donor were transduced with the indicated construct. The effector cell to target cell ratio for this experiment was 1:1. As shown in the traces, each of NK19-10, NK19-8, NK-1911, NK19-5, and NK19-12 allowed increases in the number of Nalm6 cells detected, on par with that of NK cells expressing only GFP as a control. However, each of NK19-3, NK19-9, NK19-4, NK19-13, NK19-1 and NK19-2 showed significantly less increase in Nalm6 cell number (e.g., greater cytotoxicity). In several embodiments, such constructs are therefore expressed in NK cells and used in treating B cell leukemia (or other tumor types). In several embodiments, one or more of the stimulatory domains from one of the constructs is engineered into another construct with a different stimulatory domain, which advantageously results in synergistic signaling and further enhanced cytotoxicity. By way of non-limiting example, an NK19-1 construct with an OX40 stimulatory domain is, in several embodiments, further engineered to also express a CD44 stimulatory domain in addition to OX40. By way of further non-limiting example, an NK19-1 construct with an OX40 stimulatory domain is, in several embodiments, further engineered to also express a CD44 stimulatory domain and a CD17 stimulatory domain in addition to OX40.

FIG. 9B shows corresponding cytotoxicity data for engineered NK cells from a second donor against Raji B-cell leukemia cells, 7 days post-transduction. The effector cell to target cell ratio here was also 1:1. As shown, similar to the engineered constructs activity against Nalm6 cells, several constructs allowed for increased Raji cell count. However, each of NK19-13, NK 19-4, NK19-2, NK19-3, NK19-1, and NK19-9 prevented Raji cell growth to a substantial degree, with several of the constructs resulting in nearly no Raji cell growth. In several embodiments, such constructs are therefore expressed in NK cells and used in treating B cell leukemia (or other tumor types).

FIG. 9C shows data for NK cells from Donor 1 against Nalm6 cells 14 days post-transduction. The effector to target cell ratio is 1:1. As shown, even at two weeks post-transduction, the NK19-13, NK19-4, NK19-3, NK19-2, NK19-1, and NK19-9 expressing NK cells prevented virtually all Nalm6 cell growth, indicative of their highly cytotoxic effect against tumor cells. In several embodiments, such constructs are therefore expressed in NK cells and used in treating B cell leukemia (or other tumor types). As discussed above, in several embodiments, a construct is generated that employs a combination two, three, or more of the stimulatory domains, resulting in a synergistic NK cell stimulation and enhanced cytotoxicity.

FIG. 9D shows data for NK cells from Donor 2 against Raji cells at 14 days post-transduction. The effector cell to target cell ratio was 1:2. As shown, NK cells expressing GFP only allowed growth of Raji cells essentially the same as untreated Raji cells. In contrast, each of NK19-3, NK19-1, NK19-4, NK19-13, NK19-2, and NK19-9 expressing NK cells significantly retarded the growth of Raji cells, with several of the constructs allowing little to no Raji cell growth. In several embodiments, such constructs are therefore expressed in NK cells and used in treating B cell leukemia (or other tumor types). As discussed above, in several embodiments, a construct is generated that employs a combination two, three, or more of the stimulatory domains, resulting in a synergistic NK cell stimulation and enhanced cytotoxicity.

FIG. 9E shows summary data for cytotoxicity against Nalm6 cells at an E:T ratio of 1:1 and 1:2 at 7 days post-transduction. At an E:T ratio of 1:2, all but one of the NK19 constructs was equivalent to, or more, cytotoxic than NK cells expressing GFP alone. In fact, even while at a 1:2 ratio, six of the constructs achieved ˜50% or greater cytotoxicity. When tested at an E:T of 1:1, seven of the constructs achieved ˜50% or greater cytotoxicity, however, five of the constructs (NK19-13, NK19-2, NK19-9, NK19-3, and NK19-1) yielded cytotoxicity exceeding 70%. FIG. 9F shows the corresponding data for Raji cells. All constructs tested showed enhanced cytotoxicity over GFP-expressing NK cells at both 1:2 and 1:1 E:T ratios. At an E:T of 1:2, four of the constructs exceeded 40% cytotoxicity, while at 1:1, seven constructs exceeded that kill rate. Furthermore, at a 1:1 E:T, three constructs yielded cytotoxicity of 60% or more, with the most efficacious construct achieving nearly 90% cytotoxicity. Taken together, these data demonstrated that various CD19-directed CAR constructs can not only be expressed, but are stably expressed, and are also effective an inducing cytotoxicity in multiple cancer cell types, in some cases exceeding an 80% kill rate. According to additional embodiments, CD19-targeting constructs are generated that employ combinations of two, three or more stimulatory domains, which result in further enhancements in the cytotoxicity of the NK cells expressing them. In several embodiments, CD19-directed constructs can synergistically interact with NK cells expressing receptors directed against other tumor markers, such as ligands of NKG2D (such chimeric receptor bearing NK cells are described in PCT/US2018/024650, which is incorporated by reference herein in its entirety). For example, a chimeric receptor comprising an binding domain that binds ligands of NKG2D, an OX40 stimulatory domain, and a CD3zeta signaling domain could be used in conjunction with any of the CD19-targeting constructs disclosed herein. In several embodiments, such a chimeric receptor is at least 90% identical in sequence to the nucleic acid sequence of SEQ ID NO: 145. In several embodiments, such a chimeric receptor is at least 90% identical in sequence to the amino acid sequence of SEQ ID NO: 146.

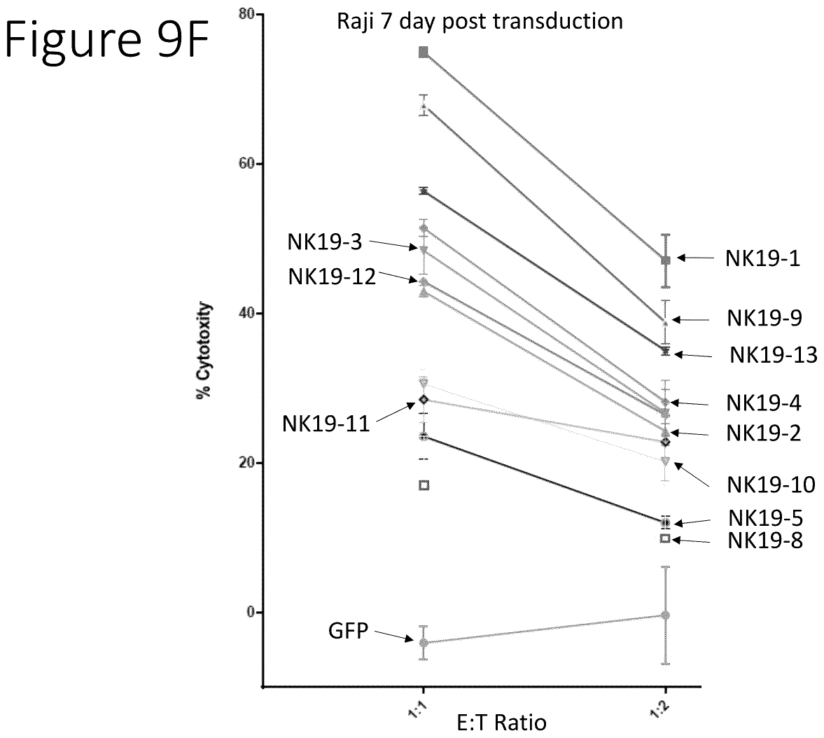

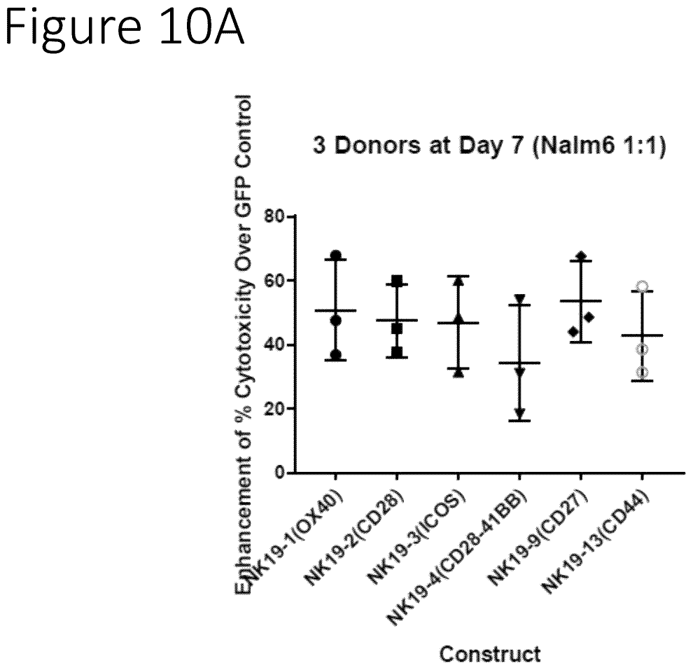

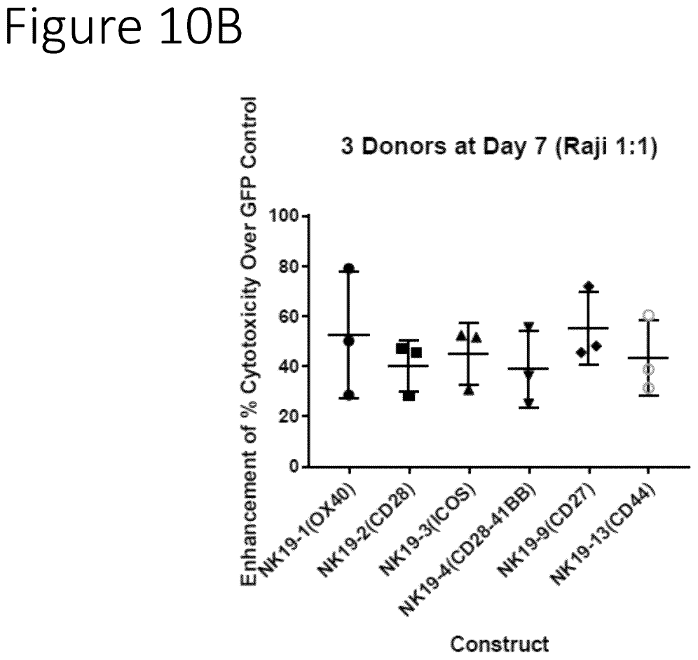

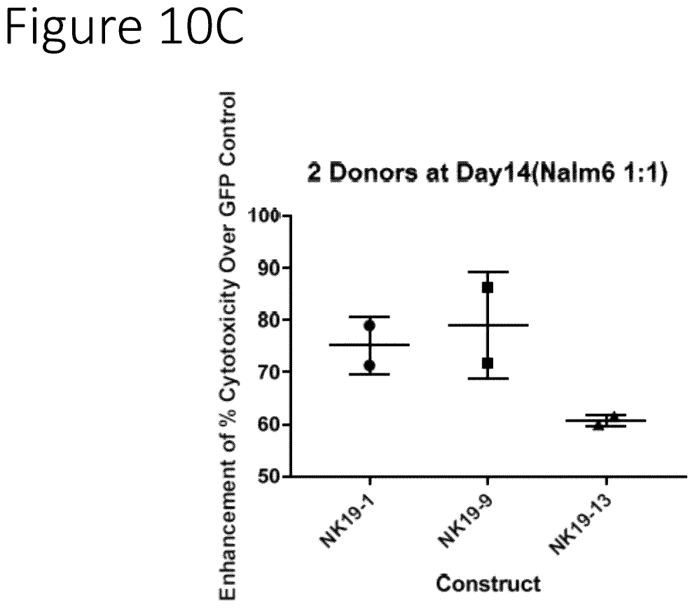

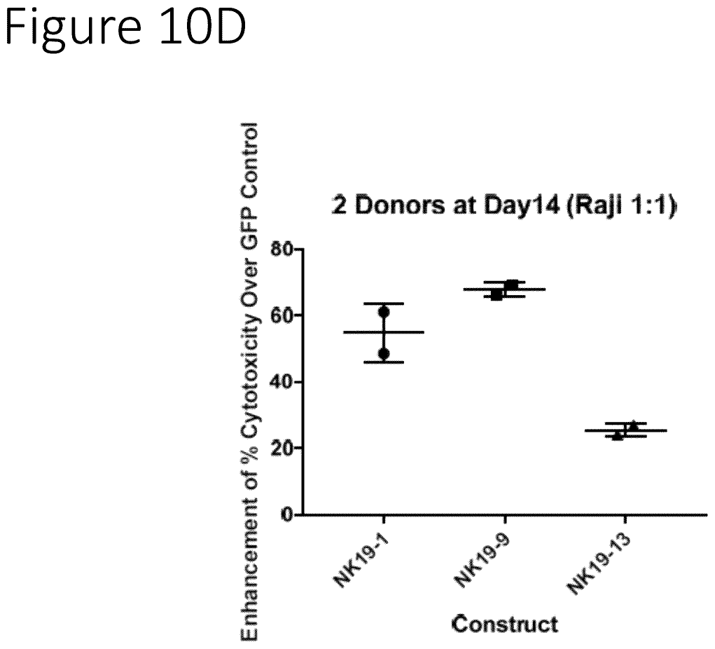

Further experiments were performed to evaluate the cytotoxicity of selected CD19-directed CAR constructs. NK cells isolated from three different donors (two donors for 14 day experiment) were transduced with vectors encoding the indicated constructs, NK19-1 (OX40 co-stimulatory domain); NK19-2 (CD28 co-stimulatory domain); NK19-3 (ICOS co-stimulatory domain); NK19-4 (CD28-41BB co-stimulatory domain); NK19-9 (CD27 co-stimulatory domain); and NK19-13 (CD44 co-stimulatory domain). Seven (n=3) or 14 (n=2) days after transduction, those engineered NK cells were co-cultured with Nalm6 or Raji cells at an E:T ratio of 1:1. Results are shown in FIG. 10A (expressed as percent enhanced cytotoxicity over NK cells expressing GFP). Consistent with the data from FIG. 9, each of the constructs tested yielded enhanced cytotoxic effects against Nalm6 cells, ranging from a mean 40% increase with NK19-4 to an overall average of about 50% increase with the other 5 constructs. FIG. 10B shows the corresponding data against Raji cells. Again, each construct outperformed NK cells expressing only GFP, with mean increases in cytotoxicity over GFP NK cells ranging from about 40% increase to about 50% increase. Using cells from two donors at 14 days post-transduction, NK19-1, NK19-9 and NK19-13 were tested on Nalm6 and Raji cells. FIG. 10C shows the calculated enhanced cytotoxicity of these constructs over GFP-expressing NK cells, where average increases of nearly 80% were seen with NK19-9 expressing NK cells, about 75% with NK19-1 expressing cells and over 60% with NK19-13 expressing cells. Similar results were obtained against Raji cells—NK19-13 expressing cells showed over a 20% improvement in cytotoxicity, NK19-1 expressing cells exhibiting nearly 60% enhanced activity and NK19-9 expressing cells showing almost 70% more cytotoxic activity against Raji cells. These results further support the embodiments disclosed herein wherein engineered NK cells expressing CD19-directed CARs are provided, as are methods for their use in treating cancer immunotherapy results in enhanced cytotoxicity against target tumor cells.

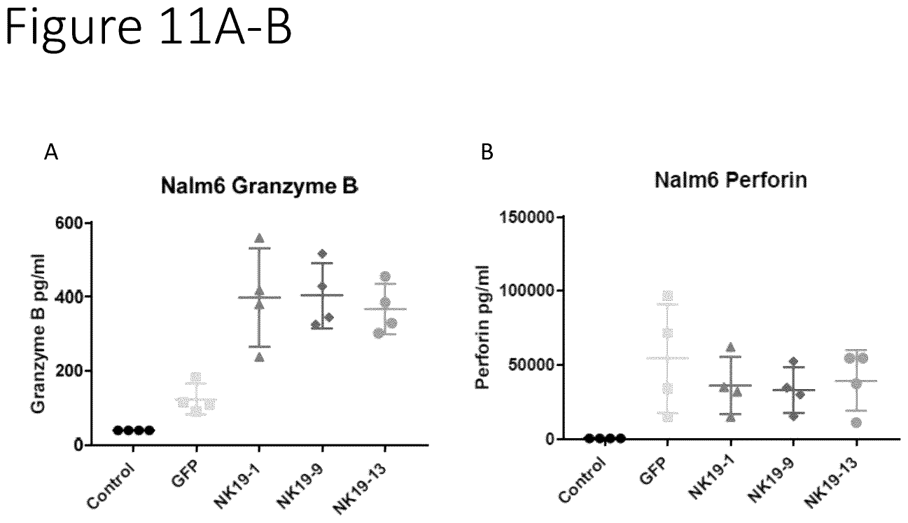

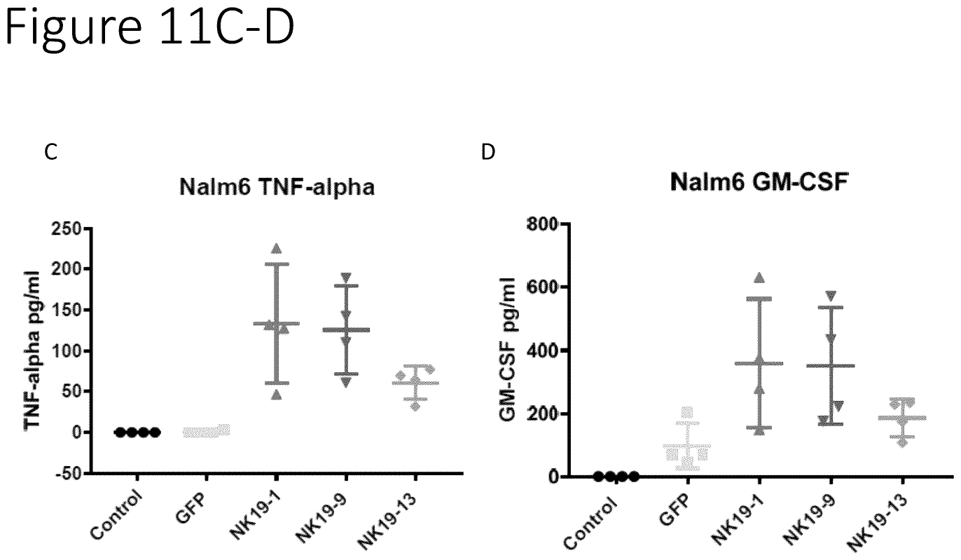

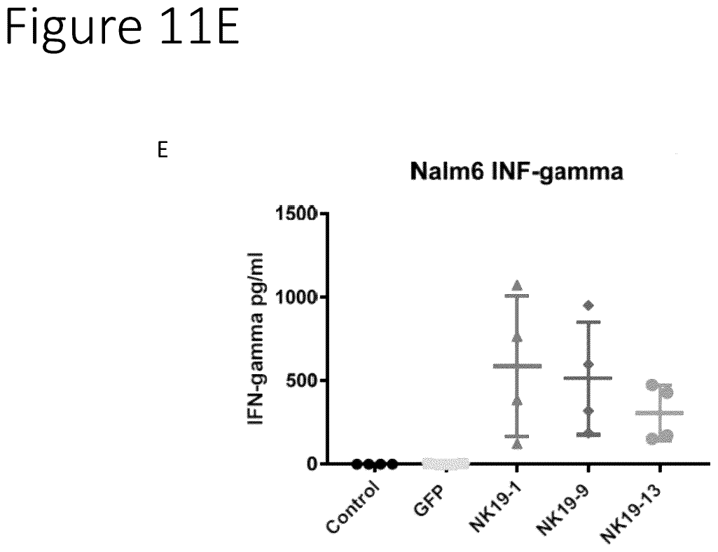

FIGS. 11A-11E depict data related to the cytokine release profiles from NK cells when cultured the Nalm6 cells, which ties into the mechanism by which NK cells control tumor and virus-infected cells—through releasing cytotoxic granules and proinflammatory cytokines. FIG. 11A shows that as compared to control, or even GFP-expressing NK cells, NK cells expressing NK19-1, NK19-9, or NK19-13 all express greater concentrations of the serine protease, Granzyme B, that is present in the granules released by NK cells. NK cells expressing these CD19-directed CARs released approximately 4 times more Granzyme B than control GFP-expressing NK cells. FIG. 11B shows data related to increased release of perforin by the engineered NK cells. Interestingly, perforin levels were not substantially elevated over the concentrations resulting from GFP-expressing NK cells (though perforin concentration was elevated over control. Perforins work in concert with Granzyme B (and other granzymes), with perforins functioning to generate pores through a cell membrane to allow granzymes to cross the membrane, then exert their protease effects on intracellular protein targets. The data raise the possibility that the perforin release while approximately the same, or reduced in connection with certain constructs, are actually more efficient at pore-formation, thus allowing the same degree of pore formation. Alternatively, if the perforins released from NK19 expressing NK cells are no more efficient at pore formation, this is offset by the elevated increase of granzyme B (and/or other granzymes). Thus, enhanced cytotoxicity is still achieved.

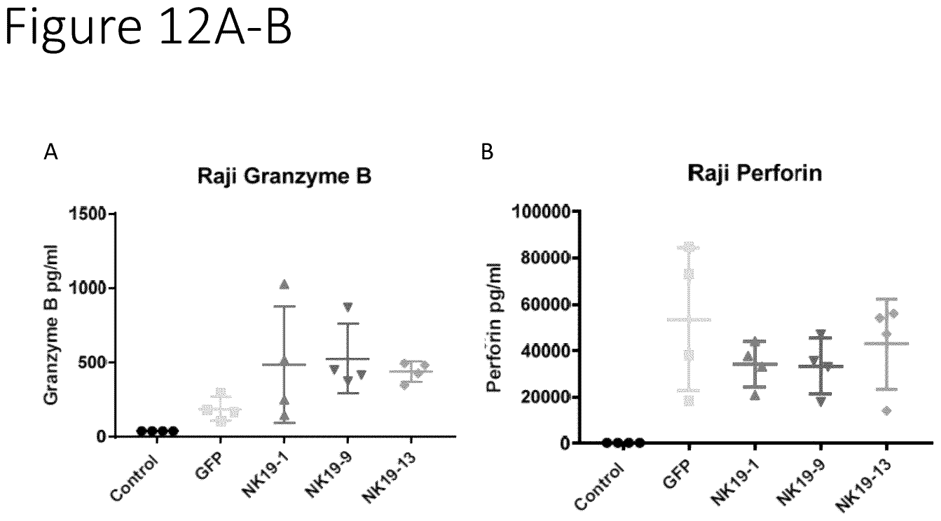

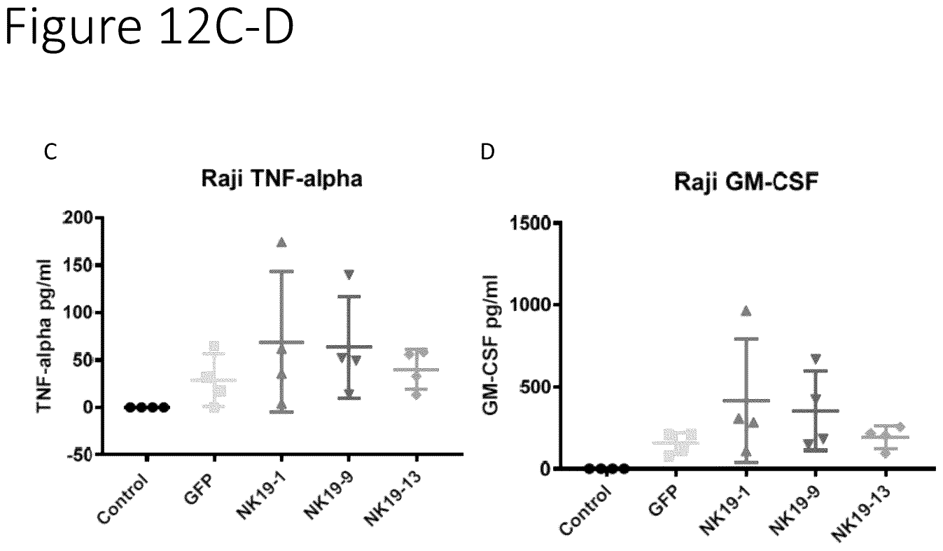

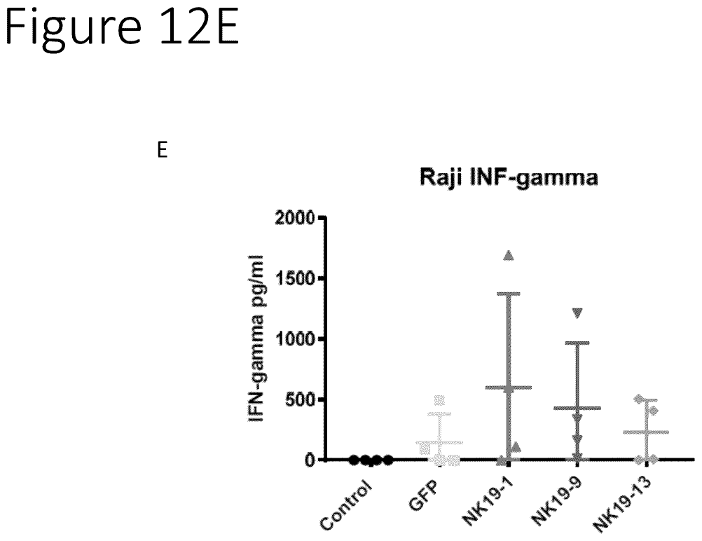

FIG. 11C shows that as compared to control, or even GFP-expressing NK cells, NK cells expressing NK19-1, NK19-9, or NK19-13 all release greater concentrations of the inflammatory cytokine TNF alpha. FIG. 11D shows similar data for GM-CSF release by NK19-expressing NK cells and FIG. 11E shows similar data for interferon gamma release. Likewise, when tested on Raji cells, similar patterns of release result, as shown in FIGS. 12A-12E. These data indicate that the NK19-expressing cells exert their cytotoxic effects, at least in part, through the increased release of inflammatory cytokines and/or cytotoxic granules. As mentioned above, in several embodiments, the engineered CARs are designed to have combinations of two, three or more co-stimulatory domains, with synergistically increased cytokine/granule release and cytotoxicity against target cancers.

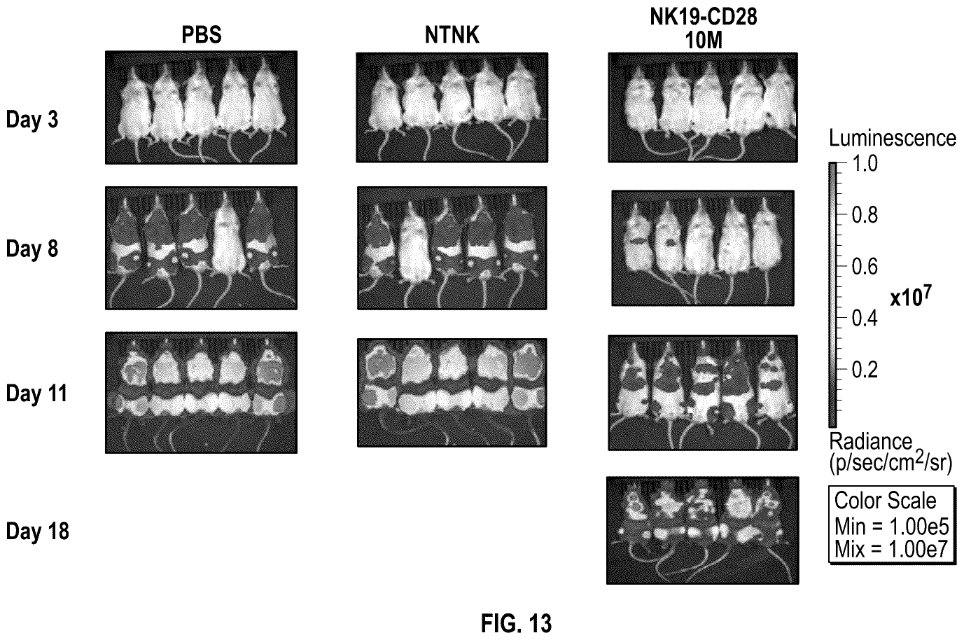

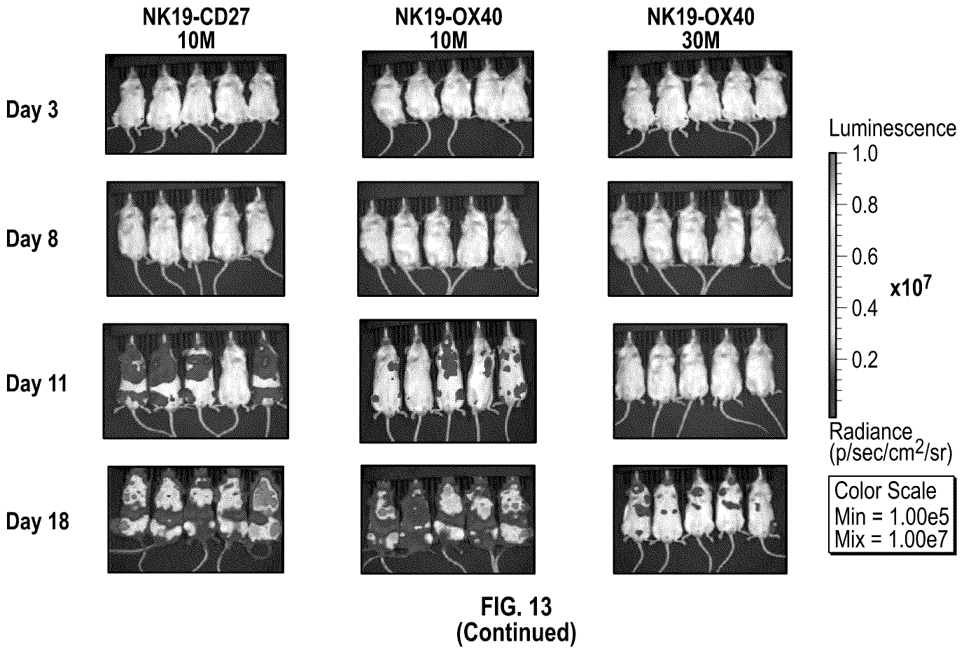

Further evidencing the enhanced effects of NK19 constructs on tumor progression, NSG mice were injected on Day 0 with 1×10⁵ Nalm6 cells (expressing a fluorescent reporter) intravenously. At Day 3, mice received either a PBS control injection, non-transduced NK cells (“NTNK”, 10M), NK19-2 expressing NK cells (10 million cells), NK19-9 expressing cells (10 million), NK19-1 expressing cells (10 million, or NK19-1 expressing cells (30 million). Fluorescent imaging to detect the Nalm6 cells was performed on Days 3, 8, 11, 18, and 25 (imaging data not shown). The data is shown in FIG. 13. As shown, the injection of NK cells expressing any NK19 variant resulted in a reduced progression of Nalm6 growth. NK19-2 expressing NK cells showed minor Nalm6 growth on Day 8, with more Nalm6 growth by Day 11, and substantial growth by Day 18 (though less than with NTNK cells). Neither NK19-9 or NK19-1 (at either dose) expressing NK cells showed detectable tumor burden on Day 8 by imaging. Mice receiving NK19-9 expressing NK cells did show some increase in Nalm6 cell growth by Day 11, with further progression by Day 18. At Day 11, mice receiving either dose of NK19-1 expressing cells did not exhibit Nalm6 cell growth. By day 18, mice receiving 10 million NK19-9 cells showed some tumor growth. However, with a 30 million cell dose, those mice receiving NK19-9 expressing NK cells showed only minor amounts of Nalm6 cell growth.

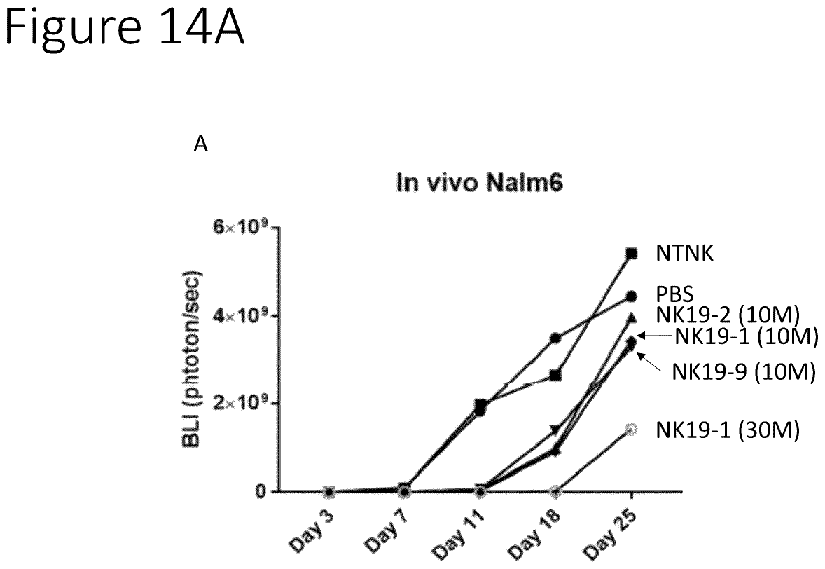

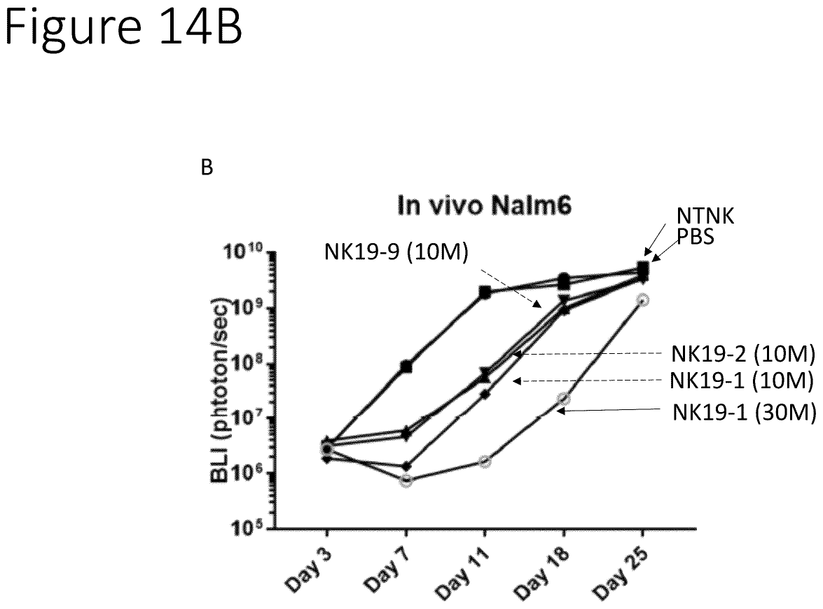

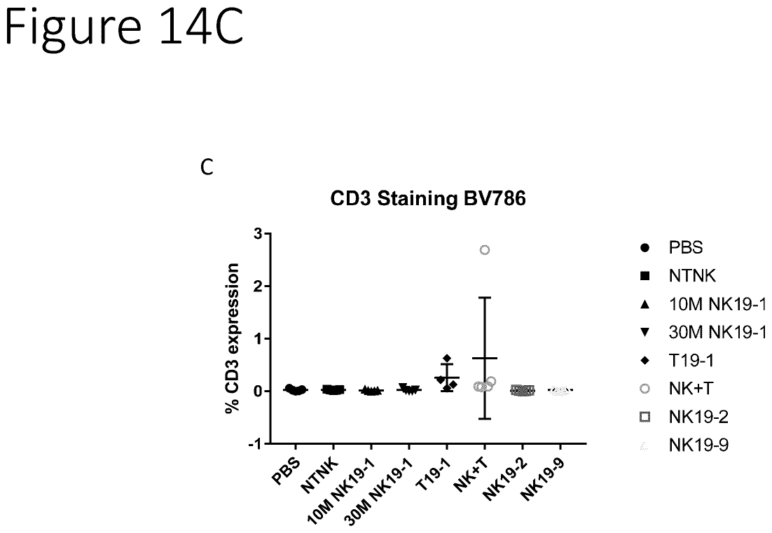

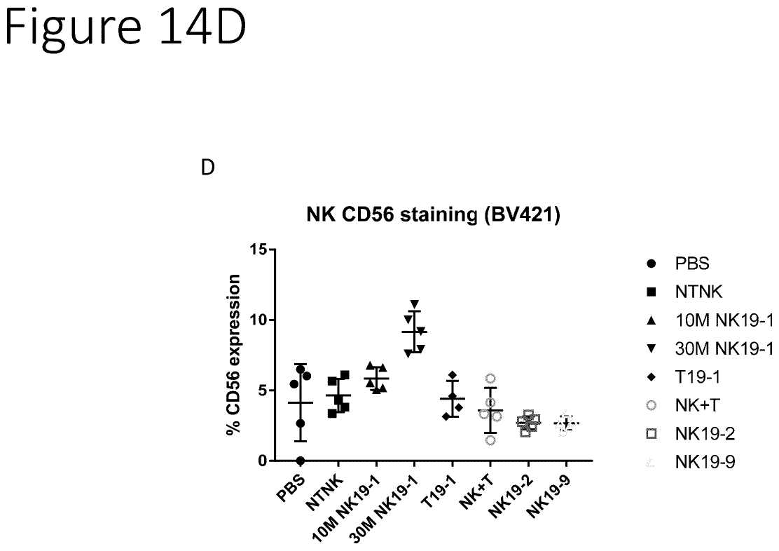

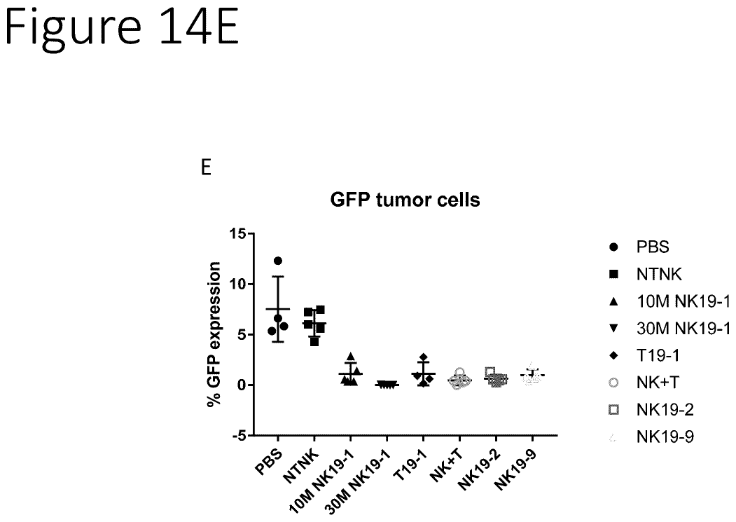

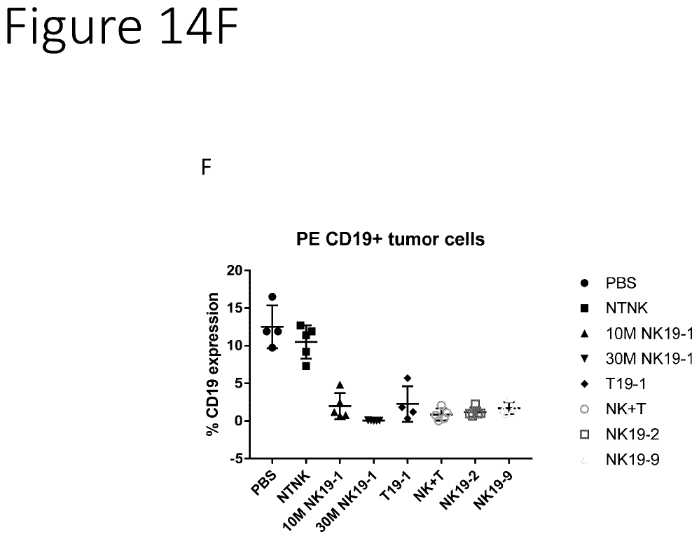

FIG. 14A depicts a line graph of the bioluminescence intensity shown detected in the mice (e.g., the fluorescent signal shown in FIG. 13, note that FIG. 13 does not show imaging data for Day 25). Consistent with the images of FIG. 13, the line graph of FIG. 14A shows increased Nalm6 cell count (as represented by increased BLI) for all groups at Day 25, with significant cell numbers detected in PBS and NTNK groups, and somewhat less for NK19-2, NK19-1 (10M) and NK19-9 groups. NK19-1 (30M) showed the smallest increase, representing that construct's ability to reduce the rate of Nalm6 progression due to cytotoxic effects on the Nalm6 cells. While each construct eventually allows some Nalm6 growth, even if modest, the tested NK19 constructs delayed the onset of that growth, as evidenced by the flat line through Day 11 for the NK19 curves. To better show that aspect, the data are replotted on a log scale Y-axis in FIG. 14B, which allows for curve separation. As displayed, the NTNK and PBS curves show an upward trend after Day 3, indicating nearly immediate Nalm6 growth. In contrast, the NK19-9, NK19-2 and both NK19-1 curves either drop, or only slightly trend upward through Day 7. At Day 11, consistent with the images in FIG. 13, Nalm6 cell growth is detected in the NK19-9, NK19-2 and NK19-1 (10M) groups. In contrast, the NK19-1 (30M) group is still approximately at baseline, reflecting the lack on any significant Nalm6 cell growth. Cell growth trends upward in the NK19-9 (30M group) on Day 18. While increases in tumor cell number occur, even with the NK19 constructs being administered, the delay of the growth onset could be advantageous in several embodiments. For example, this presents an opportunity to re-dose a patient with another dose of engineered NK cells expressing a CD19-directed construct. In several embodiments, a subsequent dose (as could the initial dose) may optionally comprise NK cells that have been edited to reduce allogenicty, for example by gene editing. Thus, in several embodiments, two, three, four or more doses of CD19-directed CAR expressing NK cells are administered. This delay in tumor cell growth presents an opportunity to dose, either serially (or concurrently) with an NK cell expressing a chimeric construct directed to a different tumor marker and/or some other variety of anti-cancer therapy (e.g., checkpoint inhibitor, antibody therapy, chemotherapy, etc.). FIG. 14C depicts data related to CD3 expression of cells transduced with the various constructs within blood samples taken from mice treated as indicated in the X axis. CD3 is a T cell marker. As indicated, but for T cells engineered to express the NK19-1 construct and a mixed population of NK cells and T cells, CD3 expression was essentially negligible. FIG. 14D depicts data related to CD56 expression, which is a marker for human NK cells. While there is some small amount of background staining, the blood samples from mice treated with NK cells expressing the indicated constructs is relatively low (as would be expected given that (i) the blood is a murine blood sample and murine cells would make the majority of the total, and (ii) CD56 is detecting only human NK cells (e.g., those administered). The data are consisting in that regard, with the 30M NK19-1 treatment group shoring markedly more CD56 expression than the other groups, and T cells/NK+ T cells groups expressing CD56 at background levels. FIG. 14E shows data related to the GFP expression by tumor cells. The blood samples were collected at −3 weeks into the in vivo experiment. As shown (consistent with the images/BLI data), the control PBS and NTNK groups exhibit higher percentages of GFP expression (e.g., a greater percentage of the total of live blood cells in the sample is tumor cells). Each of the engineered constructs shows significantly less GFP expression, based on the engineered constructs controlling/reducing tumor cell growth. FIG. 14F shows similar data to FIG. 14D, but measures CD19 expression across all the live cells in a given murine blood sample. As with GFP, the PBS and NKNT groups show CD19 expression at approximately 10-12%, meaning 10-12% of the live cells in the blood sample are tumor cells, the remainder being murine blood cells. As shown in the other experimental group, CD19 expression is much lower, reflective of the engineered CAR constructs limiting the growth of the tumor cells. These data are in accordance with embodiments disclosed herein, wherein engineered NK cells expressing CD19-directed CARs are highly cytotoxic and allow for the treatment of cancerous tumors.

### Further Evaluation of Humanized Constructs

As discussed in detail above, in several embodiments, several embodiments of the CARs disclosed herein involve the use of humanized sequences, such as in the extracellular binding moiety. In several embodiments, one or more aspects of that region is subjected to a humanization campaign. In several embodiments, one or more of the heavy and/or light chain of an antibody is humanized, which (as discussed above) can provide advantages including, but not limited to, reduced immunogenicity, increased stability, longer efficacy, increased potency, and the like.

### Example 3

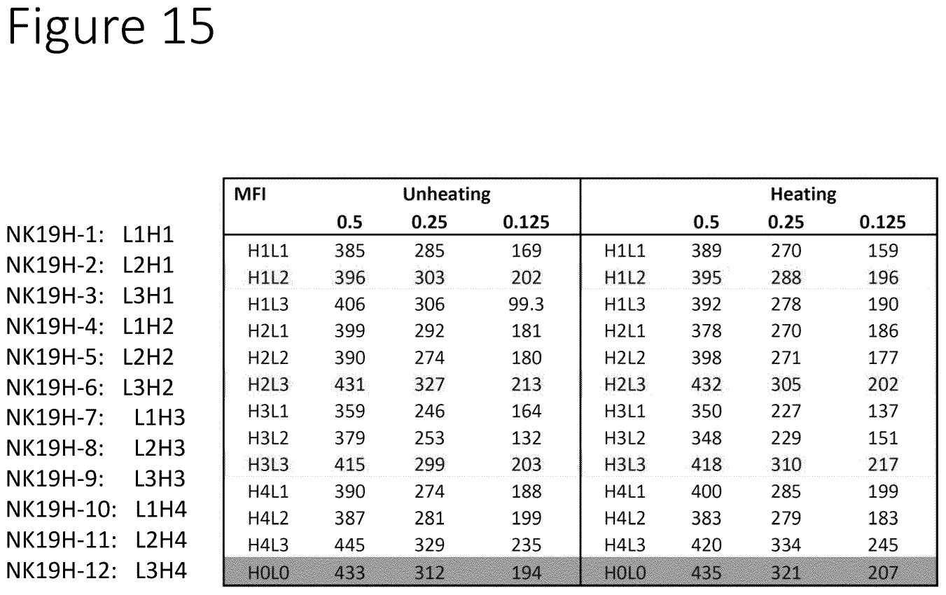

FIG. 15 shows a schematic of a series of humanized constructs according to several embodiments disclosed herein. Such constructs are designated by “H” in their identifier. By way of explanation, NK19H-1 is a humanized anti-CD19 CAR employing an scFv made up of a first light chain and a first heavy chain (‘L1H1’), while NK19H-3 employs an scFv made up of a third light chain and the first heavy chain (‘L3H1’). FIG. 15 also shows data related to the stability and aggregation of the various combinations of heavy and light chains following transient expression and secretion from 293T cells. Data are shown related to the mean fluorescence intensity detected by flow cytometry when each antibody was heated to 70° C. and then cooled back to room temperature (‘Heating’) vs. the same variant held on ice (‘Unheating’). After heat treatment, the ScFv variants are used in a flow cytometry protocol at various concentrations (0.4, 0.25, and 0.125 ug/mL). Loss of fluorescence intensity indicates that the ScFv either lost structural integrity or aggregated through the heating process. ScFv's with the best thermal stability as indicated by comparable MFI under both conditions are favored for further development, according to some embodiments.

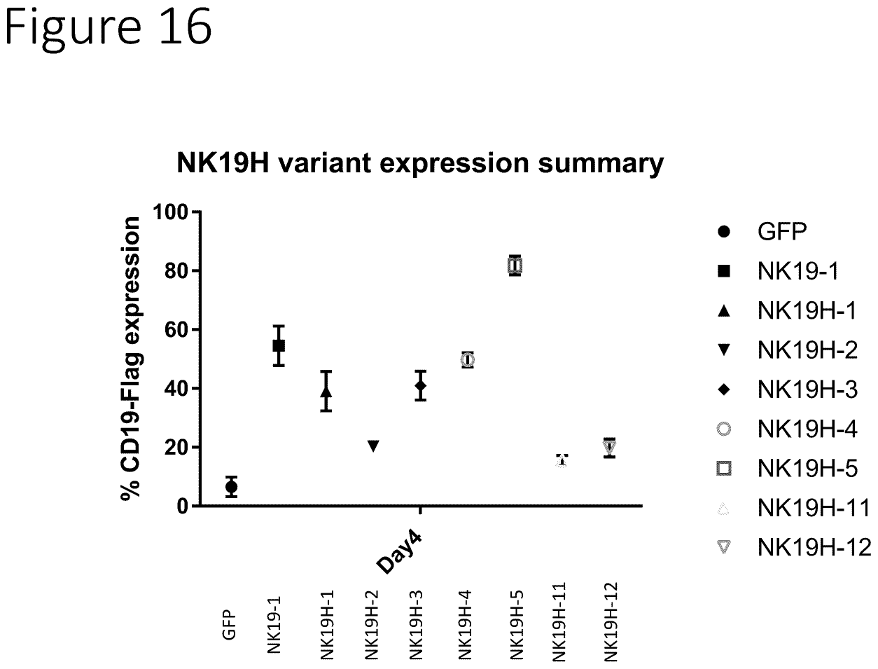

After having assessed the stability of the constructs, selected anti-CD19 CAR constructs were further evaluated. FIG. 16 shows summary data of expression of the indicated constructs by the NK cells of 3 donors (#140, #9, and #20). As depicted in the Figure, expression is evaluated by detection of CD19Flag. Data are presented as percentage of NK cells expressing CD19-Flag relative to the total number of NK cells presented. The data were collected at 4 days after transduction with the relevant virus encoding the NK19H-“X” CAR. As evidenced by the expression data, each of the selected constructs were expressed by NK cells, to varying degrees. Expression levels ranged from expression by about 20% of the total NK cells with NK19H-2, NK19H-11 and NK19H-12. Most of the other constructs were successfully expression by ˜40%-60% of the NK cells, which is on par with expression of the non-humanized NK19-1 construct. The NK19H-5 construct was expressed by over 80% of the NK cells. While certain constructs may be more efficiently expressed, those with lower expression levels were still evaluated because, as with several embodiments such constructs still exhibit significant cytotoxicity against target cells. According to several embodiments, however, those with higher expression efficiency can be advantageous, for example because a greater portion of a given NK cell preparation is useful clinically (e.g., fewer input NK cells needed to generate a clinically relevant engineered NK cell dose).

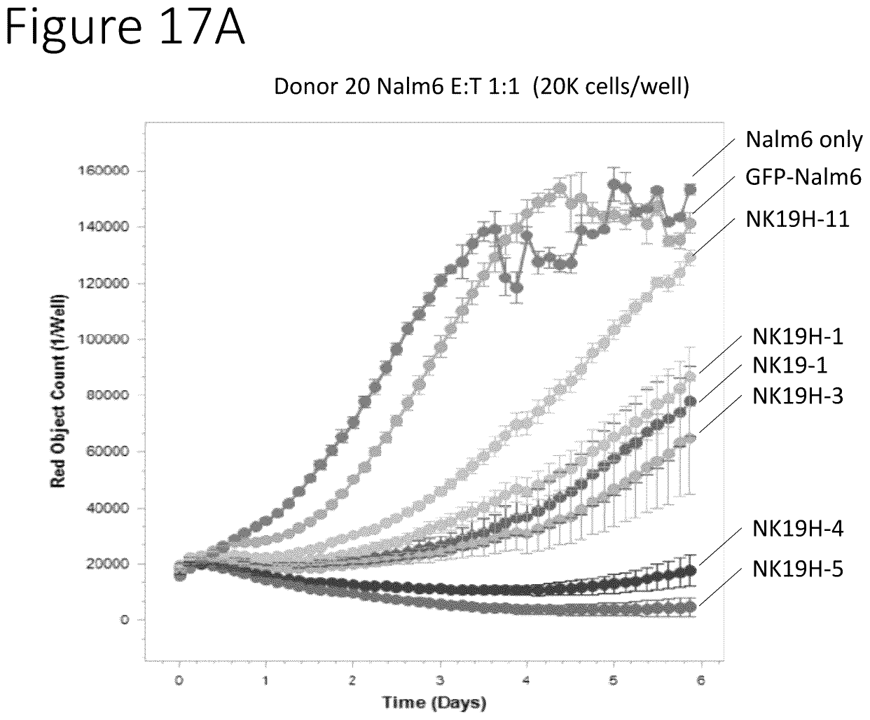

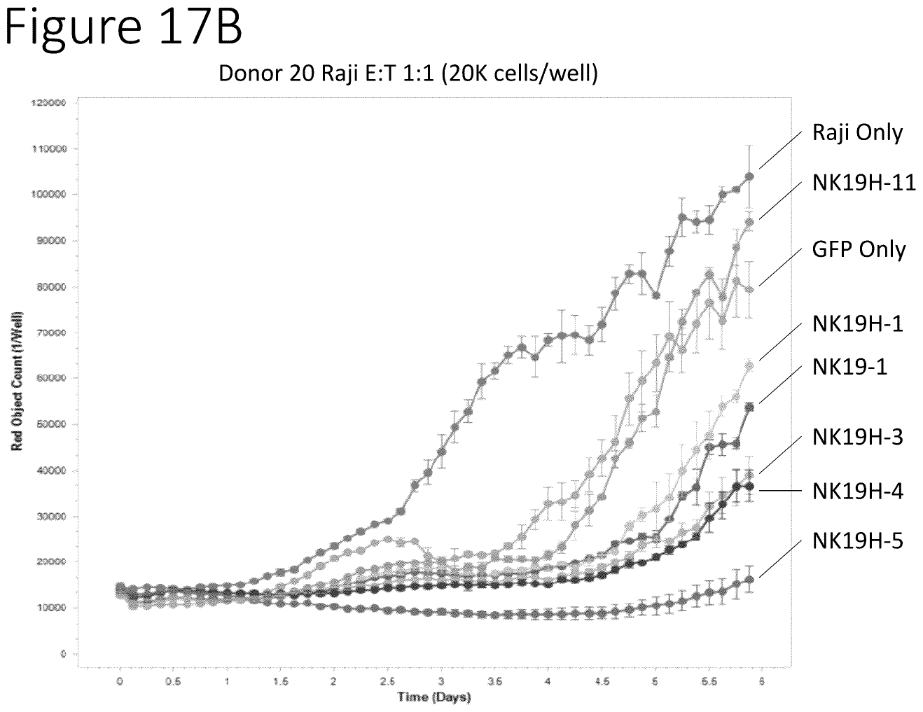

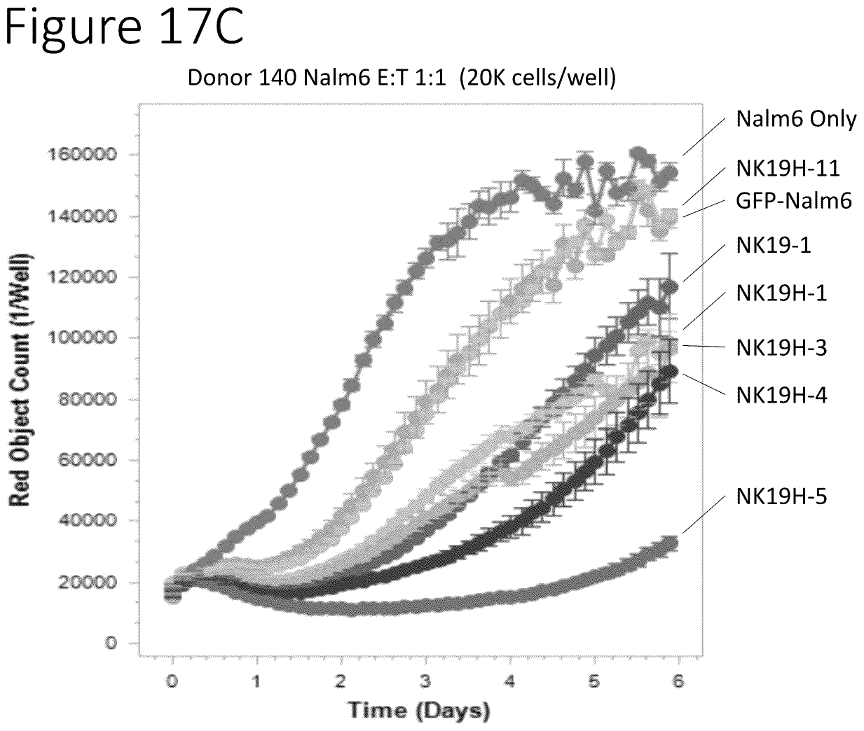

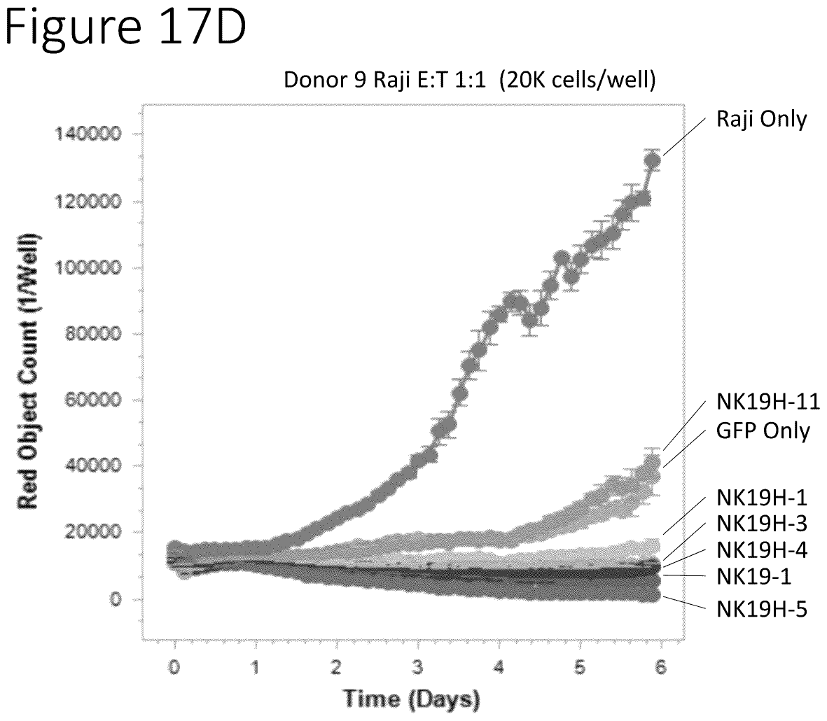

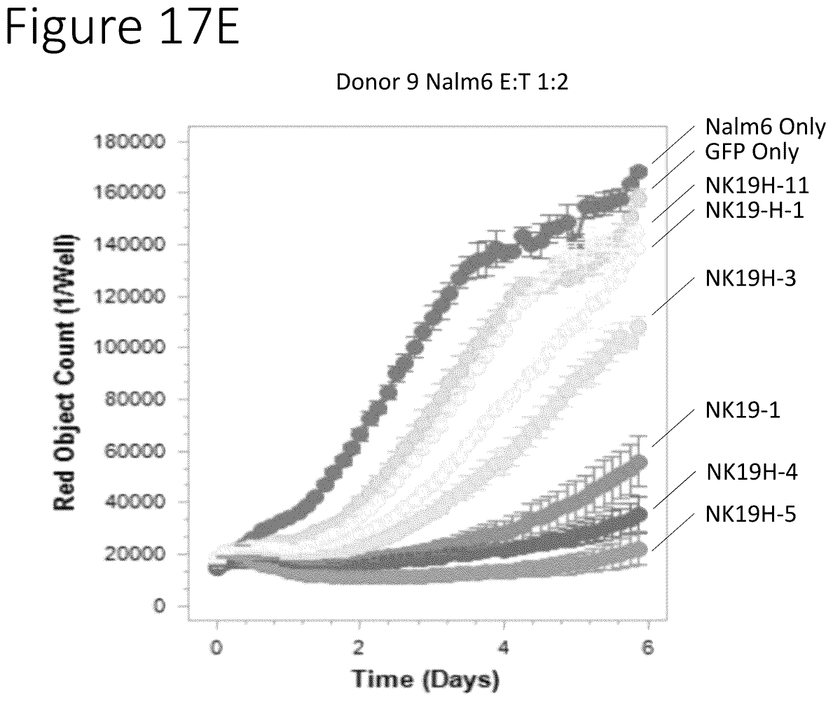

FIGS. 17A-17E depict such cytotoxicity data. FIG. 17A shows cytotoxicity of NK cells from Donor 20 and transduced with the indicated constructs against the CD19-positive Nalm6 leukemia cell line (at an E:T of 1:1; 20K cells per well). The data shows that, despite the varied expression levels, most of the NK19H constructs were able to exert cytotoxic effects against the Nalm6 target cells. NK19H-11 showed the least efficacy, allowing Nalm6 cell growth at levels just below the controls. In contrast, NK19H-1 and NK19H-3 showed cytotoxicity on par with the non-humanized NK19-1 construct, allowing for some cell growth at the later time points of co-culture. Notably NK19H-4 and NK19H-5 showed significant cytotoxicity, allowing only very limited Nalm6 growth throughout the experiment.

FIG. 17B shows NK cells from Donor 20 tested against the CD19-positive Burkitt's lymphoma cell line Raji. As with Nalm6 cells, the various humanized anti-CD19 constructs showed variable cytotoxicity against the target cells. Similar to the data of FIG. 17A, the NK19H-11 construct showed limited cytotoxicity, but all other constructs showed promising cytotoxicity against the target cells, with NK19H-1 performing on par with the non-humanized NK19-1 construct, and each of NK19H-3, 19H-4, and 19H-5 constructs inducing greater levels of cytotoxicity, limiting Raji growth until the later stages of the co-culture (with NK19H-5 allowing only very limited Raji cell growth). FIG. 17C shows corresponding Nalm6 data from Donor 140. Here, a similar pattern of efficacy was detected, with NK19H-11 allowing Nalm6 growth approximating negative controls. However, each of NK19H-1, 19H-3 and 19H-4 induced at least as much cytotoxicity as non-humanized NK19-1. Again, NK19H-5 showed significant cytotoxicity, limiting Nalm6 growth throughout the experiment. FIG. 17D shows data from Donor 9 against Raji cells. Only NK19H-11 allowed any substantial Raji cell growth. In contrast, NK cells from this donor expressed the non-humanized NK19-1 or any of the humanized NK19H-1, 19H-3, 19H-4, or 19H-5 constructs suppressed any Raji cell growth through the induced cytotoxic effects. FIG. 17E shows data for NK cells from Donor 9 against Nalm6 cells. In this experiment, the cytotoxicity of three of the humanized constructs (NK19H-11, 19H-1, and 19H-3) was limited. There is some donor to donor variability, as certain of these constructs induced cytotoxicity when expressed by NK cells of other donors. The NK19H-4 and 19H-5 constructs exhibited significant cytotoxic effects, nearly limiting Nalm6 growth to zero over the course of the experiment. Taken together, these data complement the expression data and show that, in accordance with several embodiments, humanized CAR constructs targeting CD19 are effective at killing tumor cells, even if they have variable expression efficiencies. Additionally, according to some embodiments, the expression efficiency is not correlated with cytotoxicity and even constructs with limited expression efficiency can demonstrate significant cytotoxicity.

### Example 4

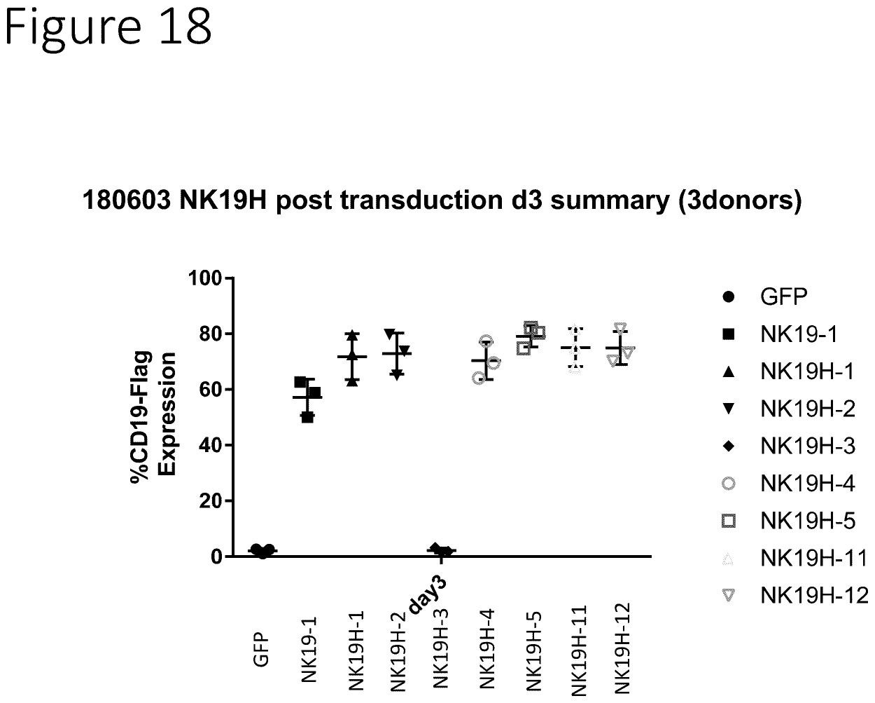

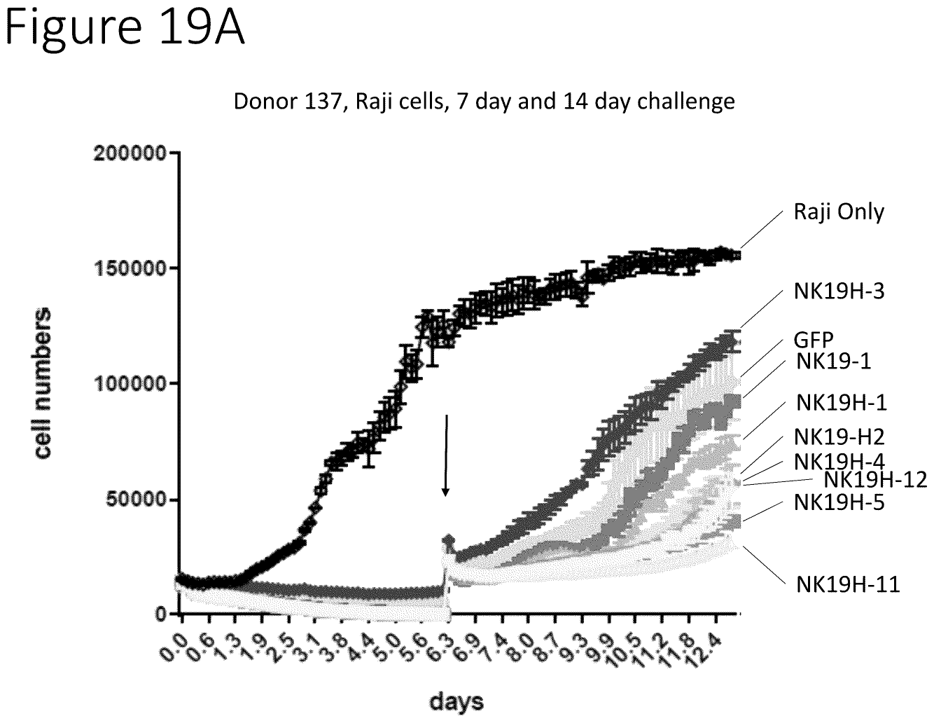

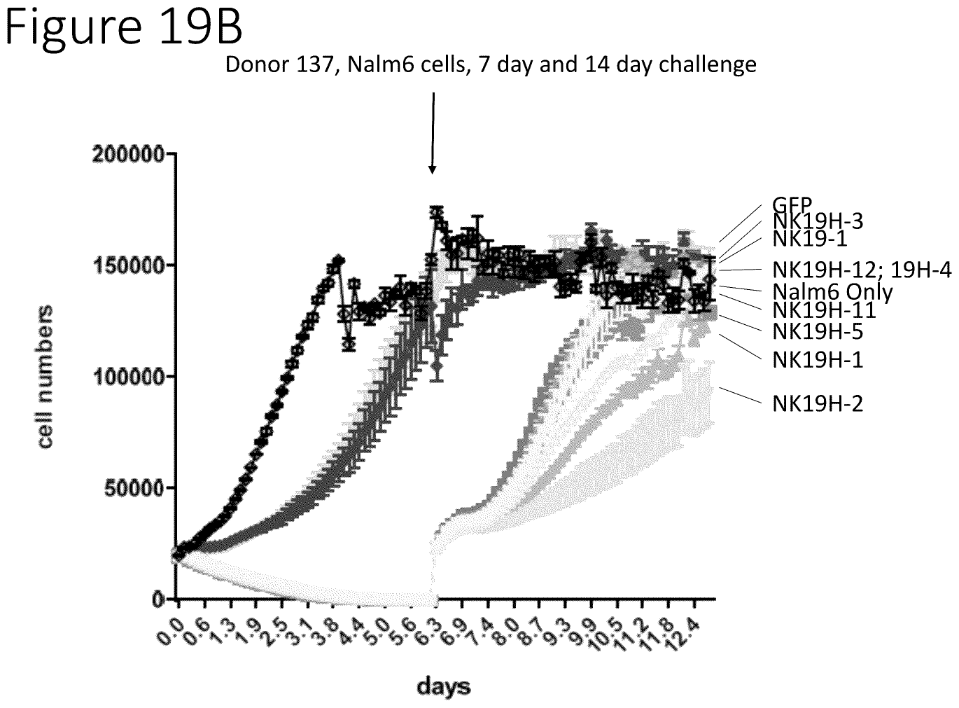

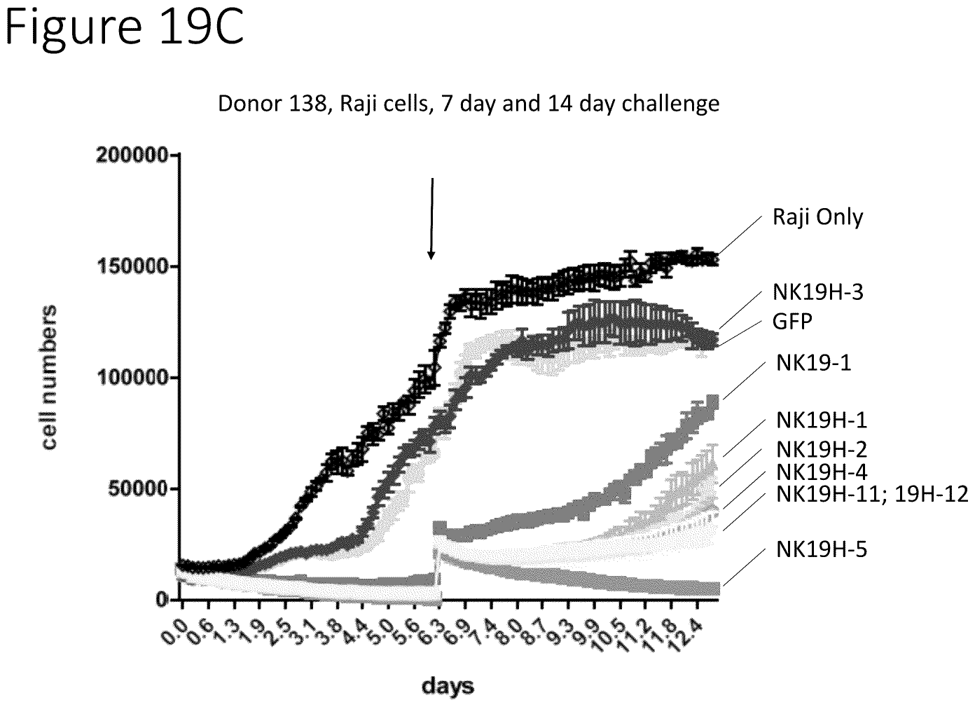

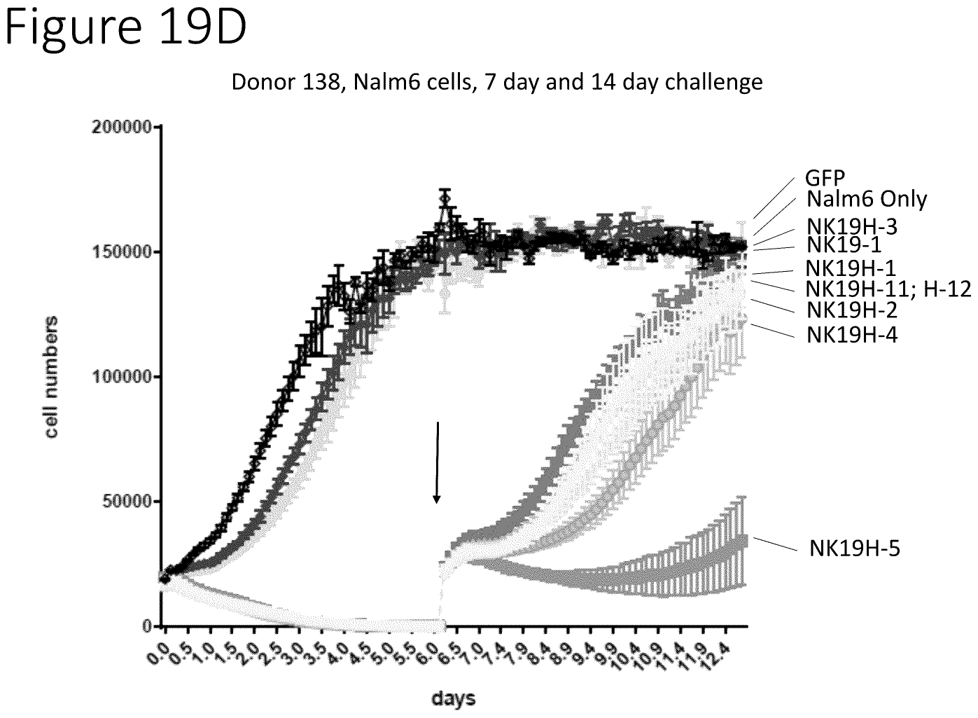

Further experiments paralleling those discussed above were performed using NK cells from additional donors. FIG. 18 shows construct expression efficiency for three donors (#945, 137 and 138) for the indicated constructs. As with the prior experiment the data presented represent the number of CD19-Flag positive cells out of the total number of NK cells evaluated. The data for these donors shows a higher overall efficiency of expression for all but one of the humanized constructs. Only NK19H-3 was expressed less than the non-humanized NK19-1 construct. With these donor NK cells, expression of the humanized constructs was detected on about 70% to 80% of the NK cells. As above, further to evaluation of expression, cytotoxicity against CD19 expression target cells was evaluated. FIG. 19A shows data related to NK cells from a donor (#137) expressing the indicated constructs and co-cultured with Raji cells. In this experiment, the engineered NK cells expressing the indicated constructs were exposed to the tumor cells at two time points, 7-days and an additional bolus of tumor cells was added at 14-days post-transduction. The arrow shows the second administration of tumor cells. As shown, untreated Raji cells expanded throughout the experiment. NK cells expressing GFP or non-humanized NK19-1 induced some cytotoxicity as shown by the reduced Raji cell growth as compared to control. NK cells expressing NKH19-3 (the humanized construct with the lowest expression efficiency) were also able to reduce Raji growth. Each of the other humanized NK constructs were able to reduce Raji cell growth compared to controls, even at 14 days post-transduction, which is indicative of the enhanced persistence of engineered NK cells disclosed herein. FIG. 19B shows corresponding data for Donor 137 NK cells against Nalm6 cells with engineered NK cells again being added at day 7 (day 0 of experiment) and day 14 post-transduction (˜day 7 of experiment). Cytotoxicity of the indicated constructs was more variable in this particular experiment. However, several humanized anti-CD19 constructs were able to produce marked cytotoxicity and reduce Nalm6 cell growth as compared to controls. These data suggest that, according to several embodiments, a more frequent dosing schedule (e.g., every 2 days, every 3 days, every 4 days, every 5 days, etc.) would be beneficial for certain subjects. Advantageously, in several embodiments, engineered NK cells as disclosed herein are allogeneic and can be readily used in more frequent dosing regimens. FIG. 19C shows corresponding data for Donor 138 against Raji cells. Here, many of the humanized constructs still had significant cytotoxic effects on the Raji cells, even with the second dose of Raji cells at 14 days post-transduction. Six of the 7 humanized constructs showed this behavior, and outperformed the non-humanized NK19-1 construct. FIG. 19D shows the corresponding data for Donor 138 against Nalm6 cells. While the additional bolus of Nalm6 led to increased Nalm6 growth in the latter stages the experiment, nearly all of the humanized constructs performed better than controls. In fact, NK19H-5-bearing NK cells were able to limit Nalm6 growth until the final few days of the experiment (after the second dose). As above, these data show that humanized anti-CD19 CAR constructs can not only be expressed by NK cells, but can also exert cytotoxic activity against target cells, with an enhanced persistence. In several embodiments, this allows multiple dosing to be separated by longer periods of time, which could be advantageous for treating cancers, while limiting potential immunogenicity (at least in part due to the humanization and/or because of the reduced frequency of administration).

### Example 5

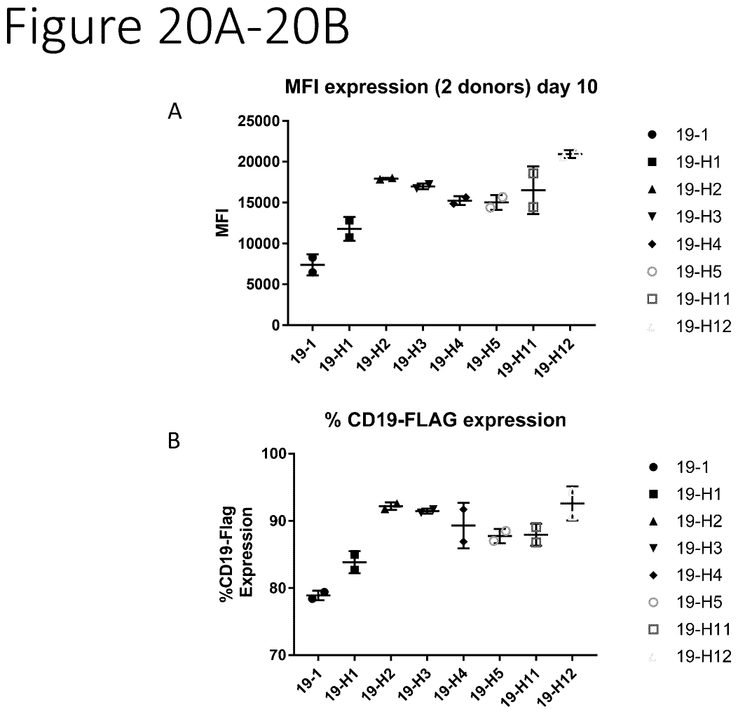

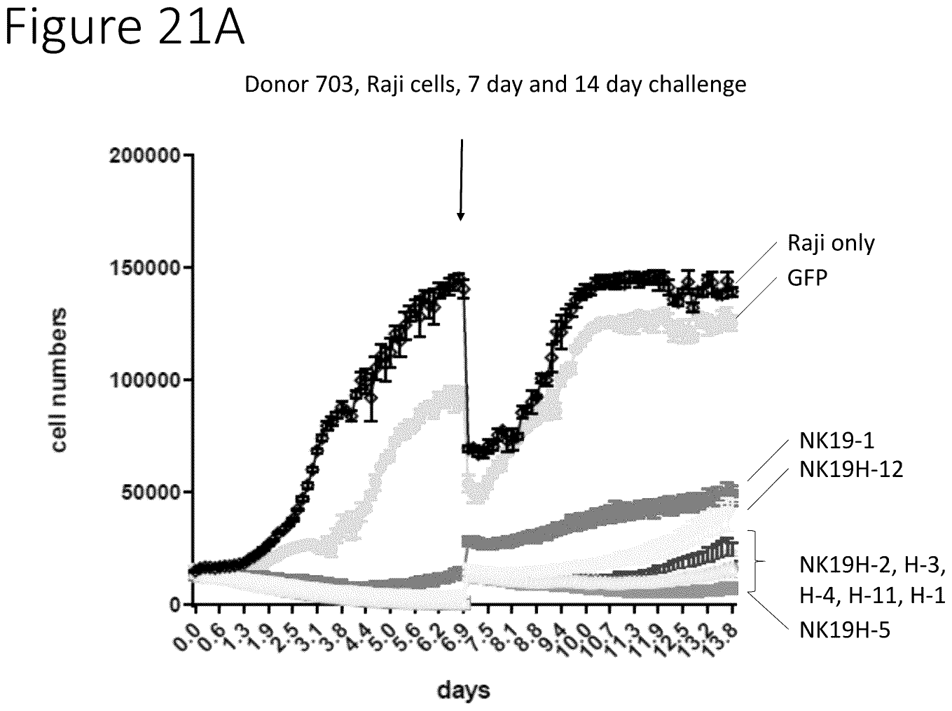

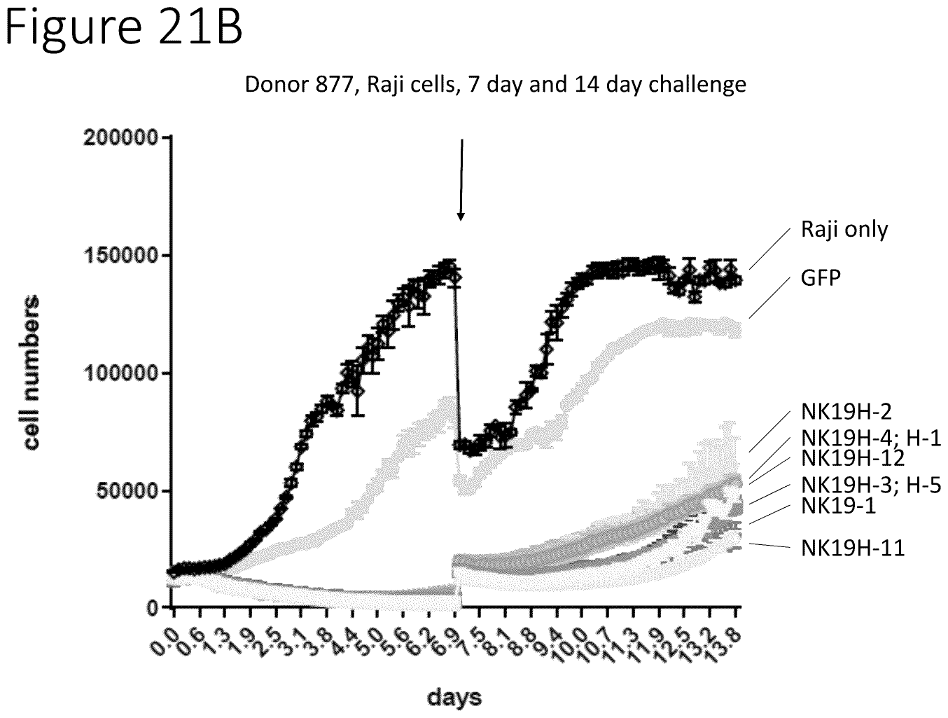

Further data for various humanized constructs in additional donors was collected. FIGS. 20A-20B show expression data for various anti-CD19 CAR constructs in NK cells from two additional donors. FIG. 20A shows the mean fluorescence intensity for NK cells at 10 days post-transduction. As indicated, as in accordance with several embodiments disclosed herein, each of the humanized anti-CD19 CAR constructs showed enhanced overall expression as compared to a non-humanized anti-CD19 CAR. FIG. 20B shows the expression data of CD19-Flag positive NK cells as a percentage of the total number of NK cells analyzed. As shown, each of the humanized anti-CD19 constructs were more efficiently expressed than the non-humanized construct (which was already expressed by almost 80% of the NK cells). The humanized CARs were expressed by approximately 85% to 95% of the NK cells, depending on the construct. FIG. 21A shows cytotoxicity data for engineered NK cells from Donor 703 (one of the two donors from FIG. 20) against Raji cells co-cultured with the NK cells at day 7 post-transduction and again at day 14 post-transduction. As shown in the Figure, Raji cells and NK cells expressing GFP alone grew robustly through day 7 and again through day 14. The non-humanized anti-CD19 CAR NK19-1 was able to suppress Raji cell growth through 7 days, and allowed relatively limited growth through 14 days. Each of the humanized anti-CD19 CAR constructs further suppressed Raji cell growth with several of the constructs nearly completely suppressing Raji cell growth. FIG. 21B shows corresponding data for cytotoxicity against Raji cells for NK cells isolated from Donor 877. These data show a similar trend to that from the prior donor. The two control groups allowed significant Raji cell growth, while each of the humanized anti-CD19 CAR constructs yielded significant cytotoxic effects.

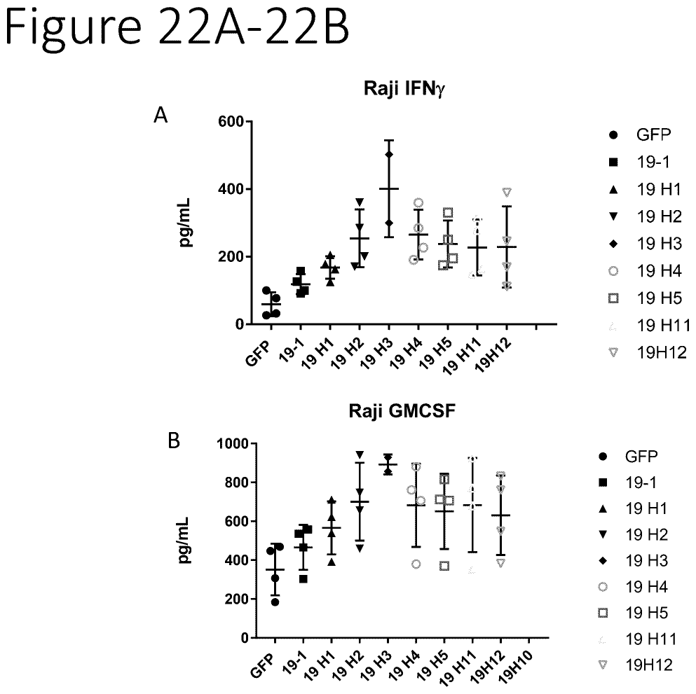

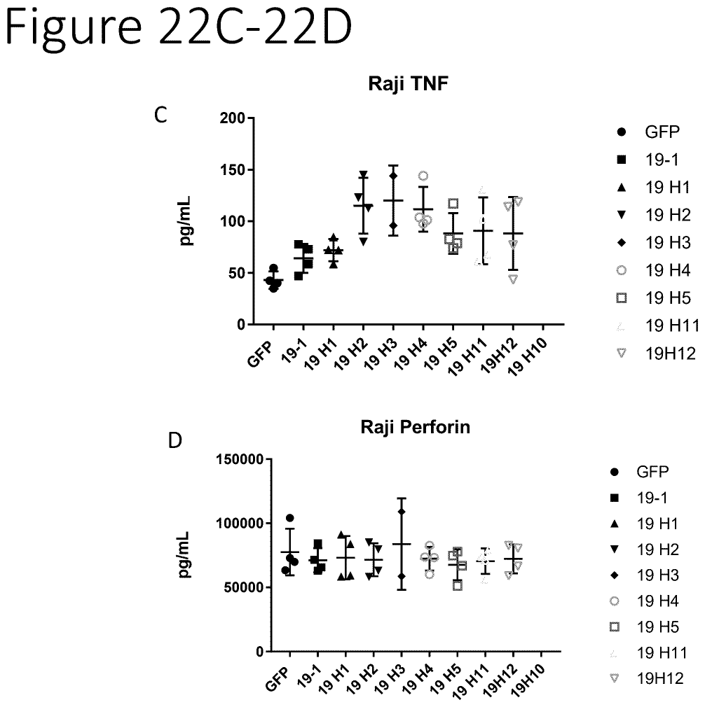

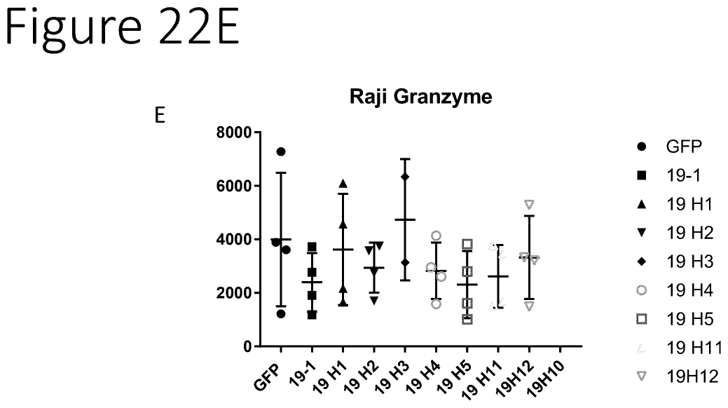

FIGS. 22A-22E show cytokine release profiles from Raji cells co-cultured with NK cells expressing the indicated constructs. FIG. 22A shows IFNg release by NK cells co-cultured with Raji cells. Each of the indicated humanized constructs resulted in increased IFNg release as compared to the non-humanized NK19-1 construct. Similarly, as shown in FIG. 22B, the humanized anti-CD19 constructs enabled the NK cells to release greater amounts of GM-CSF as compared to control NK cells. FIG. 22C show that humanized anti-CD19 CAR constructs induce elevated TNF release from the NK cells. Perforin levels were not significantly different across the constructs tested, as shown in FIG. 22D. Likewise Granzyme levels were relatively constant across the constructs. These data, taken together, indicate that according some embodiments, humanized anti-CD19 CAR constructs expressed on NK cells cause those NK cells to release greater amounts of one or more cytokines that lead to cytotoxic effects on target cells.

### Example 6

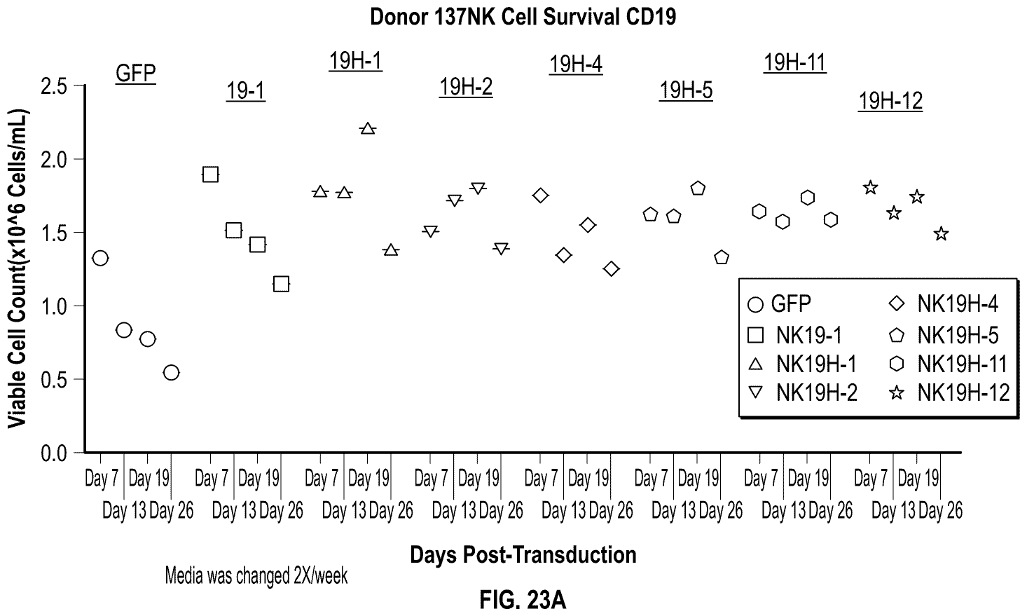

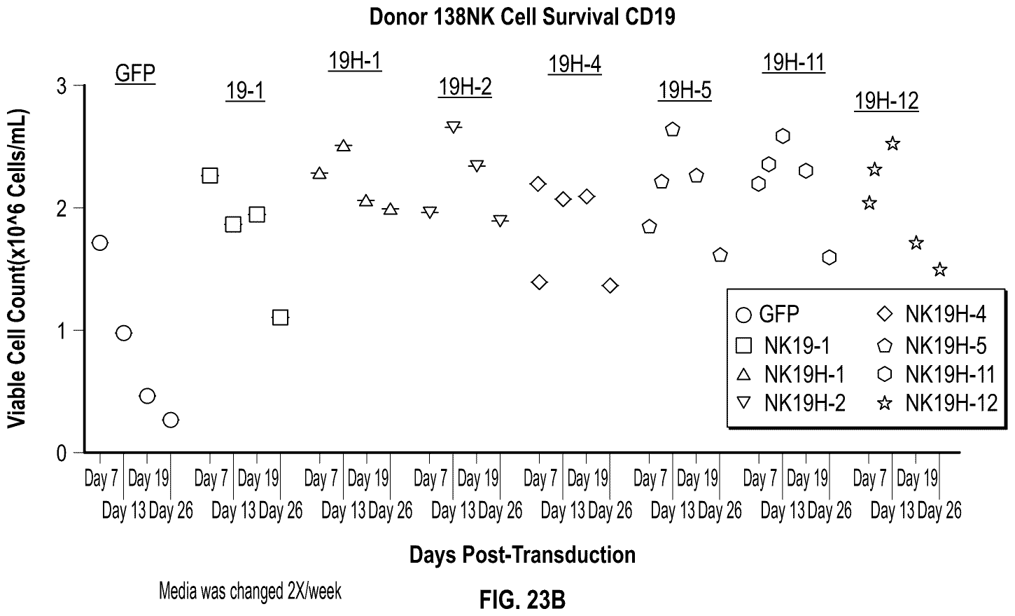

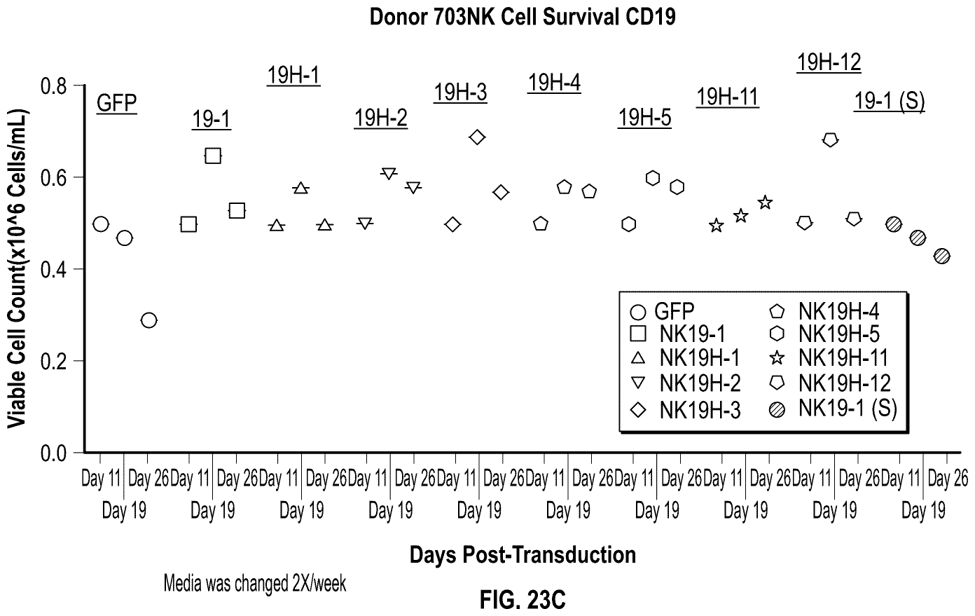

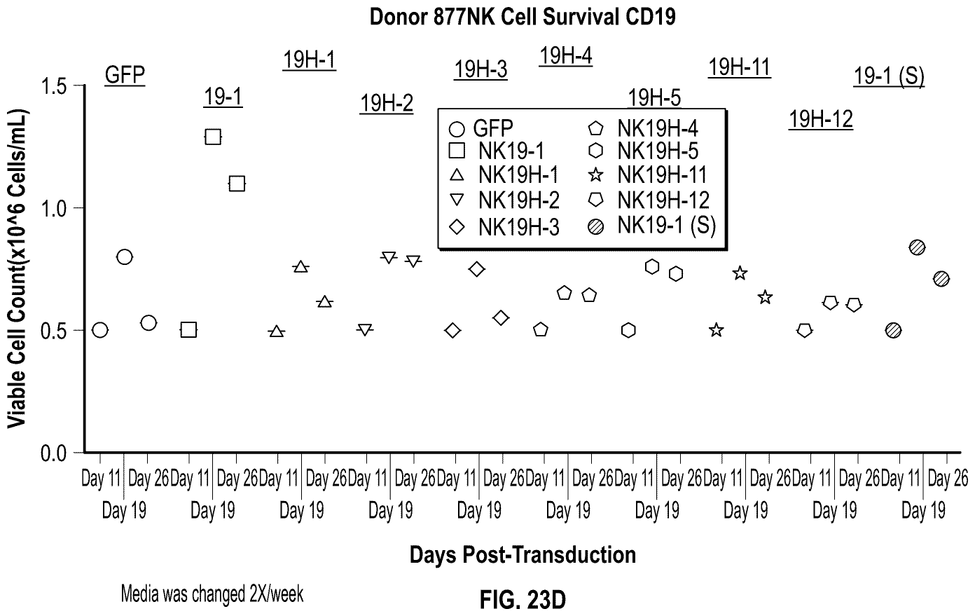

While the data presented above show that humanized anti-CD19 CARs can be expressed by NK cells and have cytotoxic effects on target tumor cells, additional experiments were performed to determine the longevity of the engineered NK cells. NK cells expressing the indicated anti-CD19 CAR constructs were cultured and the cell count was measured at various time points, out to 26 days post-transduction. FIG. 23A shows the survival data for NK cells from Donor 137. The data for the GFP-expressing NK cells show that the cell count at day 7 is higher than the cell count at any other time-point, suggesting that GFP has provided no additional longevity-inducing effects to the NK cells. Likewise, NK cells expressing non-humanized NK19-1 shows a fall off of cell number over time. While each of the humanized constructs shows some variability in terms of cell count, the trend of the data shows that expression of the humanized anti-CD19 constructs results in less NK cell death over time. A similar trend is shown in the data of FIG. 23B, which shows cell viability for NK cells from Donor 138. While the timing of analysis is modified, FIG. 23C shows data yielding a similar trend (for Donor 703), in that the expression of the humanized constructs results in longer NK cell survival over time in culture. FIG. 23D shows data for NK cells from Donor 877, where the expression of humanized anti-CD19 CAR constructs allows for reduced NK cell death over time in culture.

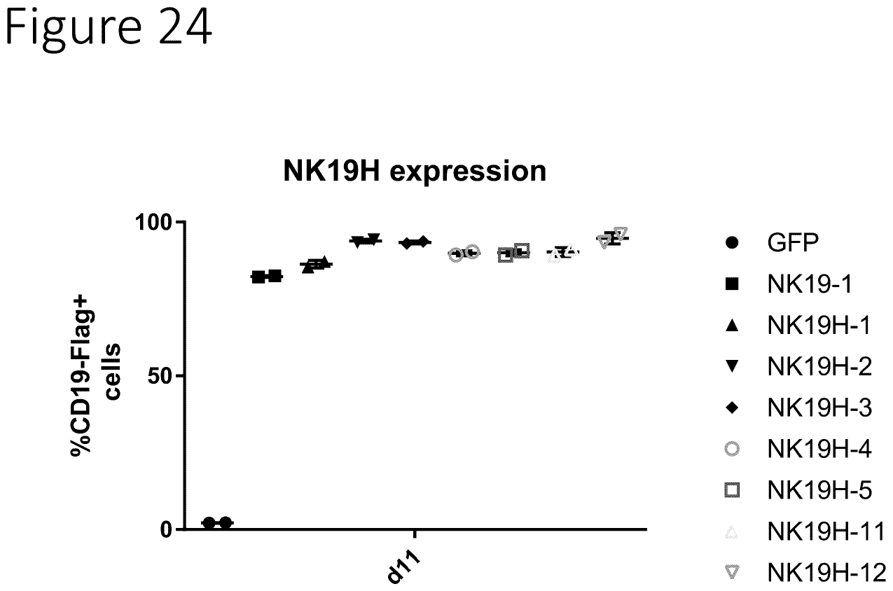

Summary data for the expression of the various anti-CD19 CAR constructs is shows in FIG. 24, which displays the expression efficiency of the indicated constructs at day 11 post-transduction. As anticipated, the GFP-transduced NK cells exhibit no CD19-Flag expression, serving as a negative control. Likewise, the non-humanized NK19-1 construct serves as a positive control. Each of the humanized constructs assessed showed enhanced expression efficiency as compared to NK19-1, with efficiencies ranging from about 85% to about 95% (e.g., 85%-95% of all the NK cells tested expressed the Flag-tagged CAR construct).

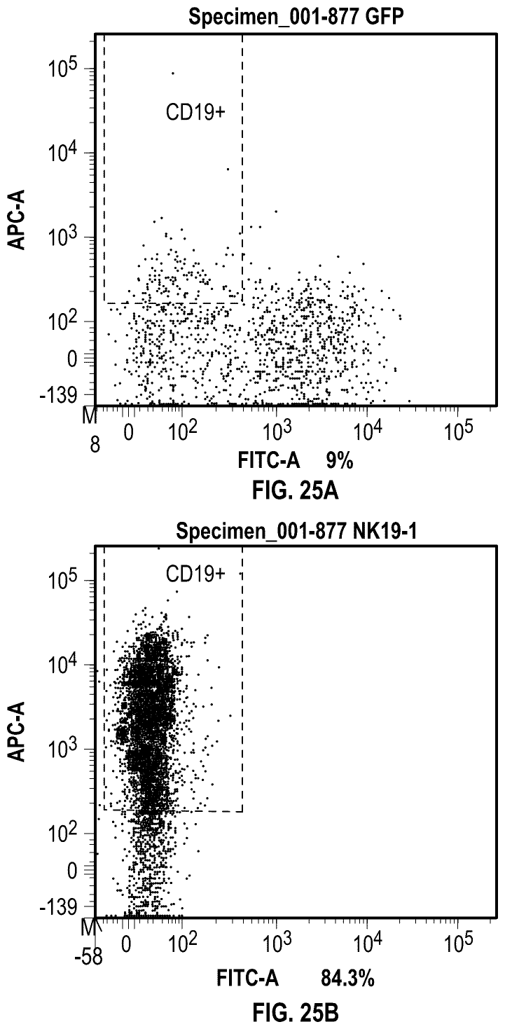

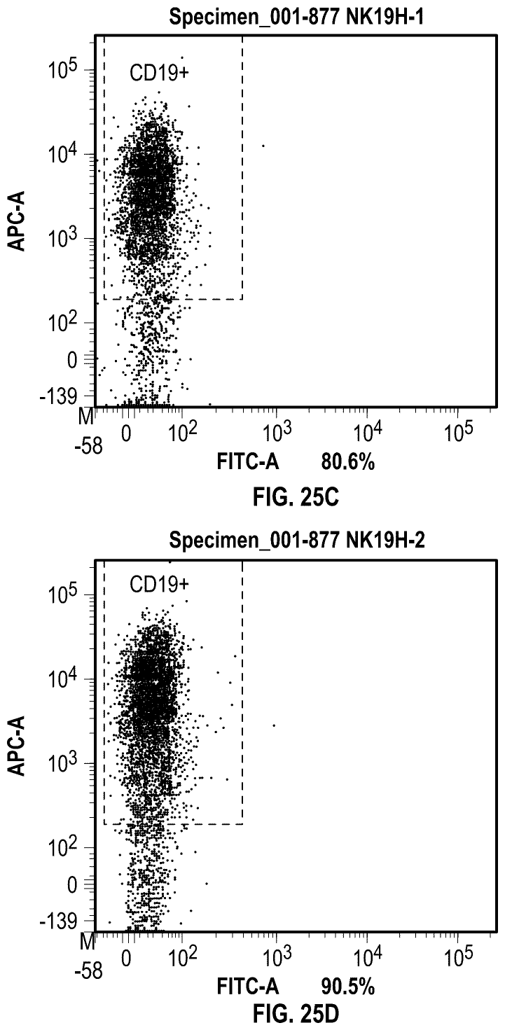

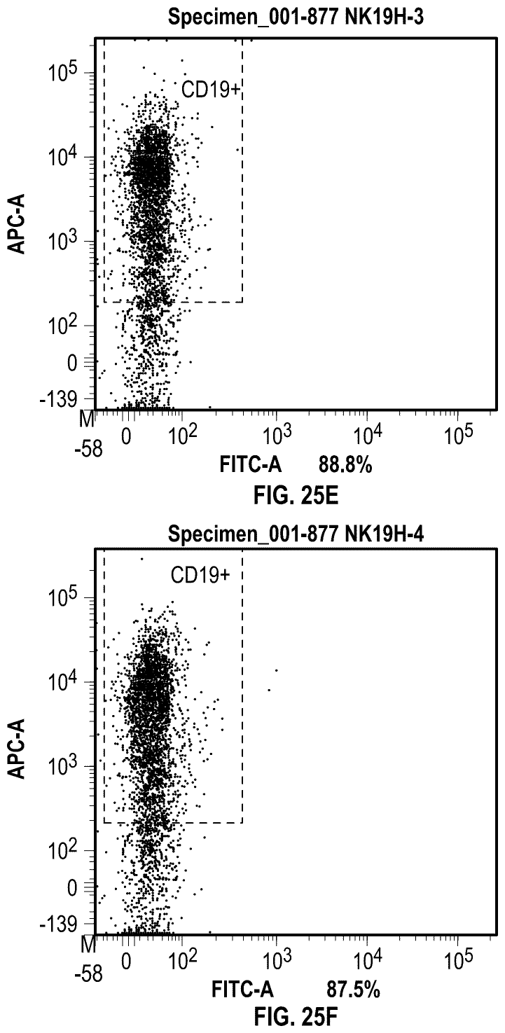

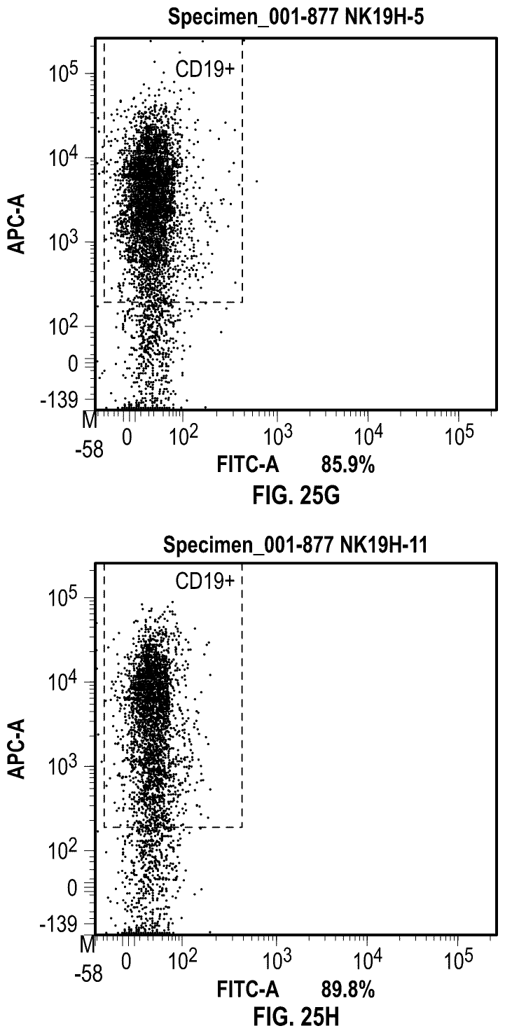

FIGS. 25A-25I show raw flow cytometry data for one donor wherein expression of CD19-Flag (indicative of CAR expression) is measured. FIG. 25A shows the GFP-control, with little to no Flag expression. FIG. 25B shows Flag detection with the NK19-1 positive control. FIGS. 25C-25I show the results of Flag detection for the indicated humanized anti-CD19 CAR constructs, with the percentage of cells expressing the constructs ranging from about 80% to about 95%. As with the experiments above, these data confirm that the humanized anti-CD19 CAR constructs can be robustly expressed by transduced NK cells.

### Example 7

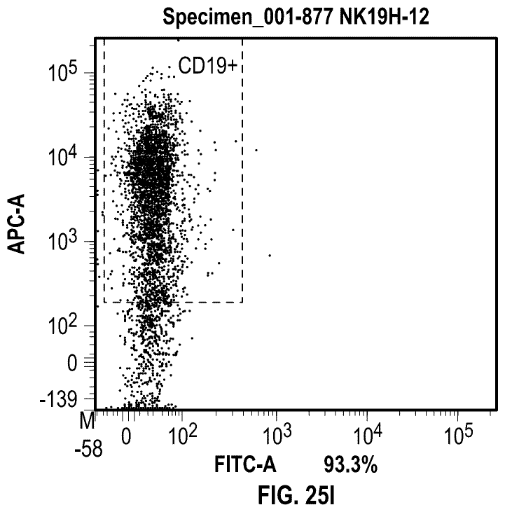

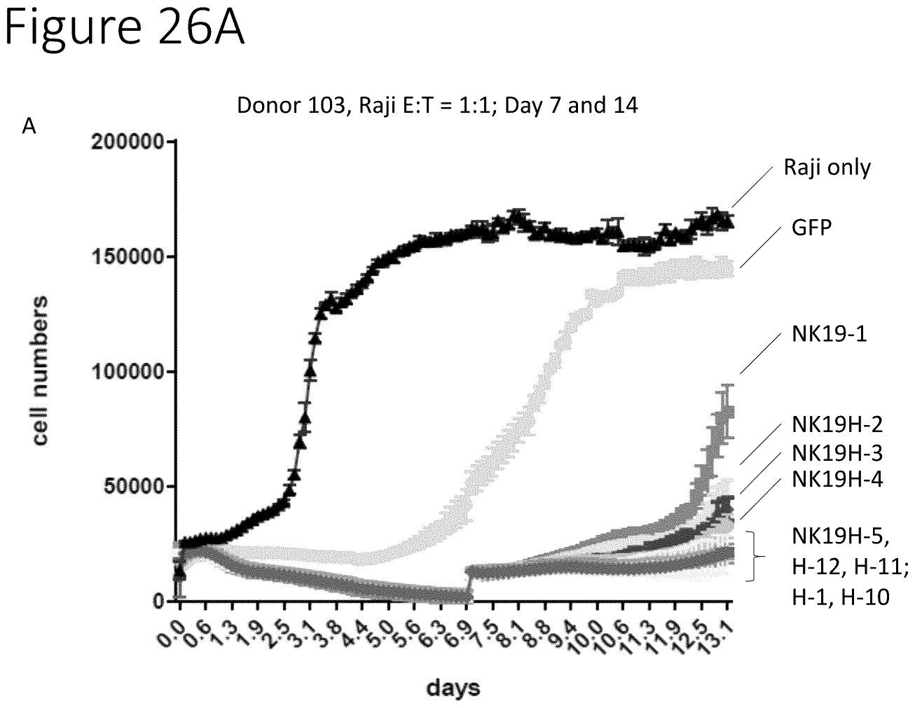

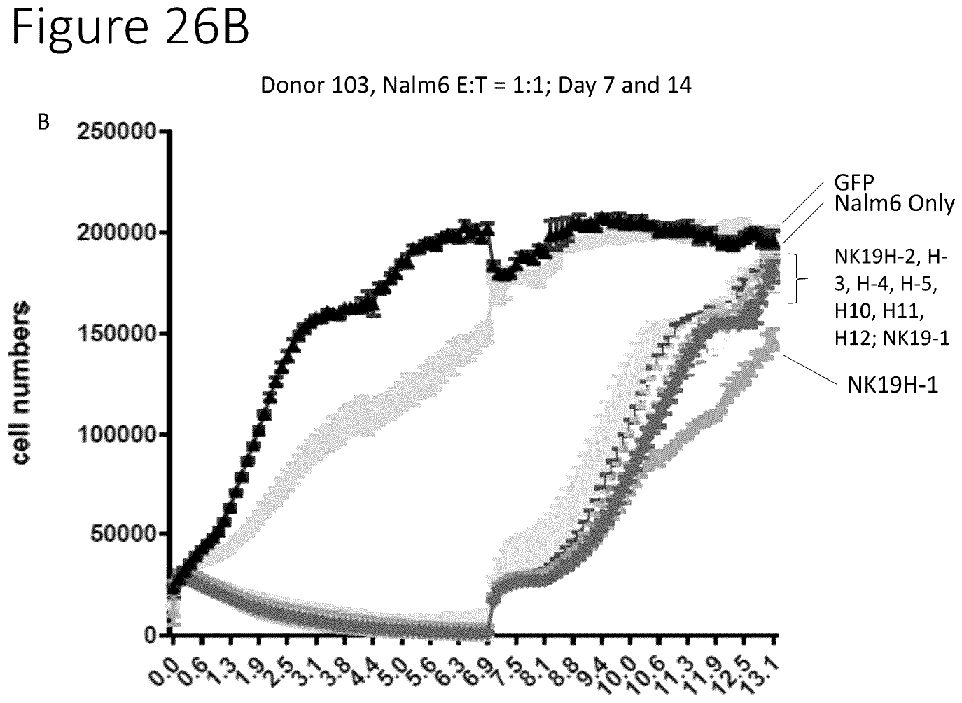

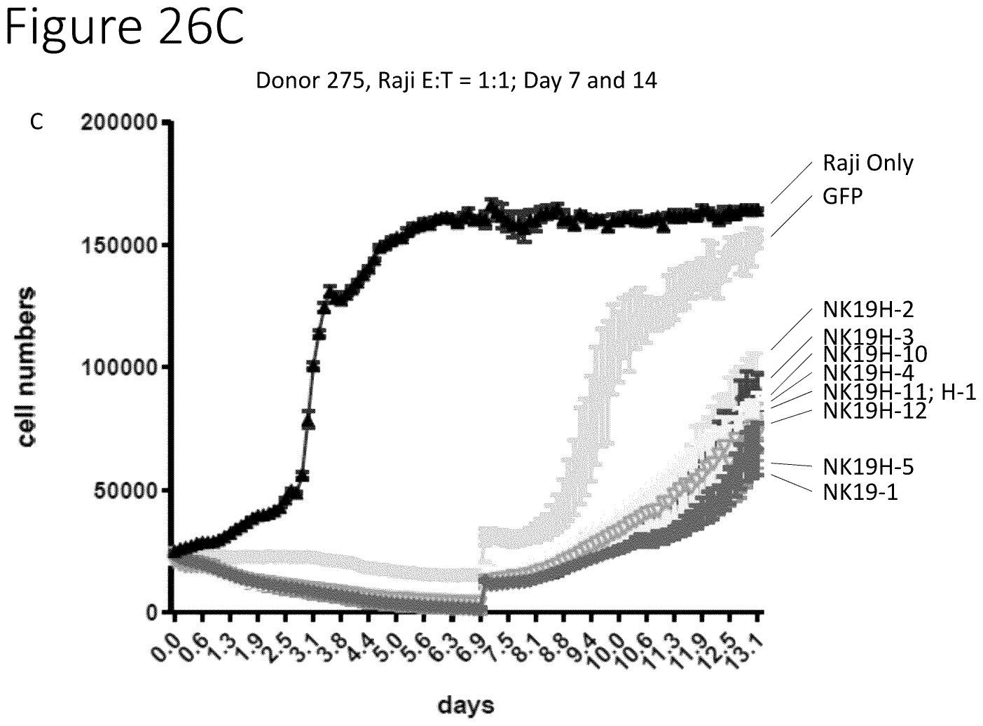

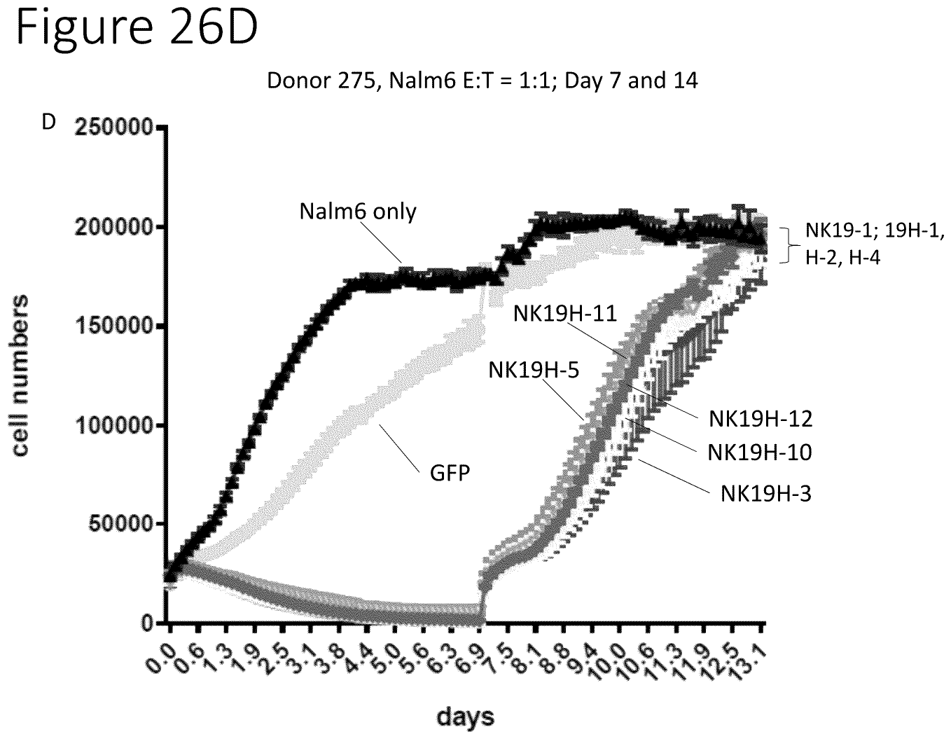

Similar to the experiments discussed above, Raji cells were exposed to NK cells from donor 103 which were transduced with the various constructs indicated (see FIG. 26A). Raji cells were co-cultured on day 7 post-transduction of the NK cells, and the NK cells were re-challenged again on day 14. As expected Raji cells alone exhibited continued growth, while GFP-transduced NK cells reduced that cell growth a small amount. In contrast, the positive control non-humanized NK19-1 construct reduced Raji cell growth until the very late stages of the experiment. Each of the humanized anti-CD19 CAR constructs yielded enhanced cytotoxicity against the Raji cells. Three of the constructs (NK19H-2, H-3, and H-4) allowed for minor Raji cell growth at the late stage of the experiment, while the other humanized constructs effectively eliminated Raji cell growth, even with the re-challenge at day 14. FIG. 26B shows corresponding data regarding the cytotoxicity of donor 103 NK cells against Nalm6 cells. As indicated in the Figure, all of the anti-CD19 CAR constructs (whether humanized or not) appeared exhibit significant cytotoxicity against the Nalm6 cells through 7 days of co-culture. However, Nalm6 growth increased for all groups with the day 14 re-challenge. While the growth curves initially were somewhat flat, Nlam6 growth later increased in rate. These data suggest that, according to some embodiments, a more frequent dosing strategy is employed to keep target cells from reaching a threshold growth rate. In several embodiments, a larger dose is given and/or a dose is given more frequently, to prevent target cell growth under a physiological equivalent context to a “re-challenge.”

FIG. 26C shows data for Raji cells (as with FIG. 26A) with NK cells from donor 275. Similar to those of FIG. 26A, each of the anti-CD19 CAR constructs showed significant cytotoxicity against Raji cells, and allowing limited growth of the target cells, even with a re-challenge. FIG. 26D shows data for Nalm6 cells (as with FIG. 26B) with NK cells from donor 275. Results for this experiment were also similar to those for donor 103, with effective control of Nalm6 cells through 7 days, but reduced cytotoxicity after re-challenge. These data indicated that dosing strategies, depending on the embodiment, are developed for a specific donor and/or for a specific target tumor type. For example, while Nalm6 cells are CD19 positive, they may express less CD19 than, for example, Raji cells, thereby accounting for the reduced cytotoxicity of NK cells from donor 103 and 275 against the Nalm6 cell line. The efficacy of the transduced NK cells against the Raji cells indicates that the NK cells are capable of cytotoxicity, even showing persistence out to nearly two weeks post-transduction. Thus, according to several embodiments, an NK cell dose and/or dosing frequency can be tailored to a given patient's tumor type, native NK cell activity, and/or the aggressiveness/stage of a cancer to allow robust and ongoing cytotoxicity against the target cells and achieving control of tumor burden.

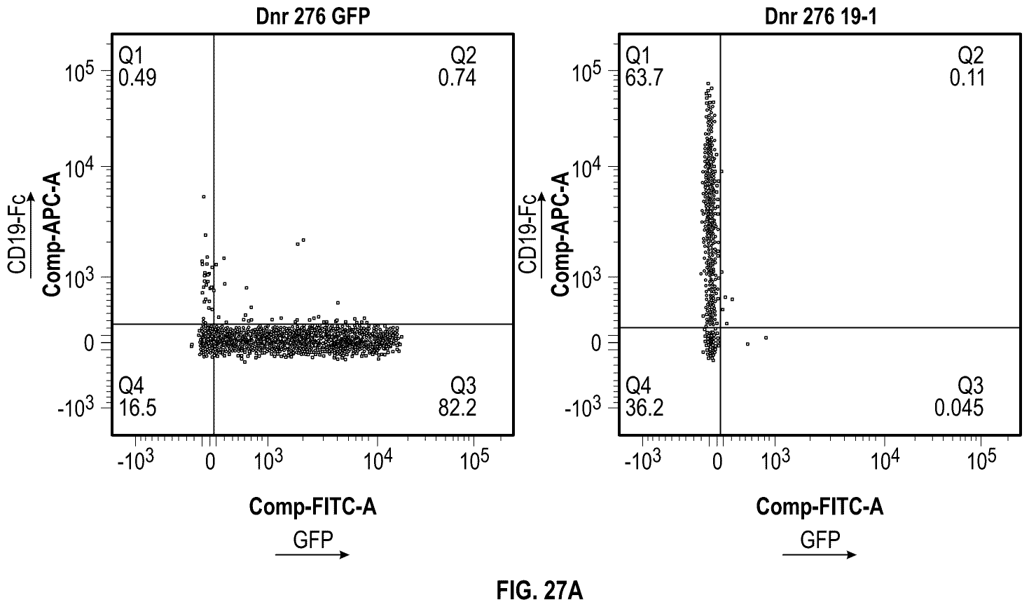

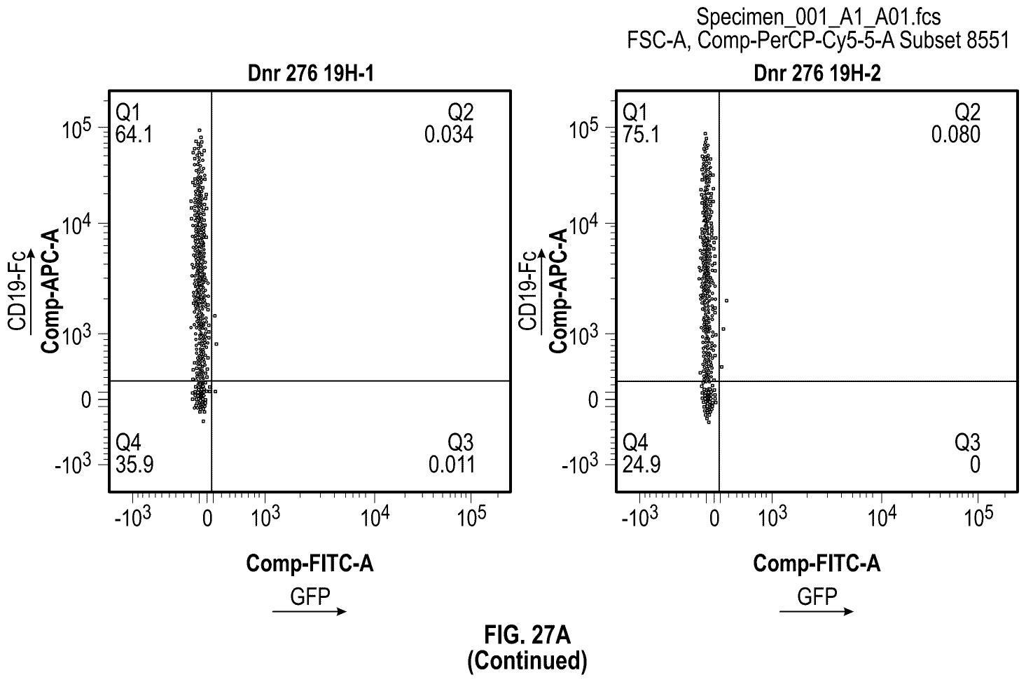

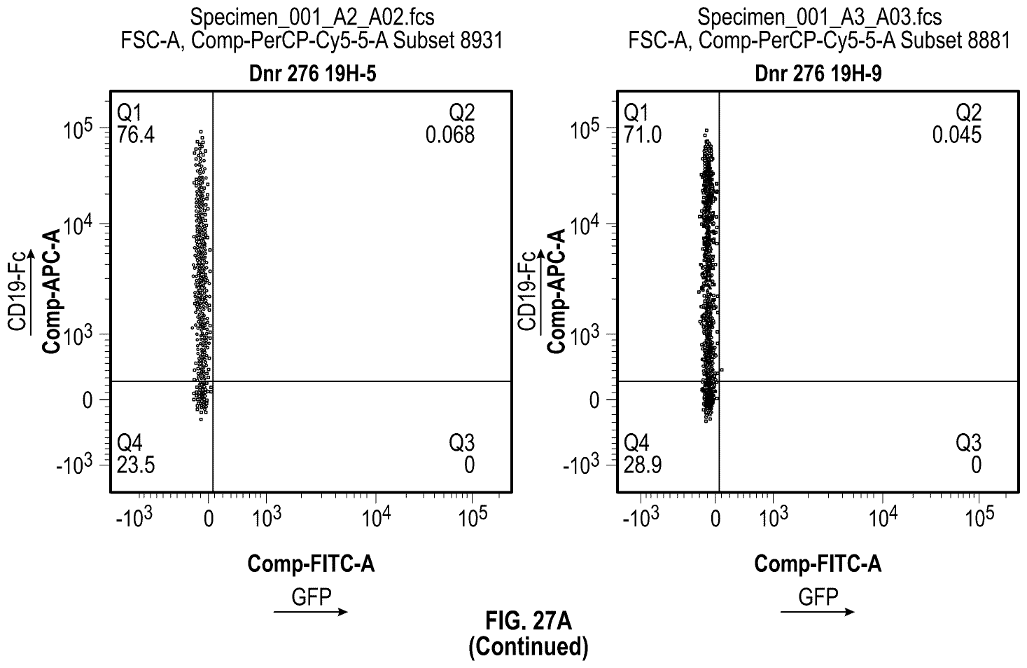

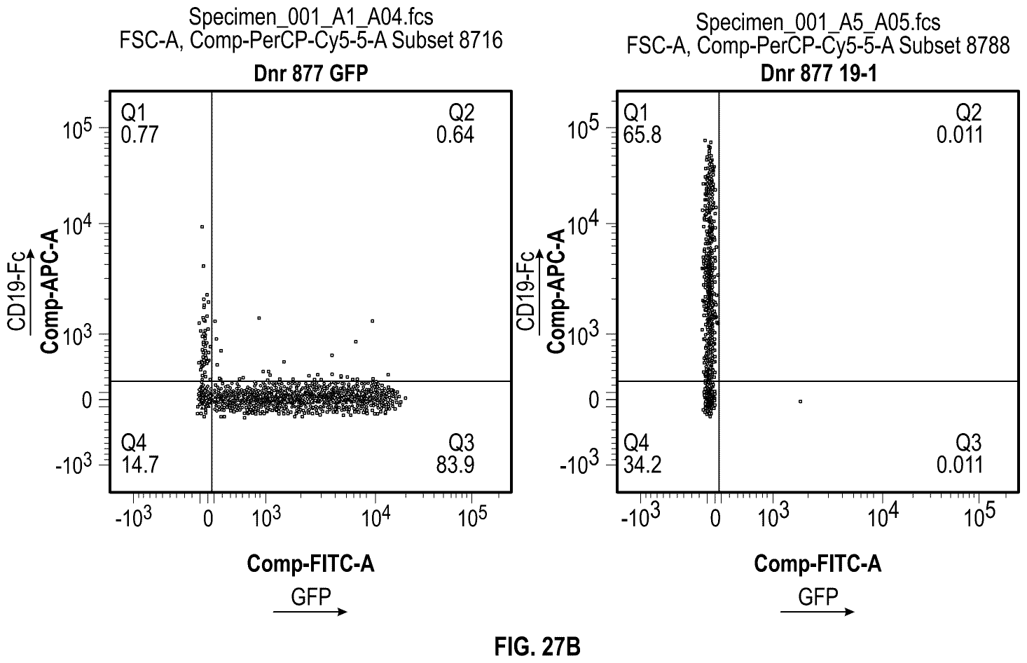

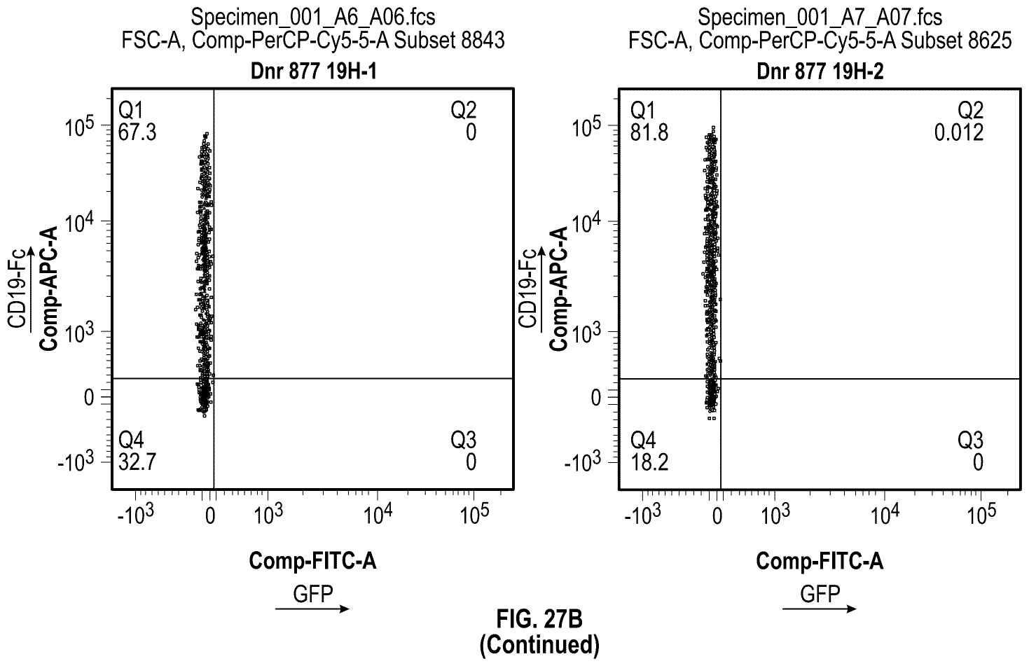

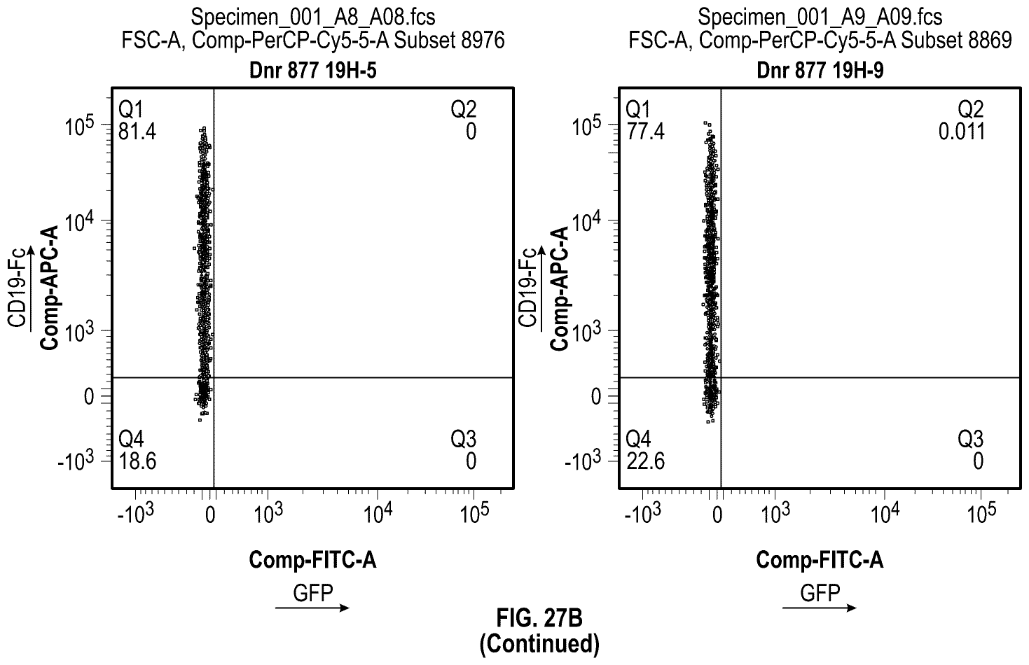

FIGS. 27A-27B show flow cytometry data that characterizes NK cells from two donors (276 in FIG. 17A and 877 in 27B) transduced with non-limiting examples of anti-CD19 CAR constructs as disclosed herein. FIGS. 27A and 27B both show that NK cells transduced with GFP express little to no CD19-Flag. Each of the other panels demonstrates that the NK cells from these donors can express not only the positive control non-humanized NK19-1 construct, but also efficiently express the selected humanized anti-CD19 CAR constructs. This is indicative of the limited impact that humanization of the anti-CD19 binder has on expression characteristics.

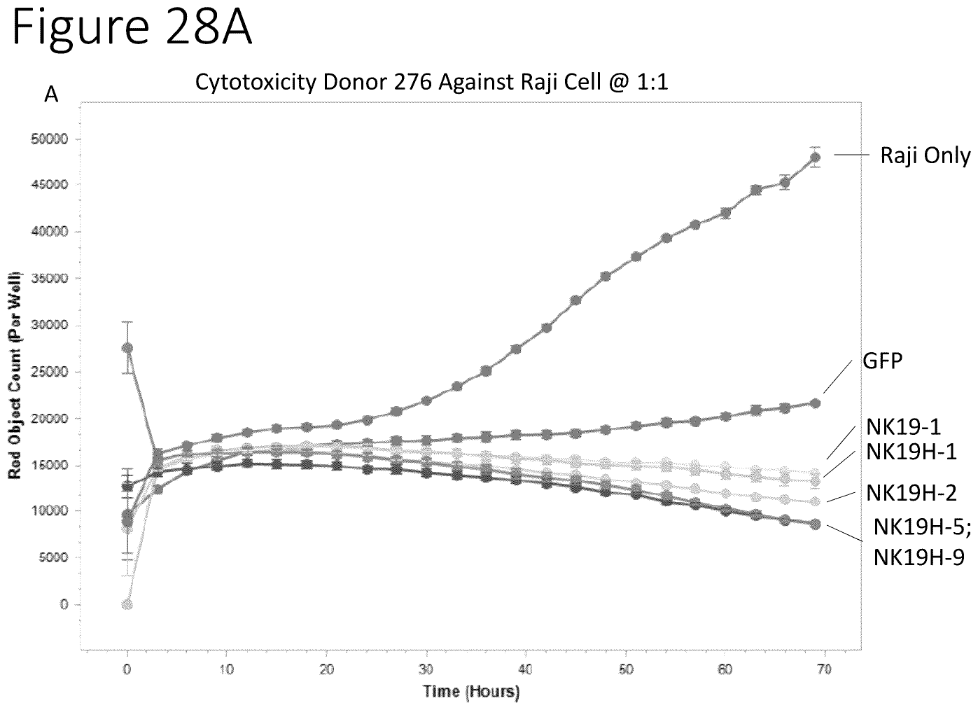

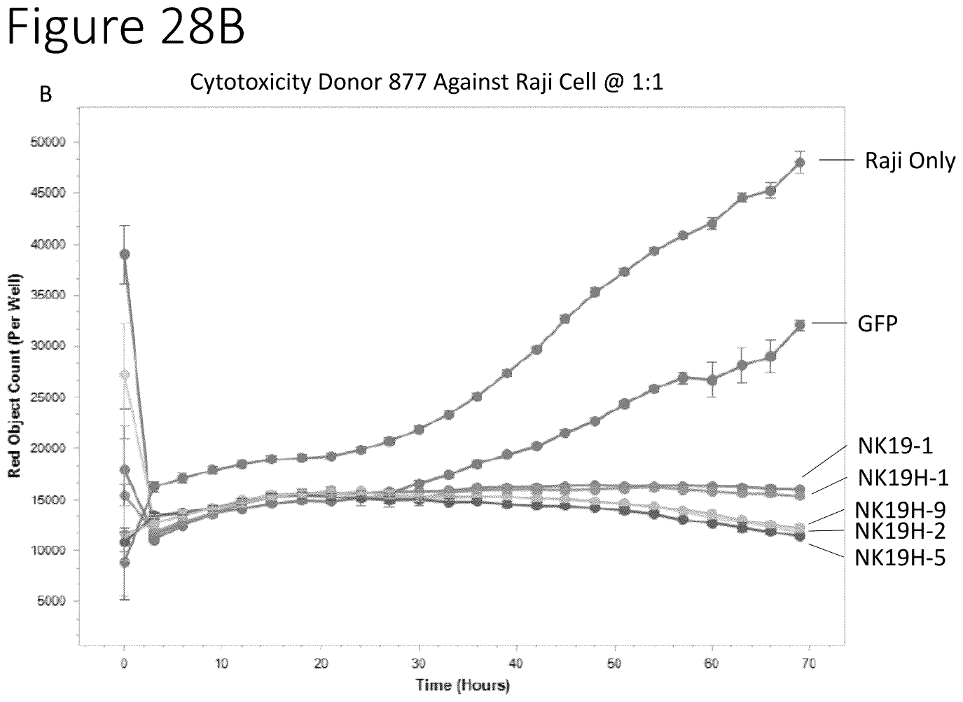

FIGS. 28A-28B show initial data for NK cell cytotoxicity of those two donors from FIG. 27 against Raji cells at an E:T ratio of 1:1. Consistent with results discussed above, each of the selected humanized anti-CD19 CAR constructs endowed transduced NK cells with significant cytotoxic potential against the target Raji cells. Each of the indicated non-limiting examples of anti-CD19 CAR constructs effectively controlled Raji cell growth (equivalent to, or enhanced as compared to, the non-humanized NK19-1 construct) with the trend of decreasing Raji cell numbers even as long as 70 hours after inception of the experiment. Similarly, FIG. 28B shows that transduced NK cells from donor 877 also effectively control Raji cell growth, equivalent to, or enhanced as compared to, the non-humanized NK19-1 construct. These data are in line with those presented for NK cells from other donors, discussed above, and indicate that, in accordance with several embodiments disclosed herein, transduction of NK cells with humanized anti-CD19 CARs enable the engineered NK cells to exert significant cytotoxic effects against target tumor cells and can effectively control growth of tumor cells. According to several embodiments, such engineered anti-CD19 NK cells (whether allogeneic or autologous) allow for robust and effective cellular immunotherapy to treat cancers.

### Example 8

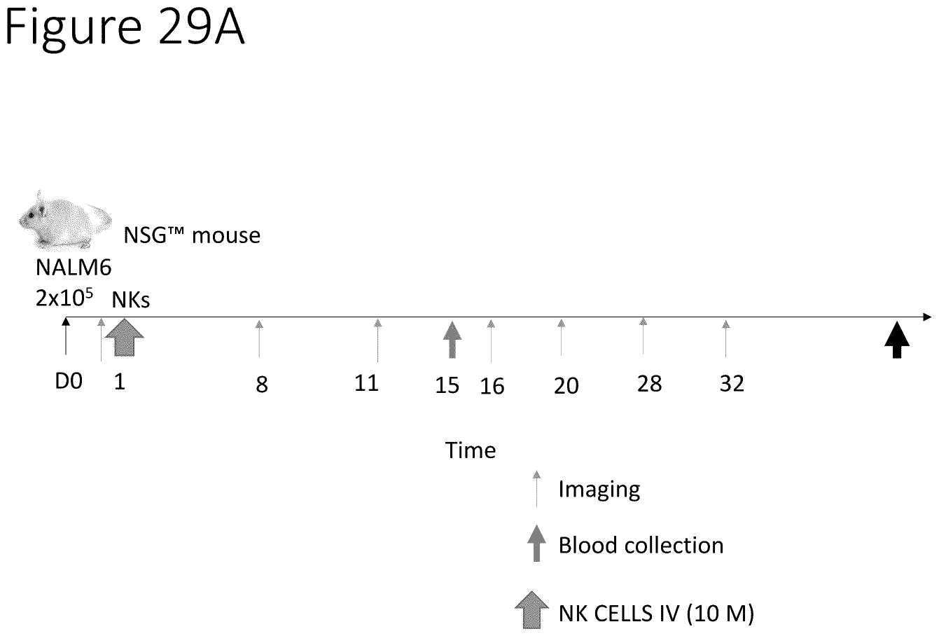

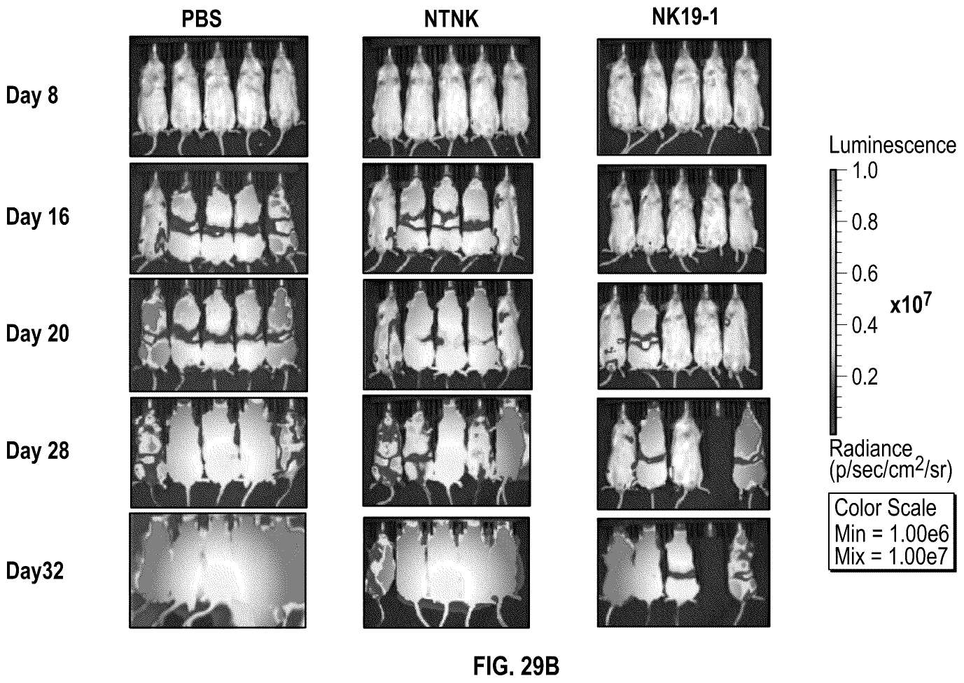

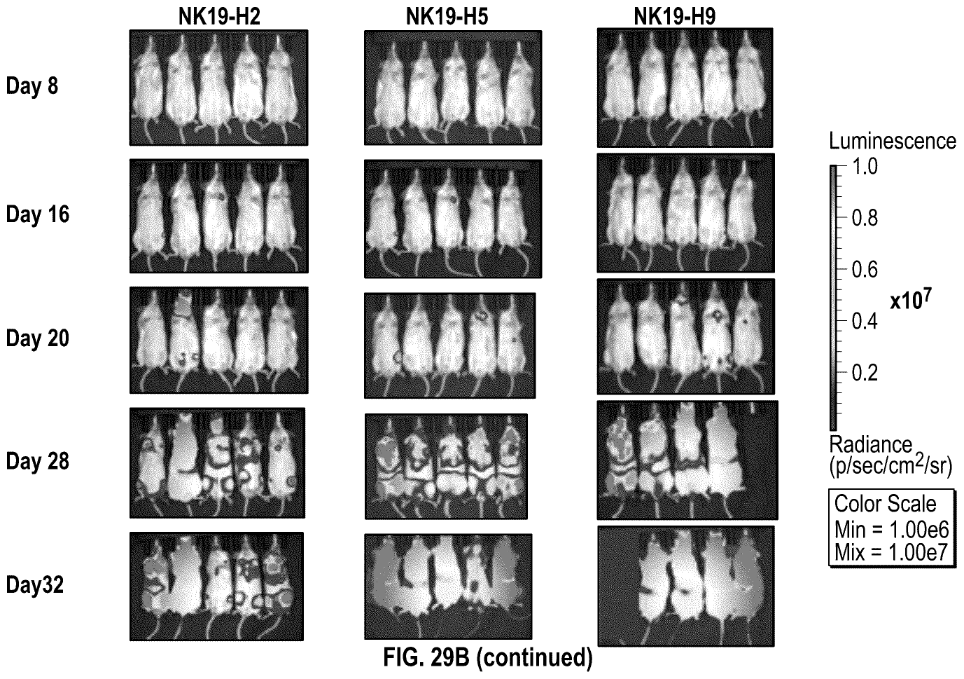

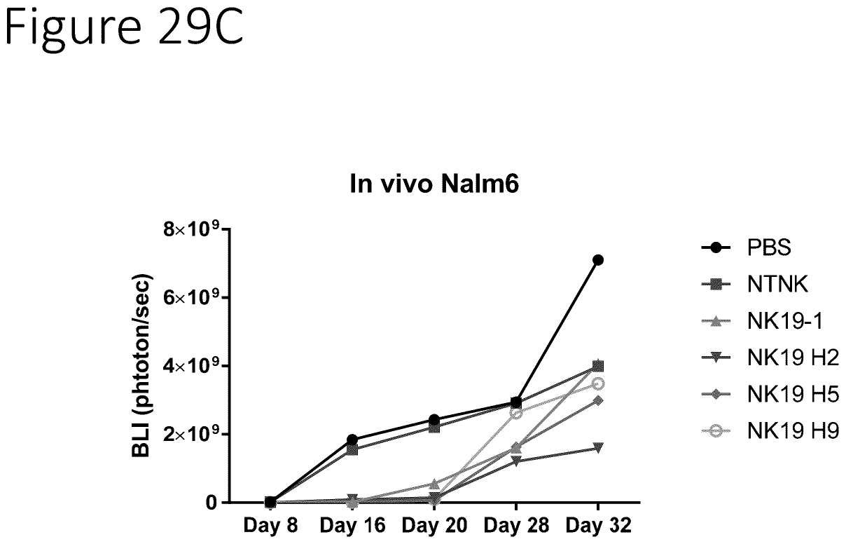

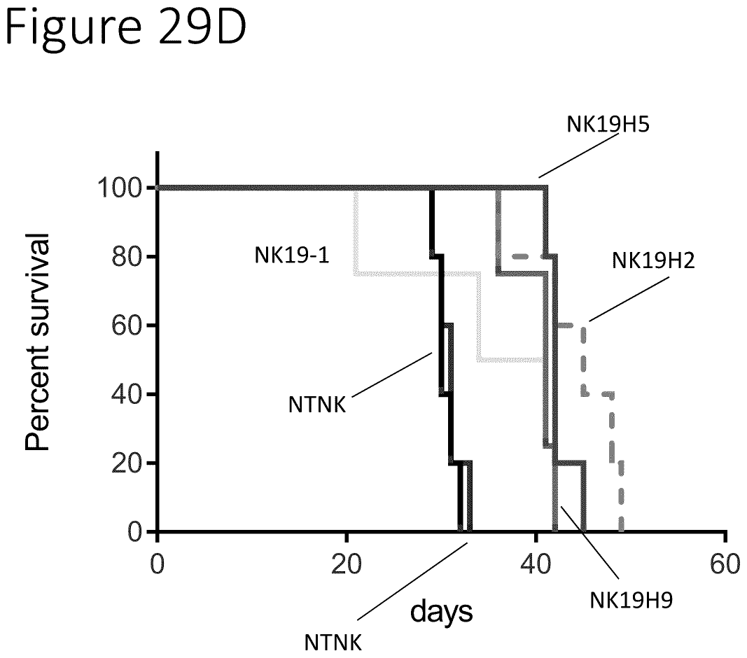

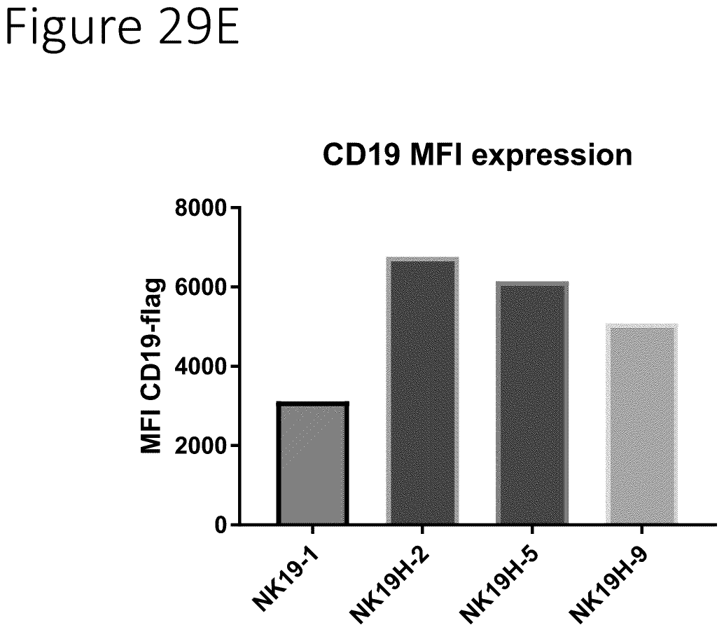

Further experiments were conducted to evaluate humanized CD19 CAR constructs as disclosed herein. FIG. 29A shows a schematic of the experimental protocol. Briefly, NSG mice were injected on Day 0 with 1×10⁵ Nalm6 cells (expressing a fluorescent reporter) intravenously. At Day 1, mice received either a PBS control injection, non-transduced K cells (“NTNK”, 10M), 10 million NK cells expressing the non-humanized NK19-1 CAR, or 10 million NK cells expressing one of various humanized CD19 CAR constructs. Blood collection and fluorescent imaging were performed as indicated. Bioluminescence data is shown in FIG. 29B. As shown, the injection of NK cells expressing any CD19 CAR construct resulted in a reduced progression of Nalm6 growth. The humanized constructs tested (NK19-H2, NK19-H5, and NK19H-9) each showed significant delay in Nalm6 growth compared to controls. FIG. 29C is a line graph depicting the measured bioluminescence over 31 days. As shown, each of the humanized constructs tested in this experiment showed reduced tumor progression as compared to controls, and slightly reduced as compared to non-humanized NK19-1. FIG. 29D shows a survival curve that reflects the reduction in tumor progression, with the mice treated with NK19-1 or any of the selected humanized constructs surviving longer than the control groups. FIG. 29E shows data related to an evaluation of the expression of each of the humanized constructs on the NK cells. As shown, each of the humanized constructs expressed more robustly than the non-humanized NK19-1 construct. In accordance with several embodiments disclosed herein, the use of a humanized construct results in a more efficacious therapeutic, at least in part due to the enhanced expression (and/or activity) of the CAR by the NK cells.

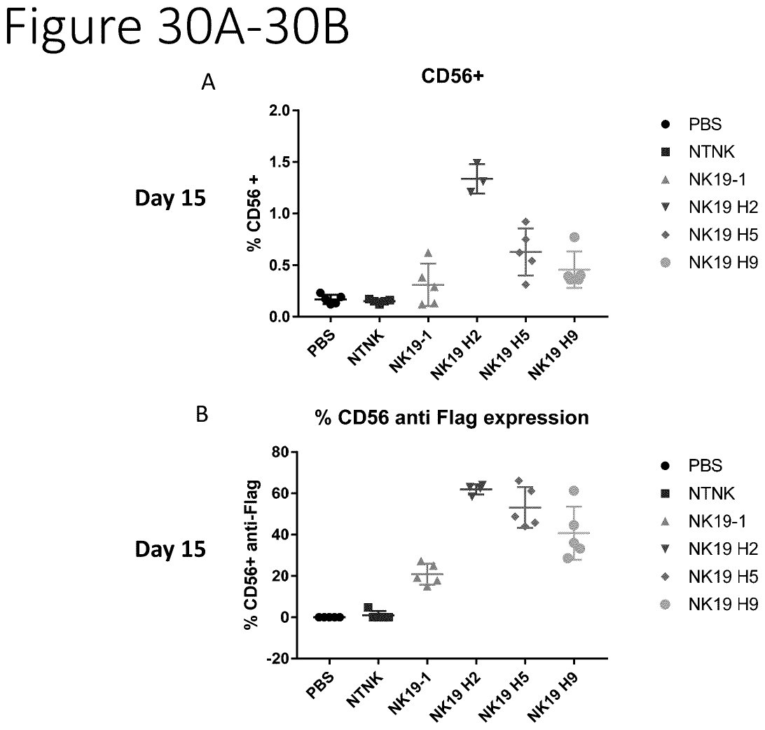

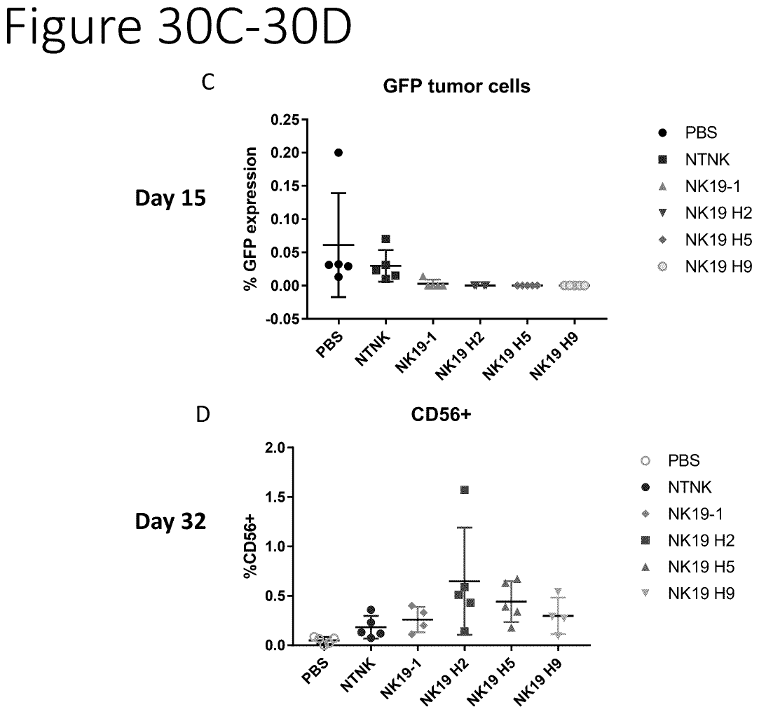

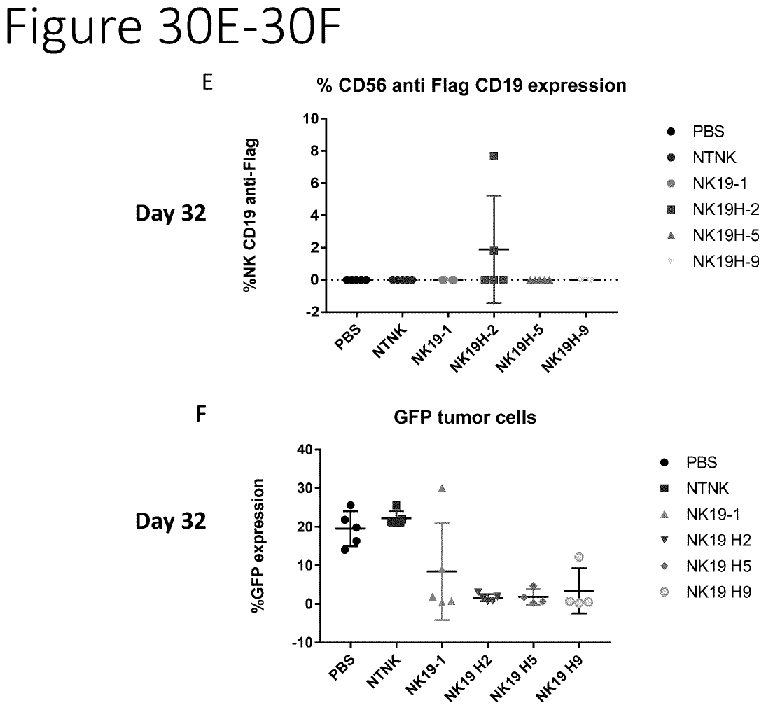

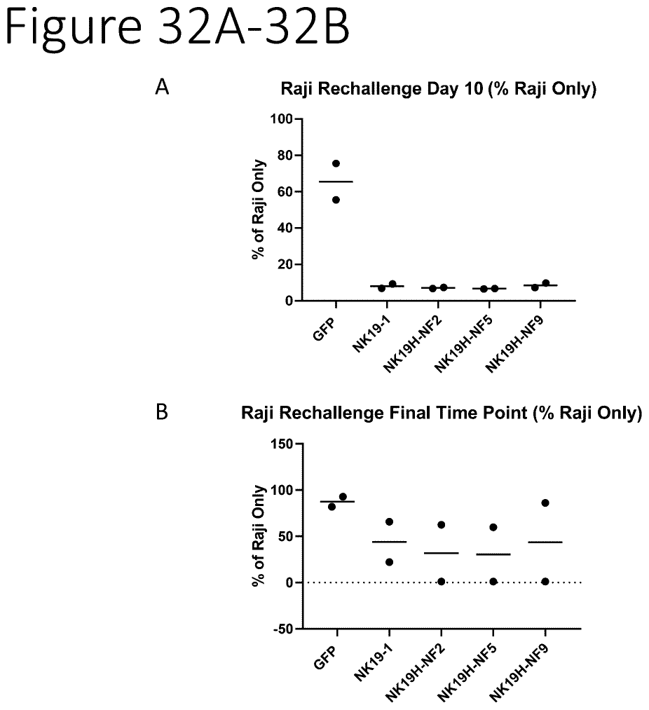

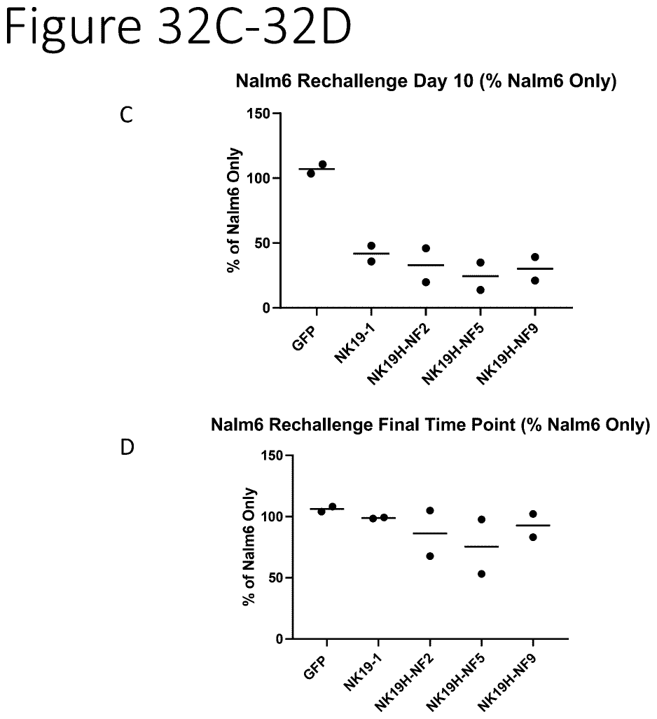

FIGS. 30A-30F relate to characteristics of cells in the blood of the mice treated with the indicated constructs at various time points during the experiment. FIG. 30A shows the percentage of human CD56+ cells in the peripheral blood of the mice, which represents the survival of the administered NK cells through the first fifteen days of the experiment. Each of the experimental groups exhibit a higher percentage of cells, with the NK19-H2 construct exhibiting the most persistence of those tested. FIG. 30B shows similar data, based on the detection of the flag expression tag used in these constructs (though, in several embodiments, no tag is used). These data show that the humanized constructs are expressed at greater levels than the non-humanized constructs. FIG. 30C shows the detection of GFP positive tumor cells after 15 days. The CAR-expressing NK cells show nearly zero GFP expression, reflective of their inhibition of Nalm6 growth at Day 15. FIGS. 30D-30F show similar data at Day 32. These data show that the NK cells expressing humanized CD19 CAR constructs make up a greater percentage of the cells present in the peripheral blood of the mice tested, which is consistent with the increased efficacy of these constructs at controlling tumor growth at later time points. FIG. 30F shows that there is little difference in expression among the humanized CD19 CAR constructs at day 32. FIG. 32F reflects the enhanced ability of NK cells expressing the humanized CD19 CAR constructs at controlling tumor growth, with the detected GFP-positive cells being lower in the humanized treatment groups, even as compared to the animals receiving NK19-1. In several embodiments, the enhanced efficacy is due, at least in part, to the enhanced expression of the humanized CD19 CAR constructs.

### Example 9

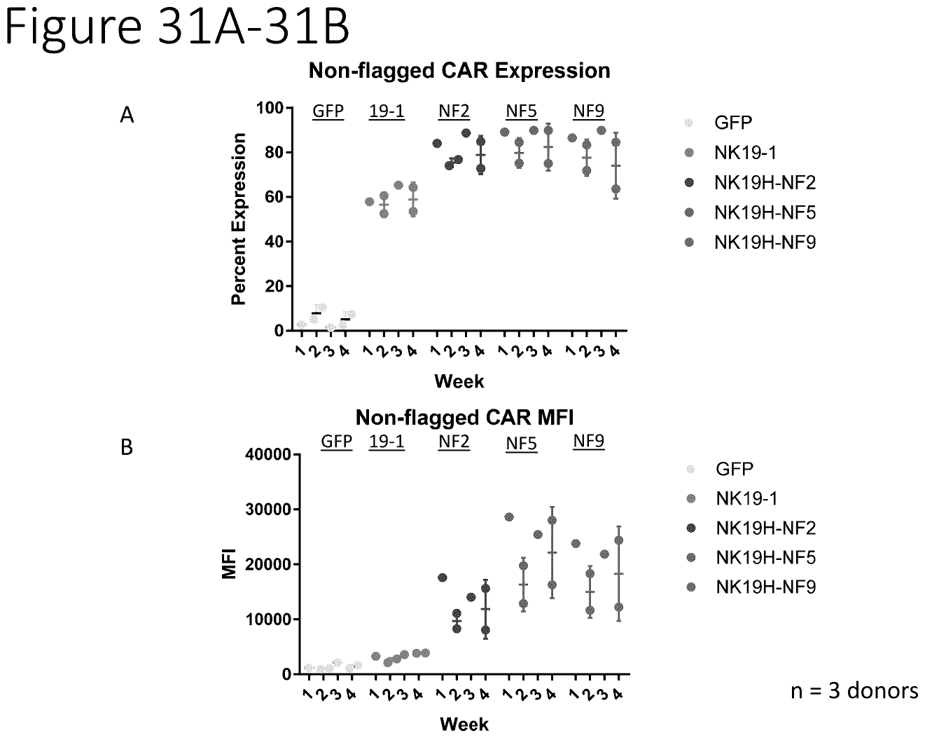

As discussed above, in some embodiments, CAR constructs comprise a detection tag. However, in several embodiments, no tag is used. Experiments were performed in order to evaluate the expression of non-flagged humanized CARS. This experiment employed NK19H-NK-2, -5 and -9 as non-limiting examples of non-flagged humanized CARs. FIGS. 31A-31B show expression data (percent expression in 31A, mean fluorescence in 31B) of the indicated construct by NK cells from three different donors, measured each week for 4 weeks. These data demonstrate that non-flagged versions of the CD19 CAR constructs as provided for herein express relatively similarly to one another, but more robustly than non-humanized constructs. Each of the non-flagged humanized constructs were expressed on at least about 70-80% of the NK cells.

### Example 10

Experiments to evaluate the cytotoxicity of NK cells expressing humanized, non-flagged CD19 CAR constructs were performed. An in vitro re-challenge assay was performed as described above, using Raji cells (FIGS. 32A-32B) or Nalm6 cells (FIGS. 32C-32D) as the target cell. FIG. 32A shows the percent of Raji cells co-cultured with the indicated treatment, measured as a percent of the number of cells measured in a Raji-only control group at day 10 of the experiment. As shown, each of the CD19 CAR constructs, whether humanized or not, and whether tagged or not, substantially eliminated Raji cell growth through Day 10. FIG. 32B shows the final time point, and there remains limited Raji cell growth in each of the experimental groups with CD19 CARs, though the data show a small trend to greater prevention of Raji growth in with the NK19H-NF-2 and NK19H-NF-5 groups. FIGS. 32C and 32D show the corresponding data using Nalm6 cells. FIG. 32C shows that, similar to Raji cells, each of the CD19 CAR constructs, whether humanized or not, and whether tagged or not, substantially eliminated Nalm6 cell growth through 10 days. At the final time point, there was Nalm6 cell growth across all groups, though the data trends to the humanized constructs being more effective at preventing growth.

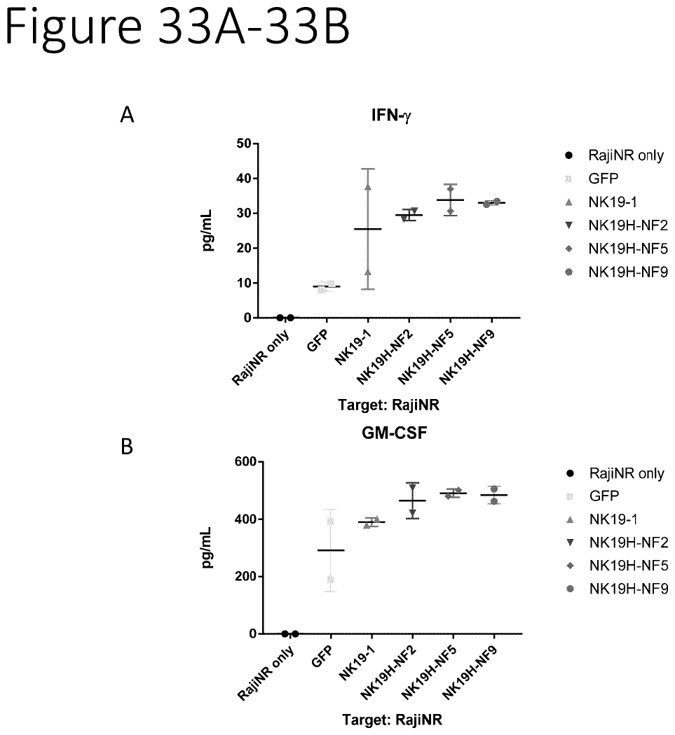

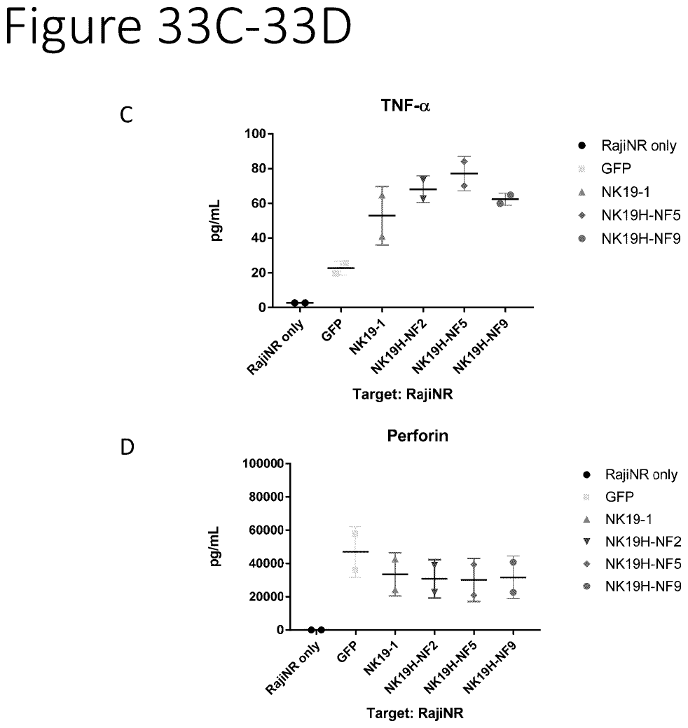

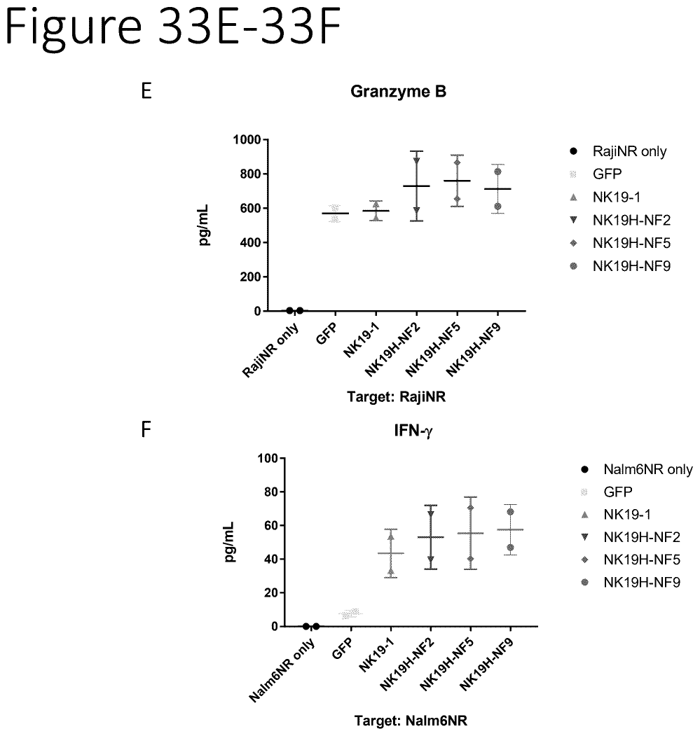

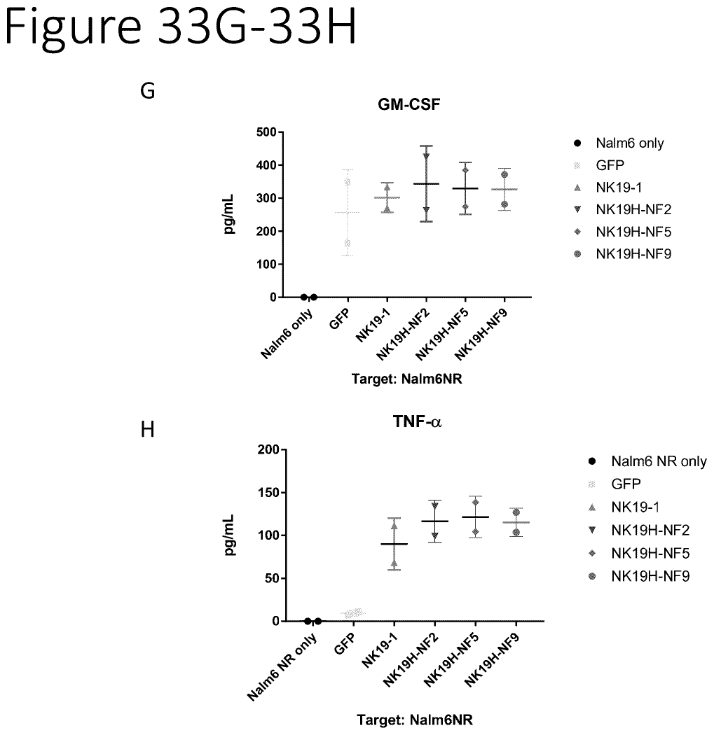

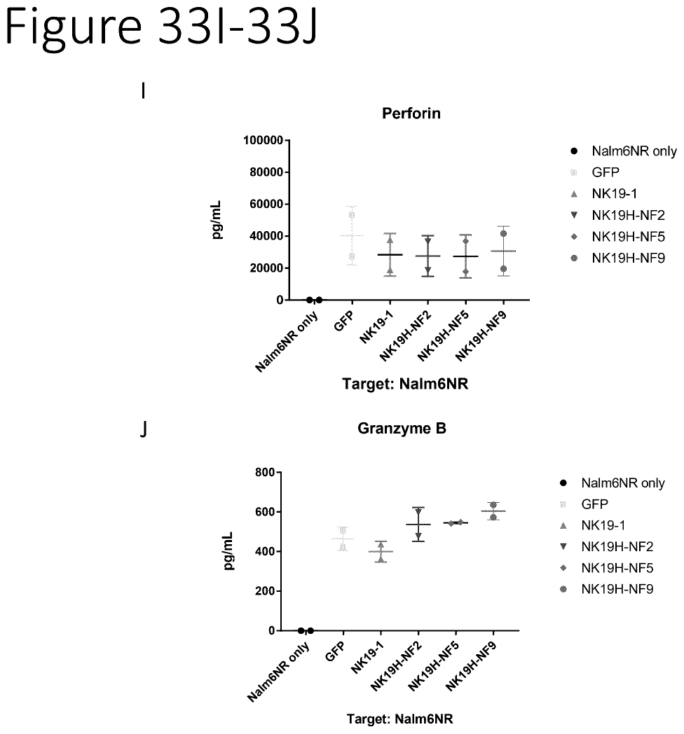

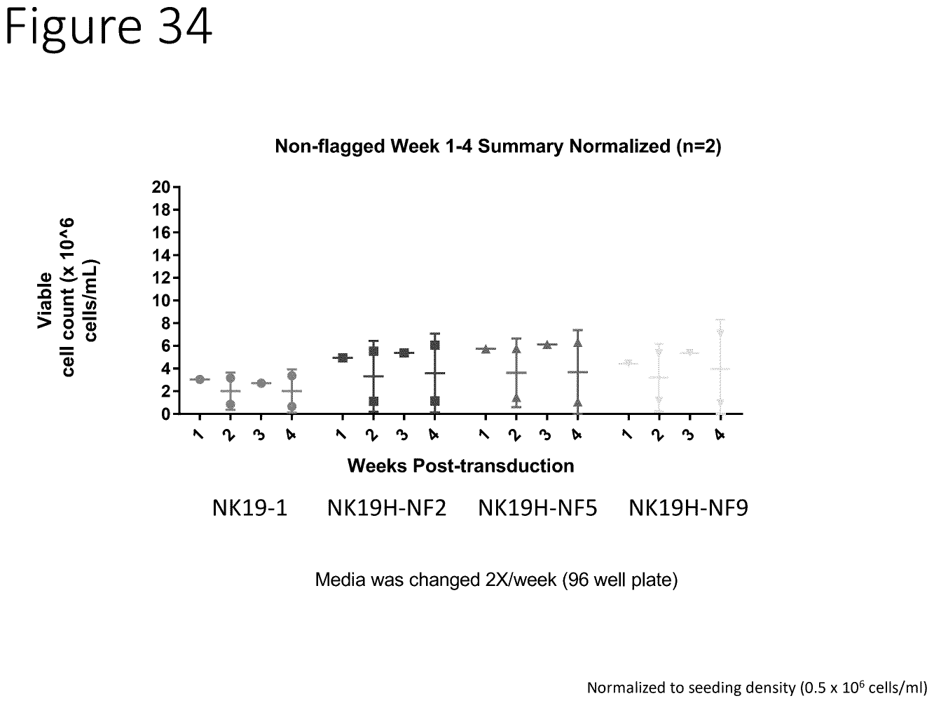

FIGS. 33A-33J relate to the detected cytokine profile for NK cells expressing the indicated constructs. As discussed above, the culture media from each treatment group was assayed for interferon gamma (33A/33F), GM-CSF (33B/33G), TNF-alpha (33C/33H), Perforin (33D/33I), and Granzyme B (33E/33J). FIGS. 33A-33E show data from the Raji cell rechallenge, while 33F-33J show data from the Nalm6 rechallenge. As shown, the non-flagged, humanized CD19 CAR expressing NK cells release similar levels of similar amount of IFN-gamma into the media, with those levels being slightly greater than the amount released by NK cells expressing the non-humanized NK19-1 CAR. Similarly, GM-CSF release was fairly consistent among the humanized, non-flagged CAR bearing NK cells, and at a level slightly about the NK19-1 expressing cells. TNF-alpha release showed a similar pattern, while perforin release was consistent among all the CD19 CAR expressing NK cells, and at a level below control. Lastly in the Raji cells, granzyme B showed a similar degree of release for the non-flagged, humanized CD19 CARs, at a level slightly above the NK19-1 expressing cells. For the most part, the data collected from the Nalm6 experiment showed similar patterns. According to several embodiments, the greater degree of release of cytotoxicity-mediating cytokines leads to a more effective therapeutic. In some embodiments, the greater degree of cytokine release is due, at least in part, to the enhanced expression of the non-flagged, humanized CD19 CAR constructs by the NK cells and/or due, at least in part, to the enhanced persistence of the NK cells expressing the non-flagged, humanized CD19 CAR constructs. Further supporting these concepts, FIG. 34 shows data related to the persistence of NK cells expressing the indicated non-flagged, humanized constructs over four weeks in culture, as compared to NK cells expressing NK19-1. While the overall cell counts appear similar among the NK cells expressing the humanized constructs, the cell counts show the same basic pattern as the cytokines, that is, slightly greater than NK cells expressing non-humanized NK19-1.

### Example 11

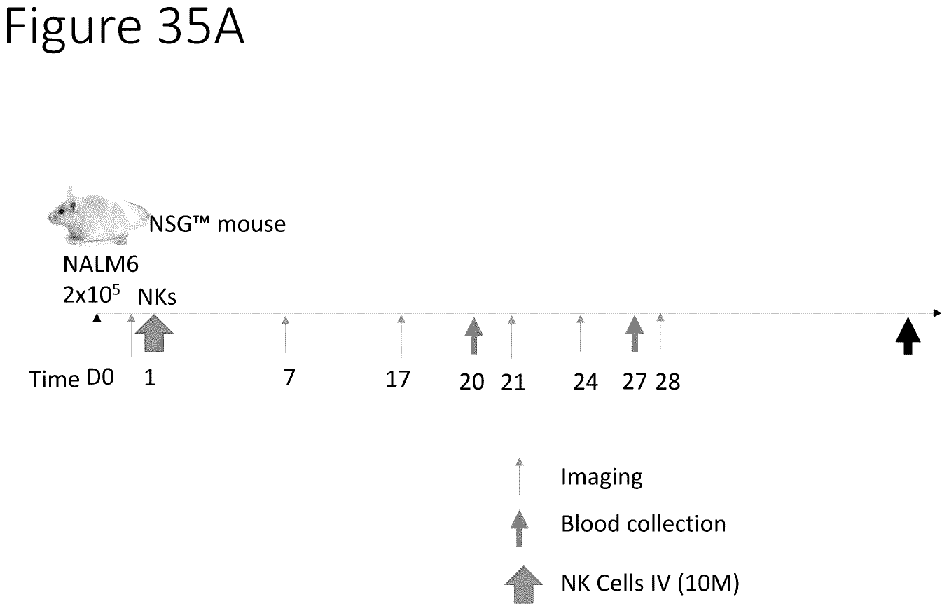

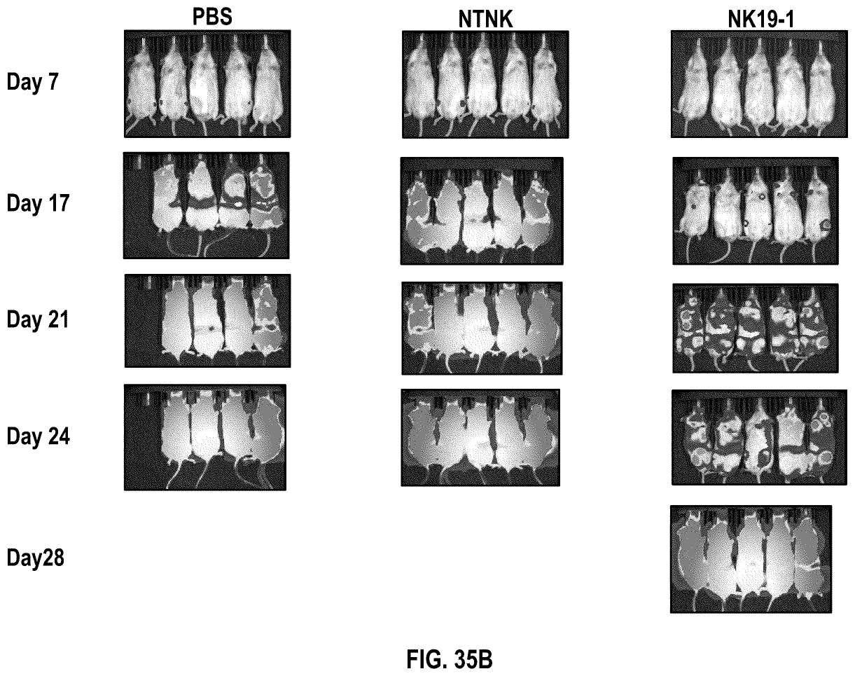

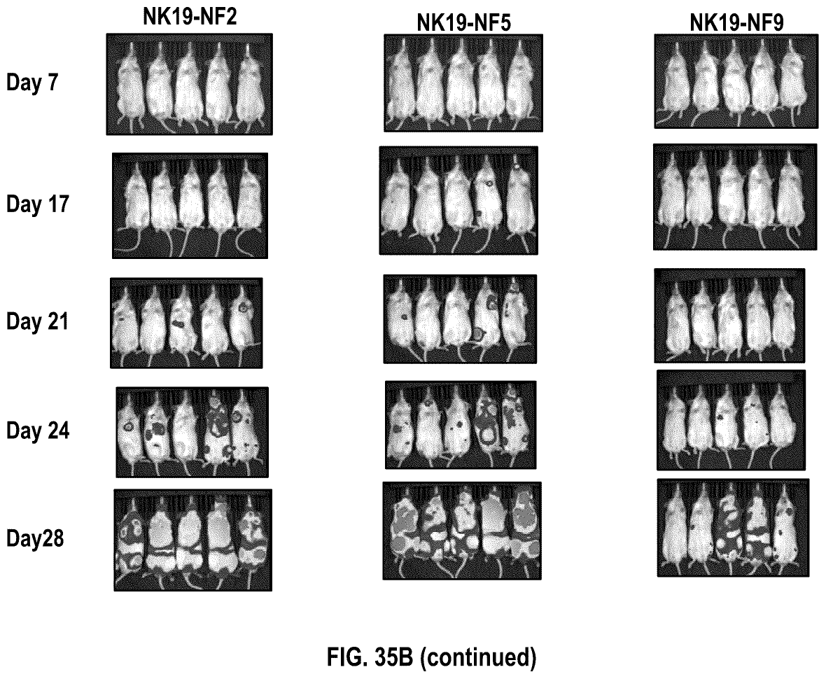

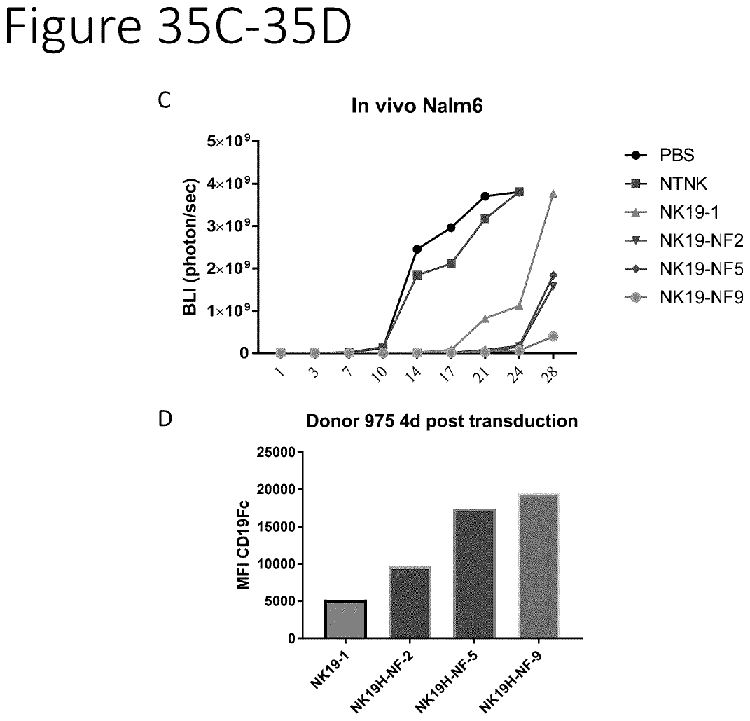

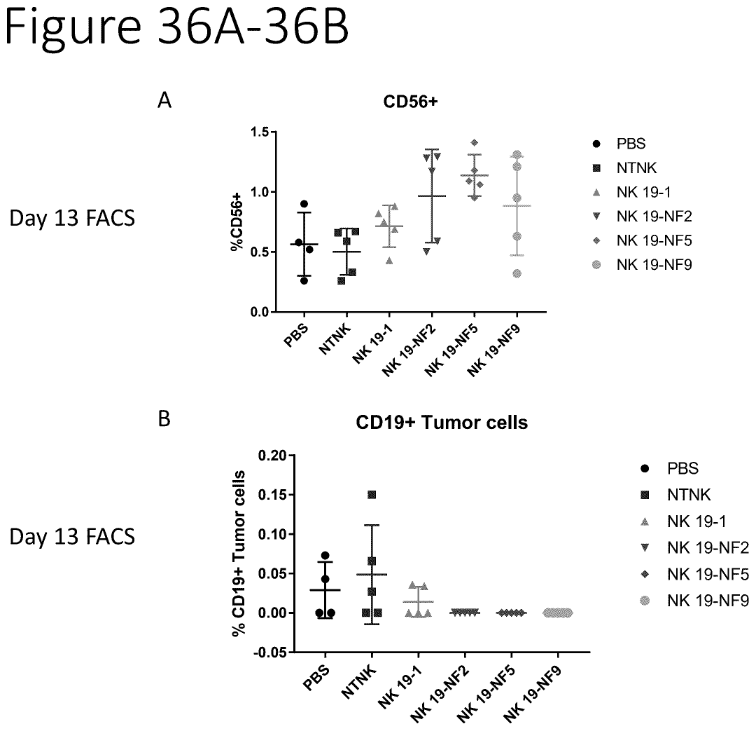

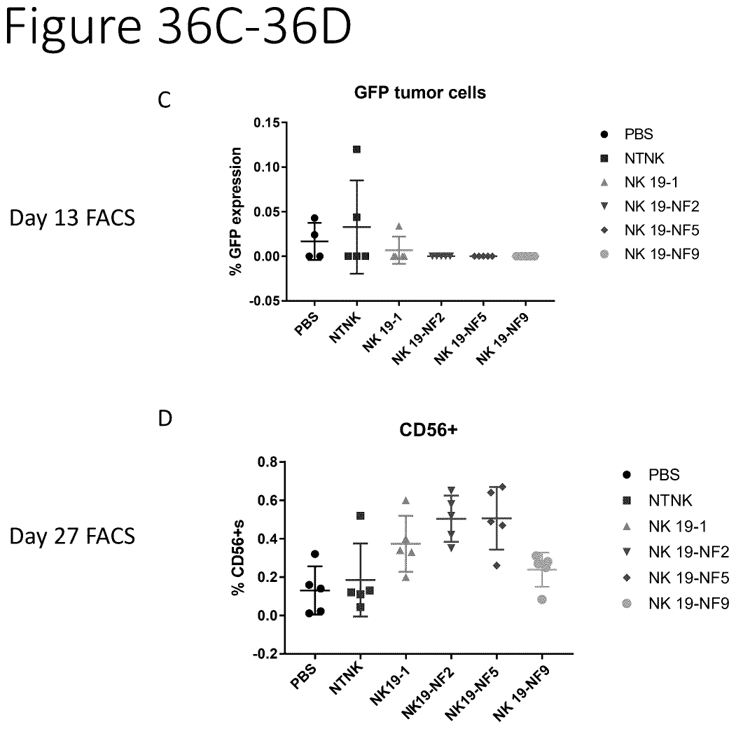

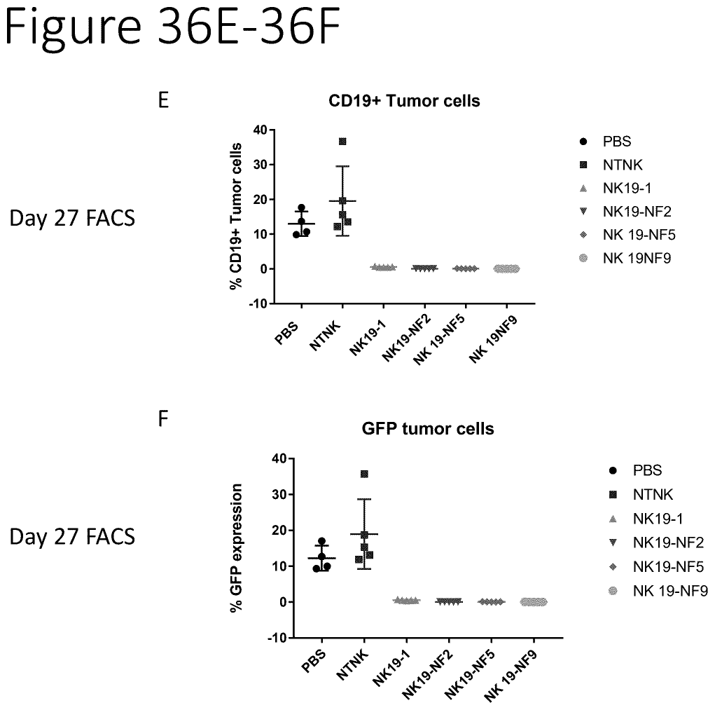

Similar to the experiments above, an in vivo assessment of the efficacy of non-flagged, humanized constructs was performed. FIG. 35A shows a schematic of the protocol used. FIG. 35B shows the in vivo bioluminescence measurements, which show that humanized, non-flagged constructs appear to slow tumor progression to a greater extent than NK cells expressing the NK19-1 construct. FIG. 35C, recapitulates the imaging data in a line graph, where the enhanced inhibition of tumor cell growth can clearly be seen when the mice received NK cells expressing any of the non-flagged, humanized CD19 CAR constructs. FIG. 35D reflects the enhanced expression of the non-flagged, humanized constructs by NK cells. FIGS. 36A-36F relate to the persistence of NK cells expressing the indicated constructs over the timeline of the experiment. FIG. 36A indicates that NK cells expressing the non-flagged, humanized CD19 CAR constructs are more persistent in vivo, making up a larger percentage of the overall CD56+ cells in the peripheral blood at Day 13. FIG. 36B demonstrates that, at Day 13, NK cells expressing any CD19 CAR inhibit tumor growth better than control, with the that NK cells expressing the non-flagged, humanized CD19 CAR constructs showing a slight advantage over the NK19-1 expressing NK cells in terms of limiting CD19+ tumor cell growth. FIG. 36C shows similar data to 36B, using GFP positive tumor cell count as the benchmark. FIG. 36D shows persistence data at day 27, wherein the NK19-NF-2 and -9 expressing NK cells have elevated population numbers compared to the other groups, indicating enhanced persistence over a longer period of time. Similar to the earlier time-point, FIGS. 36E and 36F show that the CD19-targeting CAR constructs are all effective at limiting tumor cell growth, compared to controls. According to several embodiments provided for herein, expression of a non-flagged, humanized CD19 targeting CAR by NK cells engenders those NK cells with enhanced in vivo persistence and cytotoxicity.

### Example 11

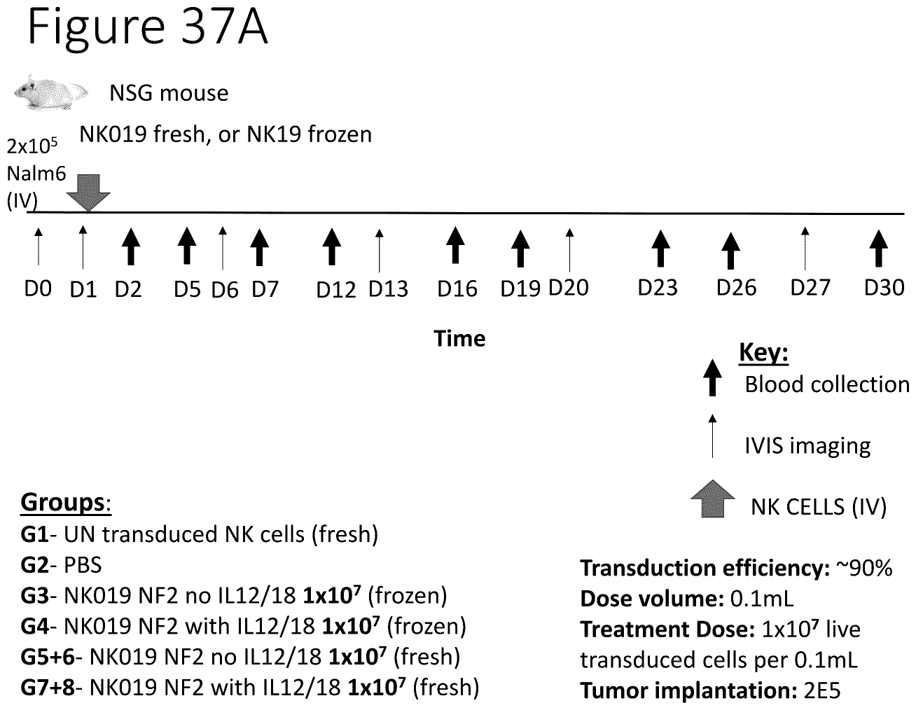

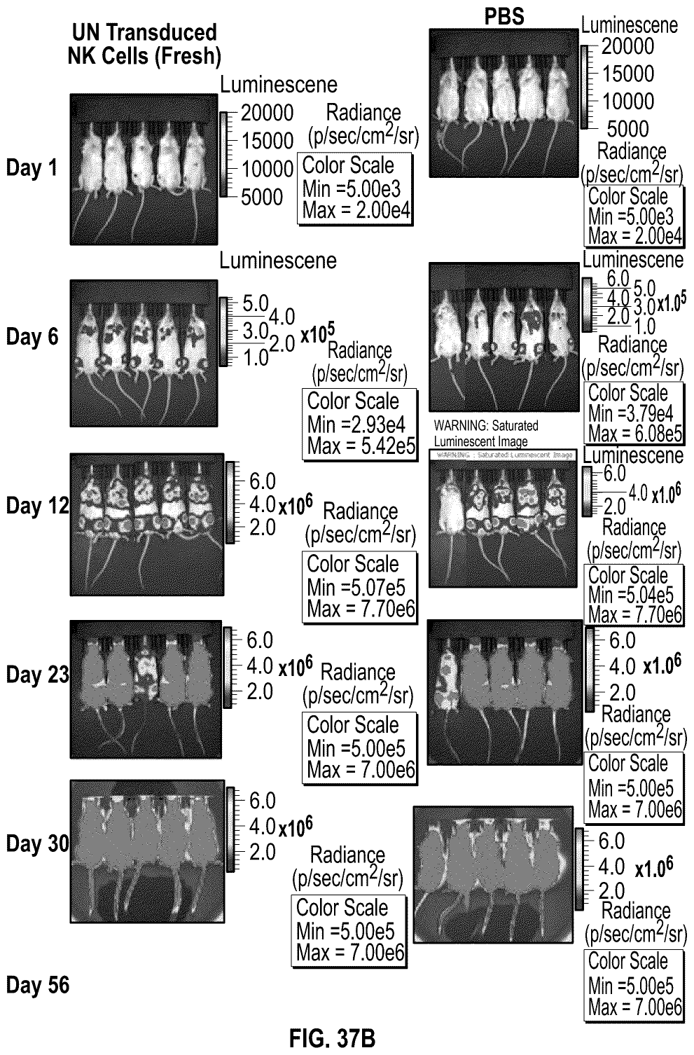

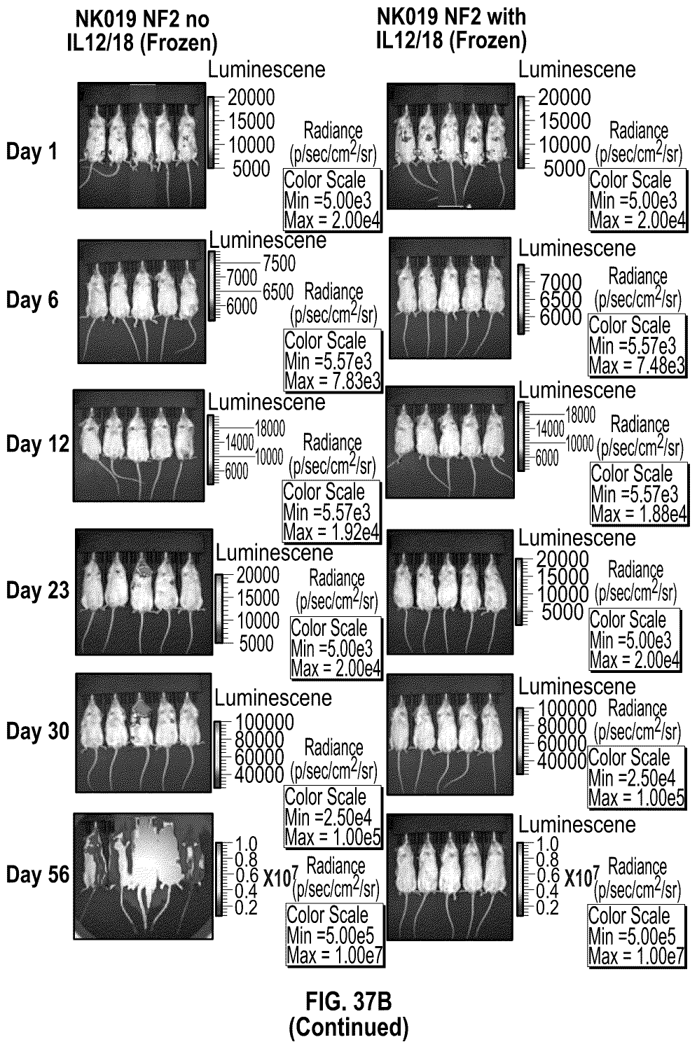

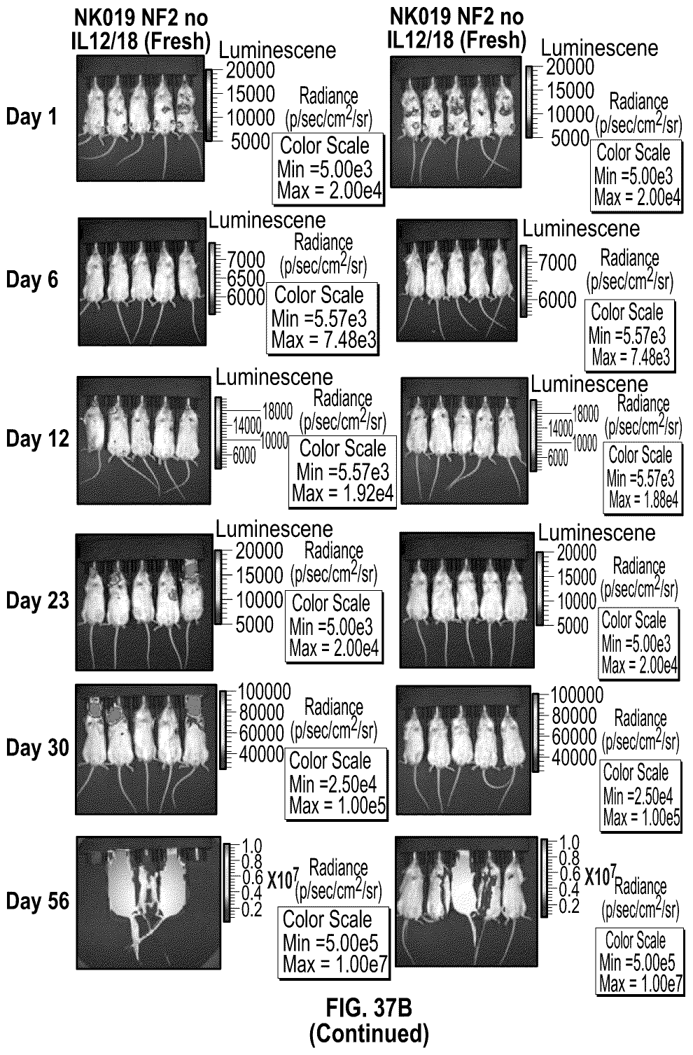

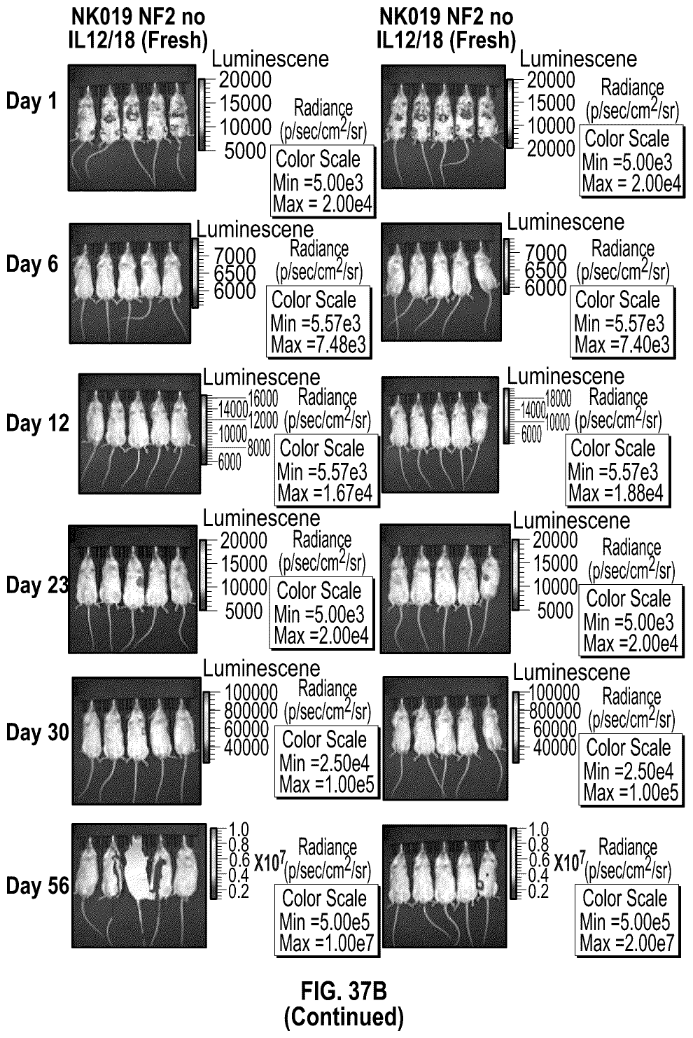

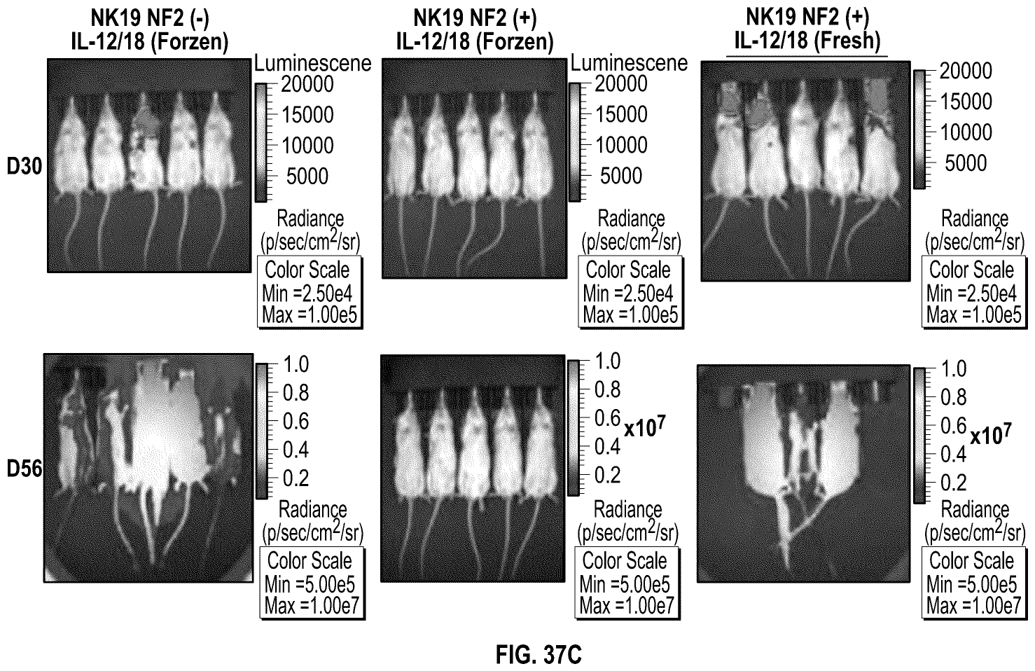

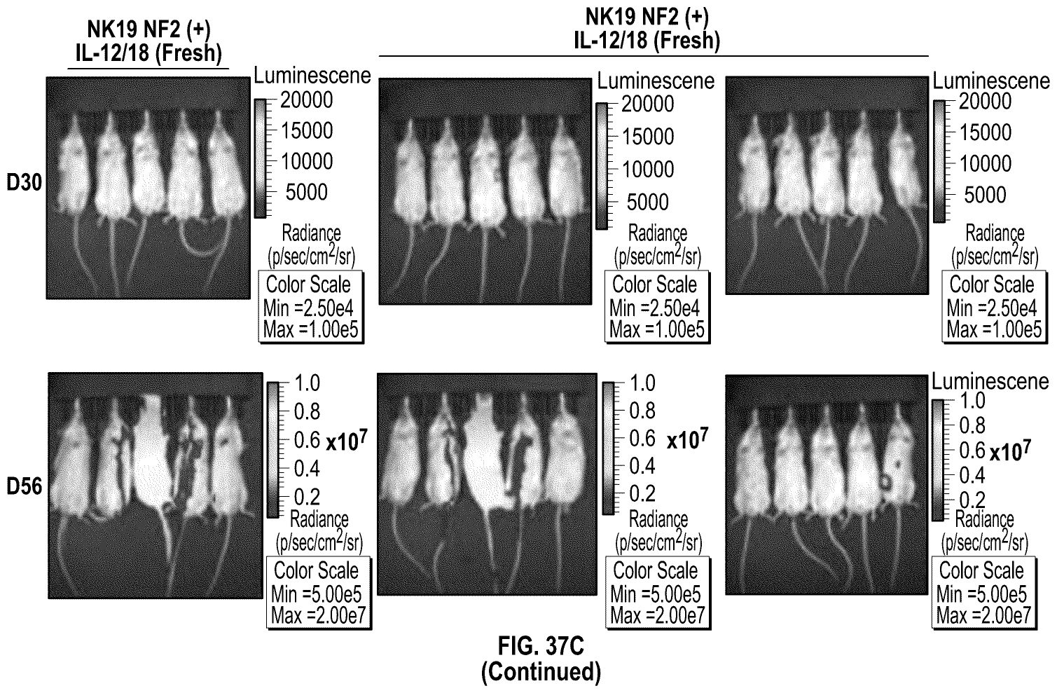

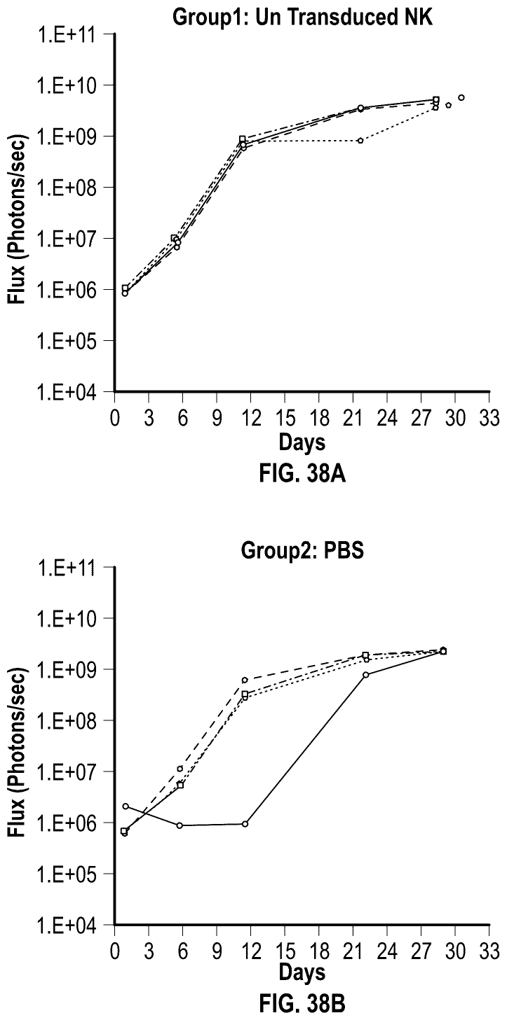

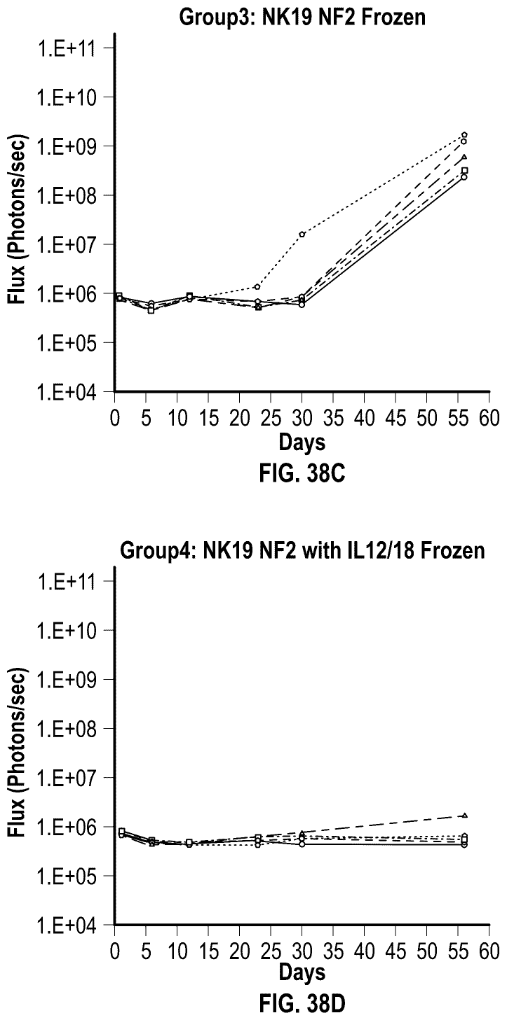

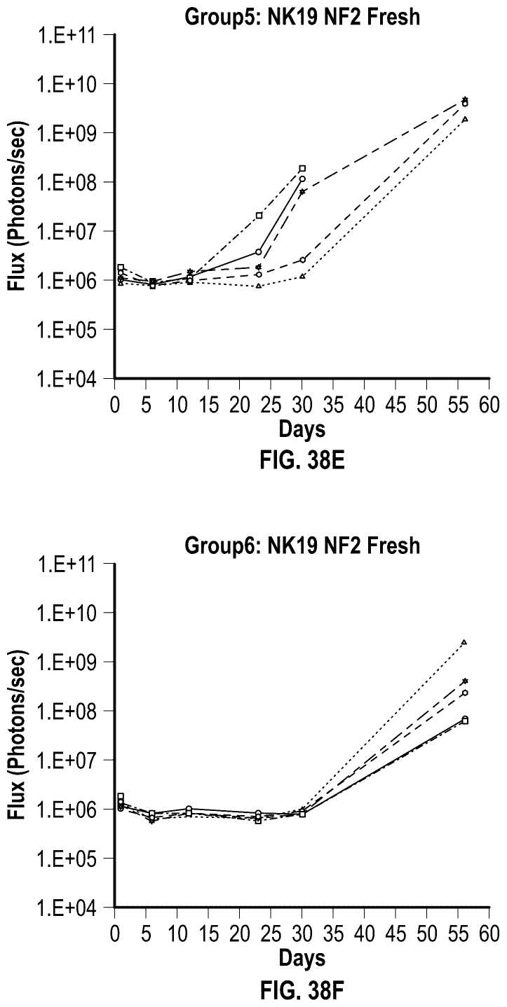

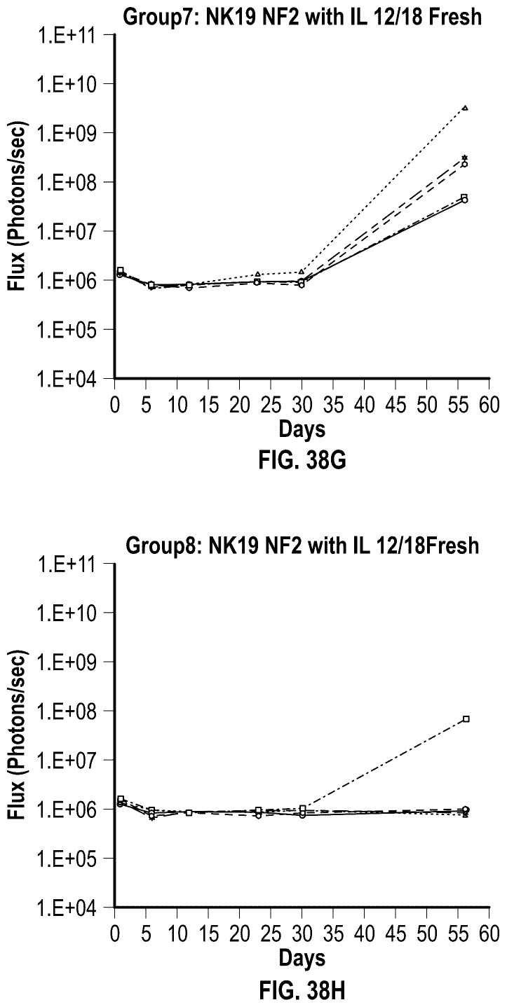

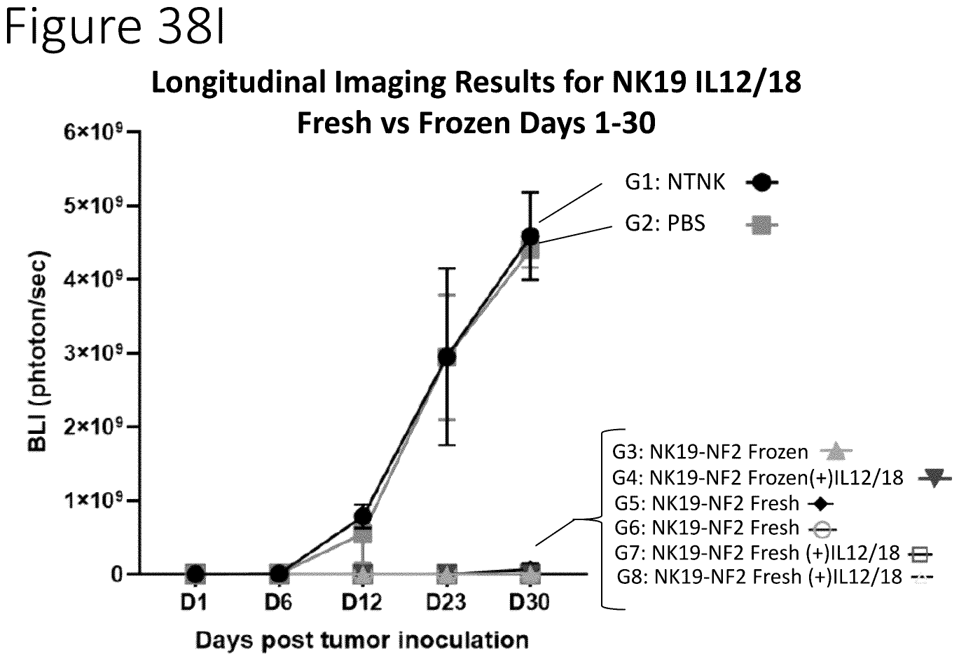

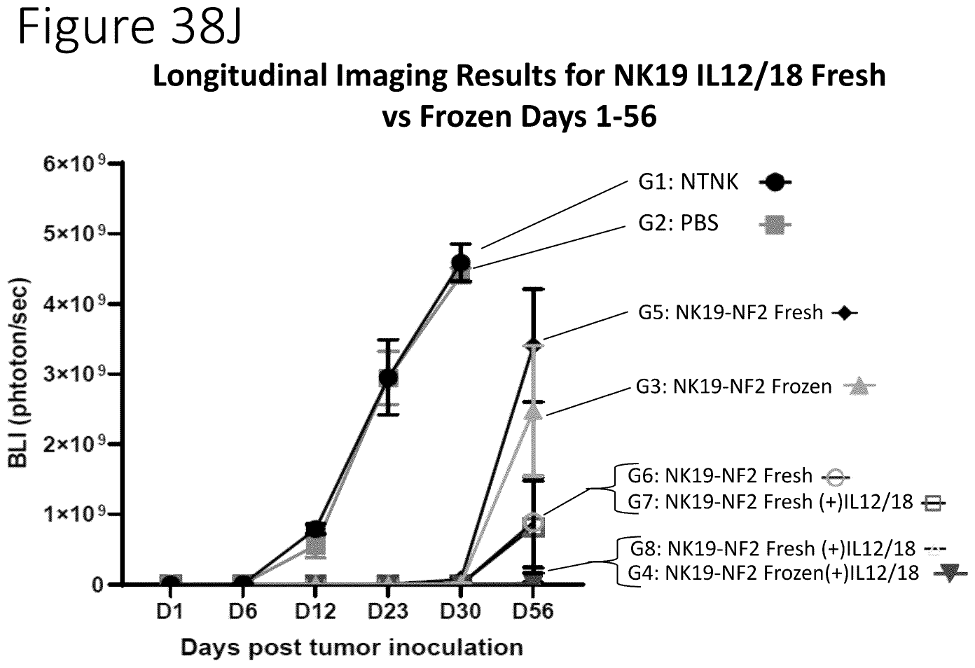

As discussed herein, in several embodiments engineered NK cells are prepared for allogeneic cell therapy. As such, in several embodiments, the engineered NK cells to be administered are prepared and then frozen for later use in a subject. Experiments were performed to determine whether the process of cryopreservation followed by thawing would adversely impact the engineered NK cells, such as by reducing their viability, persistence or cytotoxicity. FIG. 37A shows the schematic experimental protocol employed, as well as the experimental groups and other conditions used. For cells with an “IL12/IL18” designation, the cells were expanded in the presence of soluble IL12 and/or IL18, as described in in U.S. Provisional Patent Application No. 62/881,311, filed Jul. 31, 2019 and Application No. 62/932,342, filed Nov. 7, 2019, each of which is incorporated in its entirety by reference herein. FIGS. 37B and 37C shows the in vivo bioluminescence imaging from the indicated experimental groups. FIG. 38A-38H show line graphs that reflect the bioluminescence intensity over time. These data are recapitulated in FIGS. 38I, which shows the first 30 days, and FIG. 38J which shows data through 56 days. While FIG. 38I shows a clear distinction between the NK cells expressing CD19 CARs and the controls, there is nominal separation among the experimental groups. However, FIG. 38J shows data through 56 days, and there is a greater distinction of the ability of NK cells expressing the various CAR constructs and processed under the indicated conditions at inhibiting tumor cell growth. Of note is that the “pairs” of groups (same CAR construct, fresh vs. frozen) show fairly similar anti-tumor activity. This indicates, that, according to several embodiments, engineered NK cells expressing anti-CD19 CARs are effective not only when prepared and administered fresh. Additionally, according to several embodiments, engineered NK cells expressing anti-CD19 CARs are effective not only when prepared, frozen, then thawed and administered (e.g., as in an allogeneic context).

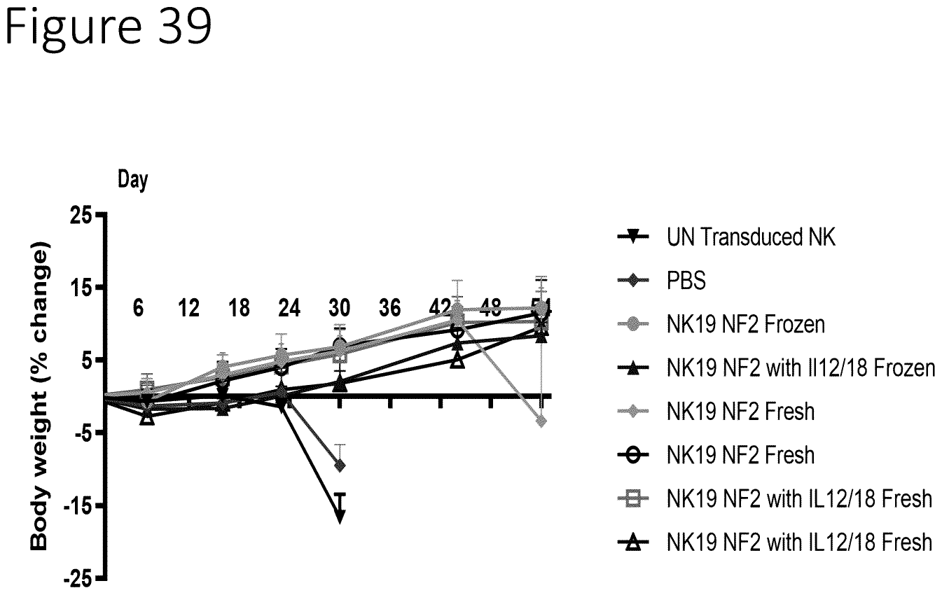

FIG. 39 shows a line graph of body mass of the mice treated with the indicated constructs over 56 days of the experiment. A reduction in body weight is correlated with increased tumor growth, e.g., progression of the tumor results in a decreased health of the mice, and corresponding loss of body weight (e.g., wasting). As shown, the control groups show substantial loss of body mass by 30 days, while experimental groups are increasing in body mass for the majority of the experiment. As with the bioluminescence data discussed above, there is a notable trend that many of the fresh versus frozen preparations exhibit substantially similar effects on body weight. According to several embodiments, engineered NK cells expressing anti-CD19 CARs are effective not only when prepared and administered fresh. Additionally, according to several embodiments, engineered NK cells expressing anti-CD19 CARs are effective not only when prepared, frozen, then thawed and administered (e.g., as in an allogeneic context).

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% sequence identity or homology includes 96%, 97%, 98%, 99%, and 100% sequence identity or homology to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

In several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

Any titles or subheadings used herein are for organization purposes and should not be used to limit the scope of embodiments disclosed herein.

1. (canceled)

2. A CD19-directed chimeric antigen receptor (CAR), the CAR comprising:
(a) an extracellular anti-CD19 binding moiety comprising a single-chain variable fragment (scFv), wherein the scFv comprises a variable heavy (VH) domain and a variable light (VL) domain,
wherein the VH domain comprises the CDR-H1, CDR-H2, and CDR-H3 of the VH domain set forth in SEQ ID NO:120, and
wherein the VL domain comprises the CDR-L1, CDR-L2, and CDR-L3 of the VL domain set forth in SEQ ID NO:118;
(b) a transmembrane domain; and
(c) an intracellular signaling domain comprising an OX40 subdomain and a CD3zeta subdomain.

3. The CD19-directed CAR of claim 2, wherein the VH domain comprises the amino acid sequence set forth in SEQ ID NO:120 and the VL domain comprises the amino acid sequence set forth in SEQ ID NO:118.

4. The CD19-directed CAR of claim 2, wherein OX40 subdomain comprises the amino acid sequence set forth in SEQ ID NO:6.

5. The CD19-directed CAR of claim 2, wherein the transmembrane domain is a CD8a transmembrane domain.

6. The CD19-directed CAR of claim 3, wherein the transmembrane domain is a CD8a transmembrane domain.

7. An immune cell expressing a CD19-directed chimeric antigen receptor (CAR), the CD19-directed CAR comprising:
(a) an extracellular anti-CD19 binding moiety comprising a single-chain variable fragment (scFv), wherein the scFv comprises a variable heavy (VH) domain and a variable light (VL) domain,
wherein the VH domain comprises the CDR-H1, CDR-H2, and CDR-H3 of the VH domain set forth in SEQ ID NO:120, and
wherein the VL domain comprises the CDR-L1, CDR-L2, and CDR-L3 of the VL domain set forth in SEQ ID NO:118;
(b) a transmembrane domain; and
(c) an intracellular signaling domain comprising an OX40 subdomain and a CD3zeta subdomain.

8. The immune cell of claim 7, wherein the immune cell is a natural killer (NK) cell.

9. The immune cell of claim 7, wherein the VH domain comprises the amino acid sequence set forth in SEQ ID NO:120 and the VL domain comprises the amino acid sequence set forth in SEQ ID NO:118.

10. The immune cell of claim 7, wherein OX40 subdomain comprises the amino acid sequence set forth in SEQ ID NO:6.

11. The immune cell of claim 7, wherein the transmembrane domain is a CD8a transmembrane domain.

12. A polynucleotide encoding a CD19-directed chimeric antigen receptor (CAR), the CD19-directed CAR comprising:
(a) an extracellular anti-CD19 binding moiety comprising a single-chain variable fragment (scFv), wherein the scFv comprises a variable heavy (VH) domain and a variable light (VL) domain,
wherein the VH domain comprises the CDR-H1, CDR-H2, and CDR-H3 of the VH domain set forth in SEQ ID NO:120, and
wherein the VL domain comprises the CDR-L1, CDR-L2, and CDR-L3 of the VL domain set forth in SEQ ID NO:118;
(b) a transmembrane domain; and
(c) an intracellular signaling domain comprising an OX40 subdomain and a CD3zeta subdomain.

13. The polynucleotide of claim 12, further encoding a membrane-bound interleukin-15 (mbIL15).

14. A vector comprising a polynucleotide encoding a CD19-directed chimeric antigen receptor (CAR), the CD19-directed CAR comprising:
(a) an extracellular anti-CD19 binding moiety comprising a single-chain variable fragment (scFv), wherein the scFv comprises a variable heavy (VH) domain and a variable light (VL) domain,
wherein the VH domain comprises the CDR-H1, CDR-H2, and CDR-H3 of the VH domain set forth in SEQ ID NO:120, and
wherein the VL domain comprises the CDR-L1, CDR-L2, and CDR-L3 of the VL domain set forth in SEQ ID NO:118;
(b) a transmembrane domain; and
(c) an intracellular signaling domain comprising an OX40 subdomain and a CD3zeta subdomain.

15. The vector of claim 14, wherein the polynucleotide further encodes a membrane-bound interleukin-15 (mbIL15).

16. The vector of claim 14, wherein the vector is a retroviral vector.

17. A method of treating a subject having a disease, the method comprising administering to a subject having a disease a population of immune cells that express a CD19-directed chimeric antigen receptor (CAR), the CD19-directed CAR comprising:
(a) an extracellular anti-CD19 binding moiety comprising a single-chain variable fragment (scFv), wherein the scFv comprises a variable heavy (VH) domain and a variable light (VL) domain,
wherein the VH domain comprises the CDR-H1, CDR-H2, and CDR-H3 of the VH domain set forth in SEQ ID NO:120, and
wherein the VL domain comprises the CDR-L1, CDR-L2, and CDR-L3 of the VL domain set forth in SEQ ID NO:118;
(b) a transmembrane domain; and
(c) an intracellular signaling domain comprising an OX40 subdomain and a CD3zeta subdomain.

18. The method of claim 17, wherein the VH domain comprises the amino acid sequence set forth in SEQ ID NO:120 and the VL domain comprises the amino acid sequence set forth in SEQ ID NO:118.

19. The method of claim 17, wherein OX40 subdomain comprises the amino acid sequence set forth in SEQ ID NO:6.

20. The method of claim 17, wherein the immune cells comprise natural killer (NK) cells.

21. The method of claim 17, wherein the immune cells further express a membrane-bound interleukin-15 (mbIL15).
